SEQUENCE LISTING (1240 sequences)

SEQ ID NO: 1 - RNA, 19 nt, Artificial Sequence
  Description of Artificial Sequence Synthetic oligonucleotide
uggccuaugagggugccgc

SEQ ID NO: 2 - RNA, 19 nt, Artificial Sequence
  Description of Artificial Sequence Synthetic oligonucleotide
ugaauaaacuccaggccua

SEQ ID NO: 3 - RNA, 19 nt, Artificial Sequence
  Description of Artificial Sequence Synthetic oligonucleotide
uuaaacuccaggccuauga

SEQ ID NO: 4 - RNA, 19 nt, Artificial Sequence
  Description of Artificial Sequence Synthetic oligonucleotide
ucgaauaaacuccaggccu

SEQ ID NO: 5 - RNA, 19 nt, Artificial Sequence
  Description of Artificial Sequence Synthetic oligonucleotide
uuccggcagcagauggcaa

SEQ ID NO: 6 - RNA, 19 nt, Artificial Sequence
  Description of Artificial Sequence Synthetic oligonucleotide
uacuccaggccuaugaggg

SEQ ID NO: 7 - RNA, 19 nt, Artificial Sequence
  Description of Artificial Sequence Synthetic oligonucleotide
uccuaugagggugccgcua

SEQ ID NO: 8 - RNA, 19 nt, Artificial Sequence
  Description of Artificial Sequence Synthetic oligonucleotide
uaggccuaugagggugccg

SEQ ID NO: 9 - RNA, 19 nt, Artificial Sequence
  Description of Artificial Sequence Synthetic oligonucleotide
uuccaggccuaugagggug

SEQ ID NO: 10 - RNA, 19 nt, Artificial Sequence
  Description of Artificial Sequence Synthetic oligonucleotide
ugcugguccucagggaacc

SEQ ID NO: 11 - RNA, 19 nt, Artificial Sequence
  Description of Artificial Sequence Synthetic oligonucleotide
uaauaaacuccaggccuau

SEQ ID NO: 12 - RNA, 19 nt, Artificial Sequence
  Description of Artificial Sequence Synthetic oligonucleotide
ucuggagcagcucagcagc

SEQ ID NO: 13 - RNA, 19 nt, Artificial Sequence
  Description of Artificial Sequence Synthetic oligonucleotide
uccgaauaaacuccaggcc

SEQ ID NO: 14 - RNA, 19 nt, Artificial Sequence
  Description of Artificial Sequence Synthetic oligonucleotide
ugguccucagggaaccagg

SEQ ID NO: 15 - RNA, 19 nt, Artificial Sequence
  Description of Artificial Sequence Synthetic oligonucleotide
uugguccucagggaaccag

SEQ ID NO: 16 - RNA, 19 nt, Artificial Sequence
  Description of Artificial Sequence Synthetic oligonucleotide
uauaaacuccaggccuaug

SEQ ID NO: 17 - RNA, 19 nt, Artificial Sequence
  Description of Artificial Sequence Synthetic oligonucleotide
ucuccaggccuaugagggu

SEQ ID NO: 18 - RNA, 19 nt, Artificial Sequence
  Description of Artificial Sequence Synthetic oligonucleotide
ucaggccuaugagggugcc

SEQ ID NO: 19 - RNA, 19 nt, Artificial Sequence
  Description of Artificial Sequence Synthetic oligonucleotide
uguccucagggaaccaggc

SEQ ID NO: 20 - RNA, 19 nt, Artificial Sequence
  Description of Artificial Sequence Synthetic oligonucleotide
ugccuaugagggugccgcu

SEQ ID NO: 21 - RNA, 19 nt, Artificial Sequence
  Description of Artificial Sequence Synthetic oligonucleotide
uuuccgaauaaacuccagg

SEQ ID NO: 22 - RNA, 19 nt, Artificial Sequence
  Description of Artificial Sequence Synthetic oligonucleotide
ugcauggcagcaggaagcg

SEQ ID NO: 23 - RNA, 19 nt, Artificial Sequence
  Description of Artificial Sequence Synthetic oligonucleotide
ugcuccggcagcagauggc

SEQ ID NO: 24 - RNA, 19 nt, Artificial Sequence
  Description of Artificial Sequence Synthetic oligonucleotide
ugccggcuccggcagcaga

SEQ ID NO: 25 - RNA, 19 nt, Artificial Sequence
  Description of Artificial Sequence Synthetic oligonucleotide
uuccucagggaaccaggcc

SEQ ID NO: 26 - RNA, 19 nt, Artificial Sequence
  Description of Artificial Sequence Synthetic oligonucleotide
ucuccggcagcagauggca

SEQ ID NO: 27 - RNA, 19 nt, Artificial Sequence
  Description of Artificial Sequence Synthetic oligonucleotide
ugccacgugggcagcagcc

SEQ ID NO: 28 - RNA, 19 nt, Artificial Sequence
  Description of Artificial Sequence Synthetic oligonucleotide
uuucaaguuacaaaagcaa

SEQ ID NO: 29 - RNA, 19 nt, Artificial Sequence
  Description of Artificial Sequence Synthetic oligonucleotide
ugcaaaacaggucuagaaa

SEQ ID NO: 30 - RNA, 19 nt, Artificial Sequence
  Description of Artificial Sequence Synthetic oligonucleotide
uacccagaauaaauaucuu

SEQ ID NO: 31 - RNA, 19 nt, Artificial Sequence
  Description of Artificial Sequence Synthetic oligonucleotide
uauaaaugucugcuugcuu

SEQ ID NO: 32 - RNA, 19 nt, Artificial Sequence
  Description of Artificial Sequence Synthetic oligonucleotide
uucaaguuacaaaagcaaa

SEQ ID NO: 33 - RNA, 19 nt, Artificial Sequence
  Description of Artificial Sequence Synthetic oligonucleotide
uaacagagaggacagaccc

SEQ ID NO: 34 - RNA, 19 nt, Artificial Sequence
  Description of Artificial Sequence Synthetic oligonucleotide
uaaauaucuucaaguuaca

SEQ ID NO: 35 - RNA, 19 nt, Artificial Sequence
  Description of Artificial Sequence Synthetic oligonucleotide
uggaaccauuuuaaagcuc

SEQ ID NO: 36 - RNA, 19 nt, Artificial Sequence
  Description of Artificial Sequence Synthetic oligonucleotide
ucaaaacaggucuagaaaa

SEQ ID NO: 37 - RNA, 19 nt, Artificial Sequence
  Description of Artificial Sequence Synthetic oligonucleotide
uaaaugcuacaaaacccag

SEQ ID NO: 38 - RNA, 19 nt, Artificial Sequence
  Description of Artificial Sequence Synthetic oligonucleotide
uccaaaagauaaaugucug

SEQ ID NO: 39 - RNA, 19 nt, Artificial Sequence
  Description of Artificial Sequence Synthetic oligonucleotide
uagcaccuggcaauggcgu

SEQ ID NO: 40 - RNA, 19 nt, Artificial Sequence
  Description of Artificial Sequence Synthetic oligonucleotide
uagaauccugccuccuugg

SEQ ID NO: 41 - RNA, 19 nt, Artificial Sequence
  Description of Artificial Sequence Synthetic oligonucleotide
uuggaucagucucugccuc

SEQ ID NO: 42 - RNA, 19 nt, Artificial Sequence
  Description of Artificial Sequence Synthetic oligonucleotide
ucccagaauaaauaucuuc

SEQ ID NO: 43 - RNA, 19 nt, Artificial Sequence
  Description of Artificial Sequence Synthetic oligonucleotide
uccagaauaaauaucuuca

SEQ ID NO: 44 - RNA, 19 nt, Artificial Sequence
  Description of Artificial Sequence Synthetic oligonucleotide
ucuucaaguuacaaaagca

SEQ ID NO: 45 - RNA, 19 nt, Artificial Sequence
  Description of Artificial Sequence Synthetic oligonucleotide
uaugcuacaaaacccagaa

SEQ ID NO: 46 - RNA, 19 nt, Artificial Sequence
  Description of Artificial Sequence Synthetic oligonucleotide
uaaaagcaaaacaggucua

SEQ ID NO: 47 - RNA, 19 nt, Artificial Sequence
  Description of Artificial Sequence Synthetic oligonucleotide
uaauaucuucaaguuacaa

SEQ ID NO: 48 - RNA, 19 nt, Artificial Sequence
  Description of Artificial Sequence Synthetic oligonucleotide
ugugacacaaagcaggugc

SEQ ID NO: 49 - RNA, 19 nt, Artificial Sequence
  Description of Artificial Sequence Synthetic oligonucleotide
uauaaauaucuucaaguua

SEQ ID NO: 50 - RNA, 19 nt, Artificial Sequence
  Description of Artificial Sequence Synthetic oligonucleotide
uaaugcuacaaaacccaga

SEQ ID NO: 51 - RNA, 19 nt, Artificial Sequence
  Description of Artificial Sequence Synthetic oligonucleotide
ucaaagcaggugcugcagu

SEQ ID NO: 52 - RNA, 19 nt, Artificial Sequence
  Description of Artificial Sequence Synthetic oligonucleotide
uagaauaaauaucuucaag

SEQ ID NO: 53 - RNA, 19 nt, Artificial Sequence
  Description of Artificial Sequence Synthetic oligonucleotide
uacaaaagcaaaacagguc

SEQ ID NO: 54 - RNA, 19 nt, Artificial Sequence
  Description of Artificial Sequence Synthetic oligonucleotide
uacaaaacccagaauaaau

SEQ ID NO: 55 - RNA, 19 nt, Artificial Sequence
  Description of Artificial Sequence Synthetic oligonucleotide
uaaagcaaaacaggucuag

SEQ ID NO: 56 - RNA, 19 nt, Artificial Sequence
  Description of Artificial Sequence Synthetic oligonucleotide
ucgaagucggugaccauga

SEQ ID NO: 57 - RNA, 19 nt, Artificial Sequence
  Description of Artificial Sequence Synthetic oligonucleotide
ucaaaagcaaaacaggucu

SEQ ID NO: 58 - RNA, 19 nt, Artificial Sequence
  Description of Artificial Sequence Synthetic oligonucleotide
ucaaguuacaaaagcaaaa

SEQ ID NO: 59 - RNA, 19 nt, Artificial Sequence
  Description of Artificial Sequence Synthetic oligonucleotide
uccuugacuuugcauucca

SEQ ID NO: 60 - RNA, 19 nt, Artificial Sequence
  Description of Artificial Sequence Synthetic oligonucleotide
uaagcguggaugcuggccu

SEQ ID NO: 61 - RNA, 19 nt, Artificial Sequence
  Description of Artificial Sequence Synthetic oligonucleotide
uuaaaugucugcuugcuug

SEQ ID NO: 62 - RNA, 19 nt, Artificial Sequence
  Description of Artificial Sequence Synthetic oligonucleotide
uaaccauuuuaaagcucag

SEQ ID NO: 63 - RNA, 19 nt, Artificial Sequence
  Description of Artificial Sequence Synthetic oligonucleotide
uaccaggccucauugauga

SEQ ID NO: 64 - RNA, 19 nt, Artificial Sequence
  Description of Artificial Sequence Synthetic oligonucleotide
uaaacccagaauaaauauc

SEQ ID NO: 65 - RNA, 19 nt, Artificial Sequence
  Description of Artificial Sequence Synthetic oligonucleotide
ugaaccauuuuaaagcuca

SEQ ID NO: 66 - RNA, 19 nt, Artificial Sequence
  Description of Artificial Sequence Synthetic oligonucleotide
ugaauaaauaucuucaagu

SEQ ID NO: 67 - RNA, 19 nt, Artificial Sequence
  Description of Artificial Sequence Synthetic oligonucleotide
uaagaggcuuggcuucaga

SEQ ID NO: 68 - RNA, 19 nt, Artificial Sequence
  Description of Artificial Sequence Synthetic oligonucleotide
ugaaccaggccucauugau

SEQ ID NO: 69 - RNA, 19 nt, Artificial Sequence
  Description of Artificial Sequence Synthetic oligonucleotide
ucagaauaaauaucuucaa

SEQ ID NO: 70 - RNA, 19 nt, Artificial Sequence
  Description of Artificial Sequence Synthetic oligonucleotide
uaagaauccugccuccuug

SEQ ID NO: 71 - RNA, 19 nt, Artificial Sequence
  Description of Artificial Sequence Synthetic oligonucleotide
uaaaacaggucuagaaaag

SEQ ID NO: 72 - RNA, 19 nt, Artificial Sequence
  Description of Artificial Sequence Synthetic oligonucleotide
uaggaagcguggaugcugg

SEQ ID NO: 73 - RNA, 19 nt, Artificial Sequence
  Description of Artificial Sequence Synthetic oligonucleotide
ucacugguugggcugaccu

SEQ ID NO: 74 - RNA, 19 nt, Artificial Sequence
  Description of Artificial Sequence Synthetic oligonucleotide
uagacaugcaggaucuugg

SEQ ID NO: 75 - RNA, 19 nt, Artificial Sequence
  Description of Artificial Sequence Synthetic oligonucleotide
ucuaggagauacaccucca

SEQ ID NO: 76 - RNA, 19 nt, Artificial Sequence
  Description of Artificial Sequence Synthetic oligonucleotide
ucuacaaaacccagaauaa

SEQ ID NO: 77 - RNA, 19 nt, Artificial Sequence
  Description of Artificial Sequence Synthetic oligonucleotide
ucagacagcaucauggcug

SEQ ID NO: 78 - RNA, 19 nt, Artificial Sequence
  Description of Artificial Sequence Synthetic oligonucleotide
uuaugcuggugucuaggag

SEQ ID NO: 79 - RNA, 19 nt, Artificial Sequence
  Description of Artificial Sequence Synthetic oligonucleotide
ugaaacuggagcagcucag

SEQ ID NO: 80 - RNA, 19 nt, Artificial Sequence
  Description of Artificial Sequence Synthetic oligonucleotide
uaggacagacccaaaagau

SEQ ID NO: 81 - RNA, 19 nt, Artificial Sequence
  Description of Artificial Sequence Synthetic oligonucleotide
ucaauggcguagacacccu

SEQ ID NO: 82 - RNA, 19 nt, Artificial Sequence
  Description of Artificial Sequence Synthetic oligonucleotide
ucaggugcugcagucgcug

SEQ ID NO: 83 - RNA, 19 nt, Artificial Sequence
  Description of Artificial Sequence Synthetic oligonucleotide
ucugccuccuugguggaga

SEQ ID NO: 84 - RNA, 19 nt, Artificial Sequence
  Description of Artificial Sequence Synthetic oligonucleotide
uagucggugaccaugaccc

SEQ ID NO: 85 - RNA, 19 nt, Artificial Sequence
  Description of Artificial Sequence Synthetic oligonucleotide
ucagccuggcauagagcag

SEQ ID NO: 86 - RNA, 19 nt, Artificial Sequence
  Description of Artificial Sequence Synthetic oligonucleotide
ucagagaggacagacccaa

SEQ ID NO: 87 - RNA, 19 nt, Artificial Sequence
  Description of Artificial Sequence Synthetic oligonucleotide
ucuggcucaguuccugcug

SEQ ID NO: 88 - RNA, 19 nt, Artificial Sequence
  Description of Artificial Sequence Synthetic oligonucleotide
ugagcuuccuggucugugu

SEQ ID NO: 89 - RNA, 19 nt, Artificial Sequence
  Description of Artificial Sequence Synthetic oligonucleotide
uaggugcugcagucgcugg

SEQ ID NO: 90 - RNA, 19 nt, Artificial Sequence
  Description of Artificial Sequence Synthetic oligonucleotide
ucuggccugucuguggaag

SEQ ID NO: 91 - RNA, 19 nt, Artificial Sequence
  Description of Artificial Sequence Synthetic oligonucleotide
uaccgccuggagcugacgg

SEQ ID NO: 92 - RNA, 19 nt, Artificial Sequence
  Description of Artificial Sequence Synthetic oligonucleotide
ucaaaagcguugugggccc

SEQ ID NO: 93 - RNA, 19 nt, Artificial Sequence
  Description of Artificial Sequence Synthetic oligonucleotide
ucacagccuggcauagagc

SEQ ID NO: 94 - RNA, 19 nt, Artificial Sequence
  Description of Artificial Sequence Synthetic oligonucleotide
ugggauuccaugcuccuug

SEQ ID NO: 95 - RNA, 19 nt, Artificial Sequence
  Description of Artificial Sequence Synthetic oligonucleotide
ugugaccaugacccugccc

SEQ ID NO: 96 - RNA, 19 nt, Artificial Sequence
  Description of Artificial Sequence Synthetic oligonucleotide
uagcaaaacaggucuagaa

SEQ ID NO: 97 - RNA, 19 nt, Artificial Sequence
  Description of Artificial Sequence Synthetic oligonucleotide
ucuggcucacuccuccagg

SEQ ID NO: 98 - RNA, 19 nt, Artificial Sequence
  Description of Artificial Sequence Synthetic oligonucleotide
ugaaguggaucagucucug

SEQ ID NO: 99 - RNA, 19 nt, Artificial Sequence
  Description of Artificial Sequence Synthetic oligonucleotide
uaugcuggugucuaggaga

SEQ ID NO: 100 - RNA, 19 nt, Artificial Sequence
  Description of Artificial Sequence Synthetic oligonucleotide
ucucaguuccugcugugug

SEQ ID NO: 101 - RNA, 19 nt, Artificial Sequence
  Description of Artificial Sequence Synthetic oligonucleotide
uuacaccuccaccaggcug

SEQ ID NO: 102 - RNA, 19 nt, Artificial Sequence
  Description of Artificial Sequence Synthetic oligonucleotide
uaucauggcugcaaugcca

SEQ ID NO: 103 - RNA, 19 nt, Artificial Sequence
  Description of Artificial Sequence Synthetic oligonucleotide
uacagagaggacagaccca

SEQ ID NO: 104 - RNA, 19 nt, Artificial Sequence
  Description of Artificial Sequence Synthetic oligonucleotide
ugaagaggcuuggcuucag

SEQ ID NO: 105 - RNA, 19 nt, Artificial Sequence
  Description of Artificial Sequence Synthetic oligonucleotide
ucccugcccucgauuuccc

SEQ ID NO: 106 - RNA, 19 nt, Artificial Sequence
  Description of Artificial Sequence Synthetic oligonucleotide
uaccaggaagccaggaaga

SEQ ID NO: 107 - RNA, 19 nt, Artificial Sequence
  Description of Artificial Sequence Synthetic oligonucleotide
ugagggagcuuccuggcac

SEQ ID NO: 108 - RNA, 19 nt, Artificial Sequence
  Description of Artificial Sequence Synthetic oligonucleotide
uaccauuuuaaagcucagc

SEQ ID NO: 109 - RNA, 19 nt, Artificial Sequence
  Description of Artificial Sequence Synthetic oligonucleotide
ucacuugcuggccugucug

SEQ ID NO: 110 - RNA, 19 nt, Artificial Sequence
  Description of Artificial Sequence Synthetic oligonucleotide
ugccacucaucuucaccag

SEQ ID NO: 111 - RNA, 19 nt, Artificial Sequence
  Description of Artificial Sequence Synthetic oligonucleotide
uuuggcagagaaguggauc

SEQ ID NO: 112 - RNA, 19 nt, Artificial Sequence
  Description of Artificial Sequence Synthetic oligonucleotide
uaguccuccucgauguagu

SEQ ID NO: 113 - RNA, 19 nt, Artificial Sequence
  Description of Artificial Sequence Synthetic oligonucleotide
uccucuggcuagaugccau

SEQ ID NO: 114 - RNA, 19 nt, Artificial Sequence
  Description of Artificial Sequence Synthetic oligonucleotide
uggagcuguguggacgcug

SEQ ID NO: 115 - RNA, 19 nt, Artificial Sequence
  Description of Artificial Sequence Synthetic oligonucleotide
ugagaaguggaucagucuc

SEQ ID NO: 116 - RNA, 19 nt, Artificial Sequence
  Description of Artificial Sequence Synthetic oligonucleotide
uagcagauggcaacggcug

SEQ ID NO: 117 - RNA, 19 nt, Artificial Sequence
  Description of Artificial Sequence Synthetic oligonucleotide
uaggccucauugaugacau

SEQ ID NO: 118 - RNA, 19 nt, Artificial Sequence
  Description of Artificial Sequence Synthetic oligonucleotide
uugcagucgcuggaggcac

SEQ ID NO: 119 - RNA, 19 nt, Artificial Sequence
  Description of Artificial Sequence Synthetic oligonucleotide
uugggucuccuccuucagc

SEQ ID NO: 120 - RNA, 19 nt, Artificial Sequence
  Description of Artificial Sequence Synthetic oligonucleotide
ucggcucggcagacagcau

SEQ ID NO: 121 - RNA, 19 nt, Artificial Sequence
  Description of Artificial Sequence Synthetic oligonucleotide
uugacaucuuuggcagaga

SEQ ID NO: 122 - RNA, 19 nt, Artificial Sequence
  Description of Artificial Sequence Synthetic oligonucleotide
ugaacgcaaggcuagcacc

SEQ ID NO: 123 - RNA, 19 nt, Artificial Sequence
  Description of Artificial Sequence Synthetic oligonucleotide
uaucuuuggcagagaagug

SEQ ID NO: 124 - RNA, 19 nt, Artificial Sequence
  Description of Artificial Sequence Synthetic oligonucleotide
uggcaggcaucgucccgga

SEQ ID NO: 125 - RNA, 19 nt, Artificial Sequence
  Description of Artificial Sequence Synthetic oligonucleotide
uggaacgcaaggcuagcac

SEQ ID NO: 126 - RNA, 19 nt, Artificial Sequence
  Description of Artificial Sequence Synthetic oligonucleotide
ugacacaaagcaggugcug

SEQ ID NO: 127 - RNA, 19 nt, Artificial Sequence
  Description of Artificial Sequence Synthetic oligonucleotide
uauaucuucaaguuacaaa

SEQ ID NO: 128 - RNA, 19 nt, Artificial Sequence
  Description of Artificial Sequence Synthetic oligonucleotide
ugugcccuucccuuggcag

SEQ ID NO: 129 - RNA, 19 nt, Artificial Sequence
  Description of Artificial Sequence Synthetic oligonucleotide
ucaaugccagccacguggg

SEQ ID NO: 130 - RNA, 19 nt, Artificial Sequence
  Description of Artificial Sequence Synthetic oligonucleotide
ugaggcuuggcuucagagc

SEQ ID NO: 131 - RNA, 19 nt, Artificial Sequence
  Description of Artificial Sequence Synthetic oligonucleotide
uggaucuuggugagguauc

SEQ ID NO: 132 - RNA, 19 nt, Artificial Sequence
  Description of Artificial Sequence Synthetic oligonucleotide
ucaacugugaugaccucgg

SEQ ID NO: 133 - RNA, 19 nt, Artificial Sequence
  Description of Artificial Sequence Synthetic oligonucleotide
uaaaugucugcuugcuugg

SEQ ID NO: 134 - RNA, 19 nt, Artificial Sequence
  Description of Artificial Sequence Synthetic oligonucleotide
uaaacaggucuagaaaagu

SEQ ID NO: 135 - RNA, 19 nt, Artificial Sequence
  Description of Artificial Sequence Synthetic oligonucleotide
uaaagcguugugggcccgg

SEQ ID NO: 136 - RNA, 19 nt, Artificial Sequence
  Description of Artificial Sequence Synthetic oligonucleotide
ucaacagagaggacagacc

SEQ ID NO: 137 - RNA, 19 nt, Artificial Sequence
  Description of Artificial Sequence Synthetic oligonucleotide
ucuccacggauccuuggcg

SEQ ID NO: 138 - RNA, 19 nt, Artificial Sequence
  Description of Artificial Sequence Synthetic oligonucleotide
uaggacugugcaggagcug

SEQ ID NO: 139 - RNA, 19 nt, Artificial Sequence
  Description of Artificial Sequence Synthetic oligonucleotide
ucaaaagauaaaugucugc

SEQ ID NO: 140 - RNA, 19 nt, Artificial Sequence
  Description of Artificial Sequence Synthetic oligonucleotide
uauccugccuccuuggugg

SEQ ID NO: 141 - RNA, 19 nt, Artificial Sequence
  Description of Artificial Sequence Synthetic oligonucleotide
ucacagcggccaaaguugg

SEQ ID NO: 142 - RNA, 19 nt, Artificial Sequence
  Description of Artificial Sequence Synthetic oligonucleotide
ucaguuccugcugugugag

SEQ ID NO: 143 - RNA, 19 nt, Artificial Sequence
  Description of Artificial Sequence Synthetic oligonucleotide
ucuggcauagagcagagua

SEQ ID NO: 144 - RNA, 19 nt, Artificial Sequence
  Description of Artificial Sequence Synthetic oligonucleotide
uccucauugaugacaucuu

SEQ ID NO: 145 - RNA, 19 nt, Artificial Sequence
  Description of Artificial Sequence Synthetic oligonucleotide
uagucgccguccucguccu

SEQ ID NO: 146 - RNA, 19 nt, Artificial Sequence
  Description of Artificial Sequence Synthetic oligonucleotide
uugggaagaauccugccuc

SEQ ID NO: 147 - RNA, 19 nt, Artificial Sequence
  Description of Artificial Sequence Synthetic oligonucleotide
ugugggugcuugacgccug

SEQ ID NO: 148 - RNA, 19 nt, Artificial Sequence
  Description of Artificial Sequence Synthetic oligonucleotide
uacacaaagcaggugcugc

SEQ ID NO: 149 - RNA, 19 nt, Artificial Sequence
  Description of Artificial Sequence Synthetic oligonucleotide
uagcaggaagcguggaugc

SEQ ID NO: 150 - RNA, 19 nt, Artificial Sequence
  Description of Artificial Sequence Synthetic oligonucleotide
uaucagucucugccucaac

SEQ ID NO: 151 - RNA, 19 nt, Artificial Sequence
  Description of Artificial Sequence Synthetic oligonucleotide
uagacagcaucauggcugc

SEQ ID NO: 152 - RNA, 19 nt, Artificial Sequence
  Description of Artificial Sequence Synthetic oligonucleotide
uucauggcugcaaugccag

SEQ ID NO: 153 - RNA, 19 nt, Artificial Sequence
  Description of Artificial Sequence Synthetic oligonucleotide
uacagccuggcauagagca

SEQ ID NO: 154 - RNA, 19 nt, Artificial Sequence
  Description of Artificial Sequence Synthetic oligonucleotide
uggcauagagcagaguaaa

SEQ ID NO: 155 - RNA, 19 nt, Artificial Sequence
  Description of Artificial Sequence Synthetic oligonucleotide
ucugguugggcugaccucg

SEQ ID NO: 156 - RNA, 19 nt, Artificial Sequence
  Description of Artificial Sequence Synthetic oligonucleotide
uagacccaaaagauaaaug

SEQ ID NO: 157 - RNA, 19 nt, Artificial Sequence
  Description of Artificial Sequence Synthetic oligonucleotide
ugguccacacagcggccaa

SEQ ID NO: 158 - RNA, 19 nt, Artificial Sequence
  Description of Artificial Sequence Synthetic oligonucleotide
uccuccaccaggcugccuc

SEQ ID NO: 159 - RNA, 19 nt, Artificial Sequence
  Description of Artificial Sequence Synthetic oligonucleotide
uaaaacagcugccaaccug

SEQ ID NO: 160 - RNA, 19 nt, Artificial Sequence
  Description of Artificial Sequence Synthetic oligonucleotide
ugaggacagacccaaaaga

SEQ ID NO: 161 - RNA, 19 nt, Artificial Sequence
  Description of Artificial Sequence Synthetic oligonucleotide
uugucugcuugcuugggug

SEQ ID NO: 162 - RNA, 19 nt, Artificial Sequence
  Description of Artificial Sequence Synthetic oligonucleotide
uaaggcaacagagaggaca

SEQ ID NO: 163 - RNA, 19 nt, Artificial Sequence
  Description of Artificial Sequence Synthetic oligonucleotide
ugugucuaggagauacacc

SEQ ID NO: 164 - RNA, 19 nt, Artificial Sequence
  Description of Artificial Sequence Synthetic oligonucleotide
uguggaucagucucugccu

SEQ ID NO: 165 - RNA, 19 nt, Artificial Sequence
  Description of Artificial Sequence Synthetic oligonucleotide
uaaacagcugccaaccugc

SEQ ID NO: 166 - RNA, 19 nt, Artificial Sequence
  Description of Artificial Sequence Synthetic oligonucleotide
ucaggcggcuugugggugc

SEQ ID NO: 167 - RNA, 19 nt, Artificial Sequence
  Description of Artificial Sequence Synthetic oligonucleotide
uggaaccaggccucauuga

SEQ ID NO: 168 - RNA, 19 nt, Artificial Sequence
  Description of Artificial Sequence Synthetic oligonucleotide
ucugccuggcucacuccuc

SEQ ID NO: 169 - RNA, 19 nt, Artificial Sequence
  Description of Artificial Sequence Synthetic oligonucleotide
uagaugagggccaucagca

SEQ ID NO: 170 - RNA, 19 nt, Artificial Sequence
  Description of Artificial Sequence Synthetic oligonucleotide
ucuuccuggucuguguucc

SEQ ID NO: 171 - RNA, 19 nt, Artificial Sequence
  Description of Artificial Sequence Synthetic oligonucleotide
uacuuugcauuccagaccu

SEQ ID NO: 172 - RNA, 19 nt, Artificial Sequence
  Description of Artificial Sequence Synthetic oligonucleotide
uuggcuagaugccauccag

SEQ ID NO: 173 - RNA, 19 nt, Artificial Sequence
  Description of Artificial Sequence Synthetic oligonucleotide
uuggcugcaaugccagcca

SEQ ID NO: 174 - RNA, 19 nt, Artificial Sequence
  Description of Artificial Sequence Synthetic oligonucleotide
ugggugugggugcuugacg

SEQ ID NO: 175 - RNA, 19 nt, Artificial Sequence
  Description of Artificial Sequence Synthetic oligonucleotide
uaaaagcguugugggcccg

SEQ ID NO: 176 - RNA, 19 nt, Artificial Sequence
  Description of Artificial Sequence Synthetic oligonucleotide
ucaccaggaagccaggaag

SEQ ID NO: 177 - RNA, 19 nt, Artificial Sequence
  Description of Artificial Sequence Synthetic oligonucleotide
ugccgggauuccaugcucc

SEQ ID NO: 178 - RNA, 19 nt, Artificial Sequence
  Description of Artificial Sequence Synthetic oligonucleotide
uccagcuccucguagucgc

SEQ ID NO: 179 - RNA, 19 nt, Artificial Sequence
  Description of Artificial Sequence Synthetic oligonucleotide
ucccgcugguccucaggga

SEQ ID NO: 180 - RNA, 19 nt, Artificial Sequence
  Description of Artificial Sequence Synthetic oligonucleotide
uugcaaugccagccacgug

SEQ ID NO: 181 - RNA, 19 nt, Artificial Sequence
  Description of Artificial Sequence Synthetic oligonucleotide
uugaugacaucuuuggcag

SEQ ID NO: 182 - RNA, 19 nt, Artificial Sequence
  Description of Artificial Sequence Synthetic oligonucleotide
ugagauacaccuccaccag

SEQ ID NO: 183 - RNA, 19 nt, Artificial Sequence
  Description of Artificial Sequence Synthetic oligonucleotide
uggugugggugcuugacgc

SEQ ID NO: 184 - RNA, 19 nt, Artificial Sequence
  Description of Artificial Sequence Synthetic oligonucleotide
ucugccucaacucggccag

SEQ ID NO: 185 - RNA, 19 nt, Artificial Sequence
  Description of Artificial Sequence Synthetic oligonucleotide
ugaagucggugaccaugac

SEQ ID NO: 186 - RNA, 19 nt, Artificial Sequence
  Description of Artificial Sequence Synthetic oligonucleotide
uguccucguccuccugcgc

SEQ ID NO: 187 - RNA, 19 nt, Artificial Sequence
  Description of Artificial Sequence Synthetic oligonucleotide
uccagccucacuguuaccc

SEQ ID NO: 188 - RNA, 19 nt, Artificial Sequence
  Description of Artificial Sequence Synthetic oligonucleotide
ugcuuuuccgaauaaacuc

SEQ ID NO: 189 - RNA, 19 nt, Artificial Sequence
  Description of Artificial Sequence Synthetic oligonucleotide
uugugagcuuggcaggcac

SEQ ID NO: 190 - RNA, 19 nt, Artificial Sequence
  Description of Artificial Sequence Synthetic oligonucleotide
uagcguggaugcuggccuc

SEQ ID NO: 191 - RNA, 19 nt, Artificial Sequence
  Description of Artificial Sequence Synthetic oligonucleotide
uaagacagaggaguccucc

SEQ ID NO: 192 - RNA, 19 nt, Artificial Sequence
  Description of Artificial Sequence Synthetic oligonucleotide
ugucugcuugcuugggugg

SEQ ID NO: 193 - RNA, 19 nt, Artificial Sequence
  Description of Artificial Sequence Synthetic oligonucleotide
uguagcaggcagcaccugg

SEQ ID NO: 194 - RNA, 19 nt, Artificial Sequence
  Description of Artificial Sequence Synthetic oligonucleotide
uuggaugcuggccucccug

SEQ ID NO: 195 - RNA, 19 nt, Artificial Sequence
  Description of Artificial Sequence Synthetic oligonucleotide
uaaaagauaaaugucugcu

SEQ ID NO: 196 - RNA, 19 nt, Artificial Sequence
  Description of Artificial Sequence Synthetic oligonucleotide
uaguccugcaaaacagcug

SEQ ID NO: 197 - RNA, 19 nt, Artificial Sequence
  Description of Artificial Sequence Synthetic oligonucleotide
ucaggaucuuggugaggua

SEQ ID NO: 198 - RNA, 19 nt, Artificial Sequence
  Description of Artificial Sequence Synthetic oligonucleotide
uagcgguggaagguggcug

SEQ ID NO: 199 - RNA, 19 nt, Artificial Sequence
  Description of Artificial Sequence Synthetic oligonucleotide
uuggcaauggcguagacac

SEQ ID NO: 200 - RNA, 19 nt, Artificial Sequence
  Description of Artificial Sequence Synthetic oligonucleotide
ucagaggaguccuccucga

SEQ ID NO: 201 - RNA, 19 nt, Artificial Sequence
  Description of Artificial Sequence Synthetic oligonucleotide
uggagcagcucagcagcuc

SEQ ID NO: 202 - RNA, 19 nt, Artificial Sequence
  Description of Artificial Sequence Synthetic oligonucleotide
ugugugagcuuggcaggca

SEQ ID NO: 203 - RNA, 19 nt, Artificial Sequence
  Description of Artificial Sequence Synthetic oligonucleotide
ucagcaccuggcaauggcg

SEQ ID NO: 204 - RNA, 19 nt, Artificial Sequence
  Description of Artificial Sequence Synthetic oligonucleotide
uucccagccucacuguuac

SEQ ID NO: 205 - RNA, 19 nt, Artificial Sequence
  Description of Artificial Sequence Synthetic oligonucleotide
ugagaaacuggagcagcuc

SEQ ID NO: 206 - RNA, 19 nt, Artificial Sequence
  Description of Artificial Sequence Synthetic oligonucleotide
uagagaaguggaucagucu

SEQ ID NO: 207 - RNA, 19 nt, Artificial Sequence
  Description of Artificial Sequence Synthetic oligonucleotide
uggucgccacucaucuuca

SEQ ID NO: 208 - RNA, 19 nt, Artificial Sequence
  Description of Artificial Sequence Synthetic oligonucleotide
ugaccagcuggcuuuuccg

SEQ ID NO: 209 - RNA, 19 nt, Artificial Sequence
  Description of Artificial Sequence Synthetic oligonucleotide
ucaugcaggaucuugguga

SEQ ID NO: 210 - RNA, 19 nt, Artificial Sequence
  Description of Artificial Sequence Synthetic oligonucleotide
uagcggccaaaguuggucc

SEQ ID NO: 211 - RNA, 19 nt, Artificial Sequence
  Description of Artificial Sequence Synthetic oligonucleotide
ucagccugugaggacgugg

SEQ ID NO: 212 - RNA, 19 nt, Artificial Sequence
  Description of Artificial Sequence Synthetic oligonucleotide
ugagcuguguggacgcugc

SEQ ID NO: 213 - RNA, 19 nt, Artificial Sequence
  Description of Artificial Sequence Synthetic oligonucleotide
ugguagcaggcagcaccug

SEQ ID NO: 214 - RNA, 19 nt, Artificial Sequence
  Description of Artificial Sequence Synthetic oligonucleotide
uugagcuccggcucggcag

SEQ ID NO: 215 - RNA, 19 nt, Artificial Sequence
  Description of Artificial Sequence Synthetic oligonucleotide
ugucuaggagauacaccuc

SEQ ID NO: 216 - RNA, 19 nt, Artificial Sequence
  Description of Artificial Sequence Synthetic oligonucleotide
uggaagcgggucccguccu

SEQ ID NO: 217 - RNA, 19 nt, Artificial Sequence
  Description of Artificial Sequence Synthetic oligonucleotide
ugcugugugagcuuggcag

SEQ ID NO: 218 - RNA, 19 nt, Artificial Sequence
  Description of Artificial Sequence Synthetic oligonucleotide
uaccagcuggcuuuuccga

SEQ ID NO: 219 - RNA, 19 nt, Artificial Sequence
  Description of Artificial Sequence Synthetic oligonucleotide
ugcaaagagguccacacag

SEQ ID NO: 220 - RNA, 19 nt, Artificial Sequence
  Description of Artificial Sequence Synthetic oligonucleotide
uaggagaccuagaggccgu

SEQ ID NO: 221 - RNA, 19 nt, Artificial Sequence
  Description of Artificial Sequence Synthetic oligonucleotide
uguccugcaaaacagcugc

SEQ ID NO: 222 - RNA, 19 nt, Artificial Sequence
  Description of Artificial Sequence Synthetic oligonucleotide
ugcaccaccacguaggugc

SEQ ID NO: 223 - RNA, 19 nt, Artificial Sequence
  Description of Artificial Sequence Synthetic oligonucleotide
uacucaagggccaggccag

SEQ ID NO: 224 - RNA, 19 nt, Artificial Sequence
  Description of Artificial Sequence Synthetic oligonucleotide
uccauuuuaaagcucagcc

SEQ ID NO: 225 - RNA, 19 nt, Artificial Sequence
  Description of Artificial Sequence Synthetic oligonucleotide
uagcguugugggcccggca

SEQ ID NO: 226 - RNA, 19 nt, Artificial Sequence
  Description of Artificial Sequence Synthetic oligonucleotide
uggcagagaaguggaucag

SEQ ID NO: 227 - RNA, 19 nt, Artificial Sequence
  Description of Artificial Sequence Synthetic oligonucleotide
ugaaaccuucuagggugug

SEQ ID NO: 228 - RNA, 19 nt, Artificial Sequence
  Description of Artificial Sequence Synthetic oligonucleotide
uagcggugaccagcacgac

SEQ ID NO: 229 - RNA, 19 nt, Artificial Sequence
  Description of Artificial Sequence Synthetic oligonucleotide
ugcagucgcuggaggcacc

SEQ ID NO: 230 - RNA, 19 nt, Artificial Sequence
  Description of Artificial Sequence Synthetic oligonucleotide
uaaaacccagaauaaauau

SEQ ID NO: 231 - RNA, 19 nt, Artificial Sequence
  Description of Artificial Sequence Synthetic oligonucleotide
uaauggcguagacacccuc

SEQ ID NO: 232 - RNA, 19 nt, Artificial Sequence
  Description of Artificial Sequence Synthetic oligonucleotide
uucuucaaguuacaaaagc

SEQ ID NO: 233 - RNA, 19 nt, Artificial Sequence
  Description of Artificial Sequence Synthetic oligonucleotide
uuuacaaaagcaaaacagg

SEQ ID NO: 234 - RNA, 19 nt, Artificial Sequence
  Description of Artificial Sequence Synthetic oligonucleotide
uaucuucaaguuacaaaag

SEQ ID NO: 235 - RNA, 19 nt, Artificial Sequence
  Description of Artificial Sequence Synthetic oligonucleotide
ucuggugucuaggagauac

SEQ ID NO: 236 - RNA, 19 nt, Artificial Sequence
  Description of Artificial Sequence Synthetic oligonucleotide
uagugcgcucugacugcga

SEQ ID NO: 237 - RNA, 19 nt, Artificial Sequence
  Description of Artificial Sequence Synthetic oligonucleotide
uugucacacuugcuggccu

SEQ ID NO: 238 - RNA, 19 nt, Artificial Sequence
  Description of Artificial Sequence Synthetic oligonucleotide
uacagaggaguccuccucg

SEQ ID NO: 239 - RNA, 19 nt, Artificial Sequence
  Description of Artificial Sequence Synthetic oligonucleotide
uguuacaaaagcaaaacag

SEQ ID NO: 240 - RNA, 19 nt, Artificial Sequence
  Description of Artificial Sequence Synthetic oligonucleotide
uaauccugccuccuuggug

SEQ ID NO: 241 - RNA, 19 nt, Artificial Sequence
  Description of Artificial Sequence Synthetic oligonucleotide
uguggaagguggcuguggu

SEQ ID NO: 242 - RNA, 19 nt, Artificial Sequence
  Description of Artificial Sequence Synthetic oligonucleotide
uggagcggguuggcugaga

SEQ ID NO: 243 - RNA, 19 nt, Artificial Sequence
  Description of Artificial Sequence Synthetic oligonucleotide
uucgccacucaucuucacc

SEQ ID NO: 244 - RNA, 19 nt, Artificial Sequence
  Description of Artificial Sequence Synthetic oligonucleotide
uagaggaguccuccucgau

SEQ ID NO: 245 - RNA, 19 nt, Artificial Sequence
  Description of Artificial Sequence Synthetic oligonucleotide
ugagcuccggcucggcaga

SEQ ID NO: 246 - RNA, 19 nt, Artificial Sequence
  Description of Artificial Sequence Synthetic oligonucleotide
uaacugugaugaccucggg

SEQ ID NO: 247 - RNA, 19 nt, Artificial Sequence
  Description of Artificial Sequence Synthetic oligonucleotide
ugauacaccuccaccaggc

SEQ ID NO: 248 - RNA, 19 nt, Artificial Sequence
  Description of Artificial Sequence Synthetic oligonucleotide
uuggcauagagcagaguaa

SEQ ID NO: 249 - RNA, 19 nt, Artificial Sequence
  Description of Artificial Sequence Synthetic oligonucleotide
ugcagcucagcagcuccuc

SEQ ID NO: 250 - RNA, 19 nt, Artificial Sequence
  Description of Artificial Sequence Synthetic oligonucleotide
uauugaugacaucuuuggc

SEQ ID NO: 251 - RNA, 15 nt, Artificial Sequence
  Description of Artificial Sequence Synthetic oligonucleotide
cacccucauaggcca

SEQ ID NO: 252 - RNA, 15 nt, Artificial Sequence
  Description of Artificial Sequence Synthetic oligonucleotide
ccuggaguuuauuca

SEQ ID NO: 253 - RNA, 15 nt, Artificial Sequence
  Description of Artificial Sequence Synthetic oligonucleotide
aggccuggaguuuaa

SEQ ID NO: 254 - RNA, 15 nt, Artificial Sequence
  Description of Artificial Sequence Synthetic oligonucleotide
cuggaguuuauucga

SEQ ID NO: 255 - RNA, 15 nt, Artificial Sequence
  Description of Artificial Sequence Synthetic oligonucleotide
caucugcugccggaa

SEQ ID NO: 256 - RNA, 15 nt, Artificial Sequence
  Description of Artificial Sequence Synthetic oligonucleotide
cauaggccuggagua

SEQ ID NO: 257 - RNA, 15 nt, Artificial Sequence
  Description of Artificial Sequence Synthetic oligonucleotide
ggcacccucauagga

SEQ ID NO: 258 - RNA, 15 nt, Artificial Sequence
  Description of Artificial Sequence Synthetic oligonucleotide
acccucauaggccua

SEQ ID NO: 259 - RNA, 15 nt, Artificial Sequence
  Description of Artificial Sequence Synthetic oligonucleotide
cucauaggccuggaa

SEQ ID NO: 260 - RNA, 15 nt, Artificial Sequence
  Description of Artificial Sequence Synthetic oligonucleotide
cccugaggaccagca

SEQ ID NO: 261 - RNA, 15 nt, Artificial Sequence
  Description of Artificial Sequence Synthetic oligonucleotide
gccuggaguuuauua

SEQ ID NO: 262 - RNA, 15 nt, Artificial Sequence
  Description of Artificial Sequence Synthetic oligonucleotide
cugagcugcuccaga

SEQ ID NO: 263 - RNA, 15 nt, Artificial Sequence
  Description of Artificial Sequence Synthetic oligonucleotide
uggaguuuauucgga

SEQ ID NO: 264 - RNA, 15 nt, Artificial Sequence
  Description of Artificial Sequence Synthetic oligonucleotide
guucccugaggacca

SEQ ID NO: 265 - RNA, 15 nt, Artificial Sequence
  Description of Artificial Sequence Synthetic oligonucleotide
uucccugaggaccaa

SEQ ID NO: 266 - RNA, 15 nt, Artificial Sequence
  Description of Artificial Sequence Synthetic oligonucleotide
ggccuggaguuuaua

SEQ ID NO: 267 - RNA, 15 nt, Artificial Sequence
  Description of Artificial Sequence Synthetic oligonucleotide
ucauaggccuggaga

SEQ ID NO: 268 - RNA, 15 nt, Artificial Sequence
  Description of Artificial Sequence Synthetic oligonucleotide
cccucauaggccuga

SEQ ID NO: 269 - RNA, 15 nt, Artificial Sequence
  Description of Artificial Sequence Synthetic oligonucleotide
gguucccugaggaca

SEQ ID NO: 270 - RNA, 15 nt, Artificial Sequence
  Description of Artificial Sequence Synthetic oligonucleotide
gcacccucauaggca

SEQ ID NO: 271 - RNA, 15 nt, Artificial Sequence
  Description of Artificial Sequence Synthetic oligonucleotide
gaguuuauucggaaa

SEQ ID NO: 272 - RNA, 15 nt, Artificial Sequence
  Description of Artificial Sequence Synthetic oligonucleotide
uccugcugccaugca

SEQ ID NO: 273 - RNA, 15 nt, Artificial Sequence
  Description of Artificial Sequence Synthetic oligonucleotide
ucugcugccggagca

SEQ ID NO: 274 - RNA, 15 nt, Artificial Sequence
  Description of Artificial Sequence Synthetic oligonucleotide
cugccggagccggca

SEQ ID NO: 275 - RNA, 15 nt, Artificial Sequence
  Description of Artificial Sequence Synthetic oligonucleotide
ugguucccugaggaa

SEQ ID NO: 276 - RNA, 15 nt, Artificial Sequence
  Description of Artificial Sequence Synthetic oligonucleotide
aucugcugccggaga

SEQ ID NO: 277 - RNA, 15 nt, Artificial Sequence
  Description of Artificial Sequence Synthetic oligonucleotide
gcugcccacguggca

SEQ ID NO: 278 - RNA, 15 nt, Artificial Sequence
  Description of Artificial Sequence Synthetic oligonucleotide
uuuuguaacuugaaa

SEQ ID NO: 279 - RNA, 15 nt, Artificial Sequence
  Description of Artificial Sequence Synthetic oligonucleotide
uagaccuguuuugca

SEQ ID NO: 280 - RNA, 15 nt, Artificial Sequence
  Description of Artificial Sequence Synthetic oligonucleotide
uauuuauucugggua

SEQ ID NO: 281 - RNA, 15 nt, Artificial Sequence
  Description of Artificial Sequence Synthetic oligonucleotide
aagcagacauuuaua

SEQ ID NO: 282 - RNA, 15 nt, Artificial Sequence
  Description of Artificial Sequence Synthetic oligonucleotide
cuuuuguaacuugaa

SEQ ID NO: 283 - RNA, 15 nt, Artificial Sequence
  Description of Artificial Sequence Synthetic oligonucleotide
cuguccucucuguua

SEQ ID NO: 284 - RNA, 15 nt, Artificial Sequence
  Description of Artificial Sequence Synthetic oligonucleotide
acuugaagauauuua

SEQ ID NO: 285 - RNA, 15 nt, Artificial Sequence
  Description of Artificial Sequence Synthetic oligonucleotide
uuuaaaaugguucca

SEQ ID NO: 286 - RNA, 15 nt, Artificial Sequence
  Description of Artificial Sequence Synthetic oligonucleotide
cuagaccuguuuuga

SEQ ID NO: 287 - RNA, 15 nt, Artificial Sequence
  Description of Artificial Sequence Synthetic oligonucleotide
guuuuguagcauuua

SEQ ID NO: 288 - RNA, 15 nt, Artificial Sequence
  Description of Artificial Sequence Synthetic oligonucleotide
cauuuaucuuuugga

SEQ ID NO: 289 - RNA, 15 nt, Artificial Sequence
  Description of Artificial Sequence Synthetic oligonucleotide
cauugccaggugcua

SEQ ID NO: 290 - RNA, 15 nt, Artificial Sequence
  Description of Artificial Sequence Synthetic oligonucleotide
ggaggcaggauucua

SEQ ID NO: 291 - RNA, 15 nt, Artificial Sequence
  Description of Artificial Sequence Synthetic oligonucleotide
cagagacugauccaa

SEQ ID NO: 292 - RNA, 15 nt, Artificial Sequence
  Description of Artificial Sequence Synthetic oligonucleotide
auauuuauucuggga

SEQ ID NO: 293 - RNA, 15 nt, Artificial Sequence
  Description of Artificial Sequence Synthetic oligonucleotide
gauauuuauucugga

SEQ ID NO: 294 - RNA, 15 nt, Artificial Sequence
  Description of Artificial Sequence Synthetic oligonucleotide
uuuguaacuugaaga

SEQ ID NO: 295 - RNA, 15 nt, Artificial Sequence
  Description of Artificial Sequence Synthetic oligonucleotide
ggguuuuguagcaua

SEQ ID NO: 296 - RNA, 15 nt, Artificial Sequence
  Description of Artificial Sequence Synthetic oligonucleotide
ccuguuuugcuuuua

SEQ ID NO: 297 - RNA, 15 nt, Artificial Sequence
  Description of Artificial Sequence Synthetic oligonucleotide
aacuugaagauauua

SEQ ID NO: 298 - RNA, 15 nt, Artificial Sequence
  Description of Artificial Sequence Synthetic oligonucleotide
cugcuuugugucaca

SEQ ID NO: 299 - RNA, 15 nt, Artificial Sequence
  Description of Artificial Sequence Synthetic oligonucleotide
uugaagauauuuaua

SEQ ID NO: 300 - RNA, 15 nt, Artificial Sequence
  Description of Artificial Sequence Synthetic oligonucleotide
gguuuuguagcauua

SEQ ID NO: 301 - RNA, 15 nt, Artificial Sequence
  Description of Artificial Sequence Synthetic oligonucleotide
cagcaccugcuuuga

SEQ ID NO: 302 - RNA, 15 nt, Artificial Sequence
  Description of Artificial Sequence Synthetic oligonucleotide
aagauauuuauucua

SEQ ID NO: 303 - RNA, 15 nt, Artificial Sequence
  Description of Artificial Sequence Synthetic oligonucleotide
uguuuugcuuuugua

SEQ ID NO: 304 - RNA, 15 nt, Artificial Sequence
  Description of Artificial Sequence Synthetic oligonucleotide
auucuggguuuugua

SEQ ID NO: 305 - RNA, 15 nt, Artificial Sequence
  Description of Artificial Sequence Synthetic oligonucleotide
accuguuuugcuuua

SEQ ID NO: 306 - RNA, 15 nt, Artificial Sequence
  Description of Artificial Sequence Synthetic oligonucleotide
ggucaccgacuucga

SEQ ID NO: 307 - RNA, 15 nt, Artificial Sequence
  Description of Artificial Sequence Synthetic oligonucleotide
cuguuuugcuuuuga

SEQ ID NO: 308 - RNA, 15 nt, Artificial Sequence
  Description of Artificial Sequence Synthetic oligonucleotide
gcuuuuguaacuuga

SEQ ID NO: 309 - RNA, 15 nt, Artificial Sequence
  Description of Artificial Sequence Synthetic oligonucleotide
augcaaagucaagga

SEQ ID NO: 310 - RNA, 15 nt, Artificial Sequence
  Description of Artificial Sequence Synthetic oligonucleotide
cagcauccacgcuua

SEQ ID NO: 311 - RNA, 15 nt, Artificial Sequence
  Description of Artificial Sequence Synthetic oligonucleotide
caagcagacauuuaa

SEQ ID NO: 312 - RNA, 15 nt, Artificial Sequence
  Description of Artificial Sequence Synthetic oligonucleotide
gcuuuaaaaugguua

SEQ ID NO: 313 - RNA, 15 nt, Artificial Sequence
  Description of Artificial Sequence Synthetic oligonucleotide
caaugaggccuggua

SEQ ID NO: 314 - RNA, 15 nt, Artificial Sequence
  Description of Artificial Sequence Synthetic oligonucleotide
uuuauucuggguuua

SEQ ID NO: 315 - RNA, 15 nt, Artificial Sequence
  Description of Artificial Sequence Synthetic oligonucleotide
cuuuaaaaugguuca

SEQ ID NO: 316 - RNA, 15 nt, Artificial Sequence
  Description of Artificial Sequence Synthetic oligonucleotide
gaagauauuuauuca

SEQ ID NO: 317 - RNA, 15 nt, Artificial Sequence
  Description of Artificial Sequence Synthetic oligonucleotide
aagccaagccucuua

SEQ ID NO: 318 - RNA, 15 nt, Artificial Sequence
  Description of Artificial Sequence Synthetic oligonucleotide
augaggccugguuca

SEQ ID NO: 319 - RNA, 15 nt, Artificial Sequence
  Description of Artificial Sequence Synthetic oligonucleotide
agauauuuauucuga

SEQ ID NO: 320 - RNA, 15 nt, Artificial Sequence
  Description of Artificial Sequence Synthetic oligonucleotide
gaggcaggauucuua

SEQ ID NO: 321 - RNA, 15 nt, Artificial Sequence
  Description of Artificial Sequence Synthetic oligonucleotide
ucuagaccuguuuua

SEQ ID NO: 322 - RNA, 15 nt, Artificial Sequence
  Description of Artificial Sequence Synthetic oligonucleotide
cauccacgcuuccua

SEQ ID NO: 323 - RNA, 15 nt, Artificial Sequence
  Description of Artificial Sequence Synthetic oligonucleotide
cagcccaaccaguga

SEQ ID NO: 324 - RNA, 15 nt, Artificial Sequence
  Description of Artificial Sequence Synthetic oligonucleotide
gauccugcaugucua

SEQ ID NO: 325 - RNA, 15 nt, Artificial Sequence
  Description of Artificial Sequence Synthetic oligonucleotide
gguguaucuccuaga

SEQ ID NO: 326 - RNA, 15 nt, Artificial Sequence
  Description of Artificial Sequence Synthetic oligonucleotide
ucuggguuuuguaga

SEQ ID NO: 327 - RNA, 15 nt, Artificial Sequence
  Description of Artificial Sequence Synthetic oligonucleotide
caugaugcugucuga

SEQ ID NO: 328 - RNA, 15 nt, Artificial Sequence
  Description of Artificial Sequence Synthetic oligonucleotide
uagacaccagcauaa

SEQ ID NO: 329 - RNA, 15 nt, Artificial Sequence
  Description of Artificial Sequence Synthetic oligonucleotide
gcugcuccaguuuca

SEQ ID NO: 330 - RNA, 15 nt, Artificial Sequence
  Description of Artificial Sequence Synthetic oligonucleotide
uuugggucuguccua

SEQ ID NO: 331 - RNA, 15 nt, Artificial Sequence
  Description of Artificial Sequence Synthetic oligonucleotide
ugucuacgccauuga

SEQ ID NO: 332 - RNA, 15 nt, Artificial Sequence
  Description of Artificial Sequence Synthetic oligonucleotide
gacugcagcaccuga

SEQ ID NO: 333 - RNA, 15 nt, Artificial Sequence
  Description of Artificial Sequence Synthetic oligonucleotide
caccaaggaggcaga

SEQ ID NO: 334 - RNA, 15 nt, Artificial Sequence
  Description of Artificial Sequence Synthetic oligonucleotide
cauggucaccgacua

SEQ ID NO: 335 - RNA, 15 nt, Artificial Sequence
  Description of Artificial Sequence Synthetic oligonucleotide
ucuaugccaggcuga

SEQ ID NO: 336 - RNA, 15 nt, Artificial Sequence
  Description of Artificial Sequence Synthetic oligonucleotide
gucuguccucucuga

SEQ ID NO: 337 - RNA, 15 nt, Artificial Sequence
  Description of Artificial Sequence Synthetic oligonucleotide
aggaacugagccaga

SEQ ID NO: 338 - RNA, 15 nt, Artificial Sequence
  Description of Artificial Sequence Synthetic oligonucleotide
agaccaggaagcuca

SEQ ID NO: 339 - RNA, 15 nt, Artificial Sequence
  Description of Artificial Sequence Synthetic oligonucleotide
cgacugcagcaccua

SEQ ID NO: 340 - RNA, 15 nt, Artificial Sequence
  Description of Artificial Sequence Synthetic oligonucleotide
cacagacaggccaga

SEQ ID NO: 341 - RNA, 15 nt, Artificial Sequence
  Description of Artificial Sequence Synthetic oligonucleotide
cagcuccaggcggua

SEQ ID NO: 342 - RNA, 15 nt, Artificial Sequence
  Description of Artificial Sequence Synthetic oligonucleotide
ccacaacgcuuuuga

SEQ ID NO: 343 - RNA, 15 nt, Artificial Sequence
  Description of Artificial Sequence Synthetic oligonucleotide
uaugccaggcuguga

SEQ ID NO: 344 - RNA, 15 nt, Artificial Sequence
  Description of Artificial Sequence Synthetic oligonucleotide
gagcauggaauccca

SEQ ID NO: 345 - RNA, 15 nt, Artificial Sequence
  Description of Artificial Sequence Synthetic oligonucleotide
agggucauggucaca

SEQ ID NO: 346 - RNA, 15 nt, Artificial Sequence
  Description of Artificial Sequence Synthetic oligonucleotide
agaccuguuuugcua

SEQ ID NO: 347 - RNA, 15 nt, Artificial Sequence
  Description of Artificial Sequence Synthetic oligonucleotide
gaggagugagccaga

SEQ ID NO: 348 - RNA, 15 nt, Artificial Sequence
  Description of Artificial Sequence Synthetic oligonucleotide
gacugauccacuuca

SEQ ID NO: 349 - RNA, 15 nt, Artificial Sequence
  Description of Artificial Sequence Synthetic oligonucleotide
cuagacaccagcaua

SEQ ID NO: 350 - RNA, 15 nt, Artificial Sequence
  Description of Artificial Sequence Synthetic oligonucleotide
cagcaggaacugaga

SEQ ID NO: 351 - RNA, 15 nt, Artificial Sequence
  Description of Artificial Sequence Synthetic oligonucleotide
cugguggagguguaa

SEQ ID NO: 352 - RNA, 15 nt, Artificial Sequence
  Description of Artificial Sequence Synthetic oligonucleotide
auugcagccaugaua

SEQ ID NO: 353 - RNA, 15 nt, Artificial Sequence
  Description of Artificial Sequence Synthetic oligonucleotide
ucuguccucucugua

SEQ ID NO: 354 - RNA, 15 nt, Artificial Sequence
  Description of Artificial Sequence Synthetic oligonucleotide
agccaagccucuuca

SEQ ID NO: 355 - RNA, 15 nt, Artificial Sequence
  Description of Artificial Sequence Synthetic oligonucleotide
aaucgagggcaggga

SEQ ID NO: 356 - RNA, 15 nt, Artificial Sequence
  Description of Artificial Sequence Synthetic oligonucleotide
ccuggcuuccuggua

SEQ ID NO: 357 - RNA, 15 nt, Artificial Sequence
  Description of Artificial Sequence Synthetic oligonucleotide
caggaagcucccuca

SEQ ID NO: 358 - RNA, 15 nt, Artificial Sequence
  Description of Artificial Sequence Synthetic oligonucleotide
agcuuuaaaauggua

SEQ ID NO: 359 - RNA, 15 nt, Artificial Sequence
  Description of Artificial Sequence Synthetic oligonucleotide
caggccagcaaguga

SEQ ID NO: 360 - RNA, 15 nt, Artificial Sequence
  Description of Artificial Sequence Synthetic oligonucleotide
ugaagaugaguggca

SEQ ID NO: 361 - RNA, 15 nt, Artificial Sequence
  Description of Artificial Sequence Synthetic oligonucleotide
cacuucucugccaaa

SEQ ID NO: 362 - RNA, 15 nt, Artificial Sequence
  Description of Artificial Sequence Synthetic oligonucleotide
caucgaggaggacua

SEQ ID NO: 363 - RNA, 15 nt, Artificial Sequence
  Description of Artificial Sequence Synthetic oligonucleotide
caucuagccagagga

SEQ ID NO: 364 - RNA, 15 nt, Artificial Sequence
  Description of Artificial Sequence Synthetic oligonucleotide
guccacacagcucca

SEQ ID NO: 365 - RNA, 15 nt, Artificial Sequence
  Description of Artificial Sequence Synthetic oligonucleotide
cugauccacuucuca

SEQ ID NO: 366 - RNA, 15 nt, Artificial Sequence
  Description of Artificial Sequence Synthetic oligonucleotide
cguugccaucugcua

SEQ ID NO: 367 - RNA, 15 nt, Artificial Sequence
  Description of Artificial Sequence Synthetic oligonucleotide
caucaaugaggccua

SEQ ID NO: 368 - RNA, 15 nt, Artificial Sequence
  Description of Artificial Sequence Synthetic oligonucleotide
cuccagcgacugcaa

SEQ ID NO: 369 - RNA, 15 nt, Artificial Sequence
  Description of Artificial Sequence Synthetic oligonucleotide
aaggaggagacccaa

SEQ ID NO: 370 - RNA, 15 nt, Artificial Sequence
  Description of Artificial Sequence Synthetic oligonucleotide
ugucugccgagccga

SEQ ID NO: 371 - RNA, 15 nt, Artificial Sequence
  Description of Artificial Sequence Synthetic oligonucleotide
ugccaaagaugucaa

SEQ ID NO: 372 - RNA, 15 nt, Artificial Sequence
  Description of Artificial Sequence Synthetic oligonucleotide
cuagccuugcguuca

SEQ ID NO: 373 - RNA, 15 nt, Artificial Sequence
  Description of Artificial Sequence Synthetic oligonucleotide
ucucugccaaagaua

SEQ ID NO: 374 - RNA, 15 nt, Artificial Sequence
  Description of Artificial Sequence Synthetic oligonucleotide
ggacgaugccugcca

SEQ ID NO: 375 - RNA, 15 nt, Artificial Sequence
  Description of Artificial Sequence Synthetic oligonucleotide
uagccuugcguucca

SEQ ID NO: 376 - RNA, 15 nt, Artificial Sequence
  Description of Artificial Sequence Synthetic oligonucleotide
accugcuuuguguca

SEQ ID NO: 377 - RNA, 15 nt, Artificial Sequence
  Description of Artificial Sequence Synthetic oligonucleotide
uaacuugaagauaua

SEQ ID NO: 378 - RNA, 15 nt, Artificial Sequence
  Description of Artificial Sequence Synthetic oligonucleotide
caagggaagggcaca

SEQ ID NO: 379 - RNA, 15 nt, Artificial Sequence
  Description of Artificial Sequence Synthetic oligonucleotide
cguggcuggcauuga

SEQ ID NO: 380 - RNA, 15 nt, Artificial Sequence
  Description of Artificial Sequence Synthetic oligonucleotide
ugaagccaagccuca

SEQ ID NO: 381 - RNA, 15 nt, Artificial Sequence
  Description of Artificial Sequence Synthetic oligonucleotide
ccucaccaagaucca

SEQ ID NO: 382 - RNA, 15 nt, Artificial Sequence
  Description of Artificial Sequence Synthetic oligonucleotide
ggucaucacaguuga

SEQ ID NO: 383 - RNA, 15 nt, Artificial Sequence
  Description of Artificial Sequence Synthetic oligonucleotide
gcaagcagacauuua

SEQ ID NO: 384 - RNA, 15 nt, Artificial Sequence
  Description of Artificial Sequence Synthetic oligonucleotide
uucuagaccuguuua

SEQ ID NO: 385 - RNA, 15 nt, Artificial Sequence
  Description of Artificial Sequence Synthetic oligonucleotide
gcccacaacgcuuua

SEQ ID NO: 386 - RNA, 15 nt, Artificial Sequence
  Description of Artificial Sequence Synthetic oligonucleotide
uguccucucuguuga

SEQ ID NO: 387 - RNA, 15 nt, Artificial Sequence
  Description of Artificial Sequence Synthetic oligonucleotide
aaggauccguggaga

SEQ ID NO: 388 - RNA, 15 nt, Artificial Sequence
  Description of Artificial Sequence Synthetic oligonucleotide
uccugcacaguccua

SEQ ID NO: 389 - RNA, 15 nt, Artificial Sequence
  Description of Artificial Sequence Synthetic oligonucleotide
acauuuaucuuuuga

SEQ ID NO: 390 - RNA, 15 nt, Artificial Sequence
  Description of Artificial Sequence Synthetic oligonucleotide
caaggaggcaggaua

SEQ ID NO: 391 - RNA, 15 nt, Artificial Sequence
  Description of Artificial Sequence Synthetic oligonucleotide
cuuuggccgcuguga

SEQ ID NO: 392 - RNA, 15 nt, Artificial Sequence
  Description of Artificial Sequence Synthetic oligonucleotide
cacagcaggaacuga

SEQ ID NO: 393 - RNA, 15 nt, Artificial Sequence
  Description of Artificial Sequence Synthetic oligonucleotide
cugcucuaugccaga

SEQ ID NO: 394 - RNA, 15 nt, Artificial Sequence
  Description of Artificial Sequence Synthetic oligonucleotide
ugucaucaaugagga

SEQ ID NO: 395 - RNA, 15 nt, Artificial Sequence
  Description of Artificial Sequence Synthetic oligonucleotide
cgaggacggcgacua

SEQ ID NO: 396 - RNA, 15 nt, Artificial Sequence
  Description of Artificial Sequence Synthetic oligonucleotide
caggauucuucccaa

SEQ ID NO: 397 - RNA, 15 nt, Artificial Sequence
  Description of Artificial Sequence Synthetic oligonucleotide
cgucaagcacccaca

SEQ ID NO: 398 - RNA, 15 nt, Artificial Sequence
  Description of Artificial Sequence Synthetic oligonucleotide
caccugcuuugugua

SEQ ID NO: 399 - RNA, 15 nt, Artificial Sequence
  Description of Artificial Sequence Synthetic oligonucleotide
ccacgcuuccugcua

SEQ ID NO: 400 - RNA, 15 nt, Artificial Sequence
  Description of Artificial Sequence Synthetic oligonucleotide
aggcagagacugaua

SEQ ID NO: 401 - RNA, 15 nt, Artificial Sequence
  Description of Artificial Sequence Synthetic oligonucleotide
ccaugaugcugucua

SEQ ID NO: 402 - RNA, 15 nt, Artificial Sequence
  Description of Artificial Sequence Synthetic oligonucleotide
cauugcagccaugaa

SEQ ID NO: 403 - RNA, 15 nt, Artificial Sequence
  Description of Artificial Sequence Synthetic oligonucleotide
cuaugccaggcugua

SEQ ID NO: 404 - RNA, 15 nt, Artificial Sequence
  Description of Artificial Sequence Synthetic oligonucleotide
cucugcucuaugcca

SEQ ID NO: 405 - RNA, 15 nt, Artificial Sequence
  Description of Artificial Sequence Synthetic oligonucleotide
gucagcccaaccaga

SEQ ID NO: 406 - RNA, 15 nt, Artificial Sequence
  Description of Artificial Sequence Synthetic oligonucleotide
uaucuuuugggucua

SEQ ID NO: 407 - RNA, 15 nt, Artificial Sequence
  Description of Artificial Sequence Synthetic oligonucleotide
ccgcuguguggacca

SEQ ID NO: 408 - RNA, 15 nt, Artificial Sequence
  Description of Artificial Sequence Synthetic oligonucleotide
cagccugguggagga

SEQ ID NO: 409 - RNA, 15 nt, Artificial Sequence
  Description of Artificial Sequence Synthetic oligonucleotide
uuggcagcuguuuua

SEQ ID NO: 410 - RNA, 15 nt, Artificial Sequence
  Description of Artificial Sequence Synthetic oligonucleotide
uugggucuguccuca

SEQ ID NO: 411 - RNA, 15 nt, Artificial Sequence
  Description of Artificial Sequence Synthetic oligonucleotide
caagcaagcagacaa

SEQ ID NO: 412 - RNA, 15 nt, Artificial Sequence
  Description of Artificial Sequence Synthetic oligonucleotide
cucucuguugccuua

SEQ ID NO: 413 - RNA, 15 nt, Artificial Sequence
  Description of Artificial Sequence Synthetic oligonucleotide
uaucuccuagacaca

SEQ ID NO: 414 - RNA, 15 nt, Artificial Sequence
  Description of Artificial Sequence Synthetic oligonucleotide
agagacugauccaca

SEQ ID NO: 415 - RNA, 15 nt, Artificial Sequence
  Description of Artificial Sequence Synthetic oligonucleotide
guuggcagcuguuua

SEQ ID NO: 416 - RNA, 15 nt, Artificial Sequence
  Description of Artificial Sequence Synthetic oligonucleotide
ccacaagccgccuga

SEQ ID NO: 417 - RNA, 15 nt, Artificial Sequence
  Description of Artificial Sequence Synthetic oligonucleotide
ugaggccugguucca

SEQ ID NO: 418 - RNA, 15 nt, Artificial Sequence
  Description of Artificial Sequence Synthetic oligonucleotide
agugagccaggcaga

SEQ ID NO: 419 - RNA, 15 nt, Artificial Sequence
  Description of Artificial Sequence Synthetic oligonucleotide
gauggcccucaucua

SEQ ID NO: 420 - RNA, 15 nt, Artificial Sequence
  Description of Artificial Sequence Synthetic oligonucleotide
cacagaccaggaaga

SEQ ID NO: 421 - RNA, 15 nt, Artificial Sequence
  Description of Artificial Sequence Synthetic oligonucleotide
cuggaaugcaaagua

SEQ ID NO: 422 - RNA, 15 nt, Artificial Sequence
  Description of Artificial Sequence Synthetic oligonucleotide
auggcaucuagccaa

SEQ ID NO: 423 - RNA, 15 nt, Artificial Sequence
  Description of Artificial Sequence Synthetic oligonucleotide
uggcauugcagccaa

SEQ ID NO: 424 - RNA, 15 nt, Artificial Sequence
  Description of Artificial Sequence Synthetic oligonucleotide
aagcacccacaccca

SEQ ID NO: 425 - RNA, 15 nt, Artificial Sequence
  Description of Artificial Sequence Synthetic oligonucleotide
cccacaacgcuuuua

SEQ ID NO: 426 - RNA, 15 nt, Artificial Sequence
  Description of Artificial Sequence Synthetic oligonucleotide
cuggcuuccugguga

SEQ ID NO: 427 - RNA, 15 nt, Artificial Sequence
  Description of Artificial Sequence Synthetic oligonucleotide
cauggaaucccggca

SEQ ID NO: 428 - RNA, 15 nt, Artificial Sequence
  Description of Artificial Sequence Synthetic oligonucleotide
cuacgaggagcugga

SEQ ID NO: 429 - RNA, 15 nt, Artificial Sequence
  Description of Artificial Sequence Synthetic oligonucleotide
ugaggaccagcggga

SEQ ID NO: 430 - RNA, 15 nt, Artificial Sequence
  Description of Artificial Sequence Synthetic oligonucleotide
uggcuggcauugcaa

SEQ ID NO: 431 - RNA, 15 nt, Artificial Sequence
  Description of Artificial Sequence Synthetic oligonucleotide
caaagaugucaucaa

SEQ ID NO: 432 - RNA, 15 nt, Artificial Sequence
  Description of Artificial Sequence Synthetic oligonucleotide
uggagguguaucuca

SEQ ID NO: 433 - RNA, 15 nt, Artificial Sequence
  Description of Artificial Sequence Synthetic oligonucleotide
caagcacccacacca

SEQ ID NO: 434 - RNA, 15 nt, Artificial Sequence
  Description of Artificial Sequence Synthetic oligonucleotide
ccgaguugaggcaga

SEQ ID NO: 435 - RNA, 15 nt, Artificial Sequence
  Description of Artificial Sequence Synthetic oligonucleotide
uggucaccgacuuca

SEQ ID NO: 436 - RNA, 15 nt, Artificial Sequence
  Description of Artificial Sequence Synthetic oligonucleotide
aggaggacgaggaca

SEQ ID NO: 437 - RNA, 15 nt, Artificial Sequence
  Description of Artificial Sequence Synthetic oligonucleotide
aacagugaggcugga

SEQ ID NO: 438 - RNA, 15 nt, Artificial Sequence
  Description of Artificial Sequence Synthetic oligonucleotide
uuauucggaaaagca

SEQ ID NO: 439 - RNA, 15 nt, Artificial Sequence
  Description of Artificial Sequence Synthetic oligonucleotide
cugccaagcucacaa

SEQ ID NO: 440 - RNA, 15 nt, Artificial Sequence
  Description of Artificial Sequence Synthetic oligonucleotide
ccagcauccacgcua

SEQ ID NO: 441 - RNA, 15 nt, Artificial Sequence
  Description of Artificial Sequence Synthetic oligonucleotide
gacuccucugucuua

SEQ ID NO: 442 - RNA, 15 nt, Artificial Sequence
  Description of Artificial Sequence Synthetic oligonucleotide
ccaagcaagcagaca

SEQ ID NO: 443 - RNA, 15 nt, Artificial Sequence
  Description of Artificial Sequence Synthetic oligonucleotide
gugcugccugcuaca

SEQ ID NO: 444 - RNA, 15 nt, Artificial Sequence
  Description of Artificial Sequence Synthetic oligonucleotide
gaggccagcauccaa

SEQ ID NO: 445 - RNA, 15 nt, Artificial Sequence
  Description of Artificial Sequence Synthetic oligonucleotide
gacauuuaucuuuua

SEQ ID NO: 446 - RNA, 15 nt, Artificial Sequence
  Description of Artificial Sequence Synthetic oligonucleotide
uguuuugcaggacua

SEQ ID NO: 447 - RNA, 15 nt, Artificial Sequence
  Description of Artificial Sequence Synthetic oligonucleotide
ucaccaagauccuga

SEQ ID NO: 448 - RNA, 15 nt, Artificial Sequence
  Description of Artificial Sequence Synthetic oligonucleotide
caccuuccaccgcua

SEQ ID NO: 449 - RNA, 15 nt, Artificial Sequence
  Description of Artificial Sequence Synthetic oligonucleotide
cuacgccauugccaa

SEQ ID NO: 450 - RNA, 15 nt, Artificial Sequence
  Description of Artificial Sequence Synthetic oligonucleotide
ggaggacuccucuga

SEQ ID NO: 451 - RNA, 15 nt, Artificial Sequence
  Description of Artificial Sequence Synthetic oligonucleotide
ugcugagcugcucca

SEQ ID NO: 452 - RNA, 15 nt, Artificial Sequence
  Description of Artificial Sequence Synthetic oligonucleotide
ugccaagcucacaca

SEQ ID NO: 453 - RNA, 15 nt, Artificial Sequence
  Description of Artificial Sequence Synthetic oligonucleotide
auugccaggugcuga

SEQ ID NO: 454 - RNA, 15 nt, Artificial Sequence
  Description of Artificial Sequence Synthetic oligonucleotide
cagugaggcugggaa

SEQ ID NO: 455 - RNA, 15 nt, Artificial Sequence
  Description of Artificial Sequence Synthetic oligonucleotide
ugcuccaguuucuca

SEQ ID NO: 456 - RNA, 15 nt, Artificial Sequence
  Description of Artificial Sequence Synthetic oligonucleotide
ugauccacuucucua

SEQ ID NO: 457 - RNA, 15 nt, Artificial Sequence
  Description of Artificial Sequence Synthetic oligonucleotide
gaugaguggcgacca

SEQ ID NO: 458 - RNA, 15 nt, Artificial Sequence
  Description of Artificial Sequence Synthetic oligonucleotide
aaagccagcugguca

SEQ ID NO: 459 - RNA, 15 nt, Artificial Sequence
  Description of Artificial Sequence Synthetic oligonucleotide
caagauccugcauga

SEQ ID NO: 460 - RNA, 15 nt, Artificial Sequence
  Description of Artificial Sequence Synthetic oligonucleotide
caacuuuggccgcua

SEQ ID NO: 461 - RNA, 15 nt, Artificial Sequence
  Description of Artificial Sequence Synthetic oligonucleotide
guccucacaggcuga

SEQ ID NO: 462 - RNA, 15 nt, Artificial Sequence
  Description of Artificial Sequence Synthetic oligonucleotide
cguccacacagcuca

SEQ ID NO: 463 - RNA, 15 nt, Artificial Sequence
  Description of Artificial Sequence Synthetic oligonucleotide
ugcugccugcuacca

SEQ ID NO: 464 - RNA, 15 nt, Artificial Sequence
  Description of Artificial Sequence Synthetic oligonucleotide
cgagccggagcucaa

SEQ ID NO: 465 - RNA, 15 nt, Artificial Sequence
  Description of Artificial Sequence Synthetic oligonucleotide
uguaucuccuagaca

SEQ ID NO: 466 - RNA, 15 nt, Artificial Sequence
  Description of Artificial Sequence Synthetic oligonucleotide
cgggacccgcuucca

SEQ ID NO: 467 - RNA, 15 nt, Artificial Sequence
  Description of Artificial Sequence Synthetic oligonucleotide
caagcucacacagca

SEQ ID NO: 468 - RNA, 15 nt, Artificial Sequence
  Description of Artificial Sequence Synthetic oligonucleotide
aaaagccagcuggua

SEQ ID NO: 469 - RNA, 15 nt, Artificial Sequence
  Description of Artificial Sequence Synthetic oligonucleotide
guggaccucuuugca

SEQ ID NO: 470 - RNA, 15 nt, Artificial Sequence
  Description of Artificial Sequence Synthetic oligonucleotide
ccucuaggucuccua

SEQ ID NO: 471 - RNA, 15 nt, Artificial Sequence
  Description of Artificial Sequence Synthetic oligonucleotide
cuguuuugcaggaca

SEQ ID NO: 472 - RNA, 15 nt, Artificial Sequence
  Description of Artificial Sequence Synthetic oligonucleotide
cuacgugguggugca

SEQ ID NO: 473 - RNA, 15 nt, Artificial Sequence
  Description of Artificial Sequence Synthetic oligonucleotide
ccuggcccuugagua

SEQ ID NO: 474 - RNA, 15 nt, Artificial Sequence
  Description of Artificial Sequence Synthetic oligonucleotide
gagcuuuaaaaugga

SEQ ID NO: 475 - RNA, 15 nt, Artificial Sequence
  Description of Artificial Sequence Synthetic oligonucleotide
gggcccacaacgcua

SEQ ID NO: 476 - RNA, 15 nt, Artificial Sequence
  Description of Artificial Sequence Synthetic oligonucleotide
uccacuucucugcca

SEQ ID NO: 477 - RNA, 15 nt, Artificial Sequence
  Description of Artificial Sequence Synthetic oligonucleotide
cccuagaagguuuca

SEQ ID NO: 478 - RNA, 15 nt, Artificial Sequence
  Description of Artificial Sequence Synthetic oligonucleotide
ugcuggucaccgcua

SEQ ID NO: 479 - RNA, 15 nt, Artificial Sequence
  Description of Artificial Sequence Synthetic oligonucleotide
ccuccagcgacugca

SEQ ID NO: 480 - RNA, 15 nt, Artificial Sequence
  Description of Artificial Sequence Synthetic oligonucleotide
uuauucuggguuuua

SEQ ID NO: 481 - RNA, 15 nt, Artificial Sequence
  Description of Artificial Sequence Synthetic oligonucleotide
gugucuacgccauua

SEQ ID NO: 482 - RNA, 15 nt, Artificial Sequence
  Description of Artificial Sequence Synthetic oligonucleotide
uuguaacuugaagaa

SEQ ID NO: 483 - RNA, 15 nt, Artificial Sequence
  Description of Artificial Sequence Synthetic oligonucleotide
uuuugcuuuuguaaa

SEQ ID NO: 484 - RNA, 15 nt, Artificial Sequence
  Description of Artificial Sequence Synthetic oligonucleotide
uguaacuugaagaua

SEQ ID NO: 485 - RNA, 15 nt, Artificial Sequence
  Description of Artificial Sequence Synthetic oligonucleotide
cuccuagacaccaga

SEQ ID NO: 486 - RNA, 15 nt, Artificial Sequence
  Description of Artificial Sequence Synthetic oligonucleotide
agucagagcgcacua

SEQ ID NO: 487 - RNA, 15 nt, Artificial Sequence
  Description of Artificial Sequence Synthetic oligonucleotide
cagcaagugugacaa

SEQ ID NO: 488 - RNA, 15 nt, Artificial Sequence
  Description of Artificial Sequence Synthetic oligonucleotide
gaggacuccucugua

SEQ ID NO: 489 - RNA, 15 nt, Artificial Sequence
  Description of Artificial Sequence Synthetic oligonucleotide
uuugcuuuuguaaca

SEQ ID NO: 490 - RNA, 15 nt, Artificial Sequence
  Description of Artificial Sequence Synthetic oligonucleotide
aaggaggcaggauua

SEQ ID NO: 491 - RNA, 15 nt, Artificial Sequence
  Description of Artificial Sequence Synthetic oligonucleotide
cagccaccuuccaca

SEQ ID NO: 492 - RNA, 15 nt, Artificial Sequence
  Description of Artificial Sequence Synthetic oligonucleotide
agccaacccgcucca

SEQ ID NO: 493 - RNA, 15 nt, Artificial Sequence
  Description of Artificial Sequence Synthetic oligonucleotide
aagaugaguggcgaa

SEQ ID NO: 494 - RNA, 15 nt, Artificial Sequence
  Description of Artificial Sequence Synthetic oligonucleotide
aggaggacuccucua

SEQ ID NO: 495 - RNA, 15 nt, Artificial Sequence
  Description of Artificial Sequence Synthetic oligonucleotide
ccgagccggagcuca

SEQ ID NO: 496 - RNA, 15 nt, Artificial Sequence
  Description of Artificial Sequence Synthetic oligonucleotide
aggucaucacaguua

SEQ ID NO: 497 - RNA, 15 nt, Artificial Sequence
  Description of Artificial Sequence Synthetic oligonucleotide
gguggagguguauca

SEQ ID NO: 498 - RNA, 15 nt, Artificial Sequence
  Description of Artificial Sequence Synthetic oligonucleotide
ucugcucuaugccaa

SEQ ID NO: 499 - RNA, 15 nt, Artificial Sequence
  Description of Artificial Sequence Synthetic oligonucleotide
agcugcugagcugca

SEQ ID NO: 500 - RNA, 15 nt, Artificial Sequence
  Description of Artificial Sequence Synthetic oligonucleotide
aagaugucaucaaua

SEQ ID NO: 501 - RNA, 19 nt, Artificial Sequence
  Description of Artificial Sequence Synthetic oligonucleotide
uucuuuggcagagaagugg

SEQ ID NO: 502 - RNA, 19 nt, Artificial Sequence
  Description of Artificial Sequence Synthetic oligonucleotide
ucaugcaggaucuugguga

SEQ ID NO: 503 - RNA, 19 nt, Artificial Sequence
  Description of Artificial Sequence Synthetic oligonucleotide
uuaaacuccaggccuauga

SEQ ID NO: 504 - RNA, 19 nt, Artificial Sequence
  Description of Artificial Sequence Synthetic oligonucleotide
ugcaaugccagccacgugg

SEQ ID NO: 505 - RNA, 19 nt, Artificial Sequence
  Description of Artificial Sequence Synthetic oligonucleotide
ugacuuugcauuccagacc

SEQ ID NO: 506 - RNA, 19 nt, Artificial Sequence
  Description of Artificial Sequence Synthetic oligonucleotide
uaaccgugcccuucccuug

SEQ ID NO: 507 - RNA, 19 nt, Artificial Sequence
  Description of Artificial Sequence Synthetic oligonucleotide
uaguggaucagucucugcc

SEQ ID NO: 508 - RNA, 19 nt, Artificial Sequence
  Description of Artificial Sequence Synthetic oligonucleotide
ucauucucgaagucgguga

SEQ ID NO: 509 - RNA, 19 nt, Artificial Sequence
  Description of Artificial Sequence Synthetic oligonucleotide
uaacuccaggccuaugagg

SEQ ID NO: 510 - RNA, 19 nt, Artificial Sequence
  Description of Artificial Sequence Synthetic oligonucleotide
uaacuucaaggccagcucc

SEQ ID NO: 511 - RNA, 19 nt, Artificial Sequence
  Description of Artificial Sequence Synthetic oligonucleotide
uaauggcguagacacccuc

SEQ ID NO: 512 - RNA, 19 nt, Artificial Sequence
  Description of Artificial Sequence Synthetic oligonucleotide
uuagaggcaggcaucgucc

SEQ ID NO: 513 - RNA, 19 nt, Artificial Sequence
  Description of Artificial Sequence Synthetic oligonucleotide
uaccuccacggauccuugg

SEQ ID NO: 514 - RNA, 19 nt, Artificial Sequence
  Description of Artificial Sequence Synthetic oligonucleotide
ucagucucugccucaacuc

SEQ ID NO: 515 - RNA, 19 nt, Artificial Sequence
  Description of Artificial Sequence Synthetic oligonucleotide
uucuuggugagguaucccc

SEQ ID NO: 516 - RNA, 19 nt, Artificial Sequence
  Description of Artificial Sequence Synthetic oligonucleotide
uagauggcaacggcuguca

SEQ ID NO: 517 - RNA, 19 nt, Artificial Sequence
  Description of Artificial Sequence Synthetic oligonucleotide
uacaccuccaccaggcugc

SEQ ID NO: 518 - RNA, 19 nt, Artificial Sequence
  Description of Artificial Sequence Synthetic oligonucleotide
uuggaagacaugcaggauc

SEQ ID NO: 519 - RNA, 19 nt, Artificial Sequence
  Description of Artificial Sequence Synthetic oligonucleotide
uaugagggugccgcuaacc

SEQ ID NO: 520 - RNA, 19 nt, Artificial Sequence
  Description of Artificial Sequence Synthetic oligonucleotide
uuucucgaagucggugacc

SEQ ID NO: 521 - RNA, 19 nt, Artificial Sequence
  Description of Artificial Sequence Synthetic oligonucleotide
uaaguggaucagucucugc

SEQ ID NO: 522 - RNA, 19 nt, Artificial Sequence
  Description of Artificial Sequence Synthetic oligonucleotide
ucacucuguaugcuggugu

SEQ ID NO: 523 - RNA, 19 nt, Artificial Sequence
  Description of Artificial Sequence Synthetic oligonucleotide
uguguggacgcugcaguug

SEQ ID NO: 524 - RNA, 19 nt, Artificial Sequence
  Description of Artificial Sequence Synthetic oligonucleotide
uguagaggcaggcaucguc

SEQ ID NO: 525 - RNA, 19 nt, Artificial Sequence
  Description of Artificial Sequence Synthetic oligonucleotide
ucuguaugcuggugucuag

SEQ ID NO: 526 - RNA, 19 nt, Artificial Sequence
  Description of Artificial Sequence Synthetic oligonucleotide
uucaaggccagcuccagca

SEQ ID NO: 527 - RNA, 19 nt, Artificial Sequence
  Description of Artificial Sequence Synthetic oligonucleotide
uaagucggugaccaugacc

SEQ ID NO: 528 - RNA, 19 nt, Artificial Sequence
  Description of Artificial Sequence Synthetic oligonucleotide
ucacacuugcuggccuguc

SEQ ID NO: 529 - RNA, 19 nt, Artificial Sequence
  Description of Artificial Sequence Synthetic oligonucleotide
ugucuguggaagcgggucc

SEQ ID NO: 530 - RNA, 19 nt, Artificial Sequence
  Description of Artificial Sequence Synthetic oligonucleotide
uuuggcagagaaguggauc

SEQ ID NO: 531 - RNA, 19 nt, Artificial Sequence
  Description of Artificial Sequence Synthetic oligonucleotide
uagaaggccauggaagaca

SEQ ID NO: 532 - RNA, 19 nt, Artificial Sequence
  Description of Artificial Sequence Synthetic oligonucleotide
uuugaggacgcggcuguac

SEQ ID NO: 533 - RNA, 19 nt, Artificial Sequence
  Description of Artificial Sequence Synthetic oligonucleotide
uuucccgguggucacucug

SEQ ID NO: 534 - RNA, 19 nt, Artificial Sequence
  Description of Artificial Sequence Synthetic oligonucleotide
uaugcuggugucuaggaga

SEQ ID NO: 535 - RNA, 19 nt, Artificial Sequence
  Description of Artificial Sequence Synthetic oligonucleotide
uggaagguggcugugguuc

SEQ ID NO: 536 - RNA, 19 nt, Artificial Sequence
  Description of Artificial Sequence Synthetic oligonucleotide
uaaacuccaggccuaugag

SEQ ID NO: 537 - RNA, 19 nt, Artificial Sequence
  Description of Artificial Sequence Synthetic oligonucleotide
uuccacgggaugcucuggg

SEQ ID NO: 538 - RNA, 19 nt, Artificial Sequence
  Description of Artificial Sequence Synthetic oligonucleotide
uuccgcuccagguuccacg

SEQ ID NO: 539 - RNA, 19 nt, Artificial Sequence
  Description of Artificial Sequence Synthetic oligonucleotide
uucagcaccaccacguagg

SEQ ID NO: 540 - RNA, 19 nt, Artificial Sequence
  Description of Artificial Sequence Synthetic oligonucleotide
uccuccucgggcacauucu

SEQ ID NO: 541 - RNA, 19 nt, Artificial Sequence
  Description of Artificial Sequence Synthetic oligonucleotide
uuuccgaauaaacuccagg

SEQ ID NO: 542 - RNA, 19 nt, Artificial Sequence
  Description of Artificial Sequence Synthetic oligonucleotide
uuuccaugcuccuugacuu

SEQ ID NO: 543 - RNA, 19 nt, Artificial Sequence
  Description of Artificial Sequence Synthetic oligonucleotide
ugcuccuugacuuugcauu

SEQ ID NO: 544 - RNA, 14 nt, Artificial Sequence
  Description of Artificial Sequence Synthetic oligonucleotide
ucucugccaaagaa

SEQ ID NO: 545 - RNA, 14 nt, Artificial Sequence
  Description of Artificial Sequence Synthetic oligonucleotide
aagauccugcauga

SEQ ID NO: 546 - RNA, 14 nt, Artificial Sequence
  Description of Artificial Sequence Synthetic oligonucleotide
ggccuggaguuuaa

SEQ ID NO: 547 - RNA, 14 nt, Artificial Sequence
  Description of Artificial Sequence Synthetic oligonucleotide
uggcuggcauugca

SEQ ID NO: 548 - RNA, 14 nt, Artificial Sequence
  Description of Artificial Sequence Synthetic oligonucleotide
ggaaugcaaaguca

SEQ ID NO: 549 - RNA, 14 nt, Artificial Sequence
  Description of Artificial Sequence Synthetic oligonucleotide
gaagggcacgguua

SEQ ID NO: 550 - RNA, 14 nt, Artificial Sequence
  Description of Artificial Sequence Synthetic oligonucleotide
agacugauccacua

SEQ ID NO: 551 - RNA, 14 nt, Artificial Sequence
  Description of Artificial Sequence Synthetic oligonucleotide
gacuucgagaauga

SEQ ID NO: 552 - RNA, 14 nt, Artificial Sequence
  Description of Artificial Sequence Synthetic oligonucleotide
uaggccuggaguua

SEQ ID NO: 553 - RNA, 14 nt, Artificial Sequence
  Description of Artificial Sequence Synthetic oligonucleotide
uggccuugaaguua

SEQ ID NO: 554 - RNA, 14 nt, Artificial Sequence
  Description of Artificial Sequence Synthetic oligonucleotide
ugucuacgccauua

SEQ ID NO: 555 - RNA, 14 nt, Artificial Sequence
  Description of Artificial Sequence Synthetic oligonucleotide
augccugccucuaa

SEQ ID NO: 556 - RNA, 14 nt, Artificial Sequence
  Description of Artificial Sequence Synthetic oligonucleotide
gauccguggaggua

SEQ ID NO: 557 - RNA, 14 nt, Artificial Sequence
  Description of Artificial Sequence Synthetic oligonucleotide
gaggcagagacuga

SEQ ID NO: 558 - RNA, 14 nt, Artificial Sequence
  Description of Artificial Sequence Synthetic oligonucleotide
uaccucaccaagaa

SEQ ID NO: 559 - RNA, 14 nt, Artificial Sequence
  Description of Artificial Sequence Synthetic oligonucleotide
gccguugccaucua

SEQ ID NO: 560 - RNA, 14 nt, Artificial Sequence
  Description of Artificial Sequence Synthetic oligonucleotide
cugguggaggugua

SEQ ID NO: 561 - RNA, 14 nt, Artificial Sequence
  Description of Artificial Sequence Synthetic oligonucleotide
ugcaugucuuccaa

SEQ ID NO: 562 - RNA, 14 nt, Artificial Sequence
  Description of Artificial Sequence Synthetic oligonucleotide
gcggcacccucaua

SEQ ID NO: 563 - RNA, 14 nt, Artificial Sequence
  Description of Artificial Sequence Synthetic oligonucleotide
ccgacuucgagaaa

SEQ ID NO: 564 - RNA, 14 nt, Artificial Sequence
  Description of Artificial Sequence Synthetic oligonucleotide
gacugauccacuua

SEQ ID NO: 565 - RNA, 14 nt, Artificial Sequence
  Description of Artificial Sequence Synthetic oligonucleotide
agcauacagaguga

SEQ ID NO: 566 - RNA, 14 nt, Artificial Sequence
  Description of Artificial Sequence Synthetic oligonucleotide
gcagcguccacaca

SEQ ID NO: 567 - RNA, 14 nt, Artificial Sequence
  Description of Artificial Sequence Synthetic oligonucleotide
ugccugccucuaca

SEQ ID NO: 568 - RNA, 14 nt, Artificial Sequence
  Description of Artificial Sequence Synthetic oligonucleotide
caccagcauacaga

SEQ ID NO: 569 - RNA, 14 nt, Artificial Sequence
  Description of Artificial Sequence Synthetic oligonucleotide
gagcuggccuugaa

SEQ ID NO: 570 - RNA, 14 nt, Artificial Sequence
  Description of Artificial Sequence Synthetic oligonucleotide
uggucaccgacuua

SEQ ID NO: 571 - RNA, 14 nt, Artificial Sequence
  Description of Artificial Sequence Synthetic oligonucleotide
gccagcaaguguga

SEQ ID NO: 572 - RNA, 14 nt, Artificial Sequence
  Description of Artificial Sequence Synthetic oligonucleotide
cgcuuccacagaca

SEQ ID NO: 573 - RNA, 14 nt, Artificial Sequence
  Description of Artificial Sequence Synthetic oligonucleotide
acuucucugccaaa

SEQ ID NO: 574 - RNA, 14 nt, Artificial Sequence
  Description of Artificial Sequence Synthetic oligonucleotide
uccauggccuucua

SEQ ID NO: 575 - RNA, 14 nt, Artificial Sequence
  Description of Artificial Sequence Synthetic oligonucleotide
gccgcguccucaaa

SEQ ID NO: 576 - RNA, 14 nt, Artificial Sequence
  Description of Artificial Sequence Synthetic oligonucleotide
ugaccaccgggaaa

SEQ ID NO: 577 - RNA, 14 nt, Artificial Sequence
  Description of Artificial Sequence Synthetic oligonucleotide
uagacaccagcaua

SEQ ID NO: 578 - RNA, 14 nt, Artificial Sequence
  Description of Artificial Sequence Synthetic oligonucleotide
acagccaccuucca

SEQ ID NO: 579 - RNA, 14 nt, Artificial Sequence
  Description of Artificial Sequence Synthetic oligonucleotide
aggccuggaguuua

SEQ ID NO: 580 - RNA, 14 nt, Artificial Sequence
  Description of Artificial Sequence Synthetic oligonucleotide
agcaucccguggaa

SEQ ID NO: 581 - RNA, 14 nt, Artificial Sequence
  Description of Artificial Sequence Synthetic oligonucleotide
aaccuggagcggaa

SEQ ID NO: 582 - RNA, 14 nt, Artificial Sequence
  Description of Artificial Sequence Synthetic oligonucleotide
gugguggugcugaa

SEQ ID NO: 583 - RNA, 14 nt, Artificial Sequence
  Description of Artificial Sequence Synthetic oligonucleotide
gugcccgaggagga

SEQ ID NO: 584 - RNA, 14 nt, Artificial Sequence
  Description of Artificial Sequence Synthetic oligonucleotide
aguuuauucggaaa

SEQ ID NO: 585 - RNA, 14 nt, Artificial Sequence
  Description of Artificial Sequence Synthetic oligonucleotide
aaggagcauggaaa

SEQ ID NO: 586 - RNA, 14 nt, Artificial Sequence
  Description of Artificial Sequence Synthetic oligonucleotide
aaagucaaggagca

SEQ ID NO: 587 - RNA, 33 nt, Artificial Sequence
  Description of Artificial Sequence Synthetic oligonucleotide
  PCS29 mxRNA (14-5-14)
ugcaaaacaggucuagaaaagaccuguuuugca

SEQ ID NO: 588 - RNA, 33 nt, Artificial Sequence
  Description of Artificial Sequence Synthetic oligonucleotide
  PCS44 mxRNA (14-5-14)
ucuucaaguuacaaaagcauuguaacuugaaga

SEQ ID NO: 589 - RNA, 33 nt, Artificial Sequence
  Description of Artificial Sequence Synthetic oligonucleotide
  PCS53 mxRNA (14-5-14)
uacaaaagcaaaacaggucguuuugcuuuugua

SEQ ID NO: 590 - DNA, 33 bp, Artificial Sequence
  Description of Artificial Sequence Synthetic oligonucleotide
  Description of Combined DNA/RNA Molecule Synthetic oligonucleotide
  PCS53 mxRNA (14-5-14)dT
tacaaaagcaaaacaggtcguuuugcuuuugua

SEQ ID NO: 591 - RNA, 19 nt, Artificial Sequence
  Description of Artificial Sequence Synthetic oligonucleotide
ucuucaaguuacaaaagca

SEQ ID NO: 592 - RNA, 19 nt, Artificial Sequence
  Description of Artificial Sequence Synthetic oligonucleotide
uaaaagcaaaacaggucua

SEQ ID NO: 593 - RNA, 19 nt, Artificial Sequence
  Description of Artificial Sequence Synthetic oligonucleotide
uacaaaagcaaaacagguc

SEQ ID NO: 594 - RNA, 19 nt, Artificial Sequence
  Description of Artificial Sequence Synthetic oligonucleotide
ugcaaaacaggucuagaaa

SEQ ID NO: 595 - RNA, 19 nt, Artificial Sequence
  Description of Artificial Sequence Synthetic oligonucleotide
ucaaaagcaaaacaggucu

SEQ ID NO: 596 - RNA, 19 nt, Artificial Sequence
  Description of Artificial Sequence Synthetic oligonucleotide
uagaauaaauaucuucaag

SEQ ID NO: 597 - RNA, 19 nt, Artificial Sequence
  Description of Artificial Sequence Synthetic oligonucleotide
uaaagcaaaacaggucuag

SEQ ID NO: 598 - RNA, 19 nt, Artificial Sequence
  Description of Artificial Sequence Synthetic oligonucleotide
uucuucaaguuacaaaagc

SEQ ID NO: 599 - RNA, 19 nt, Artificial Sequence
  Description of Artificial Sequence Synthetic oligonucleotide
ucaaguuacaaaagcaaaa

SEQ ID NO: 600 - RNA, 19 nt, Artificial Sequence
  Description of Artificial Sequence Synthetic oligonucleotide
ucaaaacaggucuagaaaa

SEQ ID NO: 601 - RNA, 19 nt, Artificial Sequence
  Description of Artificial Sequence Synthetic oligonucleotide
uauaaauaucuucaaguua

SEQ ID NO: 602 - RNA, 19 nt, Artificial Sequence
  Description of Artificial Sequence Synthetic oligonucleotide
uuuacaaaagcaaaacagg

SEQ ID NO: 603 - RNA, 19 nt, Artificial Sequence
  Description of Artificial Sequence Synthetic oligonucleotide
ugugacacaaagcaggugc

SEQ ID NO: 604 - RNA, 19 nt, Artificial Sequence
  Description of Artificial Sequence Synthetic oligonucleotide
uuucaaguuacaaaagcaa

SEQ ID NO: 605 - RNA, 19 nt, Artificial Sequence
  Description of Artificial Sequence Synthetic oligonucleotide
uaaauaucuucaaguuaca

SEQ ID NO: 606 - RNA, 19 nt, Artificial Sequence
  Description of Artificial Sequence Synthetic oligonucleotide
uauaaaugucugcuugcuu

SEQ ID NO: 607 - RNA, 19 nt, Artificial Sequence
  Description of Artificial Sequence Synthetic oligonucleotide
uccgaauaaacuccaggcc

SEQ ID NO: 608 - RNA, 19 nt, Artificial Sequence
  Description of Artificial Sequence Synthetic oligonucleotide
uggaaccauuuuaaagcuc

SEQ ID NO: 609 - RNA, 19 nt, Artificial Sequence
  Description of Artificial Sequence Synthetic oligonucleotide
uccagaauaaauaucuuca

SEQ ID NO: 610 - RNA, 19 nt, Artificial Sequence
  Description of Artificial Sequence Synthetic oligonucleotide
uauaucuucaaguuacaaa

SEQ ID NO: 611 - RNA, 19 nt, Artificial Sequence
  Description of Artificial Sequence Synthetic oligonucleotide
ucucaguuccugcugugug

SEQ ID NO: 612 - RNA, 19 nt, Artificial Sequence
  Description of Artificial Sequence Synthetic oligonucleotide
ucuacaaaacccagaauaa

SEQ ID NO: 613 - RNA, 19 nt, Artificial Sequence
  Description of Artificial Sequence Synthetic oligonucleotide
ucaaaagauaaaugucugc

SEQ ID NO: 614 - RNA, 19 nt, Artificial Sequence
  Description of Artificial Sequence Synthetic oligonucleotide
uuuccgaauaaacuccagg

SEQ ID NO: 615 - RNA, 19 nt, Artificial Sequence
  Description of Artificial Sequence Synthetic oligonucleotide
ucuggcucaguuccugcug

SEQ ID NO: 616 - RNA, 19 nt, Artificial Sequence
  Description of Artificial Sequence Synthetic oligonucleotide
ucagacagcaucauggcug

SEQ ID NO: 617 - RNA, 19 nt, Artificial Sequence
  Description of Artificial Sequence Synthetic oligonucleotide
ugggauuccaugcuccuug

SEQ ID NO: 618 - RNA, 14 nt, Artificial Sequence
  Description of Artificial Sequence Synthetic oligonucleotide
uuguaacuugaaga

SEQ ID NO: 619 - RNA, 14 nt, Artificial Sequence
  Description of Artificial Sequence Synthetic oligonucleotide
cuguuuugcuuuua

SEQ ID NO: 620 - RNA, 14 nt, Artificial Sequence
  Description of Artificial Sequence Synthetic oligonucleotide
guuuugcuuuugua

SEQ ID NO: 621 - RNA, 14 nt, Artificial Sequence
  Description of Artificial Sequence Synthetic oligonucleotide
agaccuguuuugca

SEQ ID NO: 622 - RNA, 14 nt, Artificial Sequence
  Description of Artificial Sequence Synthetic oligonucleotide
uguuuugcuuuuga

SEQ ID NO: 623 - RNA, 14 nt, Artificial Sequence
  Description of Artificial Sequence Synthetic oligonucleotide
agauauuuauucua

SEQ ID NO: 624 - RNA, 14 nt, Artificial Sequence
  Description of Artificial Sequence Synthetic oligonucleotide
ccuguuuugcuuua

SEQ ID NO: 625 - RNA, 14 nt, Artificial Sequence
  Description of Artificial Sequence Synthetic oligonucleotide
uguaacuugaagaa

SEQ ID NO: 626 - RNA, 14 nt, Artificial Sequence
  Description of Artificial Sequence Synthetic oligonucleotide
cuuuuguaacuuga

SEQ ID NO: 627 - RNA, 14 nt, Artificial Sequence
  Description of Artificial Sequence Synthetic oligonucleotide
uagaccuguuuuga

SEQ ID NO: 628 - RNA, 14 nt, Artificial Sequence
  Description of Artificial Sequence Synthetic oligonucleotide
ugaagauauuuaua

SEQ ID NO: 629 - RNA, 14 nt, Artificial Sequence
  Description of Artificial Sequence Synthetic oligonucleotide
uuugcuuuuguaaa

SEQ ID NO: 630 - RNA, 14 nt, Artificial Sequence
  Description of Artificial Sequence Synthetic oligonucleotide
ugcuuugugucaca

SEQ ID NO: 631 - RNA, 14 nt, Artificial Sequence
  Description of Artificial Sequence Synthetic oligonucleotide
uuuguaacuugaaa

SEQ ID NO: 632 - RNA, 14 nt, Artificial Sequence
  Description of Artificial Sequence Synthetic oligonucleotide
cuugaagauauuua

SEQ ID NO: 633 - RNA, 14 nt, Artificial Sequence
  Description of Artificial Sequence Synthetic oligonucleotide
agcagacauuuaua

SEQ ID NO: 634 - RNA, 14 nt, Artificial Sequence
  Description of Artificial Sequence Synthetic oligonucleotide
ggaguuuauucgga

SEQ ID NO: 635 - RNA, 14 nt, Artificial Sequence
  Description of Artificial Sequence Synthetic oligonucleotide
uuaaaaugguucca

SEQ ID NO: 636 - RNA, 14 nt, Artificial Sequence
  Description of Artificial Sequence Synthetic oligonucleotide
auauuuauucugga

SEQ ID NO: 637 - RNA, 14 nt, Artificial Sequence
  Description of Artificial Sequence Synthetic oligonucleotide
aacuugaagauaua

SEQ ID NO: 638 - RNA, 14 nt, Artificial Sequence
  Description of Artificial Sequence Synthetic oligonucleotide
agcaggaacugaga

SEQ ID NO: 639 - RNA, 14 nt, Artificial Sequence
  Description of Artificial Sequence Synthetic oligonucleotide
cuggguuuuguaga

SEQ ID NO: 640 - RNA, 14 nt, Artificial Sequence
  Description of Artificial Sequence Synthetic oligonucleotide
cauuuaucuuuuga

SEQ ID NO: 641 - RNA, 14 nt, Artificial Sequence
  Description of Artificial Sequence Synthetic oligonucleotide
aguuuauucggaaa

SEQ ID NO: 642 - RNA, 14 nt, Artificial Sequence
  Description of Artificial Sequence Synthetic oligonucleotide
ggaacugagccaga

SEQ ID NO: 643 - RNA, 14 nt, Artificial Sequence
  Description of Artificial Sequence Synthetic oligonucleotide
augaugcugucuga

SEQ ID NO: 644 - RNA, 14 nt, Artificial Sequence
  Description of Artificial Sequence Synthetic oligonucleotide
agcauggaauccca

SEQ ID NO: 645 - RNA, 19 nt, Artificial Sequence
  Description of Artificial Sequence Synthetic oligonucleotide
uggccuaugagggugccgc

SEQ ID NO: 646 - RNA, 19 nt, Artificial Sequence
  Description of Artificial Sequence Synthetic oligonucleotide
ugaauaaacuccaggccua

SEQ ID NO: 647 - RNA, 19 nt, Artificial Sequence
  Description of Artificial Sequence Synthetic oligonucleotide
uuaaacuccaggccuauga

SEQ ID NO: 648 - RNA, 19 nt, Artificial Sequence
  Description of Artificial Sequence Synthetic oligonucleotide
ucgaauaaacuccaggccu

SEQ ID NO: 649 - RNA, 19 nt, Artificial Sequence
  Description of Artificial Sequence Synthetic oligonucleotide
uuccggcagcagauggcaa

SEQ ID NO: 650 - RNA, 19 nt, Artificial Sequence
  Description of Artificial Sequence Synthetic oligonucleotide
uacuccaggccuaugaggg

SEQ ID NO: 651 - RNA, 19 nt, Artificial Sequence
  Description of Artificial Sequence Synthetic oligonucleotide
uccuaugagggugccgcua

SEQ ID NO: 652 - RNA, 19 nt, Artificial Sequence
  Description of Artificial Sequence Synthetic oligonucleotide
uaggccuaugagggugccg

SEQ ID NO: 653 - RNA, 19 nt, Artificial Sequence
  Description of Artificial Sequence Synthetic oligonucleotide
uuccaggccuaugagggug

SEQ ID NO: 654 - RNA, 19 nt, Artificial Sequence
  Description of Artificial Sequence Synthetic oligonucleotide
ugcugguccucagggaacc

SEQ ID NO: 655 - RNA, 19 nt, Artificial Sequence
  Description of Artificial Sequence Synthetic oligonucleotide
uaauaaacuccaggccuau

SEQ ID NO: 656 - RNA, 19 nt, Artificial Sequence
  Description of Artificial Sequence Synthetic oligonucleotide
ucuggagcagcucagcagc

SEQ ID NO: 657 - RNA, 19 nt, Artificial Sequence
  Description of Artificial Sequence Synthetic oligonucleotide
uccgaauaaacuccaggcc

SEQ ID NO: 658 - RNA, 19 nt, Artificial Sequence
  Description of Artificial Sequence Synthetic oligonucleotide
ugguccucagggaaccagg

SEQ ID NO: 659 - RNA, 19 nt, Artificial Sequence
  Description of Artificial Sequence Synthetic oligonucleotide
uugguccucagggaaccag

SEQ ID NO: 660 - RNA, 19 nt, Artificial Sequence
  Description of Artificial Sequence Synthetic oligonucleotide
uauaaacuccaggccuaug

SEQ ID NO: 661 - RNA, 19 nt, Artificial Sequence
  Description of Artificial Sequence Synthetic oligonucleotide
ucuccaggccuaugagggu

SEQ ID NO: 662 - RNA, 19 nt, Artificial Sequence
  Description of Artificial Sequence Synthetic oligonucleotide
ucaggccuaugagggugcc

SEQ ID NO: 663 - RNA, 19 nt, Artificial Sequence
  Description of Artificial Sequence Synthetic oligonucleotide
uguccucagggaaccaggc

SEQ ID NO: 664 - RNA, 19 nt, Artificial Sequence
  Description of Artificial Sequence Synthetic oligonucleotide
ugccuaugagggugccgcu

SEQ ID NO: 665 - RNA, 19 nt, Artificial Sequence
  Description of Artificial Sequence Synthetic oligonucleotide
uuuccgaauaaacuccagg

SEQ ID NO: 666 - RNA, 19 nt, Artificial Sequence
  Description of Artificial Sequence Synthetic oligonucleotide
ugcauggcagcaggaagcg

SEQ ID NO: 667 - RNA, 19 nt, Artificial Sequence
  Description of Artificial Sequence Synthetic oligonucleotide
ugcuccggcagcagauggc

SEQ ID NO: 668 - RNA, 19 nt, Artificial Sequence
  Description of Artificial Sequence Synthetic oligonucleotide
ugccggcuccggcagcaga

SEQ ID NO: 669 - RNA, 19 nt, Artificial Sequence
  Description of Artificial Sequence Synthetic oligonucleotide
uuccucagggaaccaggcc

SEQ ID NO: 670 - RNA, 19 nt, Artificial Sequence
  Description of Artificial Sequence Synthetic oligonucleotide
ucuccggcagcagauggca

SEQ ID NO: 671 - RNA, 19 nt, Artificial Sequence
  Description of Artificial Sequence Synthetic oligonucleotide
ugccacgugggcagcagcc

SEQ ID NO: 672 - RNA, 19 nt, Artificial Sequence
  Description of Artificial Sequence Synthetic oligonucleotide
uuucaaguuacaaaagcaa

SEQ ID NO: 673 - RNA, 19 nt, Artificial Sequence
  Description of Artificial Sequence Synthetic oligonucleotide
ugcaaaacaggucuagaaa

SEQ ID NO: 674 - RNA, 19 nt, Artificial Sequence
  Description of Artificial Sequence Synthetic oligonucleotide
uacccagaauaaauaucuu

SEQ ID NO: 675 - RNA, 19 nt, Artificial Sequence
  Description of Artificial Sequence Synthetic oligonucleotide
uauaaaugucugcuugcuu

SEQ ID NO: 676 - RNA, 19 nt, Artificial Sequence
  Description of Artificial Sequence Synthetic oligonucleotide
uucaaguuacaaaagcaaa

SEQ ID NO: 677 - RNA, 19 nt, Artificial Sequence
  Description of Artificial Sequence Synthetic oligonucleotide
uaacagagaggacagaccc

SEQ ID NO: 678 - RNA, 19 nt, Artificial Sequence
  Description of Artificial Sequence Synthetic oligonucleotide
uaaauaucuucaaguuaca

SEQ ID NO: 679 - RNA, 19 nt, Artificial Sequence
  Description of Artificial Sequence Synthetic oligonucleotide
uggaaccauuuuaaagcuc

SEQ ID NO: 680 - RNA, 19 nt, Artificial Sequence
  Description of Artificial Sequence Synthetic oligonucleotide
ucaaaacaggucuagaaaa

SEQ ID NO: 681 - RNA, 19 nt, Artificial Sequence
  Description of Artificial Sequence Synthetic oligonucleotide
uaaaugcuacaaaacccag

SEQ ID NO: 682 - RNA, 19 nt, Artificial Sequence
  Description of Artificial Sequence Synthetic oligonucleotide
uccaaaagauaaaugucug

SEQ ID NO: 683 - RNA, 19 nt, Artificial Sequence
  Description of Artificial Sequence Synthetic oligonucleotide
uagcaccuggcaauggcgu

SEQ ID NO: 684 - RNA, 19 nt, Artificial Sequence
  Description of Artificial Sequence Synthetic oligonucleotide
uagaauccugccuccuugg

SEQ ID NO: 685 - RNA, 19 nt, Artificial Sequence
  Description of Artificial Sequence Synthetic oligonucleotide
uuggaucagucucugccuc

SEQ ID NO: 686 - RNA, 19 nt, Artificial Sequence
  Description of Artificial Sequence Synthetic oligonucleotide
ucccagaauaaauaucuuc

SEQ ID NO: 687 - RNA, 19 nt, Artificial Sequence
  Description of Artificial Sequence Synthetic oligonucleotide
uccagaauaaauaucuuca

SEQ ID NO: 688 - RNA, 19 nt, Artificial Sequence
  Description of Artificial Sequence Synthetic oligonucleotide
ucuucaaguuacaaaagca

SEQ ID NO: 689 - RNA, 19 nt, Artificial Sequence
  Description of Artificial Sequence Synthetic oligonucleotide
uaugcuacaaaacccagaa

SEQ ID NO: 690 - RNA, 19 nt, Artificial Sequence
  Description of Artificial Sequence Synthetic oligonucleotide
uaaaagcaaaacaggucua

SEQ ID NO: 691 - RNA, 19 nt, Artificial Sequence
  Description of Artificial Sequence Synthetic oligonucleotide
uaauaucuucaaguuacaa

SEQ ID NO: 692 - RNA, 19 nt, Artificial Sequence
  Description of Artificial Sequence Synthetic oligonucleotide
ugugacacaaagcaggugc

SEQ ID NO: 693 - RNA, 19 nt, Artificial Sequence
  Description of Artificial Sequence Synthetic oligonucleotide
uauaaauaucuucaaguua

SEQ ID NO: 694 - RNA, 19 nt, Artificial Sequence
  Description of Artificial Sequence Synthetic oligonucleotide
uaaugcuacaaaacccaga

SEQ ID NO: 695 - RNA, 19 nt, Artificial Sequence
  Description of Artificial Sequence Synthetic oligonucleotide
ucaaagcaggugcugcagu

SEQ ID NO: 696 - RNA, 19 nt, Artificial Sequence
  Description of Artificial Sequence Synthetic oligonucleotide
uagaauaaauaucuucaag

SEQ ID NO: 697 - RNA, 19 nt, Artificial Sequence
  Description of Artificial Sequence Synthetic oligonucleotide
uacaaaagcaaaacagguc

SEQ ID NO: 698 - RNA, 19 nt, Artificial Sequence
  Description of Artificial Sequence Synthetic oligonucleotide
uacaaaacccagaauaaau

SEQ ID NO: 699 - RNA, 19 nt, Artificial Sequence
  Description of Artificial Sequence Synthetic oligonucleotide
uaaagcaaaacaggucuag

SEQ ID NO: 700 - RNA, 19 nt, Artificial Sequence
  Description of Artificial Sequence Synthetic oligonucleotide
ucgaagucggugaccauga

SEQ ID NO: 701 - RNA, 19 nt, Artificial Sequence
  Description of Artificial Sequence Synthetic oligonucleotide
ucaaaagcaaaacaggucu

SEQ ID NO: 702 - RNA, 19 nt, Artificial Sequence
  Description of Artificial Sequence Synthetic oligonucleotide
ucaaguuacaaaagcaaaa

SEQ ID NO: 703 - RNA, 19 nt, Artificial Sequence
  Description of Artificial Sequence Synthetic oligonucleotide
uccuugacuuugcauucca

SEQ ID NO: 704 - RNA, 19 nt, Artificial Sequence
  Description of Artificial Sequence Synthetic oligonucleotide
uaagcguggaugcuggccu

SEQ ID NO: 705 - RNA, 19 nt, Artificial Sequence
  Description of Artificial Sequence Synthetic oligonucleotide
uuaaaugucugcuugcuug

SEQ ID NO: 706 - RNA, 19 nt, Artificial Sequence
  Description of Artificial Sequence Synthetic oligonucleotide
uaaccauuuuaaagcucag

SEQ ID NO: 707 - RNA, 19 nt, Artificial Sequence
  Description of Artificial Sequence Synthetic oligonucleotide
uaccaggccucauugauga

SEQ ID NO: 708 - RNA, 19 nt, Artificial Sequence
  Description of Artificial Sequence Synthetic oligonucleotide
uaaacccagaauaaauauc

SEQ ID NO: 709 - RNA, 19 nt, Artificial Sequence
  Description of Artificial Sequence Synthetic oligonucleotide
ugaaccauuuuaaagcuca

SEQ ID NO: 710 - RNA, 19 nt, Artificial Sequence
  Description of Artificial Sequence Synthetic oligonucleotide
ugaauaaauaucuucaagu

SEQ ID NO: 711 - RNA, 19 nt, Artificial Sequence
  Description of Artificial Sequence Synthetic oligonucleotide
uaagaggcuuggcuucaga

SEQ ID NO: 712 - RNA, 19 nt, Artificial Sequence
  Description of Artificial Sequence Synthetic oligonucleotide
ugaaccaggccucauugau

SEQ ID NO: 713 - RNA, 19 nt, Artificial Sequence
  Description of Artificial Sequence Synthetic oligonucleotide
ucagaauaaauaucuucaa

SEQ ID NO: 714 - RNA, 19 nt, Artificial Sequence
  Description of Artificial Sequence Synthetic oligonucleotide
uaagaauccugccuccuug

SEQ ID NO: 715 - RNA, 19 nt, Artificial Sequence
  Description of Artificial Sequence Synthetic oligonucleotide
uaaaacaggucuagaaaag

SEQ ID NO: 716 - RNA, 19 nt, Artificial Sequence
  Description of Artificial Sequence Synthetic oligonucleotide
uaggaagcguggaugcugg

SEQ ID NO: 717 - RNA, 19 nt, Artificial Sequence
  Description of Artificial Sequence Synthetic oligonucleotide
ucacugguugggcugaccu

SEQ ID NO: 718 - RNA, 19 nt, Artificial Sequence
  Description of Artificial Sequence Synthetic oligonucleotide
uagacaugcaggaucuugg

SEQ ID NO: 719 - RNA, 19 nt, Artificial Sequence
  Description of Artificial Sequence Synthetic oligonucleotide
ucuaggagauacaccucca

SEQ ID NO: 720 - RNA, 19 nt, Artificial Sequence
  Description of Artificial Sequence Synthetic oligonucleotide
ucuacaaaacccagaauaa

SEQ ID NO: 721 - RNA, 19 nt, Artificial Sequence
  Description of Artificial Sequence Synthetic oligonucleotide
ucagacagcaucauggcug

SEQ ID NO: 722 - RNA, 19 nt, Artificial Sequence
  Description of Artificial Sequence Synthetic oligonucleotide
uuaugcuggugucuaggag

SEQ ID NO: 723 - RNA, 19 nt, Artificial Sequence
  Description of Artificial Sequence Synthetic oligonucleotide
ugaaacuggagcagcucag

SEQ ID NO: 724 - RNA, 19 nt, Artificial Sequence
  Description of Artificial Sequence Synthetic oligonucleotide
uaggacagacccaaaagau

SEQ ID NO: 725 - RNA, 19 nt, Artificial Sequence
  Description of Artificial Sequence Synthetic oligonucleotide
ucaauggcguagacacccu

SEQ ID NO: 726 - RNA, 19 nt, Artificial Sequence
  Description of Artificial Sequence Synthetic oligonucleotide
ucaggugcugcagucgcug

SEQ ID NO: 727 - RNA, 19 nt, Artificial Sequence
  Description of Artificial Sequence Synthetic oligonucleotide
ucugccuccuugguggaga

SEQ ID NO: 728 - RNA, 19 nt, Artificial Sequence
  Description of Artificial Sequence Synthetic oligonucleotide
uagucggugaccaugaccc

SEQ ID NO: 729 - RNA, 19 nt, Artificial Sequence
  Description of Artificial Sequence Synthetic oligonucleotide
ucagccuggcauagagcag

SEQ ID NO: 730 - RNA, 19 nt, Artificial Sequence
  Description of Artificial Sequence Synthetic oligonucleotide
ucagagaggacagacccaa

SEQ ID NO: 731 - RNA, 19 nt, Artificial Sequence
  Description of Artificial Sequence Synthetic oligonucleotide
ucuggcucaguuccugcug

SEQ ID NO: 732 - RNA, 19 nt, Artificial Sequence
  Description of Artificial Sequence Synthetic oligonucleotide
ugagcuuccuggucugugu

SEQ ID NO: 733 - RNA, 19 nt, Artificial Sequence
  Description of Artificial Sequence Synthetic oligonucleotide
uaggugcugcagucgcugg

SEQ ID NO: 734 - RNA, 19 nt, Artificial Sequence
  Description of Artificial Sequence Synthetic oligonucleotide
ucuggccugucuguggaag

SEQ ID NO: 735 - RNA, 19 nt, Artificial Sequence
  Description of Artificial Sequence Synthetic oligonucleotide
uaccgccuggagcugacgg

SEQ ID NO: 736 - RNA, 19 nt, Artificial Sequence
  Description of Artificial Sequence Synthetic oligonucleotide
ucaaaagcguugugggccc

SEQ ID NO: 737 - RNA, 19 nt, Artificial Sequence
  Description of Artificial Sequence Synthetic oligonucleotide
ucacagccuggcauagagc

SEQ ID NO: 738 - RNA, 19 nt, Artificial Sequence
  Description of Artificial Sequence Synthetic oligonucleotide
ugggauuccaugcuccuug

SEQ ID NO: 739 - RNA, 19 nt, Artificial Sequence
  Description of Artificial Sequence Synthetic oligonucleotide
ugugaccaugacccugccc

SEQ ID NO: 740 - RNA, 19 nt, Artificial Sequence
  Description of Artificial Sequence Synthetic oligonucleotide
uagcaaaacaggucuagaa

SEQ ID NO: 741 - RNA, 19 nt, Artificial Sequence
  Description of Artificial Sequence Synthetic oligonucleotide
ucuggcucacuccuccagg

SEQ ID NO: 742 - RNA, 19 nt, Artificial Sequence
  Description of Artificial Sequence Synthetic oligonucleotide
ugaaguggaucagucucug

SEQ ID NO: 743 - RNA, 19 nt, Artificial Sequence
  Description of Artificial Sequence Synthetic oligonucleotide
uaugcuggugucuaggaga

SEQ ID NO: 744 - RNA, 19 nt, Artificial Sequence
  Description of Artificial Sequence Synthetic oligonucleotide
ucucaguuccugcugugug

SEQ ID NO: 745 - RNA, 19 nt, Artificial Sequence
  Description of Artificial Sequence Synthetic oligonucleotide
uuacaccuccaccaggcug

SEQ ID NO: 746 - RNA, 19 nt, Artificial Sequence
  Description of Artificial Sequence Synthetic oligonucleotide
uaucauggcugcaaugcca

SEQ ID NO: 747 - RNA, 19 nt, Artificial Sequence
  Description of Artificial Sequence Synthetic oligonucleotide
uacagagaggacagaccca

SEQ ID NO: 748 - RNA, 19 nt, Artificial Sequence
  Description of Artificial Sequence Synthetic oligonucleotide
ugaagaggcuuggcuucag

SEQ ID NO: 749 - RNA, 19 nt, Artificial Sequence
  Description of Artificial Sequence Synthetic oligonucleotide
ucccugcccucgauuuccc

SEQ ID NO: 750 - RNA, 19 nt, Artificial Sequence
  Description of Artificial Sequence Synthetic oligonucleotide
uaccaggaagccaggaaga

SEQ ID NO: 751 - RNA, 19 nt, Artificial Sequence
  Description of Artificial Sequence Synthetic oligonucleotide
ugagggagcuuccuggcac

SEQ ID NO: 752 - RNA, 19 nt, Artificial Sequence
  Description of Artificial Sequence Synthetic oligonucleotide
uaccauuuuaaagcucagc

SEQ ID NO: 753 - RNA, 19 nt, Artificial Sequence
  Description of Artificial Sequence Synthetic oligonucleotide
ucacuugcuggccugucug

SEQ ID NO: 754 - RNA, 19 nt, Artificial Sequence
  Description of Artificial Sequence Synthetic oligonucleotide
ugccacucaucuucaccag

SEQ ID NO: 755 - RNA, 19 nt, Artificial Sequence
  Description of Artificial Sequence Synthetic oligonucleotide
uuuggcagagaaguggauc

SEQ ID NO: 756 - RNA, 19 nt, Artificial Sequence
  Description of Artificial Sequence Synthetic oligonucleotide
uaguccuccucgauguagu

SEQ ID NO: 757 - RNA, 19 nt, Artificial Sequence
  Description of Artificial Sequence Synthetic oligonucleotide
uccucuggcuagaugccau

SEQ ID NO: 758 - RNA, 19 nt, Artificial Sequence
  Description of Artificial Sequence Synthetic oligonucleotide
uggagcuguguggacgcug

SEQ ID NO: 759 - RNA, 19 nt, Artificial Sequence
  Description of Artificial Sequence Synthetic oligonucleotide
ugagaaguggaucagucuc

SEQ ID NO: 760 - RNA, 19 nt, Artificial Sequence
  Description of Artificial Sequence Synthetic oligonucleotide
uagcagauggcaacggcug

SEQ ID NO: 761 - RNA, 19 nt, Artificial Sequence
  Description of Artificial Sequence Synthetic oligonucleotide
uaggccucauugaugacau

SEQ ID NO: 762 - RNA, 19 nt, Artificial Sequence
  Description of Artificial Sequence Synthetic oligonucleotide
uugcagucgcuggaggcac

SEQ ID NO: 763 - RNA, 19 nt, Artificial Sequence
  Description of Artificial Sequence Synthetic oligonucleotide
uugggucuccuccuucagc

SEQ ID NO: 764 - RNA, 19 nt, Artificial Sequence
  Description of Artificial Sequence Synthetic oligonucleotide
ucggcucggcagacagcau

SEQ ID NO: 765 - RNA, 19 nt, Artificial Sequence
  Description of Artificial Sequence Synthetic oligonucleotide
uugacaucuuuggcagaga

SEQ ID NO: 766 - RNA, 19 nt, Artificial Sequence
  Description of Artificial Sequence Synthetic oligonucleotide
ugaacgcaaggcuagcacc

SEQ ID NO: 767 - RNA, 19 nt, Artificial Sequence
  Description of Artificial Sequence Synthetic oligonucleotide
uaucuuuggcagagaagug

SEQ ID NO: 768 - RNA, 19 nt, Artificial Sequence
  Description of Artificial Sequence Synthetic oligonucleotide
uggcaggcaucgucccgga

SEQ ID NO: 769 - RNA, 19 nt, Artificial Sequence
  Description of Artificial Sequence Synthetic oligonucleotide
uggaacgcaaggcuagcac

SEQ ID NO: 770 - RNA, 19 nt, Artificial Sequence
  Description of Artificial Sequence Synthetic oligonucleotide
ugacacaaagcaggugcug

SEQ ID NO: 771 - RNA, 19 nt, Artificial Sequence
  Description of Artificial Sequence Synthetic oligonucleotide
uauaucuucaaguuacaaa

SEQ ID NO: 772 - RNA, 19 nt, Artificial Sequence
  Description of Artificial Sequence Synthetic oligonucleotide
ugugcccuucccuuggcag

SEQ ID NO: 773 - RNA, 19 nt, Artificial Sequence
  Description of Artificial Sequence Synthetic oligonucleotide
ucaaugccagccacguggg

SEQ ID NO: 774 - RNA, 19 nt, Artificial Sequence
  Description of Artificial Sequence Synthetic oligonucleotide
ugaggcuuggcuucagagc

SEQ ID NO: 775 - RNA, 19 nt, Artificial Sequence
  Description of Artificial Sequence Synthetic oligonucleotide
uggaucuuggugagguauc

SEQ ID NO: 776 - RNA, 19 nt, Artificial Sequence
  Description of Artificial Sequence Synthetic oligonucleotide
ucaacugugaugaccucgg

SEQ ID NO: 777 - RNA, 19 nt, Artificial Sequence
  Description of Artificial Sequence Synthetic oligonucleotide
uaaaugucugcuugcuugg

SEQ ID NO: 778 - RNA, 19 nt, Artificial Sequence
  Description of Artificial Sequence Synthetic oligonucleotide
uaaacaggucuagaaaagu

SEQ ID NO: 779 - RNA, 19 nt, Artificial Sequence
  Description of Artificial Sequence Synthetic oligonucleotide
uaaagcguugugggcccgg

SEQ ID NO: 780 - RNA, 19 nt, Artificial Sequence
  Description of Artificial Sequence Synthetic oligonucleotide
ucaacagagaggacagacc

SEQ ID NO: 781 - RNA, 19 nt, Artificial Sequence
  Description of Artificial Sequence Synthetic oligonucleotide
ucuccacggauccuuggcg

SEQ ID NO: 782 - RNA, 19 nt, Artificial Sequence
  Description of Artificial Sequence Synthetic oligonucleotide
uaggacugugcaggagcug

SEQ ID NO: 783 - RNA, 19 nt, Artificial Sequence
  Description of Artificial Sequence Synthetic oligonucleotide
ucaaaagauaaaugucugc

SEQ ID NO: 784 - RNA, 19 nt, Artificial Sequence
  Description of Artificial Sequence Synthetic oligonucleotide
uauccugccuccuuggugg

SEQ ID NO: 785 - RNA, 19 nt, Artificial Sequence
  Description of Artificial Sequence Synthetic oligonucleotide
ucacagcggccaaaguugg

SEQ ID NO: 786 - RNA, 19 nt, Artificial Sequence
  Description of Artificial Sequence Synthetic oligonucleotide
ucaguuccugcugugugag

SEQ ID NO: 787 - RNA, 19 nt, Artificial Sequence
  Description of Artificial Sequence Synthetic oligonucleotide
ucuggcauagagcagagua

SEQ ID NO: 788 - RNA, 19 nt, Artificial Sequence
  Description of Artificial Sequence Synthetic oligonucleotide
uccucauugaugacaucuu

SEQ ID NO: 789 - RNA, 19 nt, Artificial Sequence
  Description of Artificial Sequence Synthetic oligonucleotide
uagucgccguccucguccu

SEQ ID NO: 790 - RNA, 19 nt, Artificial Sequence
  Description of Artificial Sequence Synthetic oligonucleotide
uugggaagaauccugccuc

SEQ ID NO: 791 - RNA, 19 nt, Artificial Sequence
  Description of Artificial Sequence Synthetic oligonucleotide
ugugggugcuugacgccug

SEQ ID NO: 792 - RNA, 19 nt, Artificial Sequence
  Description of Artificial Sequence Synthetic oligonucleotide
uacacaaagcaggugcugc

SEQ ID NO: 793 - RNA, 19 nt, Artificial Sequence
  Description of Artificial Sequence Synthetic oligonucleotide
uagcaggaagcguggaugc

SEQ ID NO: 794 - RNA, 19 nt, Artificial Sequence
  Description of Artificial Sequence Synthetic oligonucleotide
uaucagucucugccucaac

SEQ ID NO: 795 - RNA, 19 nt, Artificial Sequence
  Description of Artificial Sequence Synthetic oligonucleotide
uagacagcaucauggcugc

SEQ ID NO: 796 - RNA, 19 nt, Artificial Sequence
  Description of Artificial Sequence Synthetic oligonucleotide
uucauggcugcaaugccag

SEQ ID NO: 797 - RNA, 19 nt, Artificial Sequence
  Description of Artificial Sequence Synthetic oligonucleotide
uacagccuggcauagagca

SEQ ID NO: 798 - RNA, 19 nt, Artificial Sequence
  Description of Artificial Sequence Synthetic oligonucleotide
uggcauagagcagaguaaa

SEQ ID NO: 799 - RNA, 19 nt, Artificial Sequence
  Description of Artificial Sequence Synthetic oligonucleotide
ucugguugggcugaccucg

SEQ ID NO: 800 - RNA, 19 nt, Artificial Sequence
  Description of Artificial Sequence Synthetic oligonucleotide
uagacccaaaagauaaaug

SEQ ID NO: 801 - RNA, 19 nt, Artificial Sequence
  Description of Artificial Sequence Synthetic oligonucleotide
ugguccacacagcggccaa

SEQ ID NO: 802 - RNA, 19 nt, Artificial Sequence
  Description of Artificial Sequence Synthetic oligonucleotide
uccuccaccaggcugccuc

SEQ ID NO: 803 - RNA, 19 nt, Artificial Sequence
  Description of Artificial Sequence Synthetic oligonucleotide
uaaaacagcugccaaccug

SEQ ID NO: 804 - RNA, 19 nt, Artificial Sequence
  Description of Artificial Sequence Synthetic oligonucleotide
ugaggacagacccaaaaga

SEQ ID NO: 805 - RNA, 19 nt, Artificial Sequence
  Description of Artificial Sequence Synthetic oligonucleotide
uugucugcuugcuugggug

SEQ ID NO: 806 - RNA, 19 nt, Artificial Sequence
  Description of Artificial Sequence Synthetic oligonucleotide
uaaggcaacagagaggaca

SEQ ID NO: 807 - RNA, 19 nt, Artificial Sequence
  Description of Artificial Sequence Synthetic oligonucleotide
ugugucuaggagauacacc

SEQ ID NO: 808 - RNA, 19 nt, Artificial Sequence
  Description of Artificial Sequence Synthetic oligonucleotide
uguggaucagucucugccu

SEQ ID NO: 809 - RNA, 19 nt, Artificial Sequence
  Description of Artificial Sequence Synthetic oligonucleotide
uaaacagcugccaaccugc

SEQ ID NO: 810 - RNA, 19 nt, Artificial Sequence
  Description of Artificial Sequence Synthetic oligonucleotide
ucaggcggcuugugggugc

SEQ ID NO: 811 - RNA, 19 nt, Artificial Sequence
  Description of Artificial Sequence Synthetic oligonucleotide
uggaaccaggccucauuga

SEQ ID NO: 812 - RNA, 19 nt, Artificial Sequence
  Description of Artificial Sequence Synthetic oligonucleotide
ucugccuggcucacuccuc

SEQ ID NO: 813 - RNA, 19 nt, Artificial Sequence
  Description of Artificial Sequence Synthetic oligonucleotide
uagaugagggccaucagca

SEQ ID NO: 814 - RNA, 19 nt, Artificial Sequence
  Description of Artificial Sequence Synthetic oligonucleotide
ucuuccuggucuguguucc

SEQ ID NO: 815 - RNA, 19 nt, Artificial Sequence
  Description of Artificial Sequence Synthetic oligonucleotide
uacuuugcauuccagaccu

SEQ ID NO: 816 - RNA, 19 nt, Artificial Sequence
  Description of Artificial Sequence Synthetic oligonucleotide
uuggcuagaugccauccag

SEQ ID NO: 817 - RNA, 19 nt, Artificial Sequence
  Description of Artificial Sequence Synthetic oligonucleotide
uuggcugcaaugccagcca

SEQ ID NO: 818 - RNA, 19 nt, Artificial Sequence
  Description of Artificial Sequence Synthetic oligonucleotide
ugggugugggugcuugacg

SEQ ID NO: 819 - RNA, 19 nt, Artificial Sequence
  Description of Artificial Sequence Synthetic oligonucleotide
uaaaagcguugugggcccg

SEQ ID NO: 820 - RNA, 19 nt, Artificial Sequence
  Description of Artificial Sequence Synthetic oligonucleotide
ucaccaggaagccaggaag

SEQ ID NO: 821 - RNA, 19 nt, Artificial Sequence
  Description of Artificial Sequence Synthetic oligonucleotide
ugccgggauuccaugcucc

SEQ ID NO: 822 - RNA, 19 nt, Artificial Sequence
  Description of Artificial Sequence Synthetic oligonucleotide
uccagcuccucguagucgc

SEQ ID NO: 823 - RNA, 19 nt, Artificial Sequence
  Description of Artificial Sequence Synthetic oligonucleotide
ucccgcugguccucaggga

SEQ ID NO: 824 - RNA, 19 nt, Artificial Sequence
  Description of Artificial Sequence Synthetic oligonucleotide
uugcaaugccagccacgug

SEQ ID NO: 825 - RNA, 19 nt, Artificial Sequence
  Description of Artificial Sequence Synthetic oligonucleotide
uugaugacaucuuuggcag

SEQ ID NO: 826 - RNA, 19 nt, Artificial Sequence
  Description of Artificial Sequence Synthetic oligonucleotide
ugagauacaccuccaccag

SEQ ID NO: 827 - RNA, 19 nt, Artificial Sequence
  Description of Artificial Sequence Synthetic oligonucleotide
uggugugggugcuugacgc

SEQ ID NO: 828 - RNA, 19 nt, Artificial Sequence
  Description of Artificial Sequence Synthetic oligonucleotide
ucugccucaacucggccag

SEQ ID NO: 829 - RNA, 19 nt, Artificial Sequence
  Description of Artificial Sequence Synthetic oligonucleotide
ugaagucggugaccaugac

SEQ ID NO: 830 - RNA, 19 nt, Artificial Sequence
  Description of Artificial Sequence Synthetic oligonucleotide
uguccucguccuccugcgc

SEQ ID NO: 831 - RNA, 19 nt, Artificial Sequence
  Description of Artificial Sequence Synthetic oligonucleotide
uccagccucacuguuaccc

SEQ ID NO: 832 - RNA, 19 nt, Artificial Sequence
  Description of Artificial Sequence Synthetic oligonucleotide
ugcuuuuccgaauaaacuc

SEQ ID NO: 833 - RNA, 19 nt, Artificial Sequence
  Description of Artificial Sequence Synthetic oligonucleotide
uugugagcuuggcaggcac

SEQ ID NO: 834 - RNA, 19 nt, Artificial Sequence
  Description of Artificial Sequence Synthetic oligonucleotide
uagcguggaugcuggccuc

SEQ ID NO: 835 - RNA, 19 nt, Artificial Sequence
  Description of Artificial Sequence Synthetic oligonucleotide
uaagacagaggaguccucc

SEQ ID NO: 836 - RNA, 19 nt, Artificial Sequence
  Description of Artificial Sequence Synthetic oligonucleotide
ugucugcuugcuugggugg

SEQ ID NO: 837 - RNA, 19 nt, Artificial Sequence
  Description of Artificial Sequence Synthetic oligonucleotide
uguagcaggcagcaccugg

SEQ ID NO: 838 - RNA, 19 nt, Artificial Sequence
  Description of Artificial Sequence Synthetic oligonucleotide
uuggaugcuggccucccug

SEQ ID NO: 839 - RNA, 19 nt, Artificial Sequence
  Description of Artificial Sequence Synthetic oligonucleotide
uaaaagauaaaugucugcu

SEQ ID NO: 840 - RNA, 19 nt, Artificial Sequence
  Description of Artificial Sequence Synthetic oligonucleotide
uaguccugcaaaacagcug

SEQ ID NO: 841 - RNA, 19 nt, Artificial Sequence
  Description of Artificial Sequence Synthetic oligonucleotide
ucaggaucuuggugaggua

SEQ ID NO: 842 - RNA, 19 nt, Artificial Sequence
  Description of Artificial Sequence Synthetic oligonucleotide
uagcgguggaagguggcug

SEQ ID NO: 843 - RNA, 19 nt, Artificial Sequence
  Description of Artificial Sequence Synthetic oligonucleotide
uuggcaauggcguagacac

SEQ ID NO: 844 - RNA, 19 nt, Artificial Sequence
  Description of Artificial Sequence Synthetic oligonucleotide
ucagaggaguccuccucga

SEQ ID NO: 845 - RNA, 19 nt, Artificial Sequence
  Description of Artificial Sequence Synthetic oligonucleotide
uggagcagcucagcagcuc

SEQ ID NO: 846 - RNA, 19 nt, Artificial Sequence
  Description of Artificial Sequence Synthetic oligonucleotide
ugugugagcuuggcaggca

SEQ ID NO: 847 - RNA, 19 nt, Artificial Sequence
  Description of Artificial Sequence Synthetic oligonucleotide
ucagcaccuggcaauggcg

SEQ ID NO: 848 - RNA, 19 nt, Artificial Sequence
  Description of Artificial Sequence Synthetic oligonucleotide
uucccagccucacuguuac

SEQ ID NO: 849 - RNA, 19 nt, Artificial Sequence
  Description of Artificial Sequence Synthetic oligonucleotide
ugagaaacuggagcagcuc

SEQ ID NO: 850 - RNA, 19 nt, Artificial Sequence
  Description of Artificial Sequence Synthetic oligonucleotide
uagagaaguggaucagucu

SEQ ID NO: 851 - RNA, 19 nt, Artificial Sequence
  Description of Artificial Sequence Synthetic oligonucleotide
uggucgccacucaucuuca

SEQ ID NO: 852 - RNA, 19 nt, Artificial Sequence
  Description of Artificial Sequence Synthetic oligonucleotide
ugaccagcuggcuuuuccg

SEQ ID NO: 853 - RNA, 19 nt, Artificial Sequence
  Description of Artificial Sequence Synthetic oligonucleotide
ucaugcaggaucuugguga

SEQ ID NO: 854 - RNA, 19 nt, Artificial Sequence
  Description of Artificial Sequence Synthetic oligonucleotide
uagcggccaaaguuggucc

SEQ ID NO: 855 - RNA, 19 nt, Artificial Sequence
  Description of Artificial Sequence Synthetic oligonucleotide
ucagccugugaggacgugg

SEQ ID NO: 856 - RNA, 19 nt, Artificial Sequence
  Description of Artificial Sequence Synthetic oligonucleotide
ugagcuguguggacgcugc

SEQ ID NO: 857 - RNA, 19 nt, Artificial Sequence
  Description of Artificial Sequence Synthetic oligonucleotide
ugguagcaggcagcaccug

SEQ ID NO: 858 - RNA, 19 nt, Artificial Sequence
  Description of Artificial Sequence Synthetic oligonucleotide
uugagcuccggcucggcag

SEQ ID NO: 859 - RNA, 19 nt, Artificial Sequence
  Description of Artificial Sequence Synthetic oligonucleotide
ugucuaggagauacaccuc

SEQ ID NO: 860 - RNA, 19 nt, Artificial Sequence
  Description of Artificial Sequence Synthetic oligonucleotide
uggaagcgggucccguccu

SEQ ID NO: 861 - RNA, 19 nt, Artificial Sequence
  Description of Artificial Sequence Synthetic oligonucleotide
ugcugugugagcuuggcag

SEQ ID NO: 862 - RNA, 19 nt, Artificial Sequence
  Description of Artificial Sequence Synthetic oligonucleotide
uaccagcuggcuuuuccga

SEQ ID NO: 863 - RNA, 19 nt, Artificial Sequence
  Description of Artificial Sequence Synthetic oligonucleotide
ugcaaagagguccacacag

SEQ ID NO: 864 - RNA, 19 nt, Artificial Sequence
  Description of Artificial Sequence Synthetic oligonucleotide
uaggagaccuagaggccgu

SEQ ID NO: 865 - RNA, 19 nt, Artificial Sequence
  Description of Artificial Sequence Synthetic oligonucleotide
uguccugcaaaacagcugc

SEQ ID NO: 866 - RNA, 19 nt, Artificial Sequence
  Description of Artificial Sequence Synthetic oligonucleotide
ugcaccaccacguaggugc

SEQ ID NO: 867 - RNA, 19 nt, Artificial Sequence
  Description of Artificial Sequence Synthetic oligonucleotide
uacucaagggccaggccag

SEQ ID NO: 868 - RNA, 19 nt, Artificial Sequence
  Description of Artificial Sequence Synthetic oligonucleotide
uccauuuuaaagcucagcc

SEQ ID NO: 869 - RNA, 19 nt, Artificial Sequence
  Description of Artificial Sequence Synthetic oligonucleotide
uagcguugugggcccggca

SEQ ID NO: 870 - RNA, 19 nt, Artificial Sequence
  Description of Artificial Sequence Synthetic oligonucleotide
uggcagagaaguggaucag

SEQ ID NO: 871 - RNA, 19 nt, Artificial Sequence
  Description of Artificial Sequence Synthetic oligonucleotide
ugaaaccuucuagggugug

SEQ ID NO: 872 - RNA, 19 nt, Artificial Sequence
  Description of Artificial Sequence Synthetic oligonucleotide
uagcggugaccagcacgac

SEQ ID NO: 873 - RNA, 19 nt, Artificial Sequence
  Description of Artificial Sequence Synthetic oligonucleotide
ugcagucgcuggaggcacc

SEQ ID NO: 874 - RNA, 19 nt, Artificial Sequence
  Description of Artificial Sequence Synthetic oligonucleotide
uaaaacccagaauaaauau

SEQ ID NO: 875 - RNA, 19 nt, Artificial Sequence
  Description of Artificial Sequence Synthetic oligonucleotide
uaauggcguagacacccuc

SEQ ID NO: 876 - RNA, 19 nt, Artificial Sequence
  Description of Artificial Sequence Synthetic oligonucleotide
uucuucaaguuacaaaagc

SEQ ID NO: 877 - RNA, 19 nt, Artificial Sequence
  Description of Artificial Sequence Synthetic oligonucleotide
uuuacaaaagcaaaacagg

SEQ ID NO: 878 - RNA, 19 nt, Artificial Sequence
  Description of Artificial Sequence Synthetic oligonucleotide
uaucuucaaguuacaaaag

SEQ ID NO: 879 - RNA, 19 nt, Artificial Sequence
  Description of Artificial Sequence Synthetic oligonucleotide
ucuggugucuaggagauac

SEQ ID NO: 880 - RNA, 19 nt, Artificial Sequence
  Description of Artificial Sequence Synthetic oligonucleotide
uagugcgcucugacugcga

SEQ ID NO: 881 - RNA, 19 nt, Artificial Sequence
  Description of Artificial Sequence Synthetic oligonucleotide
uugucacacuugcuggccu

SEQ ID NO: 882 - RNA, 19 nt, Artificial Sequence
  Description of Artificial Sequence Synthetic oligonucleotide
uacagaggaguccuccucg

SEQ ID NO: 883 - RNA, 19 nt, Artificial Sequence
  Description of Artificial Sequence Synthetic oligonucleotide
uguuacaaaagcaaaacag

SEQ ID NO: 884 - RNA, 19 nt, Artificial Sequence
  Description of Artificial Sequence Synthetic oligonucleotide
uaauccugccuccuuggug

SEQ ID NO: 885 - RNA, 19 nt, Artificial Sequence
  Description of Artificial Sequence Synthetic oligonucleotide
uguggaagguggcuguggu

SEQ ID NO: 886 - RNA, 19 nt, Artificial Sequence
  Description of Artificial Sequence Synthetic oligonucleotide
uggagcggguuggcugaga

SEQ ID NO: 887 - RNA, 19 nt, Artificial Sequence
  Description of Artificial Sequence Synthetic oligonucleotide
uucgccacucaucuucacc

SEQ ID NO: 888 - RNA, 19 nt, Artificial Sequence
  Description of Artificial Sequence Synthetic oligonucleotide
uagaggaguccuccucgau

SEQ ID NO: 889 - RNA, 19 nt, Artificial Sequence
  Description of Artificial Sequence Synthetic oligonucleotide
ugagcuccggcucggcaga

SEQ ID NO: 890 - RNA, 19 nt, Artificial Sequence
  Description of Artificial Sequence Synthetic oligonucleotide
uaacugugaugaccucggg

SEQ ID NO: 891 - RNA, 19 nt, Artificial Sequence
  Description of Artificial Sequence Synthetic oligonucleotide
ugauacaccuccaccaggc

SEQ ID NO: 892 - RNA, 19 nt, Artificial Sequence
  Description of Artificial Sequence Synthetic oligonucleotide
uuggcauagagcagaguaa

SEQ ID NO: 893 - RNA, 19 nt, Artificial Sequence
  Description of Artificial Sequence Synthetic oligonucleotide
ugcagcucagcagcuccuc

SEQ ID NO: 894 - RNA, 19 nt, Artificial Sequence
  Description of Artificial Sequence Synthetic oligonucleotide
uauugaugacaucuuuggc

SEQ ID NO: 895 - RNA, 15 nt, Artificial Sequence
  Description of Artificial Sequence Synthetic oligonucleotide
cacccucauaggcca

SEQ ID NO: 896 - RNA, 15 nt, Artificial Sequence
  Description of Artificial Sequence Synthetic oligonucleotide
ccuggaguuuauuca

SEQ ID NO: 897 - RNA, 15 nt, Artificial Sequence
  Description of Artificial Sequence Synthetic oligonucleotide
aggccuggaguuuaa

SEQ ID NO: 898 - RNA, 15 nt, Artificial Sequence
  Description of Artificial Sequence Synthetic oligonucleotide
cuggaguuuauucga

SEQ ID NO: 899 - RNA, 15 nt, Artificial Sequence
  Description of Artificial Sequence Synthetic oligonucleotide
caucugcugccggaa

SEQ ID NO: 900 - RNA, 15 nt, Artificial Sequence
  Description of Artificial Sequence Synthetic oligonucleotide
cauaggccuggagua

SEQ ID NO: 901 - RNA, 15 nt, Artificial Sequence
  Description of Artificial Sequence Synthetic oligonucleotide
ggcacccucauagga

SEQ ID NO: 902 - RNA, 15 nt, Artificial Sequence
  Description of Artificial Sequence Synthetic oligonucleotide
acccucauaggccua

SEQ ID NO: 903 - RNA, 15 nt, Artificial Sequence
  Description of Artificial Sequence Synthetic oligonucleotide
cucauaggccuggaa

SEQ ID NO: 904 - RNA, 15 nt, Artificial Sequence
  Description of Artificial Sequence Synthetic oligonucleotide
cccugaggaccagca

SEQ ID NO: 905 - RNA, 15 nt, Artificial Sequence
  Description of Artificial Sequence Synthetic oligonucleotide
gccuggaguuuauua

SEQ ID NO: 906 - RNA, 15 nt, Artificial Sequence
  Description of Artificial Sequence Synthetic oligonucleotide
cugagcugcuccaga

SEQ ID NO: 907 - RNA, 15 nt, Artificial Sequence
  Description of Artificial Sequence Synthetic oligonucleotide
uggaguuuauucgga

SEQ ID NO: 908 - RNA, 15 nt, Artificial Sequence
  Description of Artificial Sequence Synthetic oligonucleotide
guucccugaggacca

SEQ ID NO: 909 - RNA, 15 nt, Artificial Sequence
  Description of Artificial Sequence Synthetic oligonucleotide
uucccugaggaccaa

SEQ ID NO: 910 - RNA, 15 nt, Artificial Sequence
  Description of Artificial Sequence Synthetic oligonucleotide
ggccuggaguuuaua

SEQ ID NO: 911 - RNA, 15 nt, Artificial Sequence
  Description of Artificial Sequence Synthetic oligonucleotide
ucauaggccuggaga

SEQ ID NO: 912 - RNA, 15 nt, Artificial Sequence
  Description of Artificial Sequence Synthetic oligonucleotide
cccucauaggccuga

SEQ ID NO: 913 - RNA, 15 nt, Artificial Sequence
  Description of Artificial Sequence Synthetic oligonucleotide
gguucccugaggaca

SEQ ID NO: 914 - RNA, 15 nt, Artificial Sequence
  Description of Artificial Sequence Synthetic oligonucleotide
gcacccucauaggca

SEQ ID NO: 915 - RNA, 15 nt, Artificial Sequence
  Description of Artificial Sequence Synthetic oligonucleotide
gaguuuauucggaaa

SEQ ID NO: 916 - RNA, 15 nt, Artificial Sequence
  Description of Artificial Sequence Synthetic oligonucleotide
uccugcugccaugca

SEQ ID NO: 917 - RNA, 15 nt, Artificial Sequence
  Description of Artificial Sequence Synthetic oligonucleotide
ucugcugccggagca

SEQ ID NO: 918 - RNA, 15 nt, Artificial Sequence
  Description of Artificial Sequence Synthetic oligonucleotide
cugccggagccggca

SEQ ID NO: 919 - RNA, 15 nt, Artificial Sequence
  Description of Artificial Sequence Synthetic oligonucleotide
ugguucccugaggaa

SEQ ID NO: 920 - RNA, 15 nt, Artificial Sequence
  Description of Artificial Sequence Synthetic oligonucleotide
aucugcugccggaga

SEQ ID NO: 921 - RNA, 15 nt, Artificial Sequence
  Description of Artificial Sequence Synthetic oligonucleotide
gcugcccacguggca

SEQ ID NO: 922 - RNA, 15 nt, Artificial Sequence
  Description of Artificial Sequence Synthetic oligonucleotide
uuuuguaacuugaaa

SEQ ID NO: 923 - RNA, 15 nt, Artificial Sequence
  Description of Artificial Sequence Synthetic oligonucleotide
uagaccuguuuugca

SEQ ID NO: 924 - RNA, 15 nt, Artificial Sequence
  Description of Artificial Sequence Synthetic oligonucleotide
uauuuauucugggua

SEQ ID NO: 925 - RNA, 15 nt, Artificial Sequence
  Description of Artificial Sequence Synthetic oligonucleotide
aagcagacauuuaua

SEQ ID NO: 926 - RNA, 15 nt, Artificial Sequence
  Description of Artificial Sequence Synthetic oligonucleotide
cuuuuguaacuugaa

SEQ ID NO: 927 - RNA, 15 nt, Artificial Sequence
  Description of Artificial Sequence Synthetic oligonucleotide
cuguccucucuguua

SEQ ID NO: 928 - RNA, 15 nt, Artificial Sequence
  Description of Artificial Sequence Synthetic oligonucleotide
acuugaagauauuua

SEQ ID NO: 929 - RNA, 15 nt, Artificial Sequence
  Description of Artificial Sequence Synthetic oligonucleotide
uuuaaaaugguucca

SEQ ID NO: 930 - RNA, 15 nt, Artificial Sequence
  Description of Artificial Sequence Synthetic oligonucleotide
cuagaccuguuuuga

SEQ ID NO: 931 - RNA, 15 nt, Artificial Sequence
  Description of Artificial Sequence Synthetic oligonucleotide
guuuuguagcauuua

SEQ ID NO: 932 - RNA, 15 nt, Artificial Sequence
  Description of Artificial Sequence Synthetic oligonucleotide
cauuuaucuuuugga

SEQ ID NO: 933 - RNA, 15 nt, Artificial Sequence
  Description of Artificial Sequence Synthetic oligonucleotide
cauugccaggugcua

SEQ ID NO: 934 - RNA, 15 nt, Artificial Sequence
  Description of Artificial Sequence Synthetic oligonucleotide
ggaggcaggauucua

SEQ ID NO: 935 - RNA, 15 nt, Artificial Sequence
  Description of Artificial Sequence Synthetic oligonucleotide
cagagacugauccaa

SEQ ID NO: 936 - RNA, 15 nt, Artificial Sequence
  Description of Artificial Sequence Synthetic oligonucleotide
auauuuauucuggga

SEQ ID NO: 937 - RNA, 15 nt, Artificial Sequence
  Description of Artificial Sequence Synthetic oligonucleotide
gauauuuauucugga

SEQ ID NO: 938 - RNA, 15 nt, Artificial Sequence
  Description of Artificial Sequence Synthetic oligonucleotide
uuuguaacuugaaga

SEQ ID NO: 939 - RNA, 15 nt, Artificial Sequence
  Description of Artificial Sequence Synthetic oligonucleotide
ggguuuuguagcaua

SEQ ID NO: 940 - RNA, 15 nt, Artificial Sequence
  Description of Artificial Sequence Synthetic oligonucleotide
ccuguuuugcuuuua

SEQ ID NO: 941 - RNA, 15 nt, Artificial Sequence
  Description of Artificial Sequence Synthetic oligonucleotide
aacuugaagauauua

SEQ ID NO: 942 - RNA, 15 nt, Artificial Sequence
  Description of Artificial Sequence Synthetic oligonucleotide
cugcuuugugucaca

SEQ ID NO: 943 - RNA, 15 nt, Artificial Sequence
  Description of Artificial Sequence Synthetic oligonucleotide
uugaagauauuuaua

SEQ ID NO: 944 - RNA, 15 nt, Artificial Sequence
  Description of Artificial Sequence Synthetic oligonucleotide
gguuuuguagcauua

SEQ ID NO: 945 - RNA, 15 nt, Artificial Sequence
  Description of Artificial Sequence Synthetic oligonucleotide
cagcaccugcuuuga

SEQ ID NO: 946 - RNA, 15 nt, Artificial Sequence
  Description of Artificial Sequence Synthetic oligonucleotide
aagauauuuauucua

SEQ ID NO: 947 - RNA, 15 nt, Artificial Sequence
  Description of Artificial Sequence Synthetic oligonucleotide
uguuuugcuuuugua

SEQ ID NO: 948 - RNA, 15 nt, Artificial Sequence
  Description of Artificial Sequence Synthetic oligonucleotide
auucuggguuuugua

SEQ ID NO: 949 - RNA, 15 nt, Artificial Sequence
  Description of Artificial Sequence Synthetic oligonucleotide
accuguuuugcuuua

SEQ ID NO: 950 - RNA, 15 nt, Artificial Sequence
  Description of Artificial Sequence Synthetic oligonucleotide
ggucaccgacuucga

SEQ ID NO: 951 - RNA, 15 nt, Artificial Sequence
  Description of Artificial Sequence Synthetic oligonucleotide
cuguuuugcuuuuga

SEQ ID NO: 952 - RNA, 15 nt, Artificial Sequence
  Description of Artificial Sequence Synthetic oligonucleotide
gcuuuuguaacuuga

SEQ ID NO: 953 - RNA, 15 nt, Artificial Sequence
  Description of Artificial Sequence Synthetic oligonucleotide
augcaaagucaagga

SEQ ID NO: 954 - RNA, 15 nt, Artificial Sequence
  Description of Artificial Sequence Synthetic oligonucleotide
cagcauccacgcuua

SEQ ID NO: 955 - RNA, 15 nt, Artificial Sequence
  Description of Artificial Sequence Synthetic oligonucleotide
caagcagacauuuaa

SEQ ID NO: 956 - RNA, 15 nt, Artificial Sequence
  Description of Artificial Sequence Synthetic oligonucleotide
gcuuuaaaaugguua

SEQ ID NO: 957 - RNA, 15 nt, Artificial Sequence
  Description of Artificial Sequence Synthetic oligonucleotide
caaugaggccuggua

SEQ ID NO: 958 - RNA, 15 nt, Artificial Sequence
  Description of Artificial Sequence Synthetic oligonucleotide
uuuauucuggguuua

SEQ ID NO: 959 - RNA, 15 nt, Artificial Sequence
  Description of Artificial Sequence Synthetic oligonucleotide
cuuuaaaaugguuca

SEQ ID NO: 960 - RNA, 15 nt, Artificial Sequence
  Description of Artificial Sequence Synthetic oligonucleotide
gaagauauuuauuca

SEQ ID NO: 961 - RNA, 15 nt, Artificial Sequence
  Description of Artificial Sequence Synthetic oligonucleotide
aagccaagccucuua

SEQ ID NO: 962 - RNA, 15 nt, Artificial Sequence
  Description of Artificial Sequence Synthetic oligonucleotide
augaggccugguuca

SEQ ID NO: 963 - RNA, 15 nt, Artificial Sequence
  Description of Artificial Sequence Synthetic oligonucleotide
agauauuuauucuga

SEQ ID NO: 964 - RNA, 15 nt, Artificial Sequence
  Description of Artificial Sequence Synthetic oligonucleotide
gaggcaggauucuua

SEQ ID NO: 965 - RNA, 15 nt, Artificial Sequence
  Description of Artificial Sequence Synthetic oligonucleotide
ucuagaccuguuuua

SEQ ID NO: 966 - RNA, 15 nt, Artificial Sequence
  Description of Artificial Sequence Synthetic oligonucleotide
cauccacgcuuccua

SEQ ID NO: 967 - RNA, 15 nt, Artificial Sequence
  Description of Artificial Sequence Synthetic oligonucleotide
cagcccaaccaguga

SEQ ID NO: 968 - RNA, 15 nt, Artificial Sequence
  Description of Artificial Sequence Synthetic oligonucleotide
gauccugcaugucua

SEQ ID NO: 969 - RNA, 15 nt, Artificial Sequence
  Description of Artificial Sequence Synthetic oligonucleotide
gguguaucuccuaga

SEQ ID NO: 970 - RNA, 15 nt, Artificial Sequence
  Description of Artificial Sequence Synthetic oligonucleotide
ucuggguuuuguaga

SEQ ID NO: 971 - RNA, 15 nt, Artificial Sequence
  Description of Artificial Sequence Synthetic oligonucleotide
caugaugcugucuga

SEQ ID NO: 972 - RNA, 15 nt, Artificial Sequence
  Description of Artificial Sequence Synthetic oligonucleotide
uagacaccagcauaa

SEQ ID NO: 973 - RNA, 15 nt, Artificial Sequence
  Description of Artificial Sequence Synthetic oligonucleotide
gcugcuccaguuuca

SEQ ID NO: 974 - RNA, 15 nt, Artificial Sequence
  Description of Artificial Sequence Synthetic oligonucleotide
uuugggucuguccua

SEQ ID NO: 975 - RNA, 15 nt, Artificial Sequence
  Description of Artificial Sequence Synthetic oligonucleotide
ugucuacgccauuga

SEQ ID NO: 976 - RNA, 15 nt, Artificial Sequence
  Description of Artificial Sequence Synthetic oligonucleotide
gacugcagcaccuga

SEQ ID NO: 977 - RNA, 15 nt, Artificial Sequence
  Description of Artificial Sequence Synthetic oligonucleotide
caccaaggaggcaga

SEQ ID NO: 978 - RNA, 15 nt, Artificial Sequence
  Description of Artificial Sequence Synthetic oligonucleotide
cauggucaccgacua

SEQ ID NO: 979 - RNA, 15 nt, Artificial Sequence
  Description of Artificial Sequence Synthetic oligonucleotide
ucuaugccaggcuga

SEQ ID NO: 980 - RNA, 15 nt, Artificial Sequence
  Description of Artificial Sequence Synthetic oligonucleotide
gucuguccucucuga

SEQ ID NO: 981 - RNA, 15 nt, Artificial Sequence
  Description of Artificial Sequence Synthetic oligonucleotide
aggaacugagccaga

SEQ ID NO: 982 - RNA, 15 nt, Artificial Sequence
  Description of Artificial Sequence Synthetic oligonucleotide
agaccaggaagcuca

SEQ ID NO: 983 - RNA, 15 nt, Artificial Sequence
  Description of Artificial Sequence Synthetic oligonucleotide
cgacugcagcaccua

SEQ ID NO: 984 - RNA, 15 nt, Artificial Sequence
  Description of Artificial Sequence Synthetic oligonucleotide
cacagacaggccaga

SEQ ID NO: 985 - RNA, 15 nt, Artificial Sequence
  Description of Artificial Sequence Synthetic oligonucleotide
cagcuccaggcggua

SEQ ID NO: 986 - RNA, 15 nt, Artificial Sequence
  Description of Artificial Sequence Synthetic oligonucleotide
ccacaacgcuuuuga

SEQ ID NO: 987 - RNA, 15 nt, Artificial Sequence
  Description of Artificial Sequence Synthetic oligonucleotide
uaugccaggcuguga

SEQ ID NO: 988 - RNA, 15 nt, Artificial Sequence
  Description of Artificial Sequence Synthetic oligonucleotide
gagcauggaauccca

SEQ ID NO: 989 - RNA, 15 nt, Artificial Sequence
  Description of Artificial Sequence Synthetic oligonucleotide
agggucauggucaca

SEQ ID NO: 990 - RNA, 15 nt, Artificial Sequence
  Description of Artificial Sequence Synthetic oligonucleotide
agaccuguuuugcua

SEQ ID NO: 991 - RNA, 15 nt, Artificial Sequence
  Description of Artificial Sequence Synthetic oligonucleotide
gaggagugagccaga

SEQ ID NO: 992 - RNA, 15 nt, Artificial Sequence
  Description of Artificial Sequence Synthetic oligonucleotide
gacugauccacuuca

SEQ ID NO: 993 - RNA, 15 nt, Artificial Sequence
  Description of Artificial Sequence Synthetic oligonucleotide
cuagacaccagcaua

SEQ ID NO: 994 - RNA, 15 nt, Artificial Sequence
  Description of Artificial Sequence Synthetic oligonucleotide
cagcaggaacugaga

SEQ ID NO: 995 - RNA, 15 nt, Artificial Sequence
  Description of Artificial Sequence Synthetic oligonucleotide
cugguggagguguaa

SEQ ID NO: 996 - RNA, 15 nt, Artificial Sequence
  Description of Artificial Sequence Synthetic oligonucleotide
auugcagccaugaua

SEQ ID NO: 997 - RNA, 15 nt, Artificial Sequence
  Description of Artificial Sequence Synthetic oligonucleotide
ucuguccucucugua

SEQ ID NO: 998 - RNA, 15 nt, Artificial Sequence
  Description of Artificial Sequence Synthetic oligonucleotide
agccaagccucuuca

SEQ ID NO: 999 - RNA, 15 nt, Artificial Sequence
  Description of Artificial Sequence Synthetic oligonucleotide
aaucgagggcaggga

SEQ ID NO: 1000 - RNA, 15 nt, Artificial Sequence
  Description of Artificial Sequence Synthetic oligonucleotide
ccuggcuuccuggua

SEQ ID NO: 1001 - RNA, 15 nt, Artificial Sequence
  Description of Artificial Sequence Synthetic oligonucleotide
caggaagcucccuca

SEQ ID NO: 1002 - RNA, 15 nt, Artificial Sequence
  Description of Artificial Sequence Synthetic oligonucleotide
agcuuuaaaauggua

SEQ ID NO: 1003 - RNA, 15 nt, Artificial Sequence
  Description of Artificial Sequence Synthetic oligonucleotide
caggccagcaaguga

SEQ ID NO: 1004 - RNA, 15 nt, Artificial Sequence
  Description of Artificial Sequence Synthetic oligonucleotide
ugaagaugaguggca

SEQ ID NO: 1005 - RNA, 15 nt, Artificial Sequence
  Description of Artificial Sequence Synthetic oligonucleotide
cacuucucugccaaa

SEQ ID NO: 1006 - RNA, 15 nt, Artificial Sequence
  Description of Artificial Sequence Synthetic oligonucleotide
caucgaggaggacua

SEQ ID NO: 1007 - RNA, 15 nt, Artificial Sequence
  Description of Artificial Sequence Synthetic oligonucleotide
caucuagccagagga

SEQ ID NO: 1008 - RNA, 15 nt, Artificial Sequence
  Description of Artificial Sequence Synthetic oligonucleotide
guccacacagcucca

SEQ ID NO: 1009 - RNA, 15 nt, Artificial Sequence
  Description of Artificial Sequence Synthetic oligonucleotide
cugauccacuucuca

SEQ ID NO: 1010 - RNA, 15 nt, Artificial Sequence
  Description of Artificial Sequence Synthetic oligonucleotide
cguugccaucugcua

SEQ ID NO: 1011 - RNA, 15 nt, Artificial Sequence
  Description of Artificial Sequence Synthetic oligonucleotide
caucaaugaggccua

SEQ ID NO: 1012 - RNA, 15 nt, Artificial Sequence
  Description of Artificial Sequence Synthetic oligonucleotide
cuccagcgacugcaa

SEQ ID NO: 1013 - RNA, 15 nt, Artificial Sequence
  Description of Artificial Sequence Synthetic oligonucleotide
aaggaggagacccaa

SEQ ID NO: 1014 - RNA, 15 nt, Artificial Sequence
  Description of Artificial Sequence Synthetic oligonucleotide
ugucugccgagccga

SEQ ID NO: 1015 - RNA, 15 nt, Artificial Sequence
  Description of Artificial Sequence Synthetic oligonucleotide
ugccaaagaugucaa

SEQ ID NO: 1016 - RNA, 15 nt, Artificial Sequence
  Description of Artificial Sequence Synthetic oligonucleotide
cuagccuugcguuca

SEQ ID NO: 1017 - RNA, 15 nt, Artificial Sequence
  Description of Artificial Sequence Synthetic oligonucleotide
ucucugccaaagaua

SEQ ID NO: 1018 - RNA, 15 nt, Artificial Sequence
  Description of Artificial Sequence Synthetic oligonucleotide
ggacgaugccugcca

SEQ ID NO: 1019 - RNA, 15 nt, Artificial Sequence
  Description of Artificial Sequence Synthetic oligonucleotide
uagccuugcguucca

SEQ ID NO: 1020 - RNA, 15 nt, Artificial Sequence
  Description of Artificial Sequence Synthetic oligonucleotide
accugcuuuguguca

SEQ ID NO: 1021 - RNA, 15 nt, Artificial Sequence
  Description of Artificial Sequence Synthetic oligonucleotide
uaacuugaagauaua

SEQ ID NO: 1022 - RNA, 15 nt, Artificial Sequence
  Description of Artificial Sequence Synthetic oligonucleotide
caagggaagggcaca

SEQ ID NO: 1023 - RNA, 15 nt, Artificial Sequence
  Description of Artificial Sequence Synthetic oligonucleotide
cguggcuggcauuga

SEQ ID NO: 1024 - RNA, 15 nt, Artificial Sequence
  Description of Artificial Sequence Synthetic oligonucleotide
ugaagccaagccuca

SEQ ID NO: 1025 - RNA, 15 nt, Artificial Sequence
  Description of Artificial Sequence Synthetic oligonucleotide
ccucaccaagaucca

SEQ ID NO: 1026 - RNA, 15 nt, Artificial Sequence
  Description of Artificial Sequence Synthetic oligonucleotide
ggucaucacaguuga

SEQ ID NO: 1027 - RNA, 15 nt, Artificial Sequence
  Description of Artificial Sequence Synthetic oligonucleotide
gcaagcagacauuua

SEQ ID NO: 1028 - RNA, 15 nt, Artificial Sequence
  Description of Artificial Sequence Synthetic oligonucleotide
uucuagaccuguuua

SEQ ID NO: 1029 - RNA, 15 nt, Artificial Sequence
  Description of Artificial Sequence Synthetic oligonucleotide
gcccacaacgcuuua

SEQ ID NO: 1030 - RNA, 15 nt, Artificial Sequence
  Description of Artificial Sequence Synthetic oligonucleotide
uguccucucuguuga

SEQ ID NO: 1031 - RNA, 15 nt, Artificial Sequence
  Description of Artificial Sequence Synthetic oligonucleotide
aaggauccguggaga

SEQ ID NO: 1032 - RNA, 15 nt, Artificial Sequence
  Description of Artificial Sequence Synthetic oligonucleotide
uccugcacaguccua

SEQ ID NO: 1033 - RNA, 15 nt, Artificial Sequence
  Description of Artificial Sequence Synthetic oligonucleotide
acauuuaucuuuuga

SEQ ID NO: 1034 - RNA, 15 nt, Artificial Sequence
  Description of Artificial Sequence Synthetic oligonucleotide
caaggaggcaggaua

SEQ ID NO: 1035 - RNA, 15 nt, Artificial Sequence
  Description of Artificial Sequence Synthetic oligonucleotide
cuuuggccgcuguga

SEQ ID NO: 1036 - RNA, 15 nt, Artificial Sequence
  Description of Artificial Sequence Synthetic oligonucleotide
cacagcaggaacuga

SEQ ID NO: 1037 - RNA, 15 nt, Artificial Sequence
  Description of Artificial Sequence Synthetic oligonucleotide
cugcucuaugccaga

SEQ ID NO: 1038 - RNA, 15 nt, Artificial Sequence
  Description of Artificial Sequence Synthetic oligonucleotide
ugucaucaaugagga

SEQ ID NO: 1039 - RNA, 15 nt, Artificial Sequence
  Description of Artificial Sequence Synthetic oligonucleotide
cgaggacggcgacua

SEQ ID NO: 1040 - RNA, 15 nt, Artificial Sequence
  Description of Artificial Sequence Synthetic oligonucleotide
caggauucuucccaa

SEQ ID NO: 1041 - RNA, 15 nt, Artificial Sequence
  Description of Artificial Sequence Synthetic oligonucleotide
cgucaagcacccaca

SEQ ID NO: 1042 - RNA, 15 nt, Artificial Sequence
  Description of Artificial Sequence Synthetic oligonucleotide
caccugcuuugugua

SEQ ID NO: 1043 - RNA, 15 nt, Artificial Sequence
  Description of Artificial Sequence Synthetic oligonucleotide
ccacgcuuccugcua

SEQ ID NO: 1044 - RNA, 15 nt, Artificial Sequence
  Description of Artificial Sequence Synthetic oligonucleotide
aggcagagacugaua

SEQ ID NO: 1045 - RNA, 15 nt, Artificial Sequence
  Description of Artificial Sequence Synthetic oligonucleotide
ccaugaugcugucua

SEQ ID NO: 1046 - RNA, 15 nt, Artificial Sequence
  Description of Artificial Sequence Synthetic oligonucleotide
cauugcagccaugaa

SEQ ID NO: 1047 - RNA, 15 nt, Artificial Sequence
  Description of Artificial Sequence Synthetic oligonucleotide
cuaugccaggcugua

SEQ ID NO: 1048 - RNA, 15 nt, Artificial Sequence
  Description of Artificial Sequence Synthetic oligonucleotide
cucugcucuaugcca

SEQ ID NO: 1049 - RNA, 15 nt, Artificial Sequence
  Description of Artificial Sequence Synthetic oligonucleotide
gucagcccaaccaga

SEQ ID NO: 1050 - RNA, 15 nt, Artificial Sequence
  Description of Artificial Sequence Synthetic oligonucleotide
uaucuuuugggucua

SEQ ID NO: 1051 - RNA, 15 nt, Artificial Sequence
  Description of Artificial Sequence Synthetic oligonucleotide
ccgcuguguggacca

SEQ ID NO: 1052 - RNA, 15 nt, Artificial Sequence
  Description of Artificial Sequence Synthetic oligonucleotide
cagccugguggagga

SEQ ID NO: 1053 - RNA, 15 nt, Artificial Sequence
  Description of Artificial Sequence Synthetic oligonucleotide
uuggcagcuguuuua

SEQ ID NO: 1054 - RNA, 15 nt, Artificial Sequence
  Description of Artificial Sequence Synthetic oligonucleotide
uugggucuguccuca

SEQ ID NO: 1055 - RNA, 15 nt, Artificial Sequence
  Description of Artificial Sequence Synthetic oligonucleotide
caagcaagcagacaa

SEQ ID NO: 1056 - RNA, 15 nt, Artificial Sequence
  Description of Artificial Sequence Synthetic oligonucleotide
cucucuguugccuua

SEQ ID NO: 1057 - RNA, 15 nt, Artificial Sequence
  Description of Artificial Sequence Synthetic oligonucleotide
uaucuccuagacaca

SEQ ID NO: 1058 - RNA, 15 nt, Artificial Sequence
  Description of Artificial Sequence Synthetic oligonucleotide
agagacugauccaca

SEQ ID NO: 1059 - RNA, 15 nt, Artificial Sequence
  Description of Artificial Sequence Synthetic oligonucleotide
guuggcagcuguuua

SEQ ID NO: 1060 - RNA, 15 nt, Artificial Sequence
  Description of Artificial Sequence Synthetic oligonucleotide
ccacaagccgccuga

SEQ ID NO: 1061 - RNA, 15 nt, Artificial Sequence
  Description of Artificial Sequence Synthetic oligonucleotide
ugaggccugguucca

SEQ ID NO: 1062 - RNA, 15 nt, Artificial Sequence
  Description of Artificial Sequence Synthetic oligonucleotide
agugagccaggcaga

SEQ ID NO: 1063 - RNA, 15 nt, Artificial Sequence
  Description of Artificial Sequence Synthetic oligonucleotide
gauggcccucaucua

SEQ ID NO: 1064 - RNA, 15 nt, Artificial Sequence
  Description of Artificial Sequence Synthetic oligonucleotide
cacagaccaggaaga

SEQ ID NO: 1065 - RNA, 15 nt, Artificial Sequence
  Description of Artificial Sequence Synthetic oligonucleotide
cuggaaugcaaagua

SEQ ID NO: 1066 - RNA, 15 nt, Artificial Sequence
  Description of Artificial Sequence Synthetic oligonucleotide
auggcaucuagccaa

SEQ ID NO: 1067 - RNA, 15 nt, Artificial Sequence
  Description of Artificial Sequence Synthetic oligonucleotide
uggcauugcagccaa

SEQ ID NO: 1068 - RNA, 15 nt, Artificial Sequence
  Description of Artificial Sequence Synthetic oligonucleotide
aagcacccacaccca

SEQ ID NO: 1069 - RNA, 15 nt, Artificial Sequence
  Description of Artificial Sequence Synthetic oligonucleotide
cccacaacgcuuuua

SEQ ID NO: 1070 - RNA, 15 nt, Artificial Sequence
  Description of Artificial Sequence Synthetic oligonucleotide
cuggcuuccugguga

SEQ ID NO: 1071 - RNA, 15 nt, Artificial Sequence
  Description of Artificial Sequence Synthetic oligonucleotide
cauggaaucccggca

SEQ ID NO: 1072 - RNA, 15 nt, Artificial Sequence
  Description of Artificial Sequence Synthetic oligonucleotide
cuacgaggagcugga

SEQ ID NO: 1073 - RNA, 15 nt, Artificial Sequence
  Description of Artificial Sequence Synthetic oligonucleotide
ugaggaccagcggga

SEQ ID NO: 1074 - RNA, 15 nt, Artificial Sequence
  Description of Artificial Sequence Synthetic oligonucleotide
uggcuggcauugcaa

SEQ ID NO: 1075 - RNA, 15 nt, Artificial Sequence
  Description of Artificial Sequence Synthetic oligonucleotide
caaagaugucaucaa

SEQ ID NO: 1076 - RNA, 15 nt, Artificial Sequence
  Description of Artificial Sequence Synthetic oligonucleotide
uggagguguaucuca

SEQ ID NO: 1077 - RNA, 15 nt, Artificial Sequence
  Description of Artificial Sequence Synthetic oligonucleotide
caagcacccacacca

SEQ ID NO: 1078 - RNA, 15 nt, Artificial Sequence
  Description of Artificial Sequence Synthetic oligonucleotide
ccgaguugaggcaga

SEQ ID NO: 1079 - RNA, 15 nt, Artificial Sequence
  Description of Artificial Sequence Synthetic oligonucleotide
uggucaccgacuuca

SEQ ID NO: 1080 - RNA, 15 nt, Artificial Sequence
  Description of Artificial Sequence Synthetic oligonucleotide
aggaggacgaggaca

SEQ ID NO: 1081 - RNA, 15 nt, Artificial Sequence
  Description of Artificial Sequence Synthetic oligonucleotide
aacagugaggcugga

SEQ ID NO: 1082 - RNA, 15 nt, Artificial Sequence
  Description of Artificial Sequence Synthetic oligonucleotide
uuauucggaaaagca

SEQ ID NO: 1083 - RNA, 15 nt, Artificial Sequence
  Description of Artificial Sequence Synthetic oligonucleotide
cugccaagcucacaa

SEQ ID NO: 1084 - RNA, 15 nt, Artificial Sequence
  Description of Artificial Sequence Synthetic oligonucleotide
ccagcauccacgcua

SEQ ID NO: 1085 - RNA, 15 nt, Artificial Sequence
  Description of Artificial Sequence Synthetic oligonucleotide
gacuccucugucuua

SEQ ID NO: 1086 - RNA, 15 nt, Artificial Sequence
  Description of Artificial Sequence Synthetic oligonucleotide
ccaagcaagcagaca

SEQ ID NO: 1087 - RNA, 15 nt, Artificial Sequence
  Description of Artificial Sequence Synthetic oligonucleotide
gugcugccugcuaca

SEQ ID NO: 1088 - RNA, 15 nt, Artificial Sequence
  Description of Artificial Sequence Synthetic oligonucleotide
gaggccagcauccaa

SEQ ID NO: 1089 - RNA, 15 nt, Artificial Sequence
  Description of Artificial Sequence Synthetic oligonucleotide
gacauuuaucuuuua

SEQ ID NO: 1090 - RNA, 15 nt, Artificial Sequence
  Description of Artificial Sequence Synthetic oligonucleotide
uguuuugcaggacua

SEQ ID NO: 1091 - RNA, 15 nt, Artificial Sequence
  Description of Artificial Sequence Synthetic oligonucleotide
ucaccaagauccuga

SEQ ID NO: 1092 - RNA, 15 nt, Artificial Sequence
  Description of Artificial Sequence Synthetic oligonucleotide
caccuuccaccgcua

SEQ ID NO: 1093 - RNA, 15 nt, Artificial Sequence
  Description of Artificial Sequence Synthetic oligonucleotide
cuacgccauugccaa

SEQ ID NO: 1094 - RNA, 15 nt, Artificial Sequence
  Description of Artificial Sequence Synthetic oligonucleotide
ggaggacuccucuga

SEQ ID NO: 1095 - RNA, 15 nt, Artificial Sequence
  Description of Artificial Sequence Synthetic oligonucleotide
ugcugagcugcucca

SEQ ID NO: 1096 - RNA, 15 nt, Artificial Sequence
  Description of Artificial Sequence Synthetic oligonucleotide
ugccaagcucacaca

SEQ ID NO: 1097 - RNA, 15 nt, Artificial Sequence
  Description of Artificial Sequence Synthetic oligonucleotide
auugccaggugcuga

SEQ ID NO: 1098 - RNA, 15 nt, Artificial Sequence
  Description of Artificial Sequence Synthetic oligonucleotide
cagugaggcugggaa

SEQ ID NO: 1099 - RNA, 15 nt, Artificial Sequence
  Description of Artificial Sequence Synthetic oligonucleotide
ugcuccaguuucuca

SEQ ID NO: 1100 - RNA, 15 nt, Artificial Sequence
  Description of Artificial Sequence Synthetic oligonucleotide
ugauccacuucucua

SEQ ID NO: 1101 - RNA, 15 nt, Artificial Sequence
  Description of Artificial Sequence Synthetic oligonucleotide
gaugaguggcgacca

SEQ ID NO: 1102 - RNA, 15 nt, Artificial Sequence
  Description of Artificial Sequence Synthetic oligonucleotide
aaagccagcugguca

SEQ ID NO: 1103 - RNA, 15 nt, Artificial Sequence
  Description of Artificial Sequence Synthetic oligonucleotide
caagauccugcauga

SEQ ID NO: 1104 - RNA, 15 nt, Artificial Sequence
  Description of Artificial Sequence Synthetic oligonucleotide
caacuuuggccgcua

SEQ ID NO: 1105 - RNA, 15 nt, Artificial Sequence
  Description of Artificial Sequence Synthetic oligonucleotide
guccucacaggcuga

SEQ ID NO: 1106 - RNA, 15 nt, Artificial Sequence
  Description of Artificial Sequence Synthetic oligonucleotide
cguccacacagcuca

SEQ ID NO: 1107 - RNA, 15 nt, Artificial Sequence
  Description of Artificial Sequence Synthetic oligonucleotide
ugcugccugcuacca

SEQ ID NO: 1108 - RNA, 15 nt, Artificial Sequence
  Description of Artificial Sequence Synthetic oligonucleotide
cgagccggagcucaa

SEQ ID NO: 1109 - RNA, 15 nt, Artificial Sequence
  Description of Artificial Sequence Synthetic oligonucleotide
uguaucuccuagaca

SEQ ID NO: 1110 - RNA, 15 nt, Artificial Sequence
  Description of Artificial Sequence Synthetic oligonucleotide
cgggacccgcuucca

SEQ ID NO: 1111 - RNA, 15 nt, Artificial Sequence
  Description of Artificial Sequence Synthetic oligonucleotide
caagcucacacagca

SEQ ID NO: 1112 - RNA, 15 nt, Artificial Sequence
  Description of Artificial Sequence Synthetic oligonucleotide
aaaagccagcuggua

SEQ ID NO: 1113 - RNA, 15 nt, Artificial Sequence
  Description of Artificial Sequence Synthetic oligonucleotide
guggaccucuuugca

SEQ ID NO: 1114 - RNA, 15 nt, Artificial Sequence
  Description of Artificial Sequence Synthetic oligonucleotide
ccucuaggucuccua

SEQ ID NO: 1115 - RNA, 15 nt, Artificial Sequence
  Description of Artificial Sequence Synthetic oligonucleotide
cuguuuugcaggaca

SEQ ID NO: 1116 - RNA, 15 nt, Artificial Sequence
  Description of Artificial Sequence Synthetic oligonucleotide
cuacgugguggugca

SEQ ID NO: 1117 - RNA, 15 nt, Artificial Sequence
  Description of Artificial Sequence Synthetic oligonucleotide
ccuggcccuugagua

SEQ ID NO: 1118 - RNA, 15 nt, Artificial Sequence
  Description of Artificial Sequence Synthetic oligonucleotide
gagcuuuaaaaugga

SEQ ID NO: 1119 - RNA, 15 nt, Artificial Sequence
  Description of Artificial Sequence Synthetic oligonucleotide
gggcccacaacgcua

SEQ ID NO: 1120 - RNA, 15 nt, Artificial Sequence
  Description of Artificial Sequence Synthetic oligonucleotide
uccacuucucugcca

SEQ ID NO: 1121 - RNA, 15 nt, Artificial Sequence
  Description of Artificial Sequence Synthetic oligonucleotide
cccuagaagguuuca

SEQ ID NO: 1122 - RNA, 15 nt, Artificial Sequence
  Description of Artificial Sequence Synthetic oligonucleotide
ugcuggucaccgcua

SEQ ID NO: 1123 - RNA, 15 nt, Artificial Sequence
  Description of Artificial Sequence Synthetic oligonucleotide
ccuccagcgacugca

SEQ ID NO: 1124 - RNA, 15 nt, Artificial Sequence
  Description of Artificial Sequence Synthetic oligonucleotide
uuauucuggguuuua

SEQ ID NO: 1125 - RNA, 15 nt, Artificial Sequence
  Description of Artificial Sequence Synthetic oligonucleotide
gugucuacgccauua

SEQ ID NO: 1126 - RNA, 15 nt, Artificial Sequence
  Description of Artificial Sequence Synthetic oligonucleotide
uuguaacuugaagaa

SEQ ID NO: 1127 - RNA, 15 nt, Artificial Sequence
  Description of Artificial Sequence Synthetic oligonucleotide
uuuugcuuuuguaaa

SEQ ID NO: 1128 - RNA, 15 nt, Artificial Sequence
  Description of Artificial Sequence Synthetic oligonucleotide
uguaacuugaagaua

SEQ ID NO: 1129 - RNA, 15 nt, Artificial Sequence
  Description of Artificial Sequence Synthetic oligonucleotide
cuccuagacaccaga

SEQ ID NO: 1130 - RNA, 15 nt, Artificial Sequence
  Description of Artificial Sequence Synthetic oligonucleotide
agucagagcgcacua

SEQ ID NO: 1131 - RNA, 15 nt, Artificial Sequence
  Description of Artificial Sequence Synthetic oligonucleotide
cagcaagugugacaa

SEQ ID NO: 1132 - RNA, 15 nt, Artificial Sequence
  Description of Artificial Sequence Synthetic oligonucleotide
gaggacuccucugua

SEQ ID NO: 1133 - RNA, 15 nt, Artificial Sequence
  Description of Artificial Sequence Synthetic oligonucleotide
uuugcuuuuguaaca

SEQ ID NO: 1134 - RNA, 15 nt, Artificial Sequence
  Description of Artificial Sequence Synthetic oligonucleotide
aaggaggcaggauua

SEQ ID NO: 1135 - RNA, 15 nt, Artificial Sequence
  Description of Artificial Sequence Synthetic oligonucleotide
cagccaccuuccaca

SEQ ID NO: 1136 - RNA, 15 nt, Artificial Sequence
  Description of Artificial Sequence Synthetic oligonucleotide
agccaacccgcucca

SEQ ID NO: 1137 - RNA, 15 nt, Artificial Sequence
  Description of Artificial Sequence Synthetic oligonucleotide
aagaugaguggcgaa

SEQ ID NO: 1138 - RNA, 15 nt, Artificial Sequence
  Description of Artificial Sequence Synthetic oligonucleotide
aggaggacuccucua

SEQ ID NO: 1139 - RNA, 15 nt, Artificial Sequence
  Description of Artificial Sequence Synthetic oligonucleotide
ccgagccggagcuca

SEQ ID NO: 1140 - RNA, 15 nt, Artificial Sequence
  Description of Artificial Sequence Synthetic oligonucleotide
aggucaucacaguua

SEQ ID NO: 1141 - RNA, 15 nt, Artificial Sequence
  Description of Artificial Sequence Synthetic oligonucleotide
gguggagguguauca

SEQ ID NO: 1142 - RNA, 15 nt, Artificial Sequence
  Description of Artificial Sequence Synthetic oligonucleotide
ucugcucuaugccaa

SEQ ID NO: 1143 - RNA, 15 nt, Artificial Sequence
  Description of Artificial Sequence Synthetic oligonucleotide
agcugcugagcugca

SEQ ID NO: 1144 - RNA, 15 nt, Artificial Sequence
  Description of Artificial Sequence Synthetic oligonucleotide
aagaugucaucaaua

SEQ ID NO: 1145 - RNA, 19 nt, Artificial Sequence
  Description of Artificial Sequence Synthetic oligonucleotide
uucuuuggcagagaagugg

SEQ ID NO: 1146 - RNA, 19 nt, Artificial Sequence
  Description of Artificial Sequence Synthetic oligonucleotide
ucaugcaggaucuugguga

SEQ ID NO: 1147 - RNA, 19 nt, Artificial Sequence
  Description of Artificial Sequence Synthetic oligonucleotide
uuaaacuccaggccuauga

SEQ ID NO: 1148 - RNA, 19 nt, Artificial Sequence
  Description of Artificial Sequence Synthetic oligonucleotide
ugcaaugccagccacgugg

SEQ ID NO: 1149 - RNA, 19 nt, Artificial Sequence
  Description of Artificial Sequence Synthetic oligonucleotide
ugacuuugcauuccagacc

SEQ ID NO: 1150 - RNA, 19 nt, Artificial Sequence
  Description of Artificial Sequence Synthetic oligonucleotide
uaaccgugcccuucccuug

SEQ ID NO: 1151 - RNA, 19 nt, Artificial Sequence
  Description of Artificial Sequence Synthetic oligonucleotide
uaguggaucagucucugcc

SEQ ID NO: 1152 - RNA, 19 nt, Artificial Sequence
  Description of Artificial Sequence Synthetic oligonucleotide
ucauucucgaagucgguga

SEQ ID NO: 1153 - RNA, 19 nt, Artificial Sequence
  Description of Artificial Sequence Synthetic oligonucleotide
uaacuccaggccuaugagg

SEQ ID NO: 1154 - RNA, 19 nt, Artificial Sequence
  Description of Artificial Sequence Synthetic oligonucleotide
uaacuucaaggccagcucc

SEQ ID NO: 1155 - RNA, 19 nt, Artificial Sequence
  Description of Artificial Sequence Synthetic oligonucleotide
uaauggcguagacacccuc

SEQ ID NO: 1156 - RNA, 19 nt, Artificial Sequence
  Description of Artificial Sequence Synthetic oligonucleotide
uuagaggcaggcaucgucc

SEQ ID NO: 1157 - RNA, 19 nt, Artificial Sequence
  Description of Artificial Sequence Synthetic oligonucleotide
uaccuccacggauccuugg

SEQ ID NO: 1158 - RNA, 19 nt, Artificial Sequence
  Description of Artificial Sequence Synthetic oligonucleotide
ucagucucugccucaacuc

SEQ ID NO: 1159 - RNA, 19 nt, Artificial Sequence
  Description of Artificial Sequence Synthetic oligonucleotide
uucuuggugagguaucccc

SEQ ID NO: 1160 - RNA, 19 nt, Artificial Sequence
  Description of Artificial Sequence Synthetic oligonucleotide
uagauggcaacggcuguca

SEQ ID NO: 1161 - RNA, 19 nt, Artificial Sequence
  Description of Artificial Sequence Synthetic oligonucleotide
uacaccuccaccaggcugc

SEQ ID NO: 1162 - RNA, 19 nt, Artificial Sequence
  Description of Artificial Sequence Synthetic oligonucleotide
uuggaagacaugcaggauc

SEQ ID NO: 1163 - RNA, 19 nt, Artificial Sequence
  Description of Artificial Sequence Synthetic oligonucleotide
uaugagggugccgcuaacc

SEQ ID NO: 1164 - RNA, 19 nt, Artificial Sequence
  Description of Artificial Sequence Synthetic oligonucleotide
uuucucgaagucggugacc

SEQ ID NO: 1165 - RNA, 19 nt, Artificial Sequence
  Description of Artificial Sequence Synthetic oligonucleotide
uaaguggaucagucucugc

SEQ ID NO: 1166 - RNA, 19 nt, Artificial Sequence
  Description of Artificial Sequence Synthetic oligonucleotide
ucacucuguaugcuggugu

SEQ ID NO: 1167 - RNA, 19 nt, Artificial Sequence
  Description of Artificial Sequence Synthetic oligonucleotide
uguguggacgcugcaguug

SEQ ID NO: 1168 - RNA, 19 nt, Artificial Sequence
  Description of Artificial Sequence Synthetic oligonucleotide
uguagaggcaggcaucguc

SEQ ID NO: 1169 - RNA, 19 nt, Artificial Sequence
  Description of Artificial Sequence Synthetic oligonucleotide
ucuguaugcuggugucuag

SEQ ID NO: 1170 - RNA, 19 nt, Artificial Sequence
  Description of Artificial Sequence Synthetic oligonucleotide
uucaaggccagcuccagca

SEQ ID NO: 1171 - RNA, 19 nt, Artificial Sequence
  Description of Artificial Sequence Synthetic oligonucleotide
uaagucggugaccaugacc

SEQ ID NO: 1172 - RNA, 19 nt, Artificial Sequence
  Description of Artificial Sequence Synthetic oligonucleotide
ucacacuugcuggccuguc

SEQ ID NO: 1173 - RNA, 19 nt, Artificial Sequence
  Description of Artificial Sequence Synthetic oligonucleotide
ugucuguggaagcgggucc

SEQ ID NO: 1174 - RNA, 19 nt, Artificial Sequence
  Description of Artificial Sequence Synthetic oligonucleotide
uuuggcagagaaguggauc

SEQ ID NO: 1175 - RNA, 19 nt, Artificial Sequence
  Description of Artificial Sequence Synthetic oligonucleotide
uagaaggccauggaagaca

SEQ ID NO: 1176 - RNA, 19 nt, Artificial Sequence
  Description of Artificial Sequence Synthetic oligonucleotide
uuugaggacgcggcuguac

SEQ ID NO: 1177 - RNA, 19 nt, Artificial Sequence
  Description of Artificial Sequence Synthetic oligonucleotide
uuucccgguggucacucug

SEQ ID NO: 1178 - RNA, 19 nt, Artificial Sequence
  Description of Artificial Sequence Synthetic oligonucleotide
uaugcuggugucuaggaga

SEQ ID NO: 1179 - RNA, 19 nt, Artificial Sequence
  Description of Artificial Sequence Synthetic oligonucleotide
uggaagguggcugugguuc

SEQ ID NO: 1180 - RNA, 19 nt, Artificial Sequence
  Description of Artificial Sequence Synthetic oligonucleotide
uaaacuccaggccuaugag

SEQ ID NO: 1181 - RNA, 19 nt, Artificial Sequence
  Description of Artificial Sequence Synthetic oligonucleotide
uuccacgggaugcucuggg

SEQ ID NO: 1182 - RNA, 19 nt, Artificial Sequence
  Description of Artificial Sequence Synthetic oligonucleotide
uuccgcuccagguuccacg

SEQ ID NO: 1183 - RNA, 19 nt, Artificial Sequence
  Description of Artificial Sequence Synthetic oligonucleotide
uucagcaccaccacguagg

SEQ ID NO: 1184 - RNA, 19 nt, Artificial Sequence
  Description of Artificial Sequence Synthetic oligonucleotide
uccuccucgggcacauucu

SEQ ID NO: 1185 - RNA, 19 nt, Artificial Sequence
  Description of Artificial Sequence Synthetic oligonucleotide
uuuccgaauaaacuccagg

SEQ ID NO: 1186 - RNA, 19 nt, Artificial Sequence
  Description of Artificial Sequence Synthetic oligonucleotide
uuuccaugcuccuugacuu

SEQ ID NO: 1187 - RNA, 19 nt, Artificial Sequence
  Description of Artificial Sequence Synthetic oligonucleotide
ugcuccuugacuuugcauu

SEQ ID NO: 1188 - RNA, 14 nt, Artificial Sequence
  Description of Artificial Sequence Synthetic oligonucleotide
ucucugccaaagaa

SEQ ID NO: 1189 - RNA, 14 nt, Artificial Sequence
  Description of Artificial Sequence Synthetic oligonucleotide
aagauccugcauga

SEQ ID NO: 1190 - RNA, 14 nt, Artificial Sequence
  Description of Artificial Sequence Synthetic oligonucleotide
ggccuggaguuuaa

SEQ ID NO: 1191 - RNA, 14 nt, Artificial Sequence
  Description of Artificial Sequence Synthetic oligonucleotide
uggcuggcauugca

SEQ ID NO: 1192 - RNA, 14 nt, Artificial Sequence
  Description of Artificial Sequence Synthetic oligonucleotide
ggaaugcaaaguca

SEQ ID NO: 1193 - RNA, 14 nt, Artificial Sequence
  Description of Artificial Sequence Synthetic oligonucleotide
gaagggcacgguua

SEQ ID NO: 1194 - RNA, 14 nt, Artificial Sequence
  Description of Artificial Sequence Synthetic oligonucleotide
agacugauccacua

SEQ ID NO: 1195 - RNA, 14 nt, Artificial Sequence
  Description of Artificial Sequence Synthetic oligonucleotide
gacuucgagaauga

SEQ ID NO: 1196 - RNA, 14 nt, Artificial Sequence
  Description of Artificial Sequence Synthetic oligonucleotide
uaggccuggaguua

SEQ ID NO: 1197 - RNA, 14 nt, Artificial Sequence
  Description of Artificial Sequence Synthetic oligonucleotide
uggccuugaaguua

SEQ ID NO: 1198 - RNA, 14 nt, Artificial Sequence
  Description of Artificial Sequence Synthetic oligonucleotide
ugucuacgccauua

SEQ ID NO: 1199 - RNA, 14 nt, Artificial Sequence
  Description of Artificial Sequence Synthetic oligonucleotide
augccugccucuaa

SEQ ID NO: 1200 - RNA, 14 nt, Artificial Sequence
  Description of Artificial Sequence Synthetic oligonucleotide
gauccguggaggua

SEQ ID NO: 1201 - RNA, 14 nt, Artificial Sequence
  Description of Artificial Sequence Synthetic oligonucleotide
gaggcagagacuga

SEQ ID NO: 1202 - RNA, 14 nt, Artificial Sequence
  Description of Artificial Sequence Synthetic oligonucleotide
uaccucaccaagaa

SEQ ID NO: 1203 - RNA, 14 nt, Artificial Sequence
  Description of Artificial Sequence Synthetic oligonucleotide
gccguugccaucua

SEQ ID NO: 1204 - RNA, 14 nt, Artificial Sequence
  Description of Artificial Sequence Synthetic oligonucleotide
cugguggaggugua

SEQ ID NO: 1205 - RNA, 14 nt, Artificial Sequence
  Description of Artificial Sequence Synthetic oligonucleotide
ugcaugucuuccaa

SEQ ID NO: 1206 - RNA, 14 nt, Artificial Sequence
  Description of Artificial Sequence Synthetic oligonucleotide
gcggcacccucaua

SEQ ID NO: 1207 - RNA, 14 nt, Artificial Sequence
  Description of Artificial Sequence Synthetic oligonucleotide
ccgacuucgagaaa

SEQ ID NO: 1208 - RNA, 14 nt, Artificial Sequence
  Description of Artificial Sequence Synthetic oligonucleotide
gacugauccacuua

SEQ ID NO: 1209 - RNA, 14 nt, Artificial Sequence
  Description of Artificial Sequence Synthetic oligonucleotide
agcauacagaguga

SEQ ID NO: 1210 - RNA, 14 nt, Artificial Sequence
  Description of Artificial Sequence Synthetic oligonucleotide
gcagcguccacaca

SEQ ID NO: 1211 - RNA, 14 nt, Artificial Sequence
  Description of Artificial Sequence Synthetic oligonucleotide
ugccugccucuaca

SEQ ID NO: 1212 - RNA, 14 nt, Artificial Sequence
  Description of Artificial Sequence Synthetic oligonucleotide
caccagcauacaga

SEQ ID NO: 1213 - RNA, 14 nt, Artificial Sequence
  Description of Artificial Sequence Synthetic oligonucleotide
gagcuggccuugaa

SEQ ID NO: 1214 - RNA, 14 nt, Artificial Sequence
  Description of Artificial Sequence Synthetic oligonucleotide
uggucaccgacuua

SEQ ID NO: 1215 - RNA, 14 nt, Artificial Sequence
  Description of Artificial Sequence Synthetic oligonucleotide
gccagcaaguguga

SEQ ID NO: 1216 - RNA, 14 nt, Artificial Sequence
  Description of Artificial Sequence Synthetic oligonucleotide
cgcuuccacagaca

SEQ ID NO: 1217 - RNA, 14 nt, Artificial Sequence
  Description of Artificial Sequence Synthetic oligonucleotide
acuucucugccaaa

SEQ ID NO: 1218 - RNA, 14 nt, Artificial Sequence
  Description of Artificial Sequence Synthetic oligonucleotide
uccauggccuucua

SEQ ID NO: 1219 - RNA, 14 nt, Artificial Sequence
  Description of Artificial Sequence Synthetic oligonucleotide
gccgcguccucaaa

SEQ ID NO: 1220 - RNA, 14 nt, Artificial Sequence
  Description of Artificial Sequence Synthetic oligonucleotide
ugaccaccgggaaa

SEQ ID NO: 1221 - RNA, 14 nt, Artificial Sequence
  Description of Artificial Sequence Synthetic oligonucleotide
uagacaccagcaua

SEQ ID NO: 1222 - RNA, 14 nt, Artificial Sequence
  Description of Artificial Sequence Synthetic oligonucleotide
acagccaccuucca

SEQ ID NO: 1223 - RNA, 14 nt, Artificial Sequence
  Description of Artificial Sequence Synthetic oligonucleotide
aggccuggaguuua

SEQ ID NO: 1224 - RNA, 14 nt, Artificial Sequence
  Description of Artificial Sequence Synthetic oligonucleotide
agcaucccguggaa

SEQ ID NO: 1225 - RNA, 14 nt, Artificial Sequence
  Description of Artificial Sequence Synthetic oligonucleotide
aaccuggagcggaa

SEQ ID NO: 1226 - RNA, 14 nt, Artificial Sequence
  Description of Artificial Sequence Synthetic oligonucleotide
gugguggugcugaa

SEQ ID NO: 1227 - RNA, 14 nt, Artificial Sequence
  Description of Artificial Sequence Synthetic oligonucleotide
gugcccgaggagga

SEQ ID NO: 1228 - RNA, 14 nt, Artificial Sequence
  Description of Artificial Sequence Synthetic oligonucleotide
aguuuauucggaaa

SEQ ID NO: 1229 - RNA, 14 nt, Artificial Sequence
  Description of Artificial Sequence Synthetic oligonucleotide
aaggagcauggaaa

SEQ ID NO: 1230 - RNA, 14 nt, Artificial Sequence
  Description of Artificial Sequence Synthetic oligonucleotide
aaagucaaggagca

SEQ ID NO: 1231 - RNA, 19 nt, Artificial Sequence
  Description of Artificial Sequence Synthetic oligonucleotide
ugcaaaacaggucuagaaa

SEQ ID NO: 1232 - RNA, 14 nt, Artificial Sequence
  Description of Artificial Sequence Synthetic oligonucleotide
agaccuguuuugca

SEQ ID NO: 1233 - RNA, 19 nt, Artificial Sequence
  Description of Artificial Sequence Synthetic oligonucleotide
ucaaaagcaaaacaggucu

SEQ ID NO: 1234 - RNA, 14 nt, Artificial Sequence
  Description of Artificial Sequence Synthetic oligonucleotide
uguuuugcuuuuga

SEQ ID NO: 1235 - RNA, 19 nt, Artificial Sequence
  Description of Artificial Sequence Synthetic oligonucleotide
uacaaaagcaaaacagguc

SEQ ID NO: 1236 - RNA, 14 nt, Artificial Sequence
  Description of Artificial Sequence Synthetic oligonucleotide
guuuugcuuuugua

SEQ ID NO: 1237 - RNA, 19 nt, Artificial Sequence
  Description of Artificial Sequence Synthetic oligonucleotide
ucuucaaguuacaaaagca

SEQ ID NO: 1238 - RNA, 14 nt, Artificial Sequence
  Description of Artificial Sequence Synthetic oligonucleotide
uuguaacuugaaga

SEQ ID NO: 1239 - RNA, 19 nt, Artificial Sequence
  Description of Artificial Sequence Synthetic oligonucleotide
ugcaaaacaggucuagaaa

SEQ ID NO: 1240 - RNA, 15 nt, Artificial Sequence
  Description of Artificial Sequence Synthetic oligonucleotide
uagaccuguuuugca

(19) United States
(12) Patent Application Publication
(10) Pub. No.: US 2023/0089502 A1
(43) Pub. Date: Mar. 23, 2023

(54) PRODUCTS AND COMPOSITIONS

(71) Applicant: Sirnaomics, Inc., Gatithersburg, MD (US)

(72) Inventor: Dmitry SAMARSKY, Gaithersburg, MD (US)

(21) Appl. No.: 17/843,657

(22) Filed: Jun. 17, 2022

Related U.S. Application Data
(60) Provisional application No. 63/211,861, filed on Jun. 17, 2021.

(51) Int. Cl. C12N 15/113; A61K 38/36; A61P 7/02; C07K 14/00; C12N 9/64

(52) U.S. Cl. CPC C12N 15/113; A61K 38/36; A61P 7/02; C07K 14/00; C12N 9/6443; C12N 2310/11; C12Y 304/21027

(57) ABSTRACT

Nucleic acid products are provided that modulate, interfere with or inhibit PCSK9 gene expression. The products include compounds that comprise at least a first region of linked nucleosides having at least a first nucleobase sequence that is at least partially complementary to at least a portion of RNA transcribed from a PCSK9 gene, wherein the first nucleobase sequence is selected from the following sequences, or a portion thereof: SEQ ID NOs 1 to 250 or 501 to 543.

### PRODUCTS AND COMPOSITIONS

This application claims priority to U.S. Provisional application Ser. No. 63/211,861, filed Jun. 17, 2021, the contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 4, 2022, is named 4690_0049 C_SequenceListing and is 288 kilobytes in size.

### FIELD

Compositions are provided that modulate, interfere with, or inhibit, proprotein convertase subtilkisin/kexin type 9 (PCSK9) gene expression, together with methods of using the compositions for the treatment, prevention or amelioration of PCSK9-associated disorders such as dyslipidemia, including hypercholesterolemia.

### BACKGROUND

Cholesterol has multiple vital functions, including the maintenance of integrity and fluidity of cell membranes. It furthermore serves a precursor for biosynthetic pathways, including those leading to steroid hormones and vitamin D. Cholesterol is present in the blood, where it occurs mainly in two forms: as component in high density lipoproteins (HDL) and in low density lipoproteins (LDL). Often the HDL cholesterol is referred as “good” or beneficial, while LDL cholesterol, in particular when present in elevated levels, presents a health risk and lead to disease.

Regulation of LDL Cholesterol

Proprotein convertase subtilkisin/kexin type 9 (PCSK9) is a serine protease involved in lipid metabolism. PCSK9 reduces the number of LDL receptors on the surface of liver cells. As a consequence, elevated amounts and/or activity of PCSK9 result in higher blood levels of “bad” LDL cholesterol. This molecular and cellular function of PCSK9 has led to its recognition as a therapeutic target molecule.

Disease

Abnormal amounts of circulating cholesterol, in particular of LDL cholesterol, also referred to as hypercholesterolemia, is a recognized disorder in itself which is inter alia owed to the fact that such abnormal amounts, in particular if they persist over extended periods of time, may result in disorder of the cardiovascular system. More specifically, excess amounts of cholesterol may be deposited on the inner walls of blood vessel which in turn may lead to clogging, wherein the clinical manifestations of such clogging include myocardial infarction, stroke and peripheral artery disease.

Treatment

Established treatments for hypercholestorolemia include the administration of statins. Statins may, however, cause side effects, and certain patients are statin-intolerant. Compounds, methods, and pharmaceutical compositions for the treatment of dyslipidemia are provided.

Double-stranded RNA (dsRNA) able to complementarily bind expressed mRNA has been shown to be able to block gene expression (Fire et al., 1998, Nature. 1998 Feb. 19;391 (6669):806-1 1 and Elbashir et al., 2001, Nature. 2001 May 24;41 1 (6836):494-8) by a mechanism that has been termed RNA interference (RNAi). Short dsRNAs direct gene-specific, post-transcriptional silencing in many organisms, including vertebrates, and have become a useful tool for studying gene function. RNAi is mediated by the RNA-induced silencing complex (RISC), a sequence-specific, multi-component nuclease that destroys messenger RNAs homologous to the silencing trigger loaded into the RISC complex. Interfering RNA (iRNA) such as siRNAs, antisense RNA, and micro-RNA are oligonucleotides that prevent the formation of proteins by gene-silencing i.e. inhibiting gene translation of the protein through degradation of mRNA molecules. Gene-silencing agents are becoming increasingly important for therapeutic applications in medicine.

According to Watts and Corey in the Journal of Pathology (2012; Vol 226, p 365-379) algorithms may be used to design nucleic acid silencing triggers, but these suffer from severe limitations. Further experimentation is needed to identify potent siRNAs, since algorithms do not take into account factors such as tertiary structure of the target mRNA or the involvement of RNA binding proteins. Accordingly, discovery of a potent nucleic acid silencing trigger with minimal off-target effects is a complex process. For the pharmaceutical development of these highly charged molecules it is necessary that they be synthesised economically, distributed to and taken up by target tissues, enter cells and function within acceptable limits of toxicity. We provide herein compounds, methods, and pharmaceutical compositions for the treatment of thromboembolic diseases as described herein, which compounds, methods and compositions comprise oligomeric compounds that modulate, in particular inhibit, gene expression by RNAi.

### SUMMARY

Nucleic acid products are provided that modulate and, in particular, interfere with or inhibit, proprotein convertase subtilkisin/kexin type 9 (PCSK9) gene expression, and associated therapeutic uses. Specific oligomeric compounds and sequences according to the disclosed embodiments are described herein. This summary is not intended to identify key features or essential features of the claimed subject matter, nor is it intended to be used to determine the scope of the claimed subject matter.

### BRIEF DESCRIPTION OF THE DRAWINGS

PCSK9-targeting constructs are labelled as P, p, PCS or PCSK9, followed by a two- or three-digit construct number, wherein numbering adheres to the numbering of the nucleobase sequences shown in the sequence listing as follows: SEQ ID NO of the nucleobase sequence of the antisense region=construct number; SEQ ID NO of the nucleobase sequence of the sense region=250+ construct number. In certain instances, such designation of PCSK9-targeting constructs is followed by information in brackets about the architecture of the construct (dsRNA vs. mxRNA; lengths of regions).

FIG. 1 shows PCSK9 knockdown as compared to negative control in a hepatoma cell line. Nucleobase sequences together with backbone (sugar, phosphate) modifications are given in Tables 1a and 1 b. No ligand attached.

FIG. 2 shows IC₅O concentrations for PCSK9 knockdown. Nucleobase sequences together with backbone (sugar, phosphate) modifications are shown in Tables 1a and 1 b. No ligand is attached.

FIG. 3 shows the concentration dependency of PCSK9 knockdown by particularly advantageous double-stranded constructs (antisense strand: 19 nt; sense strand: 14 nt) carrying a GaINAc ligand. The nucleobase sequence of the 14 nt sense strand differs from that given in the sequence listing (15 nt) in that the 5′-terminal nucleotides is removed. Backbone (sugar, phosphate) modifications are as given in Table 5. M4K4 and TMPRSS6 are negative controls (unrelated targets).

FIG. 4 shows a comparison of two types of double-stranded constructs as described herein. Antisense strand: 19 nt; sense strand: 15 nt when indicated (“(15)”; nucleobase sequence as given in the sequence listing), otherwise 14 nt. Backbone (sugar, phosphate) modifications as given in Table 5. PC1a: positive control. An inclisiran-type molecule has been used which differs from inclisiran in that the ligand is 3xGaINAc. This is for reasons of consistency with constructs as described herein which carry a 3xGaINAc ligand. M4K4 is a negative control.

FIG. 5 shows the concentration dependency of PCSK9 knockdown by selected hairpin molecules (mxRNAs) as described herein. “14-5-14” stands for a 19 nt antisense region connected to a 14 nt sense region (shortened by 1 nt as for FIG. 3), wherein the 5 3′-terminal nucleotides of the antisense region for the loop of the hairpin and the molecules contains a 14 bp duplex region formed by base pairing between the 14 5′-terminal nucleotides of the antisense region with a nucleotides of the sense region. Corresponding nucleobase sequences of the entire hairpin molecules are those set forth in SEQ ID NO: 587 to 590. Backbone (sugar, phosphate) modifications are as given in Table 6. TMPRSS6 is a negative control.

### DETAILED DESCRIPTION AND EMBODIMENTS

The following aspects are non-limiting.

Aspect 1. An oligomeric compound capable of inhibiting expression of PCSK9, wherein this compound comprises at least a first region of linked nucleosides having at least a first nucleobase sequence that is at least partially complementary to at least a portion of RNA transcribed from a PCSK9 gene, wherein the first nucleobase sequence is selected from the following sequences, or a portion thereof: sequences of Table 2a (SEQ ID NOs 1 to 250), or sequences of Table 3a (SEQ ID NOs 501 to 543), wherein the portion advantageously has a length of at least 18 nucleotides.

Particularly advantageous embodiments relate to dsRNAs on the one hand and mxRNAs on the other hand; for further details see the embodiments and their discussion further below. In addition, the antisense and sense regions disclosed herein may serve as building blocks for compounds (muRNAs) which are directed to multiple targets. The general architecture of such compounds is described in WO 2020/065602.

Aspect 2. A composition comprising an oligomeric compound according to aspect 1, and a physiologically acceptable excipient.

Aspect 3. A pharmaceutical composition comprising an oligomeric compound according to aspect 1.

Aspect 4. An oligomeric compound according to aspect 1, for use in human or veterinary medicine or therapy.

Aspect 5. An oligomeric compound according to aspect 1, for use in a method of treating a disease or disorder.

Aspect 6. A method of treating a disease or disorder comprising administration of an oligomeric compound according to aspect 1, to an individual in need of treatment.

Aspect 7. Use of an oligomeric compound according to aspect 1, for use in research as a gene function analysis tool.

Further embodiments (items) are described below by way of example only. These examples represent ways in which the disclosed compositions and methods are put into practice but the skilled artisan will recognize that they are not the only ways in which this could be achieved. It will be understood that the benefits and advantages described herein may relate to one embodiment or may relate to several embodiments. The embodiments are not limited to those that solve any or all of the stated problems or those that have any or all of the stated benefits and advantages.

Features of different aspects and embodiments may be combined as appropriate, as would be apparent to a skilled person, and may be combined with any of the aspects as described herein.

### Definitions

In many instances, the definitions, in addition to the respective definition as such, provide non-exhaustive listings of possible implementations which amount to specific embodiments. Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989.

Unless otherwise indicated, the following terms have the following meanings: As used herein, “excipient” means any compound or mixture of compounds that is added to a composition as provided herein that is suitable for delivery of an oligomeric compound.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety, phosphate-linked nucleosides also being referred to as “nucleotides”.

As used herein, “chemical modification” or “chemically modified” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA. A “naturally occurring sugar moiety” as referred to herein is also termed as an “unmodified sugar moiety”. In particular, such a “naturally occurring sugar moiety” or an “unmodified sugar moiety” as referred to herein has a —H (DNA sugar moiety) or —OH (RNA sugar moiety) at the 2′-position of the sugar moiety, especially a —H (DNA sugar moiety) at the 2′-position of the sugar moiety.

As used herein, “sugar moiety” means a naturally occurring sugar moiety or a modified sugar moiety of a nucleoside. As used herein, “modified sugar moiety” means a substituted sugar moiety or a sugar surrogate.

As used herein, “substituted sugar moiety” means a furanosyl that has been substituted. Substituted sugar moieties include, but are not limited to furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH₂CH₂OCH₃.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose). Duplexes of uniformly modified 2′-fluorinated (ribo) oligonucleotides hybridized to RNA strands are not RNase H substrates while the ara analogs retain RNase H activity.

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2 ‘-carbon and the 4’-carbon of the furanosyl.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding, more specifically hydrogen bonding, with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides can comprise a modified sugar moiety and/or a modified nucleobase.

As used herein, “bicyclic nucleoside” or “BNA” means a nucleoside comprising a bicyclic sugar moiety.

As used herein, “locked nucleic acid nucleoside” or “LNA” means a nucleoside comprising a bicyclic sugar moiety comprising a 4′-CH₂—O-2′bridge.

As used herein, “2 ‘-substituted nucleoside” means a nucleoside comprising a substituent at the 2’-position of the sugar moiety other than H or OH. Unless otherwise indicated, a 2 ‘-substituted nucleoside is not a bicyclic nucleoside.

As used herein, “deoxynucleoside” means a nucleoside comprising 2’—H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more un-modified ribonucleosides (RNA) and/or unmodified deoxyribonucleosides (DNA) and/or one or more modified nucleosides.

As used herein, “modified oligonucleotide” means an oligonucleotide comprising at least one modified nucleoside and/or at least one modified internucleoside linkage.

Advantageous modified internucleoside linkages are those which confer increased stability as compared to the naturally occurring phosphodiesters. “Stability” refers in particular to stability against hydrolysis including enzyme-catalyzed hydrolysis, enzymes including exonucleases and endonucleases.

Advantageous positions for such modified internucleoside linkages include the termini and the hairpin loop of single-stranded oligomeric compounds as described herein. For example, the internucleoside linkages connecting first and second nucleoside and second and third nucleoside counting from the 5′ terminus, and/or the internucleoside linkages connecting first and second nucleoside and second and third nucleoside counting from the 3′ terminus are modified. In addition, a linkage connecting the terminal nucleoside of the 3′ terminus with a ligand, such as GaINAc, may be modified.

As discussed above, advantageous positions are in the hairpin loop of the single-stranded oligomeric compounds. In particular, all linkages, all but one linkages or the majority of linkages in the hairpin loop are modified. As used herein, “linkages in the hairpin loop” designates the linkages between nucleosides which are not engaged in base pairing. For example, in a hairpin loop consisting of five nucleosides, there are four linkages between nucleosides which are not engaged in base pairing. Advantageously, the term “linkages in the hairpin loop” also extends to the linkages connecting the stem to the loop, i.e., those linkages which connect a base-paired nucleoside to a non-based paired nucleoside. Generally, there are two such positions in hairpins and mxRNAs as described herein.

Most advantageous is that modified internucleoside linkages are at both termini and in the hairpin loop.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage. As used herein, “modified internucleoside linkage” means any internucleoside linkage other than a naturally occurring internucleoside linkage. In particular, a “modified internucleoside linkage” as referred to herein can include a modified phosphorous linking group such as a phosphorothioate or phosphorodithioate internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom and can include naturally occurring phosphorous linking groups as present in naturally occurring RNA or DNA, such as phosphodiester linking groups, or modified phosphorous linking groups that are not generally present in naturally occurring RNA or DNA, such as phosphorothioate or phosphorodithioate linking groups. Phosphorus linking groups can therefore include without limitation, phosphodiester, phosphorothioate, phosphorodithioate, phosphonate, methylphosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “oligomeric compound” means a polymeric structure comprising two or more substructures. In certain embodiments, an oligomeric compound comprises an oligonucleotide, such as a modified oligonucletide. In certain embodiments, an oligomeric compound further comprises one or more conjugate groups and/or terminal groups and/or ligands. In certain embodiments, an oligomeric compound consists of an oligonucleotide. In certain embodiments, an oligomeric compound comprises a backbone of one or more linked monomeric sugar moieties, where each linked monomeric sugar moiety is directly or indirectly attached to a heterocyclic base moiety. In certain embodiments, oligomeric compounds may also include monomeric sugar moieties that are not linked to a heterocyclic base moiety, thereby providing abasic sites. Oligomeric compounds may be defined in terms of a nucleobase sequence only, i.e., by specifying the sequence of A, G, C, U (or T). In such a case, the structure of the sugar-phosphate backbone is not partictularly limited and may or may not comprise modified sugars and/or modified phosphates. On the other hand, oligomeric compounds may be more comprehensively defined, i.e, by specifying not only the nucleobase sequence, but also the structure of the backbone, in particular the modification status of the sugars (unmodified, 2′-OMe modified, 2′-F modified etc.) and/or of the phosphates.

As used herein, “terminal group” means one or more atom attached to either, or both, the 3′ end or the 5′ end of an oligonucleotide. In certain embodiments, a terminal group comprises one or more terminal group nucleosides.

As used herein, “conjugate” or “conjugate group” means an atom or group of atoms bound to an oligonucleotide or oligomeric compound. In certain embodiments, a conjugate group links a ligand to a modified oligonucleotide or oligomeric compound. In general, conjugate groups can modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group comprising any atom or group of atoms and which covalently link an oligonucleotide to another portion of the conjugate group. In certain embodiments, the point of attachment on the oligomeric compound is the 3 ‘-oxygen atom of the 3’-hydroxyl group of the 3′ terminal nucleoside of the oligonucleotide. In certain embodiments the point of attachment on the oligomeric compound is the 5′-oxygen atom of the 5′-hydroxyl group of the 5′ terminal nucleoside of the oligonucleotide. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and ligand portion that can comprise one or more ligands, such as a carbohydrate cluster portion, such as an N-Acetyl-Galactosamine, also referred to as “GaINAc”, cluster portion. In certain embodiments, the carbohydrate cluster portion is identified by the number and identity of the ligand. For example, in certain embodiments, the carbohydrate cluster portion comprises 2 GaINAc groups. For example, in certain embodiments, the carbohydrate cluster portion comprises 3 GaINAc groups and this is particularly advantageous. In certain embodiments, the carbohydrate cluster portion comprises 4 GaINAc groups. Such ligand portions are attached to an oligomeric compound via a cleavable moiety, such as a cleavable bond or cleavable nucleoside. The ligands can be arranged in a linear or branched configuration, such as a biantennary or triantennary configurations. A particular carbohydrate cluster has the following formula:

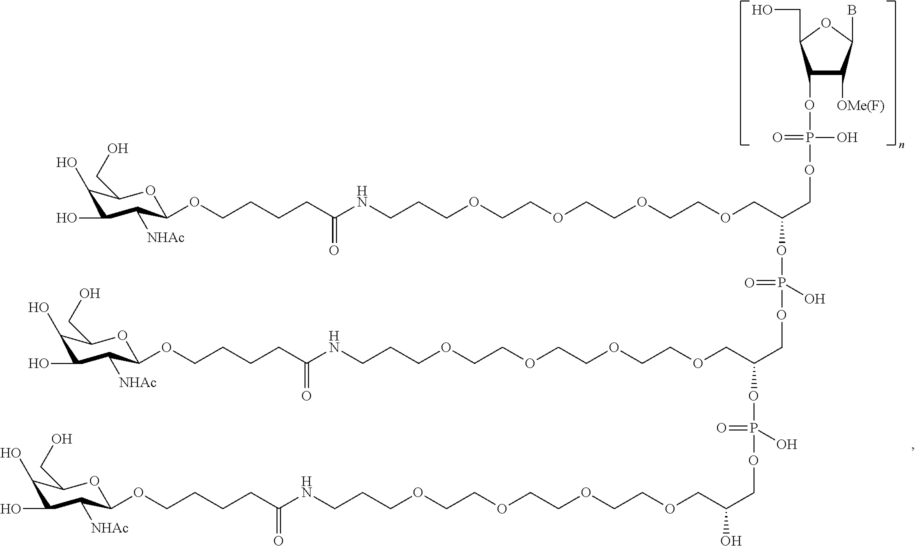

wherein in this structural formula one, two, or three phosphodiester linkages can also be substituted by phosphothionate linkages.

As used herein, “cleavable moiety” means a bond or group that is capable of being cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety comprises a group of atoms having one, two, three, four, or more than four cleavable bonds. In certain embodiments, a cleavable moiety is a phosphodiester linkage.

As used herein, “cleavable bond” means any chemical bond capable of being broken.

As used herein, “carbohydrate cluster” means a compound having one or more carbohydrate residues attached to a linker group.

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative. A carbohydrate is a biomolecule including carbon (C), hydrogen (H) and oxygen (O) atoms. Carbohydrates can include monosaccharide, disaccharides, trisaccharides, tetrasaccharides, oligosaccharides or polysaccharides, such as one or more galactose moieties, one or more lactose moieties, one or more N-Acetyl-Galactosamine moieties, and/or one or more mannose moieties. A particularly advantageous carbohydrate is N-Acetyl-Galactosamine.

As used herein, “strand” means an oligomeric compound comprising linked nucleosides.

As used herein, “single strand” or “single-stranded” means an oligomeric compound comprising linked nucleosides that are connected in a continuous sequence without a break therebetween. Such single strands may include regions of sufficient self-complementarity so as to be capable of forming a stable self-duplex in a hairpin structure.

As used herein, “hairpin” means a single stranded oligomeric compound that includes a duplex formed by base pairing between sequences in the strand that are self-complementary and opposite in directionality.

As used herein, “hairpin loop” means an unpaired loop of linked nucleosides in a hairpin that is created as a result of hybridization of the self-complementary sequences. The resulting structure looks like a loop or a U-shape.

As used herein, “directionality” means the end-to-end chemical orientation of an oligonucleotide based on the chemical convention of numbering of carbon atoms in the sugar moiety meaning that there will be a 5′-end defined by the 5′ carbon of the sugar moiety, and a 3′-end defined by the 3′ carbon of the sugar moiety. In a duplex or double stranded oligonucleotide, the respective strands run in opposite 5′ to 3′ directions to permit base pairing between them.

As used herein, “duplex” means two or more complementary strand regions, or strands, of an oligonucleotide or oligonucleotides, hybridized together by way of non-covalent, sequence-specific interaction therebetween. Most commonly, the hybridization in the duplex will be between nucleobases adenine (A) and thymine (T), and/or (A) adenine and uracil (U), and/or guanine (G) and cytosine (C). The duplex may be part of a single stranded structure, wherein self-complementarity leads to hybridization, or as a result of hybridization between respective strands in a double stranded construct.

As used herein, “double strand” or “double stranded” means a pair of oligomeric compounds that are hybridized to one another. In certain embodiments, a double-stranded oligomeric compound comprises a first and a second oligomeric compound.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a sequence to which an oligomeric compound is intended to hybridize to result in a desired activity with respect to PCSK9 expression. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In certain embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80%> complementary. In certain embodiments, complementary oligomeric compounds or regions are 90%> complementary. In certain embodiments, complementary oligomeric compounds or regions are at least 95% complementary. In certain embodiments, complementary oligomeric compounds or regions are 100% complementary.

As used herein, “self-complementarity” in reference to oligomeric compounds means a compound that may fold back on itself, creating a duplex as a result of nucleobase hybridization of internal complementary strand regions. Depending on how close together and/or how long the strand regions are, then the compound may form hairpin loops, junctions, bulges or internal loops.

As used herein, “mismatch” means a nucleobase of an oligomeric compound that is not capable of pairing with a nucleobase at a corresponding position of a target sequence, or at a corresponding position of the oligomeric compound itself when the oligomeric compound hybridizes as a result of self-complementarity, when the oligomeric compound and the target sequence and/or self-complementary regions of the oligomeric compound, are aligned. As used herein, “hybridization” means the pairing of complementary oligomeric compounds (e.g., an oligomeric compound and its target sequence). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mismatches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “type of modification” in reference to a nucleoside or a nucleoside of a “type” means the chemical modification of a nucleoside and includes modified and un-modified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified naturally occurring RNA nucleoside are “differently modified,” even though the naturally occurring nucleoside is unmodified. Likewise, DNA and RNA oligonucleotides are “differently modified,” even though both are naturally-occurring unmodified nucleosides. Nucleosides that are the same but for comprising different nucleobases are not differently modified. For example, a nucleoside comprising a 2′-OMe modified sugar moiety and an unmodified adenine nucleobase and a nucleoside comprising a 2′-OMe modified sugar moiety and an unmodified thymine nucleobase are not differently modified.

As used herein, “the same type of modifications” refers to modifications that are the same as one another, including absence of modifications. Thus, for example, two unmodified RNA nucleosides have “the same type of modification,” even though the RNA nucleosides are unmodified. Such nucleosides having the same type modification may comprise different nucleobases.

As used herein, “region” or “regions”, or “portion” or “portions”, mean a plurality of linked nucleosides that have a function or character as defined herein, in particular with reference to the claims and definitions as provided herein. Typically such regions or portions comprise at least 10, at least 11, at least 12 or at least 13 linked nucleosides. For example, such regions can comprise 13 to 20 linked nucleosides, such as 13 to 16 or 18 to 20 linked nucleosides. Typically a first region as defined herein consists essentially of 18 to 20 nucleosides and a second region as defined herein consists essentially of 13 to 16 linked nucleosides.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2 ′-position of a nucleoside other than H or OH). Substituent groups can be protected or unprotected. In certain embodiments, compounds of the present disclosure have substituents at one or at more than one position of the parent compound. Substituents may also be further substituted with other substituent groups and may be attached directly or via a linking group such as oxygen or an alkyl or hydrocarbyl group to a parent compound.

Such substituents can be present as the modification on the sugar moiety, in particular a substituent present at the 2′-position of the sugar moiety. Unless otherwise indicated, groups amenable for use as substituents include without limitation, one or more of halo, hydroxyl, alkyl, alkenyl, alkynyl, acyl, carboxyl, alkoxy, alkoxyalkylene and amino substituents. Certain substituents as described herein can represent modifications directly attached to a ring of a sugar moiety (such as a halo, such as fluoro, directly attached to a sugar ring), or a modification indirectly linked to a ring of a sugar moiety by way of an oxygen linking atom that itself is directly linked to the sugar moiety (such as an alkoxyalkylene, such as methoxyethylene, linked to an oxygen atom, overall providing an MOE substituent as described herein attached to the 2′-position of the sugar moiety).

As used herein, “alkyl,” as used herein, means a saturated straight or branched monovalent C₁₋₆ hydrocarbon radical, with methyl being a most advantageous alkyl as a substituent at the 2′-position of the sugar moiety. The alkyl group typically attaches to an oxygen linking atom at the 2′poisition of the sugar, therefore, overall providing a —Oalkyl substituent, such as an —OCH₃ substituent, on a sugar moiety of an oligomeric compound as described herein. This will be well understood be a person skilled in the art.

As used herein, “alkylene” means a saturated straight or branched divalent hydrocarbon radical of the general formula —CₙH₂ₙ— where n is 1-6. Advantageously the alkylenes are methylene or ethylene.

As used herein, “alkenyl” means a straight or branched unsaturated monovalent C₂₋₆ hydrocarbon radical, with ethenyl or propenyl being most advantageous alkenyls as a substituent at the 2′-position of the sugar moiety. As will be well understood in the art, the degree of unsaturation that is present in an alkenyl radical is the presence of at least one carbon to carbon double bond. The alkenyl group typically attaches to an oxygen linking atom at the 2′-position of the sugar, therefore, overall providing a —Oalkenyl substituent, such as an —OCH₂CH═CH₂ substituent, on a sugar moiety of an oligomeric compound as described herein. This will be well understood be a person skilled in the art.

As used herein, “alkynyl” means a straight or branched unsaturated C₂₋₆ hydrocarbon radical, with ethynyl being a most advantageous alkynyl as a substituent at the 2′-position of the sugar moiety. As will be well understood in the art, the degree of unsaturation that is present in an alkynyl radical is the presence of at least one carbon to carbon triple bond. The alkynyl group typically attaches to an oxygen linking atom at the 2′-position of the sugar, therefore, overall providing a —Oalkynyl substituent on a sugar moiety of an oligomeric compound as described herein. This will be well understood be a person skilled in the art.

As used herein, “carboxyl” is a radical having a general formula —CO₂H.

As used herein, “acyl” means a radical formed by removal of a hydroxyl group from a carboxyl radical as defined herein and has the general Formula —C(O)—X where X is typically C₁₋₆ alkyl. As used herein, “alkoxy” means a radical formed between an alkyl group, such as a C₁₋₆ alkyl group, and an oxygen atom wherein the oxygen atom is used to attach the alkoxy group either to a parent molecule (such as at the 2′-position of a sugar moiety), or to another group such as an alkylene group as defined herein. Examples of alkoxy groups include without limitation, methoxy, ethoxy, propoxy, isopropoxy, n-butoxy, sec-butoxy and tert-butoxy. Alkoxy groups as used herein may optionally include further substituent groups.

As used herein, alkoxyalkylene means an alkoxy group as defined herein that is attached to an alkylene group also as defined herein, and wherein the oxygen atom of the alkoxy group attaches to the alkylene group and the alkylene attaches to a parent molecule. The alkylene group typically attaches to an oxygen linking atom at the 2′-position of the sugar, therefore, overall providing a —Oalkylenealkoxy substituent, such as an —OCH₂CH₂OCH₃ substituent, on a sugar moiety of an oligomeric compound as described herein. This will be well understood by a person skilled in the art and is generally referred to as an MOE substituent as defined herein and as known in the art.

As used herein, “amino” includes primary, secondary and tertiary amino groups.

As used herein, “halo” and “halogen,” mean an atom selected from fluorine, chlorine, bromine and iodine.

As used herein, the term “mxRNA” is in particular understood as defined in WO 2020/044186 A2 which is incorporated by reference herein in its entirety.

It will also be understood that oligomeric compounds as described herein may have one or more non-hybridizing nucleosides at one or both ends of one or both strands (overhangs) and/or one or more internal non-hybridizing nucleosides (mismatches) provided there is sufficient complementarity to maintain hybridization under physiologically relevant conditions.

Alternatively, oligomeric compounds as described herein may be blunt ended at at least one end.

The term “comprising” is used herein to mean including the method steps or elements identified, but that such steps or elements do not comprise an exclusive list and as such there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

The following items are provided:

1. An oligomeric compound capable of inhibiting expression of PCSK9, wherein the compound comprises at least a first region of linked nucleosides having at least a first nucleobase sequence that is at least partially complementary to at least a portion of RNA transcribed from a PCSK9 gene, wherein the first nucleobase sequence is selected from the following sequences, or a portion thereof: SEQ ID NOs 1 to 250, or SEQ ID NOs 501 to 543, wherein the portion advantageously has a length of at least 18 nucleotides.

The first region is also referred to as antisense region, and the second region is also referred to as sense region. As disclosed in specific embodiments below, the two regions may be located on the same strand, advantageously in an adjacent manner. This gives rise to hairpin molecules, also referred to as mxRNAs. On the other hand, the two regions may be located on separate strands which gives rise to double-stranded RNAs (dsRNAs), wherein advantageously each strand consists of the respective region.

Moreover, the regions may serve as building blocks for muRNAs (see above). In other words, the first and the second region as defined herein may be used, in accordance with the following definition of muRNAs, as first and third regions, respectively:

A nucleic acid construct (muRNA) comprising at least:
(a) a first nucleic acid portion that is at least partially complementary to at least a first portion of an RNA which is transcribed from a PCSK9 gene;
(b) a second nucleic acid portion that is at least partially complementary to at least a second portion of an RNA which is transcribed from another gene;
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith.

Specific embodiments of and further aspects relating to muRNAs are disclosed in WO 2020/065602.

2. The oligomeric compound according to item 1, which further comprises at least a second region of linked nucleosides having at least a second nucleobase sequence that is at least partially complementary to the first nucleobase sequence and is selected from the following sequences, or a portion thereof: SEQ ID NOs 251 to 500, or SEQ ID NOs 544 to 586, wherein the portion advantageously has a length of at least 11 nucleotides.

3. The oligomeric compound according to item 1 or 2, wherein the first nucleobase sequence is selected from the following sequences, or a portion thereof: SEQ ID NOs 13, 31, 76, 127, 55, 29, 28, 53, 44, 49, 58, 94, 52, 57, 43, 36, 21, 35, 232, 87, 48, 139, 46, 233, 34, 100, and 77.

4. The oligomeric compound according to item 3, wherein the second nucleobase sequence is selected from the following sequences, or a portion thereof: SEQ ID NOs 263, 281, 326, 377, 305, 279, 278, 303, 294, 299, 308, 344, 302, 307, 293, 286, 271, 285, 482, 337, 298, 389, 296, 483, 284, 350, and 327, and wherein the portion is advantageously 14 nucleosides long and lacks the 5′-terminal nucleobase as set forth in the SEQ ID NOs.

5. The oligomeric compound according to any of items 1 to 4, wherein the first nucleobase sequence is selected from the following sequences, or a portion thereof: SEQ ID NOs 29, 44, 46, 52, 53, 55, and 57; advantageously SEQ ID NOs: 29, 44, 53 and 57, more advantageously SEQ ID NO: 29.

This embodiments defines antisense nucleobase sequences which provide for surprisingly outstanding performance. For evidence, reference is made to the Examples.

6. The oligomeric compound according to item 5, wherein the second nucleobase sequence is selected from the following sequences, or a portion thereof: SEQ ID NOs 279, 294, 296, 302, 303, 305, and 307, advantageously SEQ ID NOs: 279, 294, 303 and 307, more advantageously SEQ ID NO: 279, and wherein the portion is advantageously 14 nucleosides long and lacks the 5′-terminal nucleobase as set forth in the SEQ ID NOs.

7. The oligomeric compound according to any of items 1 to 6, wherein the first region of linked nucleosides consists essentially of 18 to 35, advantageously 18 to 20, more advantageously 18 or 19, and yet more advantageously 19 linked nucleosides.

8. The oligomeric compound according to any of items 2 to 7, wherein the second region of linked nucleosides consists essentially of 11 to 35, advantageously 11 to 20, more advantageously 13 to 16, and yet more advantageously 14 or 15, most advantageously 14 linked nucleosides.

9. The oligomeric compound according to any of items 2 to 8, which comprises at least one complementary duplex region that comprises at least a portion of the first nucleoside region directly or indirectly linked to at least a portion of the second nucleoside region, wherein advantageously the duplex region has a length of 11 to 19, more advantageously 14 to 19, and yet more advantageously 14 or 15 base pairs, most advantageously 14 base pairs, wherein optionally there is one mismatch within the duplex region.

10. The oligomeric compound according to item 9, wherein each of the first and second nucleoside regions has a 5′ to 3′ directionality thereby defining 5′ and 3′ regions respectively thereof.

11. The oligomeric compound according to item 10, wherein the 5′ region of the first nucleoside region is directly or indirectly linked to the 3′ region of the second nucleoside region, for example by complementary base pairing, and/or wherein the 3′ region of the first nucleoside region is directly or indirectly linked to the 5′ region of the second nucleoside region, wherein advantageously the 5′ terminal nucleoside of the first nucleoside region base pairs with the 3′ terminal nucleoside of the second nucleoside region.

12. The oligomeric compound according to any of items 1 to 11, which further comprises one or more ligands.

13. The oligomeric compound according to item 12, wherein the one or more ligands are conjugated to the second nucleoside region and/or the first nucleoside region.

14. The oligomeric compound according to item 13, as dependent on item 10, wherein the one or more ligands are conjugated at the 3′ region, advantageously to the 3′ end of the second nucleoside region and/or of the first nucleoside region, and/or to the 5′ end of the second nucleoside region.

15. The oligomeric compound according to any of items 12 to 14, wherein the one or more ligands are any cell directing moiety, such as lipids, carbohydrates, aptamers, vitamins and/or peptides that bind cellular membrane or a specific target on cellular surface.

16. The oligomeric compound according to item 15, wherein the one or more ligands comprise one or more carbohydrates.

17. The oligomeric compound according to item 16, wherein the one or more carbohydrates can be a monosaccharide, disaccharide, trisaccharide, tetrasaccharide, oligosaccharide or polysaccharide.

18. The oligomeric compound according to item 17, wherein the one or more carbohydrates comprise or consist of one or more hexose moieties.

19. The oligomeric compound according to item 18, wherein the one or more hexose moieties are one or more galactose moieties, one or more lactose moieties, one or more N-Acetyl-Galactosamine moieties, and/or one or more mannose moieties.

20. The oligomeric compound according to item 19, wherein the one or more carbohydrates comprise one or more N-Acetyl-Galactosamine moieties.

21. The oligomeric compound according to item 20, which comprises two or three N-Acetyl-Galactosamine moieties, advantageously three.

22. The oligomeric compound according to any of items 12 to 21, wherein the one or more ligands are attached to the oligomeric compound, advantageously to the second nucleoside region thereof, in a linear configuration, or in a branched configuration.

23. The oligomeric compound according to item 22, wherein the one or more ligands are attached to the oligomeric compound as a biantennary or triantennary configuration.

24. The oligomeric compound according to any one of items 1 to 23, wherein the compound consists of the first region of linked nucleosides and the second region of linked nucleosides. Each of the regions may constitute a separate strand, thereby giving rise to a double-stranded RNA (dsRNA). Particularly advantageous dsRNAs are those with a length of the first strand of 19 nucleosides and a length of the second region of 14 or 15, advantageously 14 nucleosides. When used for defining the length of a region or strand, the terms “nucleoside” and “nucleotide” (sometimes abbreviated “nt”) are used equivalently.

25. The oligomeric compound according to any one of items 9 to 24, wherein the oligomeric compound comprises a single strand comprising the first and second nucleoside regions, wherein the single strand dimerises whereby at least a portion of the first nucleoside region is directly or indirectly linked to at least a portion of the second nucleoside region so as to form the at least partially complementary duplex region.

The term “dimerise” in relation to the embodiment above refers to the formation of intramolecular base pairs between first and second region.

26. The oligomeric compound according to item 25, wherein the first nucleoside region has a greater number of linked nucleosides compared to the second nucleoside region, whereby the additional number of linked nucleosides of the first nucleoside region form a hairpin loop linking the first and second nucleoside regions.

Such compounds are also referred to as hairpins or mxRNAs herein.

27. The oligomeric compound according to item 26, as dependent on claim 10, whereby the hairpin loop is present at the 3′ region of the first nucleoside region, and/or wherein the hairpin loop comprises 4 or 5 linked nucleosides.

Particularly advantageous is a length of the first region of 19 nucleosides, of the second region of 14 nucleotides, and of the hairpin loop of 5 nucleotides, wherein the 5 nucleotides in the hairpin are the 5 3′-terminal nucleosides of the first region. Such molecular architecture of a hairpin or mxRNA is also designated “14-5-14” herein.

28. The oligomeric compound according to item 26 or 27, the compound has a nucleobase sequence selected from SEQ ID NOs: 587 to 590, and advantageously is selected from Table 6.

29. The oligomeric compound according to any of items 1 to 28, which comprises internucleoside linkages and wherein at least one internucleoside linkage is a modified internucleoside linkage.

Specific modified internucleoside linkages are the subject of the specific embodiments which follow. Certain modified internucleoside linkages are known in the art and described in, for example, Hu et al., Signal Transduction and Targeted Therapy (2020)5:101.

30. The oligomeric compound according to item 29, wherein the modified inter-nucleoside linkage is a phosphorothioate or phosphorodithioate internucleoside linkage.

31. The oligomeric compound according to item 30, which comprises 1 to 15 phosphorothioate or phosphorodithioate internucleoside linkages.

32. The oligomeric compound according to item 31, which comprises 7, 8, 9 or 10 phosphorothioate or phosphorodithioate internucleoside linkages.

33. The oligomeric compound according to any of items 30 to 32, as dependent on item 10, which comprises one or more phosphorothioate or phosphorodithioate internucleoside linkages at the 5′ region of the first nucleoside region.

34. The oligomeric compound according to any of items 30 to 33, as dependent on item 10, which comprises one or more phosphorothioate or phosphorodithioate internucleoside linkages at the 5′ region of the second nucleoside region.

35. The oligomeric compound according to any of items 30 to 34, as dependent on item 26, which comprises phosphorothioate or phosphorodithioate internucleoside linkages between at least two, advantageously at least three, advantageously at least four, advantageously at least five, adjacent nucleosides of the hairpin loop, dependent on the number of nucleotides present in the hairpin loop.

36. The oligomeric compound according to item 35, which comprises a phosphorothioate or phosphorodithioate internucleoside linkage between each adjacent nucleoside that is present in the hairpin loop.

37. The oligomeric compound according to any of items 1 to 36, wherein at least one nucleoside comprises a modified sugar.

Specific modified sugars are subject of the embodiments which follow. Certain modified sugars are known in the art and described in, for example, Hu et al., Signal Transduction and Targeted Therapy (2020)5:101.

38. The oligomeric compound according to item 37, wherein the modified sugar is selected from 2′ modified sugars, locked nucleic acid (LNA) sugar, (S)-constrained ethyl bicyclic nucleic acid sugar, tricyclo-DNA sugar, morpholino, unlocked nucleic acid (UNA) sugar, and glycol nucleic acid (GNA) sugar.

39. The oligomeric compound according to item 38, wherein the 2′ modified sugar is selected from 2′-O-methyl modified sugar, 2′-O-methoxyethyl modified sugar, 2′-F modified sugar, 2′-arabino-fluoro modified sugar, 2′-O-benzyl modified sugar, and 2′-O-methyl-4-pyridine modified sugar.

40. The oligomeric compound according to item 39, wherein at least one modified sugar is a 2′-O-methyl modified sugar.

41. The oligomeric compound according to item 39 or 40, wherein at least one modified sugar is a 2′-F modified sugar.

42. The oligomeric compound of item 40 or 41, wherein the sugar is ribose.

43. The oligomeric compound according to any of items 40 to 42, as dependent on item 10, wherein sugars of the nucleosides at any of positions 2 and 14 downstream from the first nucleoside of the 5′ region of the first nucleoside region, do not contain 2′-O-methyl modifications.

44. The oligomeric compound according to any of items 40 to 43, as dependent on item 10, wherein sugars of the nucleosides of the second nucleoside region, that correspond in position to any of the nucleosides of the first nucleoside region at any of positions 9 to 11 downstream from the first nucleotide of the 5′ region of the first nucleoside region, do not contain 2′-O-methyl modifications.

45. The oligomeric compound of any one of items 40 to 44, wherein the 3′ terminal position of the second nucleoside region does not contain a 2′-O-methyl modification.

46. The oligomeric compound according to item 44 or 45, wherein sugars of the nucleosides at any of positions 2 and 14 downstream from the first nucleoside of the 5′ region of the first nucleoside region, contain 2′-F modifications.

47. The oligomeric compound according to any of items 44 to 46, wherein sugars of the nucleosides of the second nucleoside region, that correspond in position to any of the nucleosides of the first nucleoside region at any of positions 9 to 11 downstream from the first nucleoside of the 5′ region of the first nucleoside region, contain 2′-F modifications.

48. The oligomeric compound of item 46 or 47, wherein the 3′ terminal position of the second nucleoside region contains a 2′-F modification.

49. The oligomeric compound according to any of items 42 to 48, as dependent on item 10, wherein one or more of the odd numbered nucleosides starting from the 5′ region of the first nucleoside region are modified, and/or wherein one or more of the even numbered nucleotides starting from the 5′ region of the first nucleoside region are modified, wherein typically the modification of the even numbered nucleotides is a second modification that is different from the modification of odd numbered nucleotides.

50. The oligomeric compound according to item 49, wherein one or more of the odd numbered nucleosides starting from the 3′ region of the second nucleoside region are modified by a modification that is different from the modification of odd numbered nucleosides of the first nucleoside region.

51. The oligomeric compound according to item 49 or 50, wherein one or more of the even numbered nucleosides starting from the 3′ region of the second nucleoside region are modified by a modification that is different from the modification of even numbered nucleosides of the first nucleoside region according to item 53.

52. The oligomeric compound according to any of items 49 to 51, wherein at least one or more of the modified even numbered nucleosides of the first nucleoside region is adjacent to at least one or more of the differently modified odd numbered nucleosides of the first nucleoside region.

53. The oligomeric compound according to any of items 49 to 52, wherein at least one or more of the modified even numbered nucleosides of the second nucleoside region is adjacent to at least one or more of the differently modified odd numbered nucleosides of the second nucleoside region.

54. The oligomeric compound according to any of items 49 to 53, wherein sugars of one or more of the odd numbered nucleosides starting from the 5′ region of the first nucleoside region are 2′-O-methyl modified sugars.

55. The oligomeric compound according to any of items 49 to 54, wherein one or more of the even numbered nucleosides starting from the 5′ region of the first nucleoside region are 2′-F modified sugars.

56. The oligomeric compound according to any of items 49 to 55, wherein sugars of one or more of the odd numbered nucleosides starting from the 3′ region of the second nucleoside region are 2′-F modified sugars.

57. The oligomeric compound according to any of items 49 to 56, wherein one or more of the even numbered nucleosides starting from the 3′ region of the second nucleoside region are 2′-O-methyl modified sugars.

58. The oligomeric compound according to any of items 37 to 57, wherein sugars of a plurality of adjacent nucleosides of the first nucleoside region are modified by a common modification.

59. The oligomeric compound according to any of items 37 to 58, wherein sugars of a plurality of adjacent nucleosides of the second nucleoside region are modified by a common modification.

60. The oligomeric compound according to any of items 49 to 59, as dependent on item 26, wherein sugars of a plurality of adjacent nucleosides of the hairpin loop are modified by a common modification.

61. The oligomeric compound according to any of items 58 to 60, wherein the common modification is a 2′-F modified sugar.

62. The oligomeric compound according to any of items 58 to 60, wherein the common modification is a 2′-O-methyl modified sugar.

63. The oligomeric compound according to item 62, wherein the plurality of adjacent 2′-O-methyl modified sugars are present in at least eight adjacent nucleosides of the first and/or second nucleoside regions.

64. The oligomeric compound according to item 62, wherein the plurality of adjacent 2′-O-methyl modified sugars are present in three or four adjacent nucleosides of the hairpin loop.

65. The oligomeric compound according to item 37, as dependent on item 26, wherein the hairpin loop comprises at least one nucleoside having a modified sugar.

66. The oligomeric compound according to item 66, wherein the at least one nucleoside is adjacent a nucleoside with a differently modified sugar.

67. The oligomeric compound according to item 66, wherein the modified sugar is a 2′-O-methyl modified sugar, and the differently modified sugar is a 2′-F modified sugar.

68. The oligomeric compound according to any of items 1 to 67, which comprises one or more nucleosides having an un-modified sugar moiety.

69. The oligomeric compound according to item 68, wherein the unmodified sugar is present in the 5′ region of the second nucleoside region.

70. The oligomeric compound according to item 68 or 69, as dependent on item 26, wherein the unmodified sugar is present in the hairpin loop.

71. The oligomeric compound according to any of items 1 to 70, wherein one or more nucleosides of the first nucleoside region and/or the second nucleoside region is an inverted nucleoside and is attached to an adjacent nucleoside via the 3′ carbon of its sugar and the 3′ carbon of the sugar of the adjacent nucleoside, and/or one or more nucleosides of the first nucleoside region and/or the second nucleoside region is an inverted nucleoside and is attached to an adjacent nucleoside via the 5′ carbon of its sugar and the 5′ carbon of the sugar of the adjacent nucleoside.

72. The oligomeric compound according to any of items 1 to 71, which is blunt ended.

73. The oligomeric compound according to any of items 1 to 71, wherein either the first or second nucleoside region has an overhang.

74. The oligomeric compound according to any one of the preceding items, wherein the first region of linked nucleotides is selected from Table 2b or Table 3b, advantageously from Table 1a, more advantageously from Table 5.

75. The oligomeric compound according to any one of the preceding items, wherein the second region of linked nucleotides is selected from Table 2d or Table 3d, advantageously from Table 1 b, more advantageously from Table 5.

76. A composition comprising an oligomeric compound according to any of items 1 to 75, and a physiologically acceptable excipient.

77. A pharmaceutical composition comprising an oligomeric compound according to any of items 1 to 75.

78. The pharmaceutical composition of item 77, further comprising a pharmaceutically acceptable excipient, diluent, antioxidant, and/or preservative.

79. The pharmaceutical composition of item 77 or 78, wherein the oligomeric compound is the only pharmaceutically active agent.

80. The pharmaceutical composition of item 79, wherein the pharmaceutical composition is to be administered to patients or individuals which are statin-intolerant and/or for whom statins are contraindicated.

81. The pharmaceutical composition of item 77 or 78, wherein the pharmaceutical composition furthermore comprises one or more further pharmaceutically active agents.

82. The pharmaceutical composition of item 81, wherein the further pharmaceutically active agent(s) is/are a further oligomeric compound which is directed to a target different from PCSK9 and/or a lipid-lowering agent distinct from the oligomeric compound, wherein the lipid-lowering agent is advantageously a statin or ezetimib.

83. The pharmaceutical composition of item 81 or 82, wherein the oligomeric compound and the further pharmaceutically active agent(s) are to be administered concomitantly or in any order.

84. An oligomeric compound according to any of items 1 to 75, for use in human or veterinary medicine or therapy.

85. An oligomeric compound according to any of items 1 to 75, for use in a method of treating a disease or disorder.

86. The compound for use of item 85, wherein the disease or disorder is a PCSK9-associated disease or disorder, or a disease or disorder requiring reduction of low-density lipoprotein (LDL) cholesterol, the disease or disorder advantageously being selected from dyslipidemia including mixed dyslipidemia, hypercholesterolemia, heterozygous familial hypercholesterolemia, non-familial hypercholesterolemia; atherosclerosis; and atherosclerotic cardiovascular disease (ASCVD) including myocardial infarction, stroke and peripheral arterial disease.

87. A method of treating a disease or disorder comprising administration of an oligomeric compound according to any of items 1 to 75, to an individual in need of treatment.

88. The method according to item 87, wherein the oligomeric compound is administered subcutaneously or intravenously to the individual.

89. Use of an oligomeric compound according to any of items 1 to 75, for use in research as a gene function analysis tool.

The following Tables show nucleobase sequences of antisense and sense strands of oligomeric compounds as described herein, and definitions of antisense and sense strands of modified oligomeric compounds (the notation including nucleobase sequence, sugar modifications, and, where applicable, modified phosphates).

The notation used is common in the art and as the following meaning:

A represents adenine;

U represents uracil;

C represents cytosine;

G represents guanine.

P represents a terminal phosphate group;

m represents a methyl modification at the 2′ position of the sugar of the underlying nucleoside;

f represents a fluoro modification at the 2′ position of the sugar of the underlying nucleoside.

r indicates an unmodified (2′-OH) ribonucleotide;

(ps) or # or * represents a phosphorothioate inter-nucleoside linkage;

i represents an inverted inter-nucleoside linkage, which can be either 3′-3′, or 5′-5′;

vp represents vinyl phosphonate;

mvp represents methyl vinyl phosphonate;

3xGaINAc or Mono-Galnac-PAHMono-Galnac-PAHMono-Galnac-PA/ represents a trivalent GaINAc, advantageously a “toothbrush” moiety as disclosed herein.

Sometimes, nucleoside are in square brackets for better reading. They do not indicate structural elements or modifications.

To the extent displayed, the presence of a 5′-terminal phosphate (“P”) is optional. On the other hand, to the extent a 5′-terminal phosphate is not displayed, its presence is optional as well. Generally, there is no requirement for a 5′-terminal phosphate in compounds to be administered to mammalian cells, since a mammalian kinase would add a 5′-terminal phosphate in case of its absence.

In Example 2, the nucleobase sequences of antisense and sense strands of 27 specific oligomeric compounds as described herein are given. These are also the subject of specific embodiments disclosed further above. For antisense sequences, the construct number coincides with the SEQ ID NO in the attached sequence listing. For sense sequences, the number of the corresponding entry in the sequence listing is construct number ±250.

Tables 1a and 1 b below shows the sugar-phosphate backbone modifications of the antisense and sense strands of the 27 constructs.

TABLE 1a

| | SEQ | |
|---|---|---|
| | ID | |
| ID | NO: | Antisense strand modified |
| P44as | 591 | [mU][#][mC][#][mU][mU][mC][mA][mA][mG][mU][mU][mA][mC][mA][fA][#]mA[#]mA[#]mG[#]mC[#]rA |
| P46as | 592 | [mU][#][mA][#][mA][mA][mA][mG][mC][mA][mA][mA][mA][mC][mA][fG][#]mG[#]mU[#]mC[#]mU[#]rA |
| P53as | 593 | [mU][#][mA][#][mC][mA][mA][mA][mA][mG][mC][mA][mA][mA][mA][fC][#]mA[#]mG[#]mG[#]mU[#]rC |
| P29as | 594 | [mU][#][mG][#][mC][mA][mA][mA][mA][mC][mA][mG][mG][mU][mC][fU][#]mA[#]mG[#]mA[#]mA[#]rA |
| P57as | 595 | [mU][#][mC][#][mA][mA][mA][mA][mG][mC][mA][mA][mA][mA][mC][fA][#]mG[#]mG[#]mU[#]mC[#]rU |
| P52as | 596 | [mU][#][mA][#][mG][mA][mA][mU][mA][mA][mA][mU][mA][mU][mC][fU][#]mU[#]mC[#]mA[#]mA[#]rG |
| P55as | 597 | [mU][#][mA][#][mA][mA][mG][mC][mA][mA][mA][mA][mC][mA][mG][fG][#]mU[#]mC[#]mU[#]mA[#]rG |
| P232as | 598 | [mU][#][mU][#][mC][mU][mU][mC][mA][mA][mG][mU][mU][mA][mC][fA][#]mA[#]mA[#]mA[#]mG[#]rC |
| P58as | 599 | [mU][#][mC][#][mA][mA][mG][mU][mU][mA][mC][mA][mA][mA][mA][fG][#]mC[#]mA[#]mA[#]mA[#]rA |
| P36as | 600 | [mU][#][mC][#][mA][mA][mA][mA][mC][mA][mG][mG][mU][mC][mU][fA][#]mG[#]mA[#]mA[#]mA[#]rA |
| P49as | 601 | [mU][#][mA][#][mU][mA][mA][mA][mU][mA][mU][mC][mU][mU][mC][fA][#]mA[#]mG[#]mU[#]mU[#]rA |
| P233as | 602 | [mU][#][mU][#][mU][mA][mC][mA][mA][mA][mA][mG][mC][mA][mA][fA][#]mA[#]mC[#]mA[#]mG[#]rG |
| P48as | 603 | [mU][#][mG][#][mU][mG][mA][mC][mA][mC][mA][mA][mA][mG][mC][fA][#]mG[#]mG[#]mU[#]mG[#]rC |
| P28as | 604 | [mU][#][mU][#][mU][mC][mA][mA][mG][mU][mU][mA][mC][mA][mA][fA][#]mA[#]mG[#]mC[#]mA[#]rA |
| P34as | 605 | [mU][#][mA][#][mA][mA][mU][mA][mU][mC][mU][mU][mC][mA][mA][fG][#]mU[#]mU[#]mA[#]mC[#]rA |
| P31as | 606 | [mU][#][mA][#][mU][mA][mA][mA][mU][mG][mU][mC][mU][mG][mC][fU][#]mU[#]mG[#]mC[#]mU[#]rU |
| P13as | 607 | [mU][#][mC][#][mC][mG][mA][mA][mU][mA][mA][mA][mC][mU][mC][fC][#]mA[#]mG[#]mG[#]mC[#]rC |
| P35as | 608 | [mU][#][mG][#][mG][mA][mA][mC][mC][mA][mU][mU][mU][mU][mA][fA][#]mA[#]mG[#]mC[#]mU[#]rC |
| P43as | 609 | [mU][#][mC][#][mC][mA][mG][mA][mA][mU][mA][mA][mA][mU][mA][fU][#]mC[#]mU[#]mU[#]mC[#]rA |
| P127as | 610 | [mU][#][mA][#][mU][mA][mU][mC][mU][mU][mC][mA][mA][mG][mU][fU][#]mA[#]mC[#]mA[#]mA[#]rA |
| P100as | 611 | [mU][#][mC][#][mU][mC][mA][mG][mU][mU][mC][mC][mU][mG][mC][fU][#]mG[#]mU[#]mG[#]mU[#]rG |
| P76as | 612 | [mU][#][mC][#][mU][mA][mC][mA][mA][mA][mA][mC][mC][mC][mA][fG][#]mA[#]mA[#]mU[#]mA[#]rA |
| P139as | 613 | [mU][#][mC][#][mA][mA][mA][mA][mG][mA][mU][mA][mA][mA][mU][fG][#]mU[#]mC[#]mU[#]mG[#]rC |
| P21as | 614 | [mU][#][mU][#][mU][mC][mC][mG][mA][mA][mU][mA][mA][mA][mC][fU][#]mC[#]mC[#]mA[#]mG[#]rG |
| P87as | 615 | [mU][#][mC][#][mU][mG][mG][mC][mU][mC][mA][mG][mU][mU][mC][fC][#]mU[#]mG[#]mC[#]mU[#]rG |
| P77as | 616 | [mU][#][mC][#][mA][mG][mA][mC][mA][mG][mC][mA][mU][mC][mA][fU][#]mG[#]mG[#]mC[#]mU[#]rG |
| P94as | 617 | [mU][#][mG][#][mG][mG][mA][mU][mU][mC][mC][mA][mU][mG][mC][fU][#]mC[#]mC[#]mU[#]mU[#]rG |

TABLE 1b

| | SEQ | |
|---|---|---|
| ID | ID NO: | Sense strand modified |
| P44s | 618 | [mu][#][fU][#][fG][fU][mA][mA][mC][mu][mu][mG][mA][mA][#][mG][#][mA][#][3XGalNac] |
| P46s | 619 | [mc][#][fU][#][fG][fU][mu][mu][mu][mG][mC][mu][mu][mU][#][mU][#][mA][#][3XGalNac] |
| P53s | 620 | [mG][#][fU][#][fU][fU][mu][mG][mC][mu][mu][mu][mu][mG][#][mU][#][mA][#][3XGalNac] |
| P29s | 621 | [mA][#][fG][#][fA][fC][mC][mu][mG][mu][mu][mu][mu][mG][#][mC][#][mA][#][3XGalNac] |
| P57s | 622 | [mu][#][fG][#][fU][fU][mu][mu][mG][mC][mu][mu][mu][mU][#][mG][#][mA][#][3XGalNac] |
| P52s | 623 | [mA][#][fG][#][fA][fU][mA][mu][mu][mu][mA][mu][mu][mC][#][mU][#][mA][#][3XGalNac] |
| P55s | 624 | [mc][#][fc][#][fU][fG][mu][mu][mu][mu][mG][mC][mu][mU][#][mU][#][mA][#][3XGalNac] |
| P232s | 625 | [mu][#][fG][#][fU][fA][mA][mC][mu][mu][mG][mA][mA][mG][#][mA][#][mA][#][3XGalNac] |
| P58s | 626 | [mc][#][fU][#][fU][fU][mu][mG][mu][mA][mA][mC][mu][mU][#][mG][#][mA][#][3XGalNac] |
| P36s | 627 | [mu][#][fA][#][fG][fA][mC][mC][mu][mG][mu][mu][mu][mU][#][mG][#][mA][#][3XGalNac] |
| P49s | 628 | [mu][#][fG][#][fA][fA][mG][mA][mu][mA][mu][mu][mu][mA][#][mU][#][mA][#][3XGalNac] |
| P233s | 629 | [mu][#][fU][#][fU][fG][mC][mu][mu][mu][mu][mG][mu][mA][#][mA][#][mA][#][3XGalNac] |
| P48s | 630 | [mu][#][fG][#][fC][fU][mu][mu][mG][mu][mG][mu][mC][mA][#][mC][#][mA][#][3XGalNac] |
| P28s | 631 | [mu][#][fU][#][fU][fG][mu][mA][mA][mC][mu][mu][mG][mA][#][mA][#][mA][#][3XGalNac] |
| P34s | 632 | [mc][#][fU][#][fU][fG][mA][mA][mG][mA][mu][mA][mu][mU][#][mU][#][mA][#][3XGalNac] |
| P31s | 633 | [mA][#][fG][#][fC][fA][mG][mA][mC][mA][mu][mu][mu][mA][#][mU][#][mA][#][3XGalNac] |
| P13s | 634 | [mG][#][fG][#][fA][fG][mu][mu][mu][mA][mu][mu][mC][mG][#][mG][#][mA][#][3XGalNac] |
| P35s | 635 | [mu][#][fU][#][fA][fA][mA][mA][mu][mG][mG][mu][mu][mC][#][mC][#][mA][#][3XGalNac] |
| P43s | 636 | [mA][#][fU][#][fA][fU][mu][mu][mA][mu][mu][mC][mu][mG][#][mG][#][mA][#][3XGalNac] |
| P127s | 637 | [mA][#][fA][#][fC][fU][mu][mG][mA][mA][mG][mA][mu][mA][#][mU][#][mA][#][3XGalNac] |
| P100s | 638 | [mA][#][fG][#][fC][fA][mG][mG][mA][mA][mC][mu][mG][mA][#][mG][#][mA][#][3XGalNac] |
| P76s | 639 | [mc][#][fU][#][fG][fG][mG][mu][mu][mu][mu][mG][mu][mA][#][mG][#][mA][#][3XGalNac] |
| P139s | 640 | [mc][#][fA][#][fU][fU][mu][mA][mu][mC][mu][mu][mu][mU][#][mG][#][mA][#][3XGalNac] |
| P21s | 641 | [mA][#][fG][#][fU][fU][mu][mA][mu][mu][mC][mG][mG][mA][#][mA][#][mA][#][3XGalNac] |
| P87s | 642 | [mG][#][fG][#][fA][fA][mC][mu][mG][mA][mG][mC][mC][mA][#][mG][#][mA][#][3XGalNac] |
| P77s | 643 | [mA][#][fU][#][fG][fA][mu][mG][mC][mu][mG][mu][mC][mU][#][mG][#][mA][#][3XGalNac] |
| P94s | 644 | [mA][#][fG][#][fC][fA][mu][mG][mG][mA][mA][mu][mC][mC][#][mC][#][mA][#][3XGalNac] |

Tables 2a to 2d below show nucleobase sequences and sugar-phosphate backbone modifications of antisense and sense strands of the 250 constructs selected in accordance with Example 1. The above-disclosed 27 oligomeric compounds have been selected from these 250 constructs. The numbering in Table 2a coincides with the number of the corresponding entry in the sequence listing. For Table 2c the following applies: entry number in the sequence listing=entry number in the Table+250.

TABLE 2a

| SEQ | |
|---|---|
| ID NO: | 250 antisense nucleobase sequences |
| 1 | UGGCCUAUGAGGGUGCCGC |
| 2 | UGAAUAAACUCCAGGCCUA |
| 3 | UUAAACUCCAGGCCUAUGA |
| 4 | UCGAAUAAACUCCAGGCCU |
| 5 | UUCCGGCAGCAGAUGGCAA |
| 6 | UACUCCAGGCCUAUGAGGG |
| 7 | UCCUAUGAGGGUGCCGCUA |
| 8 | UAGGCCUAUGAGGGUGCCG |
| 9 | UUCCAGGCCUAUGAGGGUG |
| 10 | UGCUGGUCCUCAGGGAACC |
| 11 | UAAUAAACUCCAGGCCUAU |
| 12 | UCUGGAGCAGCUCAGCAGC |
| 13 | UCCGAAUAAACUCCAGGCC |
| 14 | UGGUCCUCAGGGAACCAGG |
| 15 | UUGGUCCUCAGGGAACCAG |
| 16 | UAUAAACUCCAGGCCUAUG |
| 17 | UCUCCAGGCCUAUGAGGGU |
| 18 | UCAGGCCUAUGAGGGUGCC |
| 19 | UGUCCUCAGGGAACCAGGC |
| 20 | UGCCUAUGAGGGUGCCGCU |
| 21 | UUUCCGAAUAAACUCCAGG |
| 22 | UGCAUGGCAGCAGGAAGCG |
| 23 | UGCUCCGGCAGCAGAUGGC |
| 24 | UGCCGGCUCCGGCAGCAGA |
| 25 | UUCCUCAGGGAACCAGGCC |
| 26 | UCUCCGGCAGCAGAUGGCA |
| 27 | UGCCACGUGGGCAGCAGCC |
| 28 | UUUCAAGUUACAAAAGCAA |
| 29 | UGCAAAACAGGUCUAGAAA |
| 30 | UACCCAGAAUAAAUAUCUU |
| 31 | UAUAAAUGUCUGCUUGCUU |
| 32 | UUCAAGUUACAAAAGCAAA |
| 33 | UAACAGAGAGGACAGACCC |
| 34 | UAAAUAUCUUCAAGUUACA |
| 35 | UGGAACCAUUUUAAAGCUC |
| 36 | UCAAAACAGGUCUAGAAAA |
| 37 | UAAAUGCUACAAAACCCAG |
| 38 | UCCAAAAGAUAAAUGUCUG |
| 39 | UAGCACCUGGCAAUGGCGU |
| 40 | UAGAAUCCUGCCUCCUUGG |
| 41 | UUGGAUCAGUCUCUGCCUC |
| 42 | UCCCAGAAUAAAUAUCUUC |
| 43 | UCCAGAAUAAAUAUCUUCA |
| 44 | UCUUCAAGUUACAAAAGCA |
| 45 | UAUGCUACAAAACCCAGAA |
| 46 | UAAAAGCAAAACAGGUCUA |
| 47 | UAAUAUCUUCAAGUUACAA |
| 48 | UGUGACACAAAGCAGGUGC |
| 49 | UAUAAAUAUCUUCAAGUUA |
| 50 | UAAUGCUACAAAACCCAGA |
| 51 | UCAAAGCAGGUGCUGCAGU |
| 52 | UAGAAUAAAUAUCUUCAAG |
| 53 | UACAAAAGCAAAACAGGUC |
| 54 | UACAAAACCCAGAAUAAAU |
| 55 | UAAAGCAAAACAGGUCUAG |
| 56 | UCGAAGUCGGUGACCAUGA |
| 57 | UCAAAAGCAAAACAGGUCU |
| 58 | UCAAGUUACAAAAGCAAAA |
| 59 | UCCUUGACUUUGCAUUCCA |
| 60 | UAAGCGUGGAUGCUGGCCU |
| 61 | UUAAAUGUCUGCUUGCUUG |
| 62 | UAACCAUUUUAAAGCUCAG |
| 63 | UACCAGGCCUCAUUGAUGA |
| 64 | UAAACCCAGAAUAAAUAUC |
| 65 | UGAACCAUUUUAAAGCUCA |
| 66 | UGAAUAAAUAUCUUCAAGU |
| 67 | UAAGAGGCUUGGCUUCAGA |
| 68 | UGAACCAGGCCUCAUUGAU |
| 69 | UCAGAAUAAAUAUCUUCAA |
| 70 | UAAGAAUCCUGCCUCCUUG |
| 71 | UAAAACAGGUCUAGAAAAG |
| 72 | UAGGAAGCGUGGAUGCUGG |
| 73 | UCACUGGUUGGGCUGACCU |
| 74 | UAGACAUGCAGGAUCUUGG |
| 75 | UCUAGGAGAUACACCUCCA |
| 76 | UCUACAAAACCCAGAAUAA |
| 77 | UCAGACAGCAUCAUGGCUG |
| 78 | UUAUGCUGGUGUCUAGGAG |
| 79 | UGAAACUGGAGCAGCUCAG |
| 80 | UAGGACAGACCCAAAAGAU |
| 81 | UCAAUGGCGUAGACACCCU |
| 82 | UCAGGUGCUGCAGUCGCUG |
| 83 | UCUGCCUCCUUGGUGGAGA |
| 84 | UAGUCGGUGACCAUGACCC |
| 85 | UCAGCCUGGCAUAGAGCAG |
| 86 | UCAGAGAGGACAGACCCAA |
| 87 | UCUGGCUCAGUUCCUGCUG |
| 88 | UGAGCUUCCUGGUCUGUGU |
| 89 | UAGGUGCUGCAGUCGCUGG |
| 90 | UCUGGCCUGUCUGUGGAAG |
| 91 | UACCGCCUGGAGCUGACGG |
| 92 | UCAAAAGCGUUGUGGGCCC |
| 93 | UCACAGCCUGGCAUAGAGC |
| 94 | UGGGAUUCCAUGCUCCUUG |
| 95 | UGUGACCAUGACCCUGCCC |
| 96 | UAGCAAAACAGGUCUAGAA |
| 97 | UCUGGCUCACUCCUCCAGG |
| 98 | UGAAGUGGAUCAGUCUCUG |
| 99 | UAUGCUGGUGUCUAGGAGA |
| 100 | UCUCAGUUCCUGCUGUGUG |
| 101 | UUACACCUCCACCAGGCUG |
| 102 | UAUCAUGGCUGCAAUGCCA |
| 103 | UACAGAGAGGACAGACCCA |
| 104 | UGAAGAGGCUUGGCUUCAG |
| 105 | UCCCUGCCCUCGAUUUCCC |
| 106 | UACCAGGAAGCCAGGAAGA |
| 107 | UGAGGGAGCUUCCUGGCAC |
| 108 | UACCAUUUUAAAGCUCAGC |
| 109 | UCACUUGCUGGCCUGUCUG |
| 110 | UGCCACUCAUCUUCACCAG |
| 111 | UUUGGCAGAGAAGUGGAUC |
| 112 | UAGUCCUCCUCGAUGUAGU |
| 113 | UCCUCUGGCUAGAUGCCAU |
| 114 | UGGAGCUGUGUGGACGCUG |
| 115 | UGAGAAGUGGAUCAGUCUC |
| 116 | UAGCAGAUGGCAACGGCUG |
| 117 | UAGGCCUCAUUGAUGACAU |
| 118 | UUGCAGUCGCUGGAGGCAC |
| 119 | UUGGGUCUCCUCCUUCAGC |
| 120 | UCGGCUCGGCAGACAGCAU |
| 121 | UUGACAUCUUUGGCAGAGA |
| 122 | UGAACGCAAGGCUAGCACC |
| 123 | UAUCUUUGGCAGAGAAGUG |
| 124 | UGGCAGGCAUCGUCCCGGA |
| 125 | UGGAACGCAAGGCUAGCAC |
| 126 | UGACACAAAGCAGGUGCUG |
| 127 | UAUAUCUUCAAGUUACAAA |
| 128 | UGUGCCCUUCCCUUGGCAG |
| 129 | UCAAUGCCAGCCACGUGGG |
| 130 | UGAGGCUUGGCUUCAGAGC |
| 131 | UGGAUCUUGGUGAGGUAUC |
| 132 | UCAACUGUGAUGACCUCGG |
| 133 | UAAAUGUCUGCUUGCUUGG |
| 134 | UAAACAGGUCUAGAAAAGU |
| 135 | UAAAGCGUUGUGGGCCCGG |
| 136 | UCAACAGAGAGGACAGACC |
| 137 | UCUCCACGGAUCCUUGGCG |
| 138 | UAGGACUGUGCAGGAGCUG |
| 139 | UCAAAAGAUAAAUGUCUGC |
| 140 | UAUCCUGCCUCCUUGGUGG |
| 141 | UCACAGCGGCCAAAGUUGG |
| 142 | UCAGUUCCUGCUGUGUGAG |
| 143 | UCUGGCAUAGAGCAGAGUA |
| 144 | UCCUCAUUGAUGACAUCUU |
| 145 | UAGUCGCCGUCCUCGUCCU |
| 146 | UUGGGAAGAAUCCUGCCUC |
| 147 | UGUGGGUGCUUGACGCCUG |
| 148 | UACACAAAGCAGGUGCUGC |
| 149 | UAGCAGGAAGCGUGGAUGC |
| 150 | UAUCAGUCUCUGCCUCAAC |
| 151 | UAGACAGCAUCAUGGCUGC |
| 152 | UUCAUGGCUGCAAUGCCAG |
| 153 | UACAGCCUGGCAUAGAGCA |
| 154 | UGGCAUAGAGCAGAGUAAA |
| 155 | UCUGGUUGGGCUGACCUCG |
| 156 | UAGACCCAAAAGAUAAAUG |
| 157 | UGGUCCACACAGCGGCCAA |
| 158 | UCCUCCACCAGGCUGCCUC |
| 159 | UAAAACAGCUGCCAACCUG |
| 160 | UGAGGACAGACCCAAAAGA |
| 161 | UUGUCUGCUUGCUUGGGUG |
| 162 | UAAGGCAACAGAGAGGACA |
| 163 | UGUGUCUAGGAGAUACACC |
| 164 | UGUGGAUCAGUCUCUGCCU |
| 165 | UAAACAGCUGCCAACCUGC |
| 166 | UCAGGCGGCUUGUGGGUGC |
| 167 | UGGAACCAGGCCUCAUUGA |
| 168 | UCUGCCUGGCUCACUCCUC |
| 169 | UAGAUGAGGGCCAUCAGCA |
| 170 | UCUUCCUGGUCUGUGUUCC |
| 171 | UACUUUGCAUUCCAGACCU |
| 172 | UUGGCUAGAUGCCAUCCAG |
| 173 | UUGGCUGCAAUGCCAGCCA |
| 174 | UGGGUGUGGGUGCUUGACG |
| 175 | UAAAAGCGUUGUGGGCCCG |
| 176 | UCACCAGGAAGCCAGGAAG |
| 177 | UGCCGGGAUUCCAUGCUCC |
| 178 | UCCAGCUCCUCGUAGUCGC |
| 179 | UCCCGCUGGUCCUCAGGGA |
| 180 | UUGCAAUGCCAGCCACGUG |
| 181 | UUGAUGACAUCUUUGGCAG |
| 182 | UGAGAUACACCUCCACCAG |
| 183 | UGGUGUGGGUGCUUGACGC |
| 184 | UCUGCCUCAACUCGGCCAG |
| 185 | UGAAGUCGGUGACCAUGAC |
| 186 | UGUCCUCGUCCUCCUGCGC |
| 187 | UCCAGCCUCACUGUUACCC |
| 188 | UGCUUUUCCGAAUAAACUC |
| 189 | UUGUGAGCUUGGCAGGCAC |
| 190 | UAGCGUGGAUGCUGGCCUC |
| 191 | UAAGACAGAGGAGUCCUCC |
| 192 | UGUCUGCUUGCUUGGGUGG |
| 193 | UGUAGCAGGCAGCACCUGG |
| 194 | UUGGAUGCUGGCCUCCCUG |
| 195 | UAAAAGAUAAAUGUCUGCU |
| 196 | UAGUCCUGCAAAACAGCUG |
| 197 | UCAGGAUCUUGGUGAGGUA |
| 198 | UAGCGGUGGAAGGUGGCUG |
| 199 | UUGGCAAUGGCGUAGACAC |
| 200 | UCAGAGGAGUCCUCCUCGA |
| 201 | UGGAGCAGCUCAGCAGCUC |
| 202 | UGUGUGAGCUUGGCAGGCA |
| 203 | UCAGCACCUGGCAAUGGCG |
| 204 | UUCCCAGCCUCACUGUUAC |
| 205 | UGAGAAACUGGAGCAGCUC |
| 206 | UAGAGAAGUGGAUCAGUCU |
| 207 | UGGUCGCCACUCAUCUUCA |
| 208 | UGACCAGCUGGCUUUUCCG |
| 209 | UCAUGCAGGAUCUUGGUGA |
| 210 | UAGCGGCCAAAGUUGGUCC |
| 211 | UCAGCCUGUGAGGACGUGG |
| 212 | UGAGCUGUGUGGACGCUGC |
| 213 | UGGUAGCAGGCAGCACCUG |
| 214 | UUGAGCUCCGGCUCGGCAG |
| 215 | UGUCUAGGAGAUACACCUC |
| 216 | UGGAAGCGGGUCCCGUCCU |
| 217 | UGCUGUGUGAGCUUGGCAG |
| 218 | UACCAGCUGGCUUUUCCGA |
| 219 | UGCAAAGAGGUCCACACAG |
| 220 | UAGGAGACCUAGAGGCCGU |
| 221 | UGUCCUGCAAAACAGCUGC |
| 222 | UGCACCACCACGUAGGUGC |
| 223 | UACUCAAGGGCCAGGCCAG |
| 224 | UCCAUUUUAAAGCUCAGCC |
| 225 | UAGCGUUGUGGGCCCGGCA |
| 226 | UGGCAGAGAAGUGGAUCAG |
| 227 | UGAAACCUUCUAGGGUGUG |
| 228 | UAGCGGUGACCAGCACGAC |
| 229 | UGCAGUCGCUGGAGGCACC |
| 230 | UAAAACCCAGAAUAAAUAU |
| 231 | UAAUGGCGUAGACACCCUC |
| 232 | UUCUUCAAGUUACAAAAGC |
| 233 | UUUACAAAAGCAAAACAGG |
| 234 | UAUCUUCAAGUUACAAAAG |
| 235 | UCUGGUGUCUAGGAGAUAC |
| 236 | UAGUGCGCUCUGACUGCGA |
| 237 | UUGUCACACUUGCUGGCCU |
| 238 | UACAGAGGAGUCCUCCUCG |
| 239 | UGUUACAAAAGCAAAACAG |
| 240 | UAAUCCUGCCUCCUUGGUG |
| 241 | UGUGGAAGGUGGCUGUGGU |
| 242 | UGGAGCGGGUUGGCUGAGA |
| 243 | UUCGCCACUCAUCUUCACC |
| 244 | UAGAGGAGUCCUCCUCGAU |
| 245 | UGAGCUCCGGCUCGGCAGA |
| 246 | UAACUGUGAUGACCUCGGG |
| 247 | UGAUACACCUCCACCAGGC |
| 248 | UUGGCAUAGAGCAGAGUAA |
| 249 | UGCAGCUCAGCAGCUCCUC |
| 250 | UAUUGAUGACAUCUUUGGC |

TABLE 2b

| | SEQ ID | |
|---|---|---|
| # | NO: | 250 modified antisense strands |
| 1 | 645 | PmU.fG.mG.fC.mC.fU.mA.fU.mG.fA.mG.fG.mG.fU.mG.fC.mC.fG.mC |
| 2 | 646 | PmU.fG.mA.fA.mU.fA.mA.fA.mC.fU.mC.fC.mA.fG.mG.fC.mC.fU.mA |
| 3 | 647 | PmU.fU.mA.fA.mA.fC.mU.fC.mC.fA.mG.fG.mC.fC.mU.fA.mU.fG.mA |
| 4 | 648 | PmU.fC.mG.fA.mA.fU.mA.fA.mA.fC.mU.fC.mC.fA.mG.fG.mC.fC.mU |
| 5 | 649 | PmU.fU.mC.fC.mG.fG.mC.fA.mG.fC.mA.fG.mA.fU.mG.fG.mC.fA.mA |
| 6 | 650 | PmU.fA.mC.fU.mC.fC.mA.fG.mG.fC.mC.fU.mA.fU.mG.fA.mG.fG.mG |
| 7 | 651 | PmU.fC.mC.fU.mA.fU.mG.fA.mG.fG.mG.fU.mG.fC.mC.fG.mC.fU.mA |
| 8 | 652 | PmU.fA.mG.fG.mC.fC.mU.fA.mU.fG.mA.fG.mG.fG.mU.fG.mC.fC.mG |
| 9 | 653 | PmU.fU.mC.fC.mA.fG.mG.fC.mC.fU.mA.fU.mG.fA.mG.fG.mG.fU.mG |
| 10 | 654 | PmU.fG.mC.fU.mG.fG.mU.fC.mC.fU.mC.fA.mG.fG.mG.fA.mA.fC.mC |
| 11 | 655 | PmU.fA.mA.fU.mA.fA.mA.fC.mU.fC.mC.fA.mG.fG.mC.fC.mU.fA.mU |
| 12 | 656 | PmU.fC.mU.fG.mG.fA.mG.fC.mA.fG.mC.fU.mC.fA.mG.fC.mA.fG.mC |
| 13 | 657 | PmU.fC.mC.fG.mA.fA.mU.fA.mA.fA.mC.fU.mC.fC.mA.fG.mG.fC.mC |
| 14 | 658 | PmU.fG.mG.fU.mC.fC.mU.fC.mA.fG.mG.fG.mA.fA.mC.fC.mA.fG.mG |
| 15 | 659 | PmU.fU.mG.fG.mU.fC.mC.fU.mC.fA.mG.fG.mG.fA.mA.fC.mC.fA.mG |
| 16 | 660 | PmU.fA.mU.fA.mA.fA.mC.fU.mC.fC.mA.fG.mG.fC.mC.fU.mA.fU.mG |
| 17 | 661 | PmU.fC.mU.fC.mC.fA.mG.fG.mC.fC.mU.fA.mU.fG.mA.fG.mG.fG.mU |
| 18 | 662 | PmU.fC.mA.fG.mG.fC.mC.fU.mA.fU.mG.fA.mG.fG.mG.fU.mG.fC.mC |
| 19 | 663 | PmU.fG.mU.fC.mC.fU.mC.fA.mG.fG.mG.fA.mA.fC.mC.fA.mG.fG.mC |
| 20 | 664 | PmU.fG.mC.fC.mU.fA.mU.fG.mA.fG.mG.fG.mU.fG.mC.fC.mG.fC.mU |
| 21 | 665 | PmU.fU.mU.fC.mC.fG.mA.fA.mU.fA.mA.fA.mC.fU.mC.fC.mA.fG.mG |
| 22 | 666 | PmU.fG.mC.fA.mU.fG.mG.fC.mA.fG.mC.fA.mG.fG.mA.fA.mG.fC.mG |
| 23 | 667 | PmU.fG.mC.fU.mC.fC.mG.fG.mC.fA.mG.fC.mA.fG.mA.fU.mG.fG.mC |
| 24 | 668 | PmU.fG.mC.fC.mG.fG.mC.fU.mC.fC.mG.fG.mC.fA.mG.fC.mA.fG.mA |
| 25 | 669 | PmU.fU.mC.fC.mU.fC.mA.fG.mG.fG.mA.fA.mC.fC.mA.fG.mG.fC.mC |
| 26 | 670 | PmU.fC.mU.fC.mC.fG.mG.fC.mA.fG.mC.fA.mG.fA.mU.fG.mG.fC.mA |
| 27 | 671 | PmU.fG.mC.fC.mA.fC.mG.fU.mG.fG.mG.fC.mA.fG.mC.fA.mG.fC.mC |
| 28 | 672 | PmU.fU.mU.fC.mA.fA.mG.fU.mU.fA.mC.fA.mA.fA.mA.fG.mC.fA.mA |
| 29 | 673 | PmU.fG.mC.fA.mA.fA.mA.fC.mA.fG.mG.fU.mC.fU.mA.fG.mA.fA.mA |
| 30 | 674 | PmU.fA.mC.fC.mC.fA.mG.fA.mA.fU.mA.fA.mA.fU.mA.fU.mC.fU.mU |
| 31 | 675 | PmU.fA.mU.fA.mA.fA.mU.fG.mU.fC.mU.fG.mC.fU.mU.fG.mC.fU.mU |
| 32 | 676 | PmU.fU.mC.fA.mA.fG.mU.fU.mA.fC.mA.fA.mA.fA.mG.fC.mA.fA.mA |
| 33 | 677 | PmU.fA.mA.fC.mA.fG.mA.fG.mA.fG.mG.fA.mC.fA.mG.fA.mC.fC.mC |
| 34 | 678 | PmU.fA.mA.fA.mU.fA.mU.fC.mU.fU.mC.fA.mA.fG.mU.fU.mA.fC.mA |
| 35 | 679 | PmU.fG.mG.fA.mA.fC.mC.fA.mU.fU.mU.fU.mA.fA.mA.fG.mC.fU.mC |
| 36 | 680 | PmU.fC.mA.fA.mA.fA.mC.fA.mG.fG.mU.fC.mU.fA.mG.fA.mA.fA.mA |
| 37 | 681 | PmU.fA.mA.fA.mU.fG.mC.fU.mA.fC.mA.fA.mA.fA.mC.fC.mC.fA.mG |
| 38 | 682 | PmU.fC.mC.fA.mA.fA.mA.fG.mA.fU.mA.fA.mA.fU.mG.fU.mC.fU.mG |
| 39 | 683 | PmU.fA.mG.fC.mA.fC.mC.fU.mG.fG.mC.fA.mA.fU.mG.fG.mC.fG.mU |
| 40 | 684 | PmU.fA.mG.fA.mA.fU.mC.fC.mU.fG.mC.fC.mU.fC.mC.fU.mU.fG.mG |
| 41 | 685 | PmU.fU.mG.fG.mA.fU.mC.fA.mG.fU.mC.fU.mC.fU.mG.fC.mC.fU.mC |
| 42 | 686 | PmU.fC.mC.fC.mA.fG.mA.fA.mU.fA.mA.fA.mU.fA.mU.fC.mU.fU.mC |
| 43 | 687 | PmU.fC.mC.fA.mG.fA.mA.fU.mA.fA.mA.fU.mA.fU.mC.fU.mU.fC.mA |
| 44 | 688 | PmU.fC.mU.fU.mC.fA.mA.fG.mU.fU.mA.fC.mA.fA.mA.fA.mG.fC.mA |
| 45 | 689 | PmU.fA.mU.fG.mC.fU.mA.fC.mA.fA.mA.fA.mC.fC.mC.fA.mG.fA.mA |
| 46 | 690 | PmU.fA.mA.fA.mA.fG.mC.fA.mA.fA.mA.fC.mA.fG.mG.fU.mC.fU.mA |
| 47 | 691 | PmU.fA.mA.fU.mA.fU.mC.fU.mU.fC.mA.fA.mG.fU.mU.fA.mC.fA.mA |
| 48 | 692 | PmU.fG.mU.fG.mA.fC.mA.fC.mA.fA.mA.fG.mC.fA.mG.fG.mU.fG.mC |
| 49 | 693 | PmU.fA.mU.fA.mA.fA.mU.fA.mU.fC.mU.fU.mC.fA.mA.fG.mU.fU.mA |
| 50 | 694 | PmU.fA.mA.fU.mG.fC.mU.fA.mC.fA.mA.fA.mA.fC.mC.fC.mA.fG.mA |
| 51 | 695 | PmU.fC.mA.fA.mA.fG.mC.fA.mG.fG.mU.fG.mC.fU.mG.fC.mA.fG.mU |
| 52 | 696 | PmU.fA.mG.fA.mA.fU.mA.fA.mA.fU.mA.fU.mC.fU.mU.fC.mA.fA.mG |
| 53 | 697 | PmU.fA.mC.fA.mA.fA.mA.fG.mC.fA.mA.fA.mA.fC.mA.fG.mG.fU.mC |
| 54 | 698 | PmU.fA.mC.fA.mA.fA.mA.fC.mC.fC.mA.fG.mA.fA.mU.fA.mA.fA.mU |
| 55 | 699 | PmU.fA.mA.fA.mG.fC.mA.fA.mA.fA.mC.fA.mG.fG.mU.fC.mU.fA.mG |
| 56 | 700 | PmU.fC.mG.fA.mA.fG.mU.fC.mG.fG.mU.fG.mA.fC.mC.fA.mU.fG.mA |
| 57 | 701 | PmU.fC.mA.fA.mA.fA.mG.fC.mA.fA.mA.fA.mC.fA.mG.fG.mU.fC.mU |
| 58 | 702 | PmU.fC.mA.fA.mG.fU.mU.fA.mC.fA.mA.fA.mA.fG.mC.fA.mA.fA.mA |
| 59 | 703 | PmU.fC.mC.fU.mU.fG.mA.fC.mU.fU.mU.fG.mC.fA.mU.fU.mC.fC.mA |
| 60 | 704 | PmU.fA.mA.fG.mC.fG.mU.fG.mG.fA.mU.fG.mC.fU.mG.fG.mC.fC.mU |
| 61 | 705 | PmU.fU.mA.fA.mA.fU.mG.fU.mC.fU.mG.fC.mU.fU.mG.fC.mU.fU.mG |
| 62 | 706 | PmU.fA.mA.fC.mC.fA.mU.fU.mU.fU.mA.fA.mA.fG.mC.fU.mC.fA.mG |
| 63 | 707 | PmU.fA.mC.fC.mA.fG.mG.fC.mC.fU.mC.fA.mU.fU.mG.fA.mU.fG.mA |
| 64 | 708 | PmU.fA.mA.fA.mC.fC.mC.fA.mG.fA.mA.fU.mA.fA.mA.fU.mA.fU.mC |
| 65 | 709 | PmU.fG.mA.fA.mC.fC.mA.fU.mU.fU.mU.fA.mA.fA.mG.fC.mU.fC.mA |
| 66 | 710 | PmU.fG.mA.fA.mU.fA.mA.fA.mU.fA.mU.fC.mU.fU.mC.fA.mA.fG.mU |
| 67 | 711 | PmU.fA.mA.fG.mA.fG.mG.fC.mU.fU.mG.fG.mC.fU.mU.fC.mA.fG.mA |
| 68 | 712 | PmU.fG.mA.fA.mC.fC.mA.fG.mG.fC.mC.fU.mC.fA.mU.fU.mG.fA.mU |
| 69 | 713 | PmU.fC.mA.fG.mA.fA.mU.fA.mA.fA.mU.fA.mU.fC.mU.fU.mC.fA.mA |
| 70 | 714 | PmU.fA.mA.fG.mA.fA.mU.fC.mC.fU.mG.fC.mC.fU.mC.fC.mU.fU.mG |
| 71 | 715 | PmU.fA.mA.fA.mA.fC.mA.fG.mG.fU.mC.fU.mA.fG.mA.fA.mA.fA.mG |
| 72 | 716 | PmU.fA.mG.fG.mA.fA.mG.fC.mG.fU.mG.fG.mA.fU.mG.fC.mU.fG.mG |
| 73 | 717 | PmU.fC.mA.fC.mU.fG.mG.fU.mU.fG.mG.fG.mC.fU.mG.fA.mC.fC.mU |
| 74 | 718 | PmU.fA.mG.fA.mC.fA.mU.fG.mC.fA.mG.fG.mA.fU.mC.fU.mU.fG.mG |
| 75 | 719 | PmU.fC.mU.fA.mG.fG.mA.fG.mA.fU.mA.fC.mA.fC.mC.fU.mC.fC.mA |
| 76 | 720 | PmU.fC.mU.fA.mC.fA.mA.fA.mA.fC.mC.fC.mA.fG.mA.fA.mU.fA.mA |
| 77 | 721 | PmU.fC.mA.fG.mA.fC.mA.fG.mC.fA.mU.fC.mA.fU.mG.fG.mC.fU.mG |
| 78 | 722 | PmU.fU.mA.fU.mG.fC.mU.fG.mG.fU.mG.fU.mC.fU.mA.fG.mG.fA.mG |
| 79 | 723 | PmU.fG.mA.fA.mA.fC.mU.fG.mG.fA.mG.fC.mA.fG.mC.fU.mC.fA.mG |
| 80 | 724 | PmU.fA.mG.fG.mA.fC.mA.fG.mA.fC.mC.fC.mA.fA.mA.fA.mG.fA.mU |
| 81 | 725 | PmU.fC.mA.fA.mU.fG.mG.fC.mG.fU.mA.fG.mA.fC.mA.fC.mC.fC.mU |
| 82 | 726 | PmU.fC.mA.fG.mG.fU.mG.fC.mU.fG.mC.fA.mG.fU.mC.fG.mC.fU.mG |
| 83 | 727 | PmU.fC.mU.fG.mC.fC.mU.fC.mC.fU.mU.fG.mG.fU.mG.fG.mA.fG.mA |
| 84 | 728 | PmU.fA.mG.fU.mC.fG.mG.fU.mG.fA.mC.fC.mA.fU.mG.fA.mC.fC.mC |
| 85 | 729 | PmU.fC.mA.fG.mC.fC.mU.fG.mG.fC.mA.fU.mA.fG.mA.fG.mC.fA.mG |
| 86 | 730 | PmU.fC.mA.fG.mA.fG.mA.fG.mG.fA.mC.fA.mG.fA.mC.fC.mC.fA.mA |
| 87 | 731 | PmU.fC.mU.fG.mG.fC.mU.fC.mA.fG.mU.fU.mC.fC.mU.fG.mC.fU.mG |
| 88 | 732 | PmU.fG.mA.fG.mC.fU.mU.fC.mC.fU.mG.fG.mU.fC.mU.fG.mU.fG.mU |
| 89 | 733 | PmU.fA.mG.fG.mU.fG.mC.fU.mG.fC.mA.fG.mU.fC.mG.fC.mU.fG.mG |
| 90 | 734 | PmU.fC.mU.fG.mG.fC.mC.fU.mG.fU.mC.fU.mG.fU.mG.fG.mA.fA.mG |
| 91 | 735 | PmU.fA.mC.fC.mG.fC.mC.fU.mG.fG.mA.fG.mC.fU.mG.fA.mC.fG.mG |
| 92 | 736 | PmU.fC.mA.fA.mA.fA.mG.fC.mG.fU.mU.fG.mU.fG.mG.fG.mC.fC.mC |
| 93 | 737 | PmU.fC.mA.fC.mA.fG.mC.fC.mU.fG.mG.fC.mA.fU.mA.fG.mA.fG.mC |
| 94 | 738 | PmU.fG.mG.fG.mA.fU.mU.fC.mC.fA.mU.fG.mC.fU.mC.fC.mU.fU.mG |
| 95 | 739 | PmU.fG.mU.fG.mA.fC.mC.fA.mU.fG.mA.fC.mC.fC.mU.fG.mC.fC.mC |
| 96 | 740 | PmU.fA.mG.fC.mA.fA.mA.fA.mC.fA.mG.fG.mU.fC.mU.fA.mG.fA.mA |
| 97 | 741 | PmU.fC.mU.fG.mG.fC.mU.fC.mA.fC.mU.fC.mC.fU.mC.fC.mA.fG.mG |
| 98 | 742 | PmU.fG.mA.fA.mG.fU.mG.fG.mA.fU.mC.fA.mG.fU.mC.fU.mC.fU.mG |
| 99 | 743 | PmU.fA.mU.fG.mC.fU.mG.fG.mU.fG.mU.fC.mU.fA.mG.fG.mA.fG.mA |
| 100 | 744 | PmU.fC.mU.fC.mA.fG.mU.fU.mC.fC.mU.fG.mC.fU.mG.fU.mG.fU.mG |
| 101 | 745 | PmU.fU.mA.fC.mA.fC.mC.fU.mC.fC.mA.fC.mC.fA.mG.fG.mC.fU.mG |
| 102 | 746 | PmU.fA.mU.fC.mA.fU.mG.fG.mC.fU.mG.fC.mA.fA.mU.fG.mC.fC.mA |
| 103 | 747 | PmU.fA.mC.fA.mG.fA.mG.fA.mG.fG.mA.fC.mA.fG.mA.fC.mC.fC.mA |
| 104 | 748 | PmU.fG.mA.fA.mG.fA.mG.fG.mC.fU.mU.fG.mG.fC.mU.fU.mC.fA.mG |
| 105 | 749 | PmU.fC.mC.fC.mU.fG.mC.fC.mC.fU.mC.fG.mA.fU.mU.fU.mC.fC.mC |
| 106 | 750 | PmU.fA.mC.fC.mA.fG.mG.fA.mA.fG.mC.fC.mA.fG.mG.fA.mA.fG.mA |
| 107 | 751 | PmU.fG.mA.fG.mG.fG.mA.fG.mC.fU.mU.fC.mC.fU.mG.fG.mC.fA.mC |
| 108 | 752 | PmU.fA.mC.fC.mA.fU.mU.fU.mU.fA.mA.fA.mG.fC.mU.fC.mA.fG.mC |
| 109 | 753 | PmU.fC.mA.fC.mU.fU.mG.fC.mU.fG.mG.fC.mC.fU.mG.fU.mC.fU.mG |
| 110 | 754 | PmU.fG.mC.fC.mA.fC.mU.fC.mA.fU.mC.fU.mU.fC.mA.fC.mC.fA.mG |
| 111 | 755 | PmU.fU.mU.fG.mG.fC.mA.fG.mA.fG.mA.fA.mG.fU.mG.fG.mA.fU.mC |
| 112 | 756 | PmU.fA.mG.fU.mC.fC.mU.fC.mC.fU.mC.fG.mA.fU.mG.fU.mA.fG.mU |
| 113 | 757 | PmU.fC.mC.fU.mC.fU.mG.fG.mC.fU.mA.fG.mA.fU.mG.fC.mC.fA.mU |
| 114 | 758 | PmU.fG.mG.fA.mG.fC.mU.fG.mU.fG.mU.fG.mG.fA.mC.fG.mC.fU.mG |
| 115 | 759 | PmU.fG.mA.fG.mA.fA.mG.fU.mG.fG.mA.fU.mC.fA.mG.fU.mC.fU.mC |
| 116 | 760 | PmU.fA.mG.fC.mA.fG.mA.fU.mG.fG.mC.fA.mA.fC.mG.fG.mC.fU.mG |
| 117 | 761 | PmU.fA.mG.fG.mC.fC.mU.fC.mA.fU.mU.fG.mA.fU.mG.fA.mC.fA.mU |
| 118 | 762 | PmU.fU.mG.fC.mA.fG.mU.fC.mG.fC.mU.fG.mG.fA.mG.fG.mC.fA.mC |
| 119 | 763 | PmU.fU.mG.fG.mG.fU.mC.fU.mC.fC.mU.fC.mC.fU.mU.fC.mA.fG.mC |
| 120 | 764 | PmU.fC.mG.fG.mC.fU.mC.fG.mG.fC.mA.fG.mA.fC.mA.fG.mC.fA.mU |
| 121 | 765 | PmU.fU.mG.fA.mC.fA.mU.fC.mU.fU.mU.fG.mG.fC.mA.fG.mA.fG.mA |
| 122 | 766 | PmU.fG.mA.fA.mC.fG.mC.fA.mA.fG.mG.fC.mU.fA.mG.fC.mA.fC.mC |
| 123 | 767 | PmU.fA.mU.fC.mU.fU.mU.fG.mG.fC.mA.fG.mA.fG.mA.fA.mG.fU.mG |
| 124 | 768 | PmU.fG.mG.fC.mA.fG.mG.fC.mA.fU.mC.fG.mU.fC.mC.fC.mG.fG.mA |
| 125 | 769 | PmU.fG.mG.fA.mA.fC.mG.fC.mA.fA.mG.fG.mC.fU.mA.fG.mC.fA.mC |
| 126 | 770 | PmU.fG.mA.fC.mA.fC.mA.fA.mA.fG.mC.fA.mG.fG.mU.fG.mC.fU.mG |
| 127 | 771 | PmU.fA.mU.fA.mU.fC.mU.fU.mC.fA.mA.fG.mU.fU.mA.fC.mA.fA.mA |
| 128 | 772 | PmU.fG.mU.fG.mC.fC.mC.fU.mU.fC.mC.fC.mU.fU.mG.fG.mC.fA.mG |
| 129 | 773 | PmU.fC.mA.fA.mU.fG.mC.fC.mA.fG.mC.fC.mA.fC.mG.fU.mG.fG.mG |
| 130 | 774 | PmU.fG.mA.fG.mG.fC.mU.fU.mG.fG.mC.fU.mU.fC.mA.fG.mA.fG.mC |
| 131 | 775 | PmU.fG.mG.fA.mU.fC.mU.fU.mG.fG.mU.fG.mA.fG.mG.fU.mA.fU.mC |
| 132 | 776 | PmU.fC.mA.fA.mC.fU.mG.fU.mG.fA.mU.fG.mA.fC.mC.fU.mC.fG.mG |
| 133 | 777 | PmU.fA.mA.fA.mU.fG.mU.fC.mU.fG.mC.fU.mU.fG.mC.fU.mU.fG.mG |
| 134 | 778 | PmU.fA.mA.fA.mC.fA.mG.fG.mU.fC.mU.fA.mG.fA.mA.fA.mA.fG.mU |
| 135 | 779 | PmU.fA.mA.fA.mG.fC.mG.fU.mU.fG.mU.fG.mG.fG.mC.fC.mC.fG.mG |
| 136 | 780 | PmU.fC.mA.fA.mC.fA.mG.fA.mG.fA.mG.fG.mA.fC.mA.fG.mA.fC.mC |
| 137 | 781 | PmU.fC.mU.fC.mC.fA.mC.fG.mG.fA.mU.fC.mC.fU.mU.fG.mG.fC.mG |
| 138 | 782 | PmU.fA.mG.fG.mA.fC.mU.fG.mU.fG.mC.fA.mG.fG.mA.fG.mC.fU.mG |
| 139 | 783 | PmU.fC.mA.fA.mA.fA.mG.fA.mU.fA.mA.fA.mU.fG.mU.fC.mU.fG.mC |
| 140 | 784 | PmU.fA.mU.fC.mC.fU.mG.fC.mC.fU.mC.fC.mU.fU.mG.fG.mU.fG.mG |
| 141 | 785 | PmU.fC.mA.fC.mA.fG.mC.fG.mG.fC.mC.fA.mA.fA.mG.fU.mU.fG.mG |
| 142 | 786 | PmU.fC.mA.fG.mU.fU.mC.fC.mU.fG.mC.fU.mG.fU.mG.fU.mG.fA.mG |
| 143 | 787 | PmU.fC.mU.fG.mG.fC.mA.fU.mA.fG.mA.fG.mC.fA.mG.fA.mG.fU.mA |
| 144 | 788 | PmU.fC.mC.fU.mC.fA.mU.fU.mG.fA.mU.fG.mA.fC.mA.fU.mC.fU.mU |
| 145 | 789 | PmU.fA.mG.fU.mC.fG.mC.fC.mG.fU.mC.fC.mU.fC.mG.fU.mC.fC.mU |
| 146 | 790 | PmU.fU.mG.fG.mG.fA.mA.fG.mA.fA.mU.fC.mC.fU.mG.fC.mC.fU.mC |
| 147 | 791 | PmU.fG.mU.fG.mG.fG.mU.fG.mC.fU.mU.fG.mA.fC.mG.fC.mC.fU.mG |
| 148 | 792 | PmU.fA.mC.fA.mC.fA.mA.fA.mG.fC.mA.fG.mG.fU.mG.fC.mU.fG.mC |
| 149 | 793 | PmU.fA.mG.fC.mA.fG.mG.fA.mA.fG.mC.fG.mU.fG.mG.fA.mU.fG.mC |
| 150 | 794 | PmU.fA.mU.fC.mA.fG.mU.fC.mU.fC.mU.fG.mC.fC.mU.fC.mA.fA.mC |
| 151 | 795 | PmU.fA.mG.fA.mC.fA.mG.fC.mA.fU.mC.fA.mU.fG.mG.fC.mU.fG.mC |
| 152 | 796 | PmU.fU.mC.fA.mU.fG.mG.fC.mU.fG.mC.fA.mA.fU.mG.fC.mC.fA.mG |
| 153 | 797 | PmU.fA.mC.fA.mG.fC.mC.fU.mG.fG.mC.fA.mU.fA.mG.fA.mG.fC.mA |
| 154 | 798 | PmU.fG.mG.fC.mA.fU.mA.fG.mA.fG.mC.fA.mG.fA.mG.fU.mA.fA.mA |
| 155 | 799 | PmU.fC.mU.fG.mG.fU.mU.fG.mG.fG.mC.fU.mG.fA.mC.fC.mU.fC.mG |
| 156 | 800 | PmU.fA.mG.fA.mC.fC.mC.fA.mA.fA.mA.fG.mA.fU.mA.fA.mA.fU.mG |
| 157 | 801 | PmU.fG.mG.fU.mC.fC.mA.fC.mA.fC.mA.fG.mC.fG.mG.fC.mC.fA.mA |
| 158 | 802 | PmU.fC.mC.fU.mC.fC.mA.fC.mC.fA.mG.fG.mC.fU.mG.fC.mC.fU.mC |
| 159 | 803 | PmU.fA.mA.fA.mA.fC.mA.fG.mC.fU.mG.fC.mC.fA.mA.fC.mC.fU.mG |
| 160 | 804 | PmU.fG.mA.fG.mG.fA.mC.fA.mG.fA.mC.fC.mC.fA.mA.fA.mA.fG.mA |
| 161 | 805 | PmU.fU.mG.fU.mC.fU.mG.fC.mU.fU.mG.fC.mU.fU.mG.fG.mG.fU.mG |
| 162 | 806 | PmU.fA.mA.fG.mG.fC.mA.fA.mC.fA.mG.fA.mG.fA.mG.fG.mA.fC.mA |
| 163 | 807 | PmU.fG.mU.fG.mU.fC.mU.fA.mG.fG.mA.fG.mA.fU.mA.fC.mA.fC.mC |
| 164 | 808 | PmU.fG.mU.fG.mG.fA.mU.fC.mA.fG.mU.fC.mU.fC.mU.fG.mC.fC.mU |
| 165 | 809 | PmU.fA.mA.fA.mC.fA.mG.fC.mU.fG.mC.fC.mA.fA.mC.fC.mU.fG.mC |
| 166 | 810 | PmU.fC.mA.fG.mG.fC.mG.fG.mC.fU.mU.fG.mU.fG.mG.fG.mU.fG.mC |
| 167 | 811 | PmU.fG.mG.fA.mA.fC.mC.fA.mG.fG.mC.fC.mU.fC.mA.fU.mU.fG.mA |
| 168 | 812 | PmU.fC.mU.fG.mC.fC.mU.fG.mG.fC.mU.fC.mA.fC.mU.fC.mC.fU.mC |
| 169 | 813 | PmU.fA.mG.fA.mU.fG.mA.fG.mG.fG.mC.fC.mA.fU.mC.fA.mG.fC.mA |
| 170 | 814 | PmU.fC.mU.fU.mC.fC.mU.fG.mG.fU.mC.fU.mG.fU.mG.fU.mU.fC.mC |
| 171 | 815 | PmU.fA.mC.fU.mU.fU.mG.fC.mA.fU.mU.fC.mC.fA.mG.fA.mC.fC.mU |
| 172 | 816 | PmU.fU.mG.fG.mC.fU.mA.fG.mA.fU.mG.fC.mC.fA.mU.fC.mC.fA.mG |
| 173 | 817 | PmU.fU.mG.fG.mC.fU.mG.fC.mA.fA.mU.fG.mC.fC.mA.fG.mC.fC.mA |
| 174 | 818 | PmU.fG.mG.fG.mU.fG.mU.fG.mG.fG.mU.fG.mC.fU.mU.fG.mA.fC.mG |
| 175 | 819 | PmU.fA.mA.fA.mA.fG.mC.fG.mU.fU.mG.fU.mG.fG.mG.fC.mC.fC.mG |
| 176 | 820 | PmU.fC.mA.fC.mC.fA.mG.fG.mA.fA.mG.fC.mC.fA.mG.fG.mA.fA.mG |
| 177 | 821 | PmU.fG.mC.fC.mG.fG.mG.fA.mU.fU.mC.fC.mA.fU.mG.fC.mU.fC.mC |
| 178 | 822 | PmU.fC.mC.fA.mG.fC.mU.fC.mC.fU.mC.fG.mU.fA.mG.fU.mC.fG.mC |
| 179 | 823 | PmU.fC.mC.fC.mG.fC.mU.fG.mG.fU.mC.fC.mU.fC.mA.fG.mG.fG.mA |
| 180 | 824 | PmU.fU.mG.fC.mA.fA.mU.fG.mC.fC.mA.fG.mC.fC.mA.fC.mG.fU.mG |
| 181 | 825 | PmU.fU.mG.fA.mU.fG.mA.fC.mA.fU.mC.fU.mU.fU.mG.fG.mC.fA.mG |
| 182 | 826 | PmU.fG.mA.fG.mA.fU.mA.fC.mA.fC.mC.fU.mC.fC.mA.fC.mC.fA.mG |
| 183 | 827 | PmU.fG.mG.fU.mG.fU.mG.fG.mG.fU.mG.fC.mU.fU.mG.fA.mC.fG.mC |
| 184 | 828 | PmU.fC.mU.fG.mC.fC.mU.fC.mA.fA.mC.fU.mC.fG.mG.fC.mC.fA.mG |
| 185 | 829 | PmU.fG.mA.fA.mG.fU.mC.fG.mG.fU.mG.fA.mC.fC.mA.fU.mG.fA.mC |
| 186 | 830 | PmU.fG.mU.fC.mC.fU.mC.fG.mU.fC.mC.fU.mC.fC.mU.fG.mC.fG.mC |
| 187 | 831 | PmU.fC.mC.fA.mG.fC.mC.fU.mC.fA.mC.fU.mG.fU.mU.fA.mC.fC.mC |
| 188 | 832 | PmU.fG.mC.fU.mU.fU.mU.fC.mC.fG.mA.fA.mU.fA.mA.fA.mC.fU.mC |
| 189 | 833 | PmU.fU.mG.fU.mG.fA.mG.fC.mU.fU.mG.fG.mC.fA.mG.fG.mC.fA.mC |
| 190 | 834 | PmU.fA.mG.fC.mG.fU.mG.fG.mA.fU.mG.fC.mU.fG.mG.fC.mC.fU.mC |
| 191 | 835 | PmU.fA.mA.fG.mA.fC.mA.fG.mA.fG.mG.fA.mG.fU.mC.fC.mU.fC.mC |
| 192 | 836 | PmU.fG.mU.fC.mU.fG.mC.fU.mU.fG.mC.fU.mU.fG.mG.fG.mU.fG.mG |
| 193 | 837 | PmU.fG.mU.fA.mG.fC.mA.fG.mG.fC.mA.fG.mC.fA.mC.fC.mU.fG.mG |
| 194 | 838 | PmU.fU.mG.fG.mA.fU.mG.fC.mU.fG.mG.fC.mC.fU.mC.fC.mC.fU.mG |
| 195 | 839 | PmU.fA.mA.fA.mA.fG.mA.fU.mA.fA.mA.fU.mG.fU.mC.fU.mG.fC.mU |
| 196 | 840 | PmU.fA.mG.fU.mC.fC.mU.fG.mC.fA.mA.fA.mA.fC.mA.fG.mC.fU.mG |
| 197 | 841 | PmU.fC.mA.fG.mG.fA.mU.fC.mU.fU.mG.fG.mU.fG.mA.fG.mG.fU.mA |
| 198 | 842 | PmU.fA.mG.fC.mG.fG.mU.fG.mG.fA.mA.fG.mG.fU.mG.fG.mC.fU.mG |
| 199 | 843 | PmU.fU.mG.fG.mC.fA.mA.fU.mG.fG.mC.fG.mU.fA.mG.fA.mC.fA.mC |
| 200 | 844 | PmU.fC.mA.fG.mA.fG.mG.fA.mG.fU.mC.fC.mU.fC.mC.fU.mC.fG.mA |
| 201 | 845 | PmU.fG.mG.fA.mG.fC.mA.fG.mC.fU.mC.fA.mG.fC.mA.fG.mC.fU.mC |
| 202 | 846 | PmU.fG.mU.fG.mU.fG.mA.fG.mC.fU.mU.fG.mG.fC.mA.fG.mG.fC.mA |
| 203 | 847 | PmU.fC.mA.fG.mC.fA.mC.fC.mU.fG.mG.fC.mA.fA.mU.fG.mG.fC.mG |
| 204 | 848 | PmU.fU.mC.fC.mC.fA.mG.fC.mC.fU.mC.fA.mC.fU.mG.fU.mU.fA.mC |
| 205 | 849 | PmU.fG.mA.fG.mA.fA.mA.fC.mU.fG.mG.fA.mG.fC.mA.fG.mC.fU.mC |
| 206 | 850 | PmU.fA.mG.fA.mG.fA.mA.fG.mU.fG.mG.fA.mU.fC.mA.fG.mU.fC.mU |
| 207 | 851 | PmU.fG.mG.fU.mC.fG.mC.fC.mA.fC.mU.fC.mA.fU.mC.fU.mU.fC.mA |
| 208 | 852 | PmU.fG.mA.fC.mC.fA.mG.fC.mU.fG.mG.fC.mU.fU.mU.fU.mC.fC.mG |
| 209 | 853 | PmU.fC.mA.fU.mG.fC.mA.fG.mG.fA.mU.fC.mU.fU.mG.fG.mU.fG.mA |
| 210 | 854 | PmU.fA.mG.fC.mG.fG.mC.fC.mA.fA.mA.fG.mU.fU.mG.fG.mU.fC.mC |
| 211 | 855 | PmU.fC.mA.fG.mC.fC.mU.fG.mU.fG.mA.fG.mG.fA.mC.fG.mU.fG.mG |
| 212 | 856 | PmU.fG.mA.fG.mC.fU.mG.fU.mG.fU.mG.fG.mA.fC.mG.fC.mU.fG.mC |
| 213 | 857 | PmU.fG.mG.fU.mA.fG.mC.fA.mG.fG.mC.fA.mG.fC.mA.fC.mC.fU.mG |
| 214 | 858 | PmU.fU.mG.fA.mG.fC.mU.fC.mC.fG.mG.fC.mU.fC.mG.fG.mC.fA.mG |
| 215 | 859 | PmU.fG.mU.fC.mU.fA.mG.fG.mA.fG.mA.fU.mA.fC.mA.fC.mC.fU.mC |
| 216 | 860 | PmU.fG.mG.fA.mA.fG.mC.fG.mG.fG.mU.fC.mC.fC.mG.fU.mC.fC.mU |
| 217 | 861 | PmU.fG.mC.fU.mG.fU.mG.fU.mG.fA.mG.fC.mU.fU.mG.fG.mC.fA.mG |
| 218 | 862 | PmU.fA.mC.fC.mA.fG.mC.fU.mG.fG.mC.fU.mU.fU.mU.fC.mC.fG.mA |
| 219 | 863 | PmU.fG.mC.fA.mA.fA.mG.fA.mG.fG.mU.fC.mC.fA.mC.fA.mC.fA.mG |
| 220 | 864 | PmU.fA.mG.fG.mA.fG.mA.fC.mC.fU.mA.fG.mA.fG.mG.fC.mC.fG.mU |
| 221 | 865 | PmU.fG.mU.fC.mC.fU.mG.fC.mA.fA.mA.fA.mC.fA.mG.fC.mU.fG.mC |
| 222 | 866 | PmU.fG.mC.fA.mC.fC.mA.fC.mC.fA.mC.fG.mU.fA.mG.fG.mU.fG.mC |
| 223 | 867 | PmU.fA.mC.fU.mC.fA.mA.fG.mG.fG.mC.fC.mA.fG.mG.fC.mC.fA.mG |
| 224 | 868 | PmU.fC.mC.fA.mU.fU.mU.fU.mA.fA.mA.fG.mC.fU.mC.fA.mG.fC.mC |
| 225 | 869 | PmU.fA.mG.fC.mG.fU.mU.fG.mU.fG.mG.fG.mC.fC.mC.fG.mG.fC.mA |
| 226 | 870 | PmU.fG.mG.fC.mA.fG.mA.fG.mA.fA.mG.fU.mG.fG.mA.fU.mC.fA.mG |
| 227 | 871 | PmU.fG.mA.fA.mA.fC.mC.fU.mU.fC.mU.fA.mG.fG.mG.fU.mG.fU.mG |
| 228 | 872 | PmU.fA.mG.fC.mG.fG.mU.fG.mA.fC.mC.fA.mG.fC.mA.fC.mG.fA.mC |
| 229 | 873 | PmU.fG.mC.fA.mG.fU.mC.fG.mC.fU.mG.fG.mA.fG.mG.fC.mA.fC.mC |
| 230 | 874 | PmU.fA.mA.fA.mA.fC.mC.fC.mA.fG.mA.fA.mU.fA.mA.fA.mU.fA.mU |
| 231 | 875 | PmU.fA.mA.fU.mG.fG.mC.fG.mU.fA.mG.fA.mC.fA.mC.fC.mC.fU.mC |
| 232 | 876 | PmU.fU.mC.fU.mU.fC.mA.fA.mG.fU.mU.fA.mC.fA.mA.fA.mA.fG.mC |
| 233 | 877 | PmU.fU.mU.fA.mC.fA.mA.fA.mA.fG.mC.fA.mA.fA.mA.fC.mA.fG.mG |
| 234 | 878 | PmU.fA.mU.fC.mU.fU.mC.fA.mA.fG.mU.fU.mA.fC.mA.fA.mA.fA.mG |
| 235 | 879 | PmU.fC.mU.fG.mG.fU.mG.fU.mC.fU.mA.fG.mG.fA.mG.fA.mU.fA.mC |
| 236 | 880 | PmU.fA.mG.fU.mG.fC.mG.fC.mU.fC.mU.fG.mA.fC.mU.fG.mC.fG.mA |
| 237 | 881 | PmU.fU.mG.fU.mC.fA.mC.fA.mC.fU.mU.fG.mC.fU.mG.fG.mC.fC.mU |
| 238 | 882 | PmU.fA.mC.fA.mG.fA.mG.fG.mA.fG.mU.fC.mC.fU.mC.fC.mU.fC.mG |
| 239 | 883 | PmU.fG.mU.fU.mA.fC.mA.fA.mA.fA.mG.fC.mA.fA.mA.fA.mC.fA.mG |
| 240 | 884 | PmU.fA.mA.fU.mC.fC.mU.fG.mC.fC.mU.fC.mC.fU.mU.fG.mG.fU.mG |
| 241 | 885 | PmU.fG.mU.fG.mG.fA.mA.fG.mG.fU.mG.fG.mC.fU.mG.fU.mG.fG.mU |
| 242 | 886 | PmU.fG.mG.fA.mG.fC.mG.fG.mG.fU.mU.fG.mG.fC.mU.fG.mA.fG.mA |
| 243 | 887 | PmU.fU.mC.fG.mC.fC.mA.fC.mU.fC.mA.fU.mC.fU.mU.fC.mA.fC.mC |
| 244 | 888 | PmU.fA.mG.fA.mG.fG.mA.fG.mU.fC.mC.fU.mC.fC.mU.fC.mG.fA.mU |
| 245 | 889 | PmU.fG.mA.fG.mC.fU.mC.fC.mG.fG.mC.fU.mC.fG.mG.fC.mA.fG.mA |
| 246 | 890 | PmU.fA.mA.fC.mU.fG.mU.fG.mA.fU.mG.fA.mC.fC.mU.fC.mG.fG.mG |
| 247 | 891 | PmU.fG.mA.fU.mA.fC.mA.fC.mC.fU.mC.fC.mA.fC.mC.fA.mG.fG.mC |
| 248 | 892 | PmU.fU.mG.fG.mC.fA.mU.fA.mG.fA.mG.fC.mA.fG.mA.fG.mU.fA.mA |
| 249 | 893 | PmU.fG.mC.fA.mG.fC.mU.fC.mA.fG.mC.fA.mG.fC.mU.fC.mC.fU.mC |
| 250 | 894 | PmU.fA.mU.fU.mG.fA.mU.fG.mA.fC.mA.fU.mC.fU.mU.fU.mG.fG.mC |

TABLE 2c

| | SEQ ID | |
|---|---|---|
| # | NO: | 250 sense nucleobase sequences |
| 1 | 251 | CACCCUCAUAGGCCA |
| 2 | 252 | CCUGGAGUUUAUUCA |
| 3 | 253 | AGGCCUGGAGUUUAA |
| 4 | 254 | CUGGAGUUUAUUCGA |
| 5 | 255 | CAUCUGCUGCCGGAA |
| 6 | 256 | CAUAGGCCUGGAGUA |
| 7 | 257 | GGCACCCUCAUAGGA |
| 8 | 258 | ACCCUCAUAGGCCUA |
| 9 | 259 | CUCAUAGGCCUGGAA |
| 10 | 260 | CCCUGAGGACCAGCA |
| 11 | 261 | GCCUGGAGUUUAUUA |
| 12 | 262 | CUGAGCUGCUCCAGA |
| 13 | 263 | UGGAGUUUAUUCGGA |
| 14 | 264 | GUUCCCUGAGGACCA |
| 15 | 265 | UUCCCUGAGGACCAA |
| 16 | 266 | GGCCUGGAGUUUAUA |
| 17 | 267 | UCAUAGGCCUGGAGA |
| 18 | 268 | CCCUCAUAGGCCUGA |
| 19 | 269 | GGUUCCCUGAGGACA |
| 20 | 270 | GCACCCUCAUAGGCA |
| 21 | 271 | GAGUUUAUUCGGAAA |
| 22 | 272 | UCCUGCUGCCAUGCA |
| 23 | 273 | UCUGCUGCCGGAGCA |
| 24 | 274 | CUGCCGGAGCCGGCA |
| 25 | 275 | UGGUUCCCUGAGGAA |
| 26 | 276 | AUCUGCUGCCGGAGA |
| 27 | 277 | GCUGCCCACGUGGCA |
| 28 | 278 | UUUUGUAACUUGAAA |
| 29 | 279 | UAGACCUGUUUUGCA |
| 30 | 280 | UAUUUAUUCUGGGUA |
| 31 | 281 | AAGCAGACAUUUAUA |
| 32 | 282 | CUUUUGUAACUUGAA |
| 33 | 283 | CUGUCCUCUCUGUUA |
| 34 | 284 | ACUUGAAGAUAUUUA |
| 35 | 285 | UUUAAAAUGGUUCCA |
| 36 | 286 | CUAGACCUGUUUUGA |
| 37 | 287 | GUUUUGUAGCAUUUA |
| 38 | 288 | CAUUUAUCUUUUGGA |
| 39 | 289 | CAUUGCCAGGUGCUA |
| 40 | 290 | GGAGGCAGGAUUCUA |
| 41 | 291 | CAGAGACUGAUCCAA |
| 42 | 292 | AUAUUUAUUCUGGGA |
| 43 | 293 | GAUAUUUAUUCUGGA |
| 44 | 294 | UUUGUAACUUGAAGA |
| 45 | 295 | GGGUUUUGUAGCAUA |
| 46 | 296 | CCUGUUUUGCUUUUA |
| 47 | 297 | AACUUGAAGAUAUUA |
| 48 | 298 | CUGCUUUGUGUCACA |
| 49 | 299 | UUGAAGAUAUUUAUA |
| 50 | 300 | GGUUUUGUAGCAUUA |
| 51 | 301 | CAGCACCUGCUUUGA |
| 52 | 302 | AAGAUAUUUAUUCUA |
| 53 | 303 | UGUUUUGCUUUUGUA |
| 54 | 304 | AUUCUGGGUUUUGUA |
| 55 | 305 | ACCUGUUUUGCUUUA |
| 56 | 306 | GGUCACCGACUUCGA |
| 57 | 307 | CUGUUUUGCUUUUGA |
| 58 | 308 | GCUUUUGUAACUUGA |
| 59 | 309 | AUGCAAAGUCAAGGA |
| 60 | 310 | CAGCAUCCACGCUUA |
| 61 | 311 | CAAGCAGACAUUUAA |
| 62 | 312 | GCUUUAAAAUGGUUA |
| 63 | 313 | CAAUGAGGCCUGGUA |
| 64 | 314 | UUUAUUCUGGGUUUA |
| 65 | 315 | CUUUAAAAUGGUUCA |
| 66 | 316 | GAAGAUAUUUAUUCA |
| 67 | 317 | AAGCCAAGCCUCUUA |
| 68 | 318 | AUGAGGCCUGGUUCA |
| 69 | 319 | AGAUAUUUAUUCUGA |
| 70 | 320 | GAGGCAGGAUUCUUA |
| 71 | 321 | UCUAGACCUGUUUUA |
| 72 | 322 | CAUCCACGCUUCCUA |
| 73 | 323 | CAGCCCAACCAGUGA |
| 74 | 324 | GAUCCUGCAUGUCUA |
| 75 | 325 | GGUGUAUCUCCUAGA |
| 76 | 326 | UCUGGGUUUUGUAGA |
| 77 | 327 | CAUGAUGCUGUCUGA |
| 78 | 328 | UAGACACCAGCAUAA |
| 79 | 329 | GCUGCUCCAGUUUCA |
| 80 | 330 | UUUGGGUCUGUCCUA |
| 81 | 331 | UGUCUACGCCAUUGA |
| 82 | 332 | GACUGCAGCACCUGA |
| 83 | 333 | CACCAAGGAGGCAGA |
| 84 | 334 | CAUGGUCACCGACUA |
| 85 | 335 | UCUAUGCCAGGCUGA |
| 86 | 336 | GUCUGUCCUCUCUGA |
| 87 | 337 | AGGAACUGAGCCAGA |
| 88 | 338 | AGACCAGGAAGCUCA |
| 89 | 339 | CGACUGCAGCACCUA |
| 90 | 340 | CACAGACAGGCCAGA |
| 91 | 341 | CAGCUCCAGGCGGUA |
| 92 | 342 | CCACAACGCUUUUGA |
| 93 | 343 | UAUGCCAGGCUGUGA |
| 94 | 344 | GAGCAUGGAAUCCCA |
| 95 | 345 | AGGGUCAUGGUCACA |
| 96 | 346 | AGACCUGUUUUGCUA |
| 97 | 347 | GAGGAGUGAGCCAGA |
| 98 | 348 | GACUGAUCCACUUCA |
| 99 | 349 | CUAGACACCAGCAUA |
| 100 | 350 | CAGCAGGAACUGAGA |
| 101 | 351 | CUGGUGGAGGUGUAA |
| 102 | 352 | AUUGCAGCCAUGAUA |
| 103 | 353 | UCUGUCCUCUCUGUA |
| 104 | 354 | AGCCAAGCCUCUUCA |
| 105 | 355 | AAUCGAGGGCAGGGA |
| 106 | 356 | CCUGGCUUCCUGGUA |
| 107 | 357 | CAGGAAGCUCCCUCA |
| 108 | 358 | AGCUUUAAAAUGGUA |
| 109 | 359 | CAGGCCAGCAAGUGA |
| 110 | 360 | UGAAGAUGAGUGGCA |
| 111 | 361 | CACUUCUCUGCCAAA |
| 112 | 362 | CAUCGAGGAGGACUA |
| 113 | 363 | CAUCUAGCCAGAGGA |
| 114 | 364 | GUCCACACAGCUCCA |
| 115 | 365 | CUGAUCCACUUCUCA |
| 116 | 366 | CGUUGCCAUCUGCUA |
| 117 | 367 | CAUCAAUGAGGCCUA |
| 118 | 368 | CUCCAGCGACUGCAA |
| 119 | 369 | AAGGAGGAGACCCAA |
| 120 | 370 | UGUCUGCCGAGCCGA |
| 121 | 371 | UGCCAAAGAUGUCAA |
| 122 | 372 | CUAGCCUUGCGUUCA |
| 123 | 373 | UCUCUGCCAAAGAUA |
| 124 | 374 | GGACGAUGCCUGCCA |
| 125 | 375 | UAGCCUUGCGUUCCA |
| 126 | 376 | ACCUGCUUUGUGUCA |
| 127 | 377 | UAACUUGAAGAUAUA |
| 128 | 378 | CAAGGGAAGGGCACA |
| 129 | 379 | CGUGGCUGGCAUUGA |
| 130 | 380 | UGAAGCCAAGCCUCA |
| 131 | 381 | CCUCACCAAGAUCCA |
| 132 | 382 | GGUCAUCACAGUUGA |
| 133 | 383 | GCAAGCAGACAUUUA |
| 134 | 384 | UUCUAGACCUGUUUA |
| 135 | 385 | GCCCACAACGCUUUA |
| 136 | 386 | UGUCCUCUCUGUUGA |
| 137 | 387 | AAGGAUCCGUGGAGA |
| 138 | 388 | UCCUGCACAGUCCUA |
| 139 | 389 | ACAUUUAUCUUUUGA |
| 140 | 390 | CAAGGAGGCAGGAUA |
| 141 | 391 | CUUUGGCCGCUGUGA |
| 142 | 392 | CACAGCAGGAACUGA |
| 143 | 393 | CUGCUCUAUGCCAGA |
| 144 | 394 | UGUCAUCAAUGAGGA |
| 145 | 395 | CGAGGACGGCGACUA |
| 146 | 396 | CAGGAUUCUUCCCAA |
| 147 | 397 | CGUCAAGCACCCACA |
| 148 | 398 | CACCUGCUUUGUGUA |
| 149 | 399 | CCACGCUUCCUGCUA |
| 150 | 400 | AGGCAGAGACUGAUA |
| 151 | 401 | CCAUGAUGCUGUCUA |
| 152 | 402 | CAUUGCAGCCAUGAA |
| 153 | 403 | CUAUGCCAGGCUGUA |
| 154 | 404 | CUCUGCUCUAUGCCA |
| 155 | 405 | GUCAGCCCAACCAGA |
| 156 | 406 | UAUCUUUUGGGUCUA |
| 157 | 407 | CCGCUGUGUGGACCA |
| 158 | 408 | CAGCCUGGUGGAGGA |
| 159 | 409 | UUGGCAGCUGUUUUA |
| 160 | 410 | UUGGGUCUGUCCUCA |
| 161 | 411 | CAAGCAAGCAGACAA |
| 162 | 412 | CUCUCUGUUGCCUUA |
| 163 | 413 | UAUCUCCUAGACACA |
| 164 | 414 | AGAGACUGAUCCACA |
| 165 | 415 | GUUGGCAGCUGUUUA |
| 166 | 416 | CCACAAGCCGCCUGA |
| 167 | 417 | UGAGGCCUGGUUCCA |
| 168 | 418 | AGUGAGCCAGGCAGA |
| 169 | 419 | GAUGGCCCUCAUCUA |
| 170 | 420 | CACAGACCAGGAAGA |
| 171 | 421 | CUGGAAUGCAAAGUA |
| 172 | 422 | AUGGCAUCUAGCCAA |
| 173 | 423 | UGGCAUUGCAGCCAA |
| 174 | 424 | AAGCACCCACACCCA |
| 175 | 425 | CCCACAACGCUUUUA |
| 176 | 426 | CUGGCUUCCUGGUGA |
| 177 | 427 | CAUGGAAUCCCGGCA |
| 178 | 428 | CUACGAGGAGCUGGA |
| 179 | 429 | UGAGGACCAGCGGGA |
| 180 | 430 | UGGCUGGCAUUGCAA |
| 181 | 431 | CAAAGAUGUCAUCAA |
| 182 | 432 | UGGAGGUGUAUCUCA |
| 183 | 433 | CAAGCACCCACACCA |
| 184 | 434 | CCGAGUUGAGGCAGA |
| 185 | 435 | UGGUCACCGACUUCA |
| 186 | 436 | AGGAGGACGAGGACA |
| 187 | 437 | AACAGUGAGGCUGGA |
| 188 | 438 | UUAUUCGGAAAAGCA |
| 189 | 439 | CUGCCAAGCUCACAA |
| 190 | 440 | CCAGCAUCCACGCUA |
| 191 | 441 | GACUCCUCUGUCUUA |
| 192 | 442 | CCAAGCAAGCAGACA |
| 193 | 443 | GUGCUGCCUGCUACA |
| 194 | 444 | GAGGCCAGCAUCCAA |
| 195 | 445 | GACAUUUAUCUUUUA |
| 196 | 446 | UGUUUUGCAGGACUA |
| 197 | 447 | UCACCAAGAUCCUGA |
| 198 | 448 | CACCUUCCACCGCUA |
| 199 | 449 | CUACGCCAUUGCCAA |
| 200 | 450 | GGAGGACUCCUCUGA |
| 201 | 451 | UGCUGAGCUGCUCCA |
| 202 | 452 | UGCCAAGCUCACACA |
| 203 | 453 | AUUGCCAGGUGCUGA |
| 204 | 454 | CAGUGAGGCUGGGAA |
| 205 | 455 | UGCUCCAGUUUCUCA |
| 206 | 456 | UGAUCCACUUCUCUA |
| 207 | 457 | GAUGAGUGGCGACCA |
| 208 | 458 | AAAGCCAGCUGGUCA |
| 209 | 459 | CAAGAUCCUGCAUGA |
| 210 | 460 | CAACUUUGGCCGCUA |
| 211 | 461 | GUCCUCACAGGCUGA |
| 212 | 462 | CGUCCACACAGCUCA |
| 213 | 463 | UGCUGCCUGCUACCA |
| 214 | 464 | CGAGCCGGAGCUCAA |
| 215 | 465 | UGUAUCUCCUAGACA |
| 216 | 466 | CGGGACCCGCUUCCA |
| 217 | 467 | CAAGCUCACACAGCA |
| 218 | 468 | AAAAGCCAGCUGGUA |
| 219 | 469 | GUGGACCUCUUUGCA |
| 220 | 470 | CCUCUAGGUCUCCUA |
| 221 | 471 | CUGUUUUGCAGGACA |
| 222 | 472 | CUACGUGGUGGUGCA |
| 223 | 473 | CCUGGCCCUUGAGUA |
| 224 | 474 | GAGCUUUAAAAUGGA |
| 225 | 475 | GGGCCCACAACGCUA |
| 226 | 476 | UCCACUUCUCUGCCA |
| 227 | 477 | CCCUAGAAGGUUUCA |
| 228 | 478 | UGCUGGUCACCGCUA |
| 229 | 479 | CCUCCAGCGACUGCA |
| 230 | 480 | UUAUUCUGGGUUUUA |
| 231 | 481 | GUGUCUACGCCAUUA |
| 232 | 482 | UUGUAACUUGAAGAA |
| 233 | 483 | UUUUGCUUUUGUAAA |
| 234 | 484 | UGUAACUUGAAGAUA |
| 235 | 485 | CUCCUAGACACCAGA |
| 236 | 486 | AGUCAGAGCGCACUA |
| 237 | 487 | CAGCAAGUGUGACAA |
| 238 | 488 | GAGGACUCCUCUGUA |
| 239 | 489 | UUUGCUUUUGUAACA |
| 240 | 490 | AAGGAGGCAGGAUUA |
| 241 | 491 | CAGCCACCUUCCACA |
| 242 | 492 | AGCCAACCCGCUCCA |
| 243 | 493 | AAGAUGAGUGGCGAA |
| 244 | 494 | AGGAGGACUCCUCUA |
| 245 | 495 | CCGAGCCGGAGCUCA |
| 246 | 496 | AGGUCAUCACAGUUA |
| 247 | 497 | GGUGGAGGUGUAUCA |
| 248 | 498 | UCUGCUCUAUGCCAA |
| 249 | 499 | AGCUGCUGAGCUGCA |
| 250 | 500 | AAGAUGUCAUCAAUA |

TABLE 2d

| | SEQ ID | |
|---|---|---|
| # | NO: | 250 modified sense strands |
| 1 | 895 | fC.mA.fC.mC.fC.mU.fC.mA.fU.mA.fG.mG.fC.mC.fA |
| 2 | 896 | fC.mC.fU.mG.fG.mA.fG.mU.fU.mU.fA.mU.fU.mC.fA |
| 3 | 897 | fA.mG.fG.mC.fC.mU.fG.mG.fA.mG.fU.mU.fU.mA.fA |
| 4 | 898 | fC.mU.fG.mG.fA.mG.fU.mU.fU.mA.fU.mU.fC.mG.fA |
| 5 | 899 | fC.mA.fU.mC.fU.mG.fC.mU.fG.mC.fC.mG.fG.mA.fA |
| 6 | 900 | f.mA.fU.mA.fG.mG.fC.mC.fU.mG.fG.mA.fG.mU.fA |
| 7 | 901 | fG.mG.fC.mA.fC.mC.fC.mU.fC.mA.fU.mA.fG.mG.fA |
| 8 | 902 | fA.mC.fC.mC.fU.mC.fA.mU.fA.mG.fG.mC.fC.mU.fA |
| 9 | 903 | fC.mU.fC.mA.fU.mA.fG.mG.fC.mC.fU.mG.fG.mA.fA |
| 10 | 904 | fC.mC.fC.mU.fG.mA.fG.mG.fA.mC.fC.mA.fG.mC.fA |
| 11 | 905 | fG.mC.fC.mU.fG.mG.fA.mG.fU.mU.fU.mA.fU.mU.fA |
| 12 | 906 | fC.mU.fG.mA.fG.mC.fU.mG.fC.mU.fC.mC.fA.mG.fA |
| 13 | 907 | fU.mG.fG.mA.fG.mU.fU.mU.fA.mU.fU.mC.fG.mG.fA |
| 14 | 908 | fG.mU.fU.mC.fC.mC.fU.mG.fA.mG.fG.mA.fC.mC.fA |
| 15 | 909 | fU.mU.fC.mC.fC.mU.fG.mA.fG.mG.fA.mC.fC.mA.fA |
| 16 | 910 | fG.mG.fC.mC.fU.mG.fG.mA.fG.mU.fU.mU.fA.mU.fA |
| 17 | 911 | fU.mC.fA.mU.fA.mG.fG.mC.fC.mU.fG.mG.fA.mG.fA |
| 18 | 912 | fC.mC.fC.mU.fC.mA.fU.mA.fG.mG.fC.mC.fU.mG.fA |
| 19 | 913 | fG.mG.fU.mU.fC.mC.fC.mU.fG.mA.fG.mG.fA.mC.fA |
| 20 | 914 | fG.mC.fA.mC.fC.mC.fU.mC.fA.mU.fA.mG.fG.mC.fA |
| 21 | 915 | fG.mA.fG.mU.fU.mU.fA.mU.fU.mC.fG.mG.fA.mA.fA |
| 22 | 916 | fU.mC.fC.mU.fG.mC.fU.mG.fC.mC.fA.mU.fG.mC.fA |
| 23 | 917 | fU.mC.fU.mG.fC.mU.fG.mC.fC.mG.fG.mA.fG.mC.fA |
| 24 | 918 | fC.mU.fG.mC.fC.mG.fG.mA.fG.mC.fC.mG.fG.mC.fA |
| 25 | 919 | fU.mG.fG.mU.fU.mC.fC.mC.fU.mG.fA.mG.fG.mA.fA |
| 26 | 920 | fA.mU.fC.mU.fG.mC.fU.mG.fC.mC.fG.mG.fA.mG.fA |
| 27 | 921 | fG.mC.fU.mG.fC.mC.fC.mA.fC.mG.fU.mG.fG.mC.fA |
| 28 | 922 | fU.mU.fU.mU.fG.mU.fA.mA.fC.mU.fU.mG.fA.mA.fA |
| 29 | 923 | fU.mA.fG.mA.fC.mC.fU.mG.fU.mU.fU.mU.fG.mC.fA |
| 30 | 924 | fU.mA.fU.mU.fU.mA.fU.mU.fC.mU.fG.mG.fG.mU.fA |
| 31 | 925 | fA.mA.fG.mC.fA.mG.fA.mC.fA.mU.fU.mU.fA.mU.fA |
| 32 | 926 | fC.mU.fU.mU.fU.mG.fU.mA.fA.mC.fU.mU.fG.mA.fA |
| 33 | 927 | fC.mU.fG.mU.fC.mC.fU.mC.fU.mC.fU.mG.fU.mU.fA |
| 34 | 928 | fA.mC.fU.mU.fG.mA.fA.mG.fA.mU.fA.mU.fU.mU.fA |
| 35 | 929 | fU.mU.fU.mA.fA.mA.fA.mU.fG.mG.fU.mU.fC.mC.fA |
| 36 | 930 | fC.mU.fA.mG.fA.mC.fC.mU.fG.mU.fU.mU.fU.mG.fA |
| 37 | 931 | fG.mU.fU.mU.fU.mG.fU.mA.fG.mC.fA.mU.fU.mU.fA |
| 38 | 932 | fC.mA.fU.mU.fU.mA.fU.mC.fU.mU.fU.mU.fG.mG.fA |
| 39 | 933 | fC.mA.fU.mU.fG.mC.fC.mA.fG.mG.fU.mG.fC.mU.fA |
| 40 | 934 | fG.mG.fA.mG.fG.mC.fA.mG.fG.mA.fU.mU.fC.mU.fA |
| 41 | 935 | fC.mA.fG.mA.fG.mA.fC.mU.fG.mA.fU.mC.fC.mA.fA |
| 42 | 936 | fA.mU.fA.mU.fU.mU.fA.mU.fU.mC.fU.mG.fG.mG.fA |
| 43 | 937 | fG.mA.fU.mA.fU.mU.fU.mA.fU.mU.fC.mU.fG.mG.fA |
| 44 | 938 | fU.mU.fU.mG.fU.mA.fA.mC.fU.mU.fG.mA.fA.mG.fA |
| 45 | 939 | fG.mG.fG.mU.fU.mU.fU.mG.fU.mA.fG.mC.fA.mU.fA |
| 46 | 940 | fC.mC.fU.mG.fU.mU.fU.mU.fG.mC.fU.mU.fU.mU.fA |
| 47 | 941 | fA.mA.fC.mU.fU.mG.fA.mA.fG.mA.fU.mA.fU.mU.fA |
| 48 | 942 | fC.mU.fG.mC.fU.mU.fU.mG.fU.mG.fU.mC.fA.mC.fA |
| 49 | 943 | fU.mU.fG.mA.fA.mG.fA.mU.fA.mU.fU.mU.fA.mU.fA |
| 50 | 944 | fG.mG.fU.mU.fU.mU.fG.mU.fA.mG.fC.mA.fU.mU.fA |
| 51 | 945 | fC.mA.fG.mC.fA.mC.fC.mU.fG.mC.fU.mU.fU.mG.fA |
| 52 | 946 | fA.mA.fG.mA.fU.mA.fU.mU.fU.mA.fU.mU.fC.mU.fA |
| 53 | 947 | fU.mG.fU.mU.fU.mU.fG.mC.fU.mU.fU.mU.fG.mU.fA |
| 54 | 948 | fA.mU.fU.mC.fU.mG.fG.mG.fU.mU.fU.mU.fG.mU.fA |
| 55 | 949 | fA.mC.fC.mU.fG.mU.fU.mU.fU.mG.fC.mU.fU.mU.fA |
| 56 | 950 | fG.mG.fU.mC.fA.mC.fC.mG.fA.mC.fU.mU.fC.mG.fA |
| 57 | 951 | fC.mU.fG.mU.fU.mU.fU.mG.fC.mU.fU.mU.fU.mG.fA |
| 58 | 952 | fG.mC.fU.mU.fU.mU.fG.mU.fA.mA.fC.mU.fU.mG.fA |
| 59 | 953 | fA.mU.fG.mC.fA.mA.fA.mG.fU.mC.fA.mA.fG.mG.fA |
| 60 | 954 | fC.mA.fG.mC.fA.mU.fC.mC.fA.mC.fG.mC.fU.mU.fA |
| 61 | 955 | fC.mA.fA.mG.fC.mA.fG.mA.fC.mA.fU.mU.fU.mA.fA |
| 62 | 956 | fG.mC.fU.mU.fU.mA.fA.mA.fA.mU.fG.mG.fU.mU.fA |
| 63 | 957 | fC.mA.fA.mU.fG.mA.fG.mG.fC.mC.fU.mG.fG.mU.fA |
| 64 | 958 | fU.mU.fU.mA.fU.mU.fC.mU.fG.mG.fG.mU.fU.mU.fA |
| 65 | 959 | fC.mU.fU.mU.fA.mA.fA.mA.fU.mG.fG.mU.fU.mC.fA |
| 66 | 960 | fG.mA.fA.mG.fA.mU.fA.mU.fU.mU.fA.mU.fU.mC.fA |
| 67 | 961 | fA.mA.fG.mC.fC.mA.fA.mG.fC.mC.fU.mC.fU.mU.fA |
| 68 | 962 | fA.mU.fG.mA.fG.mG.fC.mC.fU.mG.fG.mU.fU.mC.fA |
| 69 | 963 | fA.mG.fA.mU.fA.mU.fU.mU.fA.mU.fU.mC.fU.mG.fA |
| 70 | 964 | fG.mA.fG.mG.fC.mA.fG.mG.fA.mU.fU.mC.fU.mU.fA |
| 71 | 965 | fU.mC.fU.mA.fG.mA.fC.mC.fU.mG.fU.mU.fU.mU.fA |
| 72 | 966 | fC.mA.fU.mC.fC.mA.fC.mG.fC.mU.fU.mC.fC.mU.fA |
| 73 | 967 | fC.mA.fG.mC.fC.mC.fA.mA.fC.mC.fA.mG.fU.mG.fA |
| 74 | 968 | fG.mA.fU.mC.fC.mU.fG.mC.fA.mU.fG.mU.fC.mU.fA |
| 75 | 969 | fG.mG.fU.mG.fU.mA.fU.mC.fU.mC.fC.mU.fA.mG.fA |
| 76 | 970 | fU.mC.fU.mG.fG.mG.fU.mU.fU.mU.fG.mU.fA.mG.fA |
| 77 | 971 | fC.mA.fU.mG.fA.mU.fG.mC.fU.mG.fU.mC.fU.mG.fA |
| 78 | 972 | fU.mA.fG.mA.fC.mA.fC.mC.fA.mG.fC.mA.fU.mA.fA |
| 79 | 973 | fG.mC.fU.mG.fC.mU.fC.mC.fA.mG.fU.mU.fU.mC.fA |
| 80 | 974 | fU.mU.fU.mG.fG.mG.fU.mC.fU.mG.fU.mC.fC.mU.fA |
| 81 | 975 | fU.mG.fU.mC.fU.mA.fC.mG.fC.mC.fA.mU.fU.mG.fA |
| 82 | 976 | fG.mA.fC.mU.fG.mC.fA.mG.fC.mA.fC.mC.fU.mG.fA |
| 83 | 977 | fC.mA.fC.mC.fA.mA.fG.mG.fA.mG.fG.mC.fA.mG.fA |
| 84 | 978 | fC.mA.fU.mG.fG.mU.fC.mA.fC.mC.fG.mA.fC.mU.fA |
| 85 | 979 | fU.mC.fU.mA.fU.mG.fC.mC.fA.mG.fG.mC.fU.mG.fA |
| 86 | 980 | fG.mU.fC.mU.fG.mU.fC.mC.fU.mC.fU.mC.fU.mG.fA |
| 87 | 981 | fA.mG.fG.mA.fA.mC.fU.mG.fA.mG.fC.mC.fA.mG.fA |
| 88 | 982 | fA.mG.fA.mC.fC.mA.fG.mG.fA.mA.fG.mC.fU.mC.fA |
| 89 | 983 | fC.mG.fA.mC.fU.mG.fC.mA.fG.mC.fA.mC.fC.mU.fA |
| 90 | 984 | fC.mA.fC.mA.fG.mA.fC.mA.fG.mG.fC.mC.fA.mG.fA |
| 91 | 985 | fC.mA.fG.mC.fU.mC.fC.mA.fG.mG.fC.mG.fG.mU.fA |
| 92 | 986 | fC.mC.fA.mC.fA.mA.fC.mG.fC.mU.fU.mU.fU.mG.fA |
| 93 | 987 | fU.mA.fU.mG.fC.mC.fA.mG.fG.mC.fU.mG.fU.mG.fA |
| 94 | 988 | fG.mA.fG.mC.fA.mU.fG.mG.fA.mA.fU.mC.fC.mC.fA |
| 95 | 989 | fA.mG.fG.mG.fU.mC.fA.mU.fG.mG.fU.mC.fA.mC.fA |
| 96 | 990 | fA.mG.fA.mC.fC.mU.fG.mU.fU.mU.fU.mG.fC.mU.fA |
| 97 | 991 | fG.mA.fG.mG.fA.mG.fU.mG.fA.mG.fC.mC.fA.mG.fA |
| 98 | 992 | fG.mA.fC.mU.fG.mA.fU.mC.fC.mA.fC.mU.fU.mC.fA |
| 99 | 993 | fC.mU.fA.mG.fA.mC.fA.mC.fC.mA.fG.mC.fA.mU.fA |
| 100 | 994 | fC.mA.fG.mC.fA.mG.fG.mA.fA.mC.fU.mG.fA.mG.fA |
| 101 | 995 | fC.mU.fG.mG.fU.mG.fG.mA.fG.mG.fU.mG.fU.mA.fA |
| 102 | 996 | fA.mU.fU.mG.fC.mA.fG.mC.fC.mA.fU.mG.fA.mU.fA |
| 103 | 997 | fU.mC.fU.mG.fU.mC.fC.mU.fC.mU.fC.mU.fG.mU.fA |
| 104 | 998 | fA.mG.fC.mC.fA.mA.fG.mC.fC.mU.fC.mU.fU.mC.fA |
| 105 | 999 | fA.mA.fU.mC.fG.mA.fG.mG.fG.mC.fA.mG.fG.mG.fA |
| 106 | 1000 | fC.mC.fU.mG.fG.mC.fU.mU.fC.mC.fU.mG.fG.mU.fA |
| 107 | 1001 | fC.mA.fG.mG.fA.mA.fG.mC fU.mC.fC.mC fU.mC.fA |
| 108 | 1002 | fA.mG.fC.mU.fU.mU.fA.mA.fA.mA.fU.mG.fG.mU.fA |
| 109 | 1003 | fC.mA.fG.mG.fC.mC.fA.mG.fC.mA.fA.mG.fU.mG.fA |
| 110 | 1004 | fU.mG.fA.mA.fG.mA.fU.mG.fA.mG.fU.mG.fG.mC.fA |
| 111 | 1005 | fC.mA.fC.mU.fU.mC.fU.mC.fU.mG.fC.mC.fA.mA.fA |
| 112 | 1006 | fC.mA.fU.mC.fG.mA.fG.mG.fA.mG.fG.mA.fC.mU.fA |
| 113 | 1007 | fC.mA.fU.mC.fU.mA.fG.mC.fC.mA.fG.mA.fG.mG.fA |
| 114 | 1008 | fG.mU.fC.mC.fA.mC.fA.mC.fA.mG.fC.mU.fC.mC.fA |
| 115 | 1009 | fC.mU.fG.mA.fU.mC.fC.mA.fC.mU.fU.mC.fU.mC.fA |
| 116 | 1010 | fC.mG.fU.mU.fG.mC.fC.mA.fU.mC.fU.mG.fC.mU.fA |
| 117 | 1011 | fC.mA.fU.mC.fA.mA.fU.mG.fA.mG.fG.mC.fC.mU.fA |
| 118 | 1012 | fC.mU.fC.mC.fA.mG.fC.mG.fA.mC.fU.mG.fC.mA.fA |
| 119 | 1013 | fA.mA.fG.mG.fA.mG.fG.mA.fG.mA.fC.mC.fC.mA.fA |
| 120 | 1014 | fU.mG.fU.mC.fU.mG.fC.mC.fG.mA.fG.mC.fC.mG.fA |
| 121 | 1015 | fU.mG.fC.mC.fA.mA.fA.mG.fA.mU.fG.mU.fC.mA.fA |
| 122 | 1016 | fC.mU.fA.mG.fC.mC.fU.mU.fG.mC.fG.mU.fU.mC.fA |
| 123 | 1017 | fU.mC.fU.mC.fU.mG.fC.mC.fA.mA.fA.mG.fA.mU.fA |
| 124 | 1018 | fG.mG.fA.mC.fG.mA.fU.mG.fC.mC.fU.mG.fC.mC.fA |
| 125 | 1019 | fU.mA.fG.mC.fC.mU.fU.mG.fC.mG.fU.mU.fC.mC.fA |
| 126 | 1020 | fA.mC.fC.mU.fG.mC.fU.mU.fU.mG.fU.mG.fU.mC.fA |
| 127 | 1021 | fU.mA.fA.mC.fU.mU.fG.mA.fA.mG.fA.mU.fA.mU.fA |
| 128 | 1022 | fC.mA.fA.mG.fG.mG.fA.mA.fG.mG.fG.mC.fA.mC.fA |
| 129 | 1023 | fC.mG.fU.mG.fG.mC.fU.mG.fG.mC.fA.mU.fU.mG.fA |
| 130 | 1024 | fU.mG.fA.mA.fG.mC.fC.mA.fA.mG.fC.mC.fU.mC.fA |
| 131 | 1025 | fC.mC.fU.mC.fA.mC.fC.mA.fA.mG.fA.mU.fC.mC.fA |
| 132 | 1026 | fG.mG.fU.mC.fA.mU.fC.mA.fC.mA.fG.mU.fU.mG.fA |
| 133 | 1027 | fG.mC.fA.mA.fG.mC.fA.mG.fA.mC.fA.mU.fU.mU.fA |
| 134 | 1028 | fU.mU.fC.mU.fA.mG.fA.mC.fC.mU.fG.mU.fU.mU.fA |
| 135 | 1029 | fG.mC.fC.mC.fA.mC.fA.mA.fC.mG.fC.mU.fU.mU.fA |
| 136 | 1030 | fU.mG.fU.mC.fC.mU.fC.mU.fC.mU.fG.mU.fU.mG.fA |
| 137 | 1031 | fA.mA.fG.mG.fA.mU.fC.mC.fG.mU.fG.mG.fA.mG.fA |
| 138 | 1032 | fU.mC.fC.mU.fG.mC.fA.mC.fA.mG.fU.mC.fC.mU.fA |
| 139 | 1033 | fA.mC.fA.mU.fU.mU.fA.mU.fC.mU.fU.mU.fU.mG.fA |
| 140 | 1034 | fC.mA.fA.mG.fG.mA.fG.mG.fC.mA.fG.mG.fA.mU.fA |
| 141 | 1035 | fC.mU.fU.mU.fG.mG.fC.mC.fG.mC.fU.mG.fU.mG.fA |
| 142 | 1036 | fC.mA.fC.mA.fG.mC.fA.mG.fG.mA.fA.mC.fU.mG.fA |
| 143 | 1037 | fC.mU.fG.mC.fU.mC.fU.mA.fU.mG.fC.mC.fA.mG.fA |
| 144 | 1038 | fU.mG.fU.mC.fA.mU.fC.mA.fA.mU.fG.mA.fG.mG.fA |
| 145 | 1039 | f.mG.fA.mG.fG.mA.fC.mG.fG.mC.fG.mA.fC.mU.fA |
| 146 | 1040 | fC.mA.fG.mG.fA.mU.fU.mC.fU.mU.fC.mC.fC.mA.fA |
| 147 | 1041 | fC.mG.fU.mC.fA.mA.fG.mC.fA.mC.fC.mC.fA.mC.fA |
| 148 | 1042 | fC.mA.fC.mC.fU.mG.fC.mU.fU.mU.fG.mU.fG.mU.fA |
| 149 | 1043 | fC.mC.fA.mC.fG.mC.fU.mU.fC.mC.fU.mG.fC.mU.fA |
| 150 | 1044 | fA.mG.fG.mC.fA.mG.fA.mG.fA.mC.fU.mG.fA.mU.fA |
| 151 | 1045 | fC.mC.fA.mU.fG.mA.fU.mG.fC.mU.fG.mU.fC.mU.fA |
| 152 | 1046 | fC.mA.fU.mU.fG.mC.fA.mG.fC.mC.fA.mU.fG.mA.fA |
| 153 | 1047 | fC.mU.fA.mU.fG.mC.fC.mA.fG.mG.fC.mU.fG.mU.fA |
| 154 | 1048 | fC.mU.fC.mU.fG.mC.fU.mC.fU.mA.fU.mG.fC.mC.fA |
| 155 | 1049 | fG.mU.fC.mA.fG.mC.fC.mC.fA.mA.fC.mC.fA.mG.fA |
| 156 | 1050 | fU.mA.fU.mC.fU.mU.fU.mU.fG.mG.fG.mU.fC.mU.fA |
| 157 | 1051 | fC.mC.fG.mC.fU.mG.fU.mG.fU.mG.fG.mA.fC.mC.fA |
| 158 | 1052 | fC.mA.fG.mC.fC.mU.fG.mG.fU.mG.fG.mA.fG.mG.fA |
| 159 | 1053 | fU.mU.fG.mG.fC.mA.fG.mC.fU.mG.fU.mU.fU.mU.fA |
| 160 | 1054 | fU.mU.fG.mG.fG.mU.fC.mU.fG.mU.fC.mC.fU.mC.fA |
| 161 | 1055 | fC.mA.fA.mG.fC.mA.fA.mG.fC.mA.fG.mA.fC.mA.fA |
| 162 | 1056 | fC.mU.fC.mU.fC.mU.fG.mU.fU.mG.fC.mC.fU.mU.fA |
| 163 | 1057 | fU.mA.fU.mC.fU.mC.fC.mU.fA.mG.fA.mC.fA.mC.fA |
| 164 | 1058 | fA.mG.fA.mG.fA.mC.fU.mG.fA.mU.fC.mC.fA.mC.fA |
| 165 | 1059 | fG.mU.fU.mG.fG.mC.fA.mG.fC.mU.fG.mU.fU.mU.fA |
| 166 | 1060 | fC.mC.fA.mC.fA.mA.fG.mC.fC.mG.fC.mC.fU.mG.fA |
| 167 | 1061 | fU.mG.fA.mG.fG.mC.fC.mU.fG.mG.fU.mU.fC.mC.fA |
| 168 | 1062 | fA.mG.fU.mG.fA.mG.fC.mC.fA.mG.fG.mC.fA.mG.fA |
| 169 | 1063 | fG.mA.fU.mG.fG.mC.fC.mC.fU.mC.fA.mU.fC.mU.fA |
| 170 | 1064 | fC.mA.fC.mA.fG.mA.fC.mC.fA.mG.fG.mA.fA.mG.fA |
| 171 | 1065 | fC.mU.fG.mG.fA.mA.fU.mG.fC.mA.fA.mA.fG.mU.fA |
| 172 | 1066 | fA.mU.fG.mG.fC.mA.fU.mC.fU.mA.fG.mC.fC.mA.fA |
| 173 | 1067 | fU.mG.fG.mC.fA.mU.fU.mG.fC.mA.fG.mC.fC.mA.fA |
| 174 | 1068 | fA.mA.fG.mC.fA.mC.fC.mC.fA.mC.fA.mC.fC.mC.fA |
| 175 | 1069 | fC.mC.fC.mA.fC.mA.fA.mC.fG.mC.fU.mU.fU.mU.fA |
| 176 | 1070 | fC.mU.fG.mG.fC.mU.fU.mC.fC.mU.fG.mG.fU.mG.fA |
| 177 | 1071 | fC.mA.fU.mG.fG.mA.fA.mU.fC.mC.fC.mG.fG.mC.fA |
| 178 | 1072 | fC.mU.fA.mC.fG.mA.fG.mG.fA.mG.fC.mU.fG.mG.fA |
| 179 | 1073 | fU.mG.fA.mG.fG.mA.fC.mC.fA.mG.fC.mG.fG.mG.fA |
| 180 | 1074 | fU.mG.fG.mC.fU.mG.fG.mC.fA.mU.fU.mG.fC.mA.fA |
| 181 | 1075 | fC.mA.fA.mA.fG.mA.fU.mG.fU.mC.fA.mU.fC.mA.fA |
| 182 | 1076 | fU.mG.fG.mA.fG.mG.fU.mG.fU.mA.fU.mC.fU.mC.fA |
| 183 | 1077 | fC.mA.fA.mG.fC.mA.fC.mC.fC.mA.fC.mA.fC.mC.fA |
| 184 | 1078 | fC.mC.fG.mA.fG.mU.fU.mG.fA.mG.fG.mC.fA.mG.fA |
| 185 | 1079 | fU.mG.fG.mU.fC.mA.fC.mC.fG.mA.fC.mU.fU.mC.fA |
| 186 | 1080 | fA.mG.fG.mA.fG.mG.fA.mC.fG.mA.fG.mG.fA.mC.fA |
| 187 | 1081 | fA.mA.fC.mA.fG.mU.fG.mA.fG.mG.fC.mU.fG.mG.fA |
| 188 | 1082 | fU.mU.fA.mU.fU.mC.fG.mG.fA.mA.fA.mA.fG.mC.fA |
| 189 | 1083 | fC.mU.fG.mC.fC.mA.fA.mG.fC.mU.fC.mA.fC.mA.fA |
| 190 | 1084 | fC.mC.fA.mG.fC.mA.fU.mC.fC.mA.fC.mG.fC.mU.fA |
| 191 | 1085 | fG.mA.fC.mU.fC.mC.fU.mC.fU.mG.fU.mC.fU.mU.fA |
| 192 | 1086 | fC.mC.fA.mA.fG.mC.fA.mA.fG.mC.fA.mG.fA.mC.fA |
| 193 | 1087 | fG.mU.fG.mC.fU.mG.fC.mC.fU.mG.fC.mU.fA.mC.fA |
| 194 | 1088 | fG.mA.fG.mG.fC.mC.fA.mG.fC.mA.fU.mC.fC.mA.fA |
| 195 | 1089 | fG.mA.fC.mA.fU.mU.fU.mA.fU.mC.fU.mU.fU.mU.fA |
| 196 | 1090 | fU.mG.fU.mU.fU.mU.fG.mC.fA.mG.fG.mA.fC.mU.fA |
| 197 | 1091 | fU.mC.fA.mC.fC.mA.fA.mG.fA.mU.fC.mC.fU.mG.fA |
| 198 | 1092 | fC.mA.fC.mC.fU.mU.fC.mC.fA.mC.fC.mG.fC.mU.fA |
| 199 | 1093 | fC.mU.fA.mC.fG.mC.fC.mA.fU.mU.fG.mC.fC.mA.fA |
| 200 | 1094 | fG.mG.fA.mG.fG.mA.fC.mU.fC.mC.fU.mC.fU.mG.fA |
| 201 | 1095 | fU.mG.fC.mU.fG.mA.fG.mC.fU.mG.fC.mU.fC.mC.fA |
| 202 | 1096 | fU.mG.fC.mC.fA.mA.fG.mC.fU.mC.fA.mC.fA.mC.fA |
| 203 | 1097 | fA.mU.fU.mG.fC.mC.fA.mG.fG.mU.fG.mC.fU.mG.fA |
| 204 | 1098 | fC.mA.fG.mU.fG.mA.fG.mG.fC.mU.fG.mG.fG.mA.fA |
| 205 | 1099 | fU.mG.fC.mU.fC.mC.fA.mG.fU.mU.fU.mC.fU.mC.fA |
| 206 | 1100 | fU.mG.fA.mU.fC.mC.fA.mC.fU.mU.fC.mU.fC.mU.fA |
| 207 | 1101 | fG.mA.fU.mG.fA.mG.fU.mG.fG.mC.fG.mA.fC.mC.fA |
| 208 | 1102 | fA.mA.fA.mG.fC.mC.fA.mG.fC.mU.fG.mG.fU.mC.fA |
| 209 | 1103 | fC.mA.fA.mG.fA.mU.fC.mC.fU.mG.fC.mA.fU.mG.fA |
| 210 | 1104 | fC.mA.fA.mC.fU.mU.fU.mG.fG.mC.fC.mG.fC.mU.fA |
| 211 | 1105 | fG.mU.fC.mC.fU.mC.fA.mC.fA.mG.fG.mC.fU.mG.fA |
| 212 | 1106 | fC.mG.fU.mC.fC.mA.fC.mA.fC.mA.fG.mC.fU.mC.fA |
| 213 | 1107 | fU.mG.fC.mU.fG.mC.fC.mU.fG.mC.fU.mA.fC.mC.fA |
| 214 | 1108 | fC.mG.fA.mG.fC.mC.fG.mG.fA.mG.fC.mU.fC.mA.fA |
| 215 | 1109 | fU.mG.fU.mA.fU.mC.fU.mC.fC.mU.fA.mG.fA.mC.fA |
| 216 | 1110 | fC.mG.fG.mG.fA.mC.fC.mC.fG.mC.fU.mU.fC.mC.fA |
| 217 | 1111 | fC.mA.fA.mG.fC.mU.fC.mA.fC.mA.fC.mA.fG.mC.fA |
| 218 | 1112 | fA.mA.fA.mA.fG.mC.fC.mA.fG.mC.fU.mG.fG.mU.fA |
| 219 | 1113 | fG.mU.fG.mG.fA.mC.fC.mU.fC.mU.fU.mU.fG.mC.fA |
| 220 | 1114 | fC.mC.fU.mC.fU.mA.fG.mG.fU.mC.fU.mC.fC.mU.fA |
| 221 | 1115 | fC.mU.fG.mU.fU.mU.fU.mG.fC.mA.fG.mG.fA.mC.fA |
| 222 | 1116 | fC.mU.fA.mC.fG.mU.fG.mG.fU.mG.fG.mU.fG.mC.fA |
| 223 | 1117 | fC.mC.fU.mG.fG.mC.fC.mC.fU.mU.fG.mA.fG.mU.fA |
| 224 | 1118 | fG.mA.fG.mC.fU.mU.fU.mA.fA.mA.fA.mU.fG.mG.fA |
| 225 | 1119 | fG.mG.fG.mC.fC.mC.fA.mC.fA.mA.fC.mG.fC.mU.fA |
| 226 | 1120 | fU.mC.fC.mA.fC.mU.fU.mC.fU.mC.fU.mG.fC.mC.fA |
| 227 | 1121 | fC.mC.fC.mU.fA.mG.fA.mA.fG.mG.fU.mU.fU.mC.fA |
| 228 | 1122 | fU.mG.fC.mU.fG.mG.fU.mC.fA.mC.fC.mG.fC.mU.fA |
| 229 | 1123 | fC.mC.fU.mC.fC.mA.fG.mC.fG.mA.fC.mU.fG.mC.fA |
| 230 | 1124 | fU.mU.fA.mU.fU.mC.fU.mG.fG.mG.fU.mU.fU.mU.fA |
| 231 | 1125 | fG.mU.fG.mU.fC.mU.fA.mC.fG.mC.fC.mA.fU.mU.fA |
| 232 | 1126 | fU.mU.fG.mU.fA.mA.fC.mU.fU.mG.fA.mA.fG.mA.fA |
| 233 | 1127 | fU.mU.fU.mU.fG.mC.fU.mU.fU.mU.fG.mU.fA.mA.fA |
| 234 | 1128 | fU.mG.fU.mA.fA.mC.fU.mU.fG.mA.fA.mG.fA.mU.fA |
| 235 | 1129 | fC.mU.fC.mC.fU.mA.fG.mA.fC.mA.fC.mC.fA.mG.fA |
| 236 | 1130 | fA.mG.fU.mC.fA.mG.fA.mG.fC.mG.fC.mA.fC.mU.fA |
| 237 | 1131 | fC.mA.fG.mC.fA.mA.fG.mU.fG.mU.fG.mA.fC.mA.fA |
| 238 | 1132 | fG.mA.fG.mG.fA.mC.fU.mC.fC.mU.fC.mU.fG.mU.fA |
| 239 | 1133 | fU.mU.fU.mG.fC.mU.fU.mU.fU.mG.fU.mA.fA.mC.fA |
| 240 | 1134 | fA.mA.fG.mG.fA.mG.fG.mC.fA.mG.fG.mA.fU.mU.fA |
| 241 | 1135 | fC.mA.fG.mC.fC.mA.fC.mC.fU.mU.fC.mC.fA.mC.fA |
| 242 | 1136 | fA.mG.fC.mC.fA.mA.fC.mC.fC.mG.fC.mU.fC.mC.fA |
| 243 | 1137 | fA.mA.fG.mA.fU.mG.fA.mG.fU.mG.fG.mC.fG.mA.fA |
| 244 | 1138 | fA.mG.fG.mA.fG.mG.fA.mC.fU.mC.fC.mU.fC.mU.fA |
| 245 | 1139 | fC.mC.fG.mA.fG.mC.fC.mG.fG.mA.fG.mC.fU.mC.fA |
| 246 | 1140 | fA.mG.fG.mU.fC.mA.fU.mC.fA.mC.fA.mG.fU.mU.fA |
| 247 | 1141 | fG.mG.fU.mG.fG.mA.fG.mG.fU.mG.fU.mA.fU.mC.fA |
| 248 | 1142 | fU.mC.fU.mG.fC.mU.fC.mU.fA.mU.fG.mC.fC.mA.fA |
| 249 | 1143 | fA.mG.fC.mU.fG.mC.fU.mG.fA.mG.fC.mU.fG.mC.fA |
| 250 | 1144 | fA.mA.fG.mA.fU.mG.fU.mC.fA.mU.fC.mA.fA.mU.fA |

Tables 3a to 3d below show nucleobase sequences and sugar-phosphate backbone modifications of antisense and sense strands of further 43 constructs as described herein. For corresponding entries in the sequence listing, the following applies: entry number in Table 3a+500=entry number in the sequence listing; entry number in Table 3c+543=entry number in the sequence listing.

TABLE 3a

| | SEQ ID | |
|---|---|---|
| # | NO: | 43 antisense nucleobase sequences |
| 1 | 501 | UUCUUUGGCAGAGAAGUGG |
| 2 | 502 | UCAUGCAGGAUCUUGGUGA |
| 3 | 503 | UUAAACUCCAGGCCUAUGA |
| 4 | 504 | UGCAAUGCCAGCCACGUGG |
| 5 | 505 | UGACUUUGCAUUCCAGACC |
| 6 | 506 | UAACCGUGCCCUUCCCUUG |
| 7 | 507 | UAGUGGAUCAGUCUCUGCC |
| 8 | 508 | UCAUUCUCGAAGUCGGUGA |
| 9 | 509 | UAACUCCAGGCCUAUGAGG |
| 10 | 510 | UAACUUCAAGGCCAGCUCC |
| 11 | 511 | UAAUGGCGUAGACACCCUC |
| 12 | 512 | UUAGAGGCAGGCAUCGUCC |
| 13 | 513 | UACCUCCACGGAUCCUUGG |
| 14 | 514 | UCAGUCUCUGCCUCAACUC |
| 15 | 515 | UUCUUGGUGAGGUAUCCCC |
| 16 | 516 | UAGAUGGCAACGGCUGUCA |
| 17 | 517 | UACACCUCCACCAGGCUGC |
| 18 | 518 | UUGGAAGACAUGCAGGAUC |
| 19 | 519 | UAUGAGGGUGCCGCUAACC |
| 20 | 520 | UUUCUCGAAGUCGGUGACC |
| 21 | 521 | UAAGUGGAUCAGUCUCUGC |
| 22 | 522 | UCACUCUGUAUGCUGGUGU |
| 23 | 523 | UGUGUGGACGCUGCAGUUG |
| 24 | 524 | UGUAGAGGCAGGCAUCGUC |
| 25 | 525 | UCUGUAUGCUGGUGUCUAG |
| 26 | 526 | UUCAAGGCCAGCUCCAGCA |
| 27 | 527 | UAAGUCGGUGACCAUGACC |
| 28 | 528 | UCACACUUGCUGGCCUGUC |
| 29 | 529 | UGUCUGUGGAAGCGGGUCC |
| 30 | 530 | UUUGGCAGAGAAGUGGAUC |
| 31 | 531 | UAGAAGGCCAUGGAAGACA |
| 32 | 532 | UUUGAGGACGCGGCUGUAC |
| 33 | 533 | UUUCCCGGUGGUCACUCUG |
| 34 | 534 | UAUGCUGGUGUCUAGGAGA |
| 35 | 535 | UGGAAGGUGGCUGUGGUUC |
| 36 | 536 | UAAACUCCAGGCCUAUGAG |
| 37 | 537 | UUCCACGGGAUGCUCUGGG |
| 38 | 538 | UUCCGCUCCAGGUUCCACG |
| 39 | 539 | UUCAGCACCACCACGUAGG |
| 40 | 540 | UCCUCCUCGGGCACAUUCU |
| 41 | 541 | UUUCCGAAUAAACUCCAGG |
| 42 | 542 | UUUCCAUGCUCCUUGACUU |
| 43 | 543 | UGCUCCUUGACUUUGCAUU |

TABLE 3b

| | SEQ | |
|---|---|---|
| | ID | |
| # | NO: | 43 modified antisense strands |
| 1 | 1145 | [mU][#][fU][#][mC][fU][mU][fU][mG][fG][mC][fA][mG][fA][mG] |
| | | [fA][#][mA][#][fG][#][mU][#][fG][#][rG] |
| 2 | 1146 | [mU][#][fC][#][mA][fU][mG][fC][mA][fG][mG][fA][mU][fC][mU] |
| | | [fU][#][mG][#][fG][#][mU][#][fG][#][rA] |
| 3 | 1147 | [mU][#][fU][#][mA][fA][mA][fC][mU][fC][mC][fA][mG][fG][mC] |
| | | [fC][#][mU][#][fA][#][mU][#][fG][#][rA] |
| 4 | 1148 | [mU][#][fG][#][mC][fA][mA][fU][mG][fC][mC][fA][mG][fC][mC] |
| | | [fA][#][mC][#][fG][#][mU][#][fG][#][rG] |
| 5 | 1149 | [mU][#][fG][#][mA][fC][mU][fU][mU][fG][mC][fA][mU][fU][mC] |
| | | [fC][#][mA][#][fG][#][mA][#][fC][#][rC] |
| 6 | 1150 | [mU][#][fA][#][mA][fC][mC][fG][mU][fG][mC][fC][mC][fU][mU] |
| | | [fC][#][mC][#][fC][#][mU][#][fU][#][rG] |
| 7 | 1151 | [mU][#][fA][#][mG][fU][mG][fG][mA][fU][mC][fA][mG][fU][mC] |
| | | [fU][#][mC][#][fU][#][mG][#][fC][#][rC] |
| 8 | 1152 | [mU][#][fC][#][mA][fU][mU][fC][mU][fC][mG][fA][mA][fG][mU] |
| | | [fC][#][mG][#][fG][#][mU][#][fG][#][rA] |
| 9 | 1153 | [mU][#][fA][#][mA][fC][mU][fC][mC][fA][mG][fG][mC][fC][mU] |
| | | [fA][#][mU][#][fG][#][mA][#][fG][#][rG] |
| 10 | 1154 | [mU][#][fA][#][mA][fC][mU][fU][mC][fA][mA][fG][mG][fC][mC] |
| | | [fA][#][mG][#][fC][#][mU][#][fC][#][rC] |
| 11 | 1155 | [mU][#][fA][#][mA][fU][mG][fG][mC][fG][mU][fA][mG][fA][mC] |
| | | [fA][#][mC][#][fC][#][mC][#][fU][#][rC] |
| 12 | 1156 | [mU][#][fU][#][mA][fG][mA][fG][mG][fC][mA][fG][mG][fC][mA] |
| | | [fU][#][mC][#][fG][#][mU][#][fC][#][rC] |
| 13 | 1157 | [mU][#][fA][#][mC][fC][mU][fC][mC][fA][mC][fG][mG][fA][mU] |
| | | [fC][#][mC][#][fU][#][mU][#][fG][#][rG] |
| 14 | 1158 | [mU][#][fC][#][mA][fG][mU][fC][mU][fC][mU][fG][mC][fC][mU] |
| | | [fC][#][mA][#][fA][#][mC][#][fU][#][rC] |
| 15 | 1159 | [mU][#][fU][#][mC][fU][mU][fG][mG][fU][mG][fA][mG][fG][mU] |
| | | [fA][#][mU][#][fC][#][mC][#][fC][#][rC] |
| 16 | 1160 | [mU][#][fA][#][mG][fA][mU][fG][mG][fC][mA][fA][mC][fG][mG][fC][#][mU][#][fG][#][mU][#][fC][#][rA] |
| 17 | 1161 | [mU][#][fA][#][mC][fA][mC][fC][mU][fC][mC][fA][mC][fC][mA][fG][#][mG][#][fC][#][mU][#][fG][#][rC] |
| 18 | 1162 | [mU][#][fU][#][mG][fG][mA][fA][mG][fA][mC][fA][mU][fG][mC][fA][#][mG][#][fG][#][mA][#][fU][#][rC] |
| 19 | 1163 | [mU][#][fA][#][mU][fG][mA][fG][mG][fG][mU][fG][mC][fC][mG][fC][#][mU][#][fA][#][mA][#][fC][#][rC] |
| 20 | 1164 | [mU][#][fU][#][mU][fC][mU][fC][mG][fA][mA][fG][mU][fC][mG][fG][#][mU][#][fG][#][mA][#][fC][#][rC] |
| 21 | 1165 | [mU][#][fA][#][mA][fG][mU][fG][mG][fA][mU][fC][mA][fG][mU][fC][#][mU][#][fC][#][mU][#][fG][#][rC] |
| 22 | 1166 | [mU][#][fC][#][mA][fC][mU][fC][mU][fG][mU][fA][mU][fG][mC][fU][#][mG][#][fG][#][mU][#][fG][#][rU] |
| 23 | 1167 | [mU][#][fG][#][mU][fG][mU][fG][mG][fA][mC][fG][mC][fU][mG][fC][#][mA][#][fG][#][mU][#][fU][#][rG] |
| 24 | 1168 | [mU][#][fG][#][mU][fA][mG][fA][mG][fG][mC][fA][mG][fG][mC][fA][#][mU][#][fC][#][mG][#][fU][#][rC] |
| 25 | 1169 | [mU][#][fC][#][mU][fG][mU][fA][mU][fG][mC][fU][mG][fG][mU][fG][#][mU][#][fC][#][mU][#][fA][#][rG] |
| 26 | 1170 | [mU][#][fU][#][mC][fA][mA][fG][mG][fC][mC][fA][mG][fC][mU][fC][#][mC][#][fA][#][mG][#][fC][#][rA] |
| 27 | 1171 | [mU][#][fA][#][mA][fG][mU][fC][mG][fG][mU][fG][mA][fC][mC][fA][#][mU][#][fG][#][mA][#][fC][#][rC] |
| 28 | 1172 | [mU][#][fC][#][mA][fC][mA][fC][mU][fU][mG][fC][mU][fG][mG][fC][#][mC][#][fU][#][mG][#][fU][#][rC] |
| 29 | 1173 | [mU][#][fG][#][mU][fC][mU][fG][mU][fG][mG][fA][mA][fG][mC][fG][#][mG][#][fG][#][mU][#][fC][#][rC] |
| 30 | 1174 | [mU][#][fU][#][mU][fG][mG][fC][mA][fG][mA][fG][mA][fA][mG][fU][#][mG][#][fG][#][mA][#][fU][#][rC] |
| 31 | 1175 | [mU][#][fA][#][mG][fA][mA][fG][mG][fC][mC][fA][mU][fG][mG][fA][#][mA][#][fG][#][mA][#][fC][#][rA] |
| 32 | 1176 | [mU][#][fU][#][mU][fG][mA][fG][mG][fA][mC][fG][mC][fG][mG][fC][#][mU][#][fG][#][mU][#][fA][#][rC] |
| 33 | 1177 | [mU][#][fU][#][mU][fC][mC][fC][mG][fG][mU][fG][mG][fU][mC][fA][#][mC][#][fU][#][mC][#][fU][#][rG] |
| 34 | 1178 | [mU][#][fA][#][mU][fG][mC][fU][mG][fG][mU][fG][mU][fC][mU][fA][#][mG][#][fG][#][mA][#][fG][#][rA] |
| 35 | 1179 | [mU][#][fG][#][mG][fA][mA][fG][mG][fU][mG][fG][mC][fU][mG][fU][#][mG][#][fG][#][mU][#][fU][#][rC] |
| 36 | 1180 | [mU][#][fA][#][mA][fA][mC][fU][mC][fC][mA][fG][mG][fC][mC][fU][#][mA][#][fU][#][mG][#][fA][#][rG] |
| 37 | 1181 | [mU][#][fU][#][mC][fC][mA][fC][mG][fG][mG][fA][mU][fG][mC][fU][#][mC][#][fU][#][mG][#][fG][#][rG] |
| 38 | 1182 | [mU][#][fU][#][mC][fC][mG][fC][mU][fC][mC][fA][mG][fG][mU][fU][#][mC][#][fC][#][mA][#][fC][#][rG] |
| 39 | 1183 | [mU][#][fU][#][mC][fA][mG][fC][mA][fC][mC][fA][mC][fC][mA][fC][#][mG][#][fU][#][mA][#][fG][#][rG] |
| 40 | 1184 | [mU][#][fC][#][mC][fU][mC][fC][mU][fC][mG][fG][mG][fC][mA][fC][#][mA][#][fU][#][mU][#][fC][#][rU] |
| 41 | 1185 | [mU][#][fU][#][mU][fC][mC][fG][mA][fA][mU][fA][mA][fA][mC] |
| | | [fU][#][mC][#][fC][#][mA][#][fG][#][rG] |
| 42 | 1186 | [mU][#][fU][#][mU][fC][mC][fA][mU][fG][mC][fU][mC][fC][mU] |
| | | [fU][#][mG][#][fA][#][mC][#][fU][#][rU] |
| 43 | 1187 | [mU][#][fG][#][mC][fU][mC][fC][mU][fU][mG][fA][mC][fU][mU] |
| | | [fU][#][mG][#][fC][#][mA][#][fU][#][rU] |

TABLE 3c

| | SEQ | 43 sense nucleobase |
|---|---|---|
| # | ID NO: | sequences |
| 1 | 544 | UCUCUGCCAAAGAA |
| 2 | 545 | AAGAUCCUGCAUGA |
| 3 | 546 | GGCCUGGAGUUUAA |
| 4 | 547 | UGGCUGGCAUUGCA |
| 5 | 548 | GGAAUGCAAAGUCA |
| 6 | 549 | GAAGGGCACGGUUA |
| 7 | 550 | AGACUGAUCCACUA |
| 8 | 551 | GACUUCGAGAAUGA |
| 9 | 552 | UAGGCCUGGAGUUA |
| 10 | 553 | UGGCCUUGAAGUUA |
| 11 | 554 | UGUCUACGCCAUUA |
| 12 | 555 | AUGCCUGCCUCUAA |
| 13 | 556 | GAUCCGUGGAGGUA |
| 14 | 557 | GAGGCAGAGACUGA |
| 15 | 558 | UACCUCACCAAGAA |
| 16 | 559 | GCCGUUGCCAUCUA |
| 17 | 560 | CUGGUGGAGGUGUA |
| 18 | 561 | UGCAUGUCUUCCAA |
| 19 | 562 | GCGGCACCCUCAUA |
| 20 | 563 | CCGACUUCGAGAAA |
| 21 | 564 | GACUGAUCCACUUA |
| 22 | 565 | AGCAUACAGAGUGA |
| 23 | 566 | GCAGCGUCCACACA |
| 24 | 567 | UGCCUGCCUCUACA |
| 25 | 568 | CACCAGCAUACAGA |
| 26 | 569 | GAGCUGGCCUUGAA |
| 27 | 570 | UGGUCACCGACUUA |
| 28 | 571 | GCCAGCAAGUGUGA |
| 29 | 572 | CGCUUCCACAGACA |
| 30 | 573 | ACUUCUCUGCCAAA |
| 31 | 574 | UCCAUGGCCUUCUA |
| 32 | 575 | GCCGCGUCCUCAAA |
| 33 | 576 | UGACCACCGGGAAA |
| 34 | 577 | UAGACACCAGCAUA |
| 35 | 578 | ACAGCCACCUUCCA |
| 36 | 579 | AGGCCUGGAGUUUA |
| 37 | 580 | AGCAUCCCGUGGAA |
| 38 | 581 | AACCUGGAGCGGAA |
| 39 | 582 | GUGGUGGUGCUGAA |
| 40 | 583 | GUGCCCGAGGAGGA |
| 41 | 584 | AGUUUAUUCGGAAA |
| 42 | 585 | AAGGAGCAUGGAAA |
| 43 | 586 | AAAGUCAAGGAGCA |

TABLE 3d

| | SEQ | |
|---|---|---|
| | ID | |
| # | NO: | 43 modified sense strands |
| 1 | 1188 | [mU][#][fC][#][mU][fC][mU][fG][mC][fC][mA][fA][mA][fG][#][mA][#][fA][#][3XGalNac] |
| 2 | 1189 | [mA][#][fA][#][mG][fA][mU][fC][mC][fU][mG][fC][mA][fU][#][mG][#][fA][#][3XGalNac] |
| 3 | 1190 | [mG][#][fG][#][mC][fC][mU][fG][mG][fA][mG][fU][mU][fU][#][mA][#][fA][#][3XGalNac] |
| 4 | 1191 | [mU][#][fG][#][mG][fC][mU][fG][mG][fC][mA][fU][mU][fG][#][mC][#][fA][#][3XGalNac] |
| 5 | 1192 | [mG][#][fG][#][mA][fA][mU][fG][mC][fA][mA][fA][mG][fU][#][mC][#][fA][#][3XGalNac] |
| 6 | 1193 | [mG][#][fA][#][mA][fG][mG][fG][mC][fA][mC][fG][mG][fU][#][mU][#][fA][#][3XGalNac] |
| 7 | 1194 | [mA][#][fG][#][mA][fC][mU][fG][mA][fU][mC][fC][mA][fC][#][mU][#][fA][#][3XGalNac] |
| 8 | 1195 | [mG][#][fA][#][mC][fU][mU][fC][mG][fA][mG][fA][mA][fU][#][mG][#][fA][#][3XGalNac] |
| 9 | 1196 | [mU][#][fA][#][mG][fG][mC][fC][mU][fG][mG][fA][mG][fU][#][mU][#][fA][#][3XGalNac] |
| 10 | 1197 | [mU][#][fG][#][mG][fC][mC][fU][mU][fG][mA][fA][mG][fU][#][mU][#][fA][#][3XGalNac] |
| 11 | 1198 | [mU][#][fG][#][mU][fC][mU][fA][mC][fG][mC][fC][mA][fU][#][mU][#][fA][#][3XGalNac] |
| 12 | 1199 | [mA][#][fU][#][mG][fC][mC][fU][mG][fC][mC][fU][mC][fU][#][mA][#][fA][#][3XGalNac] |
| 13 | 1200 | [mG][#][fA][#][mU][fC][mC][fG][mU][fG][mG][fA][mG][fG][#][mU][#][fA][#][3XGalNac] |
| 14 | 1201 | [mG][#][fA][#][mG][fG][mC][fA][mG][fA][mG][fA][mC][fU][#][mG][#][fA][#][3XGalNac] |
| 15 | 1202 | [mU][#][fA][#][mC][fC][mU][fC][mA][fC][mC][fA][mA][fG][#][mA][#][fA][#][3XGalNac] |
| 16 | 1203 | [mG][#][fC][#][mC][fG][mU][fU][mG][fC][mC][fA][mU][fC][#][mU][#][fA][#][3XGalNac] |
| 17 | 1204 | [mC][#][fU][#][mG][fG][mU][fG][mG][fA][mG][fG][mU][fG][#][mU][#][fA][#][3XGalNac] |
| 18 | 1205 | [mU][#][fG][#][mC][fA][mU][fG][mU][fC][mU][fU][mC][fC][#][mA][#][fA][#][3XGalNac] |
| 19 | 1206 | [mG][#][fC][#][mG][fG][mC][fA][mC][fC][mC][fU][mC][fA][#][mU][#][fA][#][3XGalNac] |
| 20 | 1207 | [mC][#][fC][#][mG][fA][mC][fU][mU][fC][mG][fA][mG][fA][#][mA][#][fA][#][3XGalNac] |
| 21 | 1208 | [mG][#][fA][#][mC][fU][mG][fA][mU][fC][mC][fA][mC][fU][#][mU][#][fA][#][3XGalNac] |
| 22 | 1209 | [mA][#][fG][#][mC][fA][mU][fA][mC][fA][mG][fA][mG][fU][#][mG][#][fA][#][3XGalNac] |
| 23 | 1210 | [mG][#][fC][#][mA][fG][mC][fG][mU][fC][mC][fA][mC][fA][#][mC][#][fA][#][3XGalNac] |
| 24 | 1211 | [mU][#][fG][#][mC][fC][mU][fG][mC][fC][mU][fC][mU][fA][#][mC][#][fA][#][3XGalNac] |
| 25 | 1212 | [mC][#][fA][#][mC][fC][mA][fG][mC][fA][mU][fA][mC][fA][#][mG][#][fA][#][3XGalNac] |
| 26 | 1213 | [mG][#][fA][#][mG][fC][mU][fG][mG][fC][mC][fU][mU][fG][#][mA][#][fA][#][3XGalNac] |
| 27 | 1214 | [mU][#][fG][#][mG][fU][mC][fA][mC][fC][mG][fA][mC][fU][#][mU][#][fA][#][3XGalNac] |
| 28 | 1215 | [mG][#][fC][#][mC][fA][mG][fC][mA][fA][mG][fU][mG][fU][#][mG][#][fA][#][3XGalNac] |
| 29 | 1216 | [mC][#][fG][#][mC][fU][mU][fC][mC][fA][mC][fA][mG][fA][#][mC][#][fA][#][3XGalNac] |
| 30 | 1217 | [mA][#][fC][#][mU][fU][mC][fU][mC][fU][mG][fC][mC][fA][#][mA][#][fA][#][3XGalNac] |
| 31 | 1218 | [mU][#][fC][#][mC][fA][mU][fG][mG][fC][mC][fU][mU][fC][#][mU][#][fA][#][3XGalNac] |
| 32 | 1219 | [mG][#][fC][#][mC][fG][mC][fG][mU][fC][mC][fU][mC][fA][#][mA][#][fA][#][3XGalNac] |
| 33 | 1220 | [mU][#][fG][#][mA][fC][mC][fA][mC][fC][mG][fG][mG][fA][#][mA][#][fA][#][3XGalNac] |
| 34 | 1221 | [mU][#][fA][#][mG][fA][mC][fA][mC][fC][mA][fG][mC][fA][#][mU][#][fA][#][3XGalNac] |
| 35 | 1222 | [mA][#][fC][#][mA][fG][mC][fC][mA][fC][mC][fU][mU][fC][#][mC][#][fA][#][3XGalNac] |
| 36 | 1223 | [mA][#][fG][#][mG][fC][mC][fU][mG][fG][mA][fG][mU][fU][#][mU][#][fA][#][3XGalNac] |
| 37 | 1224 | [mA][#][fG][#][mC][fA][mU][fC][mC][fC][mG][fU][mG][fG][#][mA][#][fA][#][3XGalNac] |
| 38 | 1225 | [mA][#][fA][#][mC][fC][mU][fG][mG][fA][mG][fC][mG][fG][#][mA][#][fA][#][3XGalNac] |
| 39 | 1226 | [mG][#][fU][#][mG][fG][mU][fG][mG][fU][mG][fC][mU][fG][#][mA][#][fA][#][3XGalNac] |
| 40 | 1227 | [mG][#][fU][#][mG][fC][mC][fC][mG][fA][mG][fG][mA][fG][#][mG][#][fA][#][3XGalNac] |
| 41 | 1228 | [mA][#][fG][#][mU][fU][mU][fA][mU][fU][mC][fG][mG][fA][#][mA][#][fA][#][3XGalNac] |
| 42 | 1229 | [mA][#][fA][#][mG][fG][mA][fG][mC][fA][mU][fG][mG][fA][#][mA][#][fA][#][3XGalNac] |
| 43 | 1230 | [mA][#][fA][#][mA][fG][mU][fC][mA][fA][mG][fG][mA][fG][#][mC][#][fA][#][3XGalNac] |

It should also be noted that the scope of the disclosed embodiments extends to sequences that correspond to those in the Tables above, and wherein the 5′ nucleoside of the antisense (guide) strand (first region as defined in the claims herein) can include any nucleobase that can be present in an RNA molecule, in other words can be any of adenine (A), uracil (U), guanine (G) or cytosine (C). Additionally, the scope of the present embodiments extends to sequences that correspond to those in Table 1a or Table 1 b, and wherein the 3′ nucleoside of the sense (passenger) strand (second region as defined in the claims herein) can include any nucleobase that can be present in an RNA molecule, in other words can be any of adenine (A), uracil (U), guanine (G) or cytosine (C), advantageously however a nucleobase that is complementary to the 5′ nucleobase of the antisense (guide) strand (first region as defined in the claims herein). While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the Examples described above may be combined with aspects of any of the other Examples described to form further Examples.

It will be understood that the above description of a particular embodiment is given by way of example only and that various modifications may be made by those skilled in the art. What has been described above includes Examples of one or more embodiments. It is, of course, not possible to describe every conceivable modification and alteration of the above compounds, compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

### EXAMPLES

The following Examples illustrate certain embodiments of the present disclosure and are not limiting. Moreover, where specific embodiments are provided, the inventors have contemplated generic application of those specific embodiments. For example, disclosure of an oligonucleotide having a particular motif or modification patterns provides reasonable support for additional oligonucleotides having the same or similar motif or modification patterns.

### Example 1

Materials and Methods

Cell Culture:

HepG2 (ATCC cat. 85011430) cells were maintained by biweekly passing in EMEM supplemented with 10% FBS, 20 mM L-glutamine, 10 mM HEPES pH 7.2, 1 mM sodium pyruvate, 1xMEM non-essential amino acids, and 1xPen/Strep (EMEM complete).

PCSK9 Target identification and duplex preparation:

Targets to PCSK9 were identified by bioinformatic analysis on human PCSK9 mRNA sequence (refseq NM_174936.3). 250 targets were selected for synthesis as asymmetric duplexes (15 sense, 19 antisense). Compounds were dissolved to 50 uM in molecular biology grade water and annealed by heating at 95 C for 5 minutes followed by gradual cooling to room temperature.

PCSK9-Primary Screen:

On the day of transfection, HepG2 cells were collected by trypsinization, counted, and seeded in 96 well tissue culture treated plates at 10,000 cells per well in 50 uL complete EMEM with 20% FBS. Cells were allowed to rest for 4 hours before transfection with 2 pmoles of each respective PCSK9 duplex in triplicate via RNAiMax (ThermoFisher). In brief, 8 pmoles of each duplex were diluted in 100 uL OptiMEM and mixed gently with 0.8 uL of RNAiMax in 100 uL OptiMEM to make 200 uL total complex. 50 uL of each RNAiMax complexed duplex was added to each respective triplicate well of HepG2 cells for a final mixture of 20 nM duplex in a volume of 100 uL, 50/50 EMEM/OptiMEM at 10% FBS.

72 hours post transfection, cells were harvested and RNA isolated using the PureLink Pro 96 total RNA Purification Kit (ThermoFisher, 12173011A) according to the manufacturer protocol. Harvested RNA was assayed for PCSK9 expression via Taqman qPCR using the Luna Universal Probe One-Step RT-qPCR Kit (NEB, E3006). Two separate qPCR assays were performed for each sample using two separate PCSK9 Taqman probe sets (Hs00545399_m1-FAM and Hs03037355_m1-FAM) multiplexed with a common GAPDH VIC probe (ThermoFisher, 4326317E). Thermocycling and data acquisition was performed with an Applied Biosystems QuantStudio 3 Real-Time PCR System.

PCSK9-Secondary Screen:

Based on data from the primary screen, the best performing 27 PCSK9 duplexes were tested in dose curves and IC50 values have been determined. As before, HepG2 cells were collected by trypsinization and seeded in 96 well tissue culture plates at 10,000 cells per well in 50 uL complete EMEM with 20% FBS and allowed to rest for 4 hours. Transfection complexes were formed by gently mixing 36 pmoles of each duplex in 180 uL OptiMEM with 2.16 uL RNAiMax in 180 uL OptiMEM to make 360 uL total complex. A two fold dilution series was then performed with basal OptiMEM. 50 uL of each dilution was added to respective triplicates of HepG2 cells to make a final dilution series of 50 nM down to 0.32 nM in a volume of 100 uL, 50/50 EMEM/OptiMEM at 10% FBS.

72 hours post transfection, cells were harvested and RNA isolated using the PureLink Pro 96 total RNA Purification Kit (ThermoFisher, 12173011A) according to the manufacturer protocol. Harvested RNA was assayed for PCSK9 expression via Taqman qPCR using the Luna Universal Probe One-Step RT-qPCR Kit (NEB, E3006). A single qPCR assay was performed for each sample using PCSK9 Taqman probe set Hs00545399_m1-FAM multiplexed with a common GAPDH VIC probe (ThermoFisher, 4326317E). Thermocycling and data acquisition was performed with an Applied Biosystems QuantStudio 3 Real-Time PCR System.

### Example 2

Results obtained by performing methods of Example 1

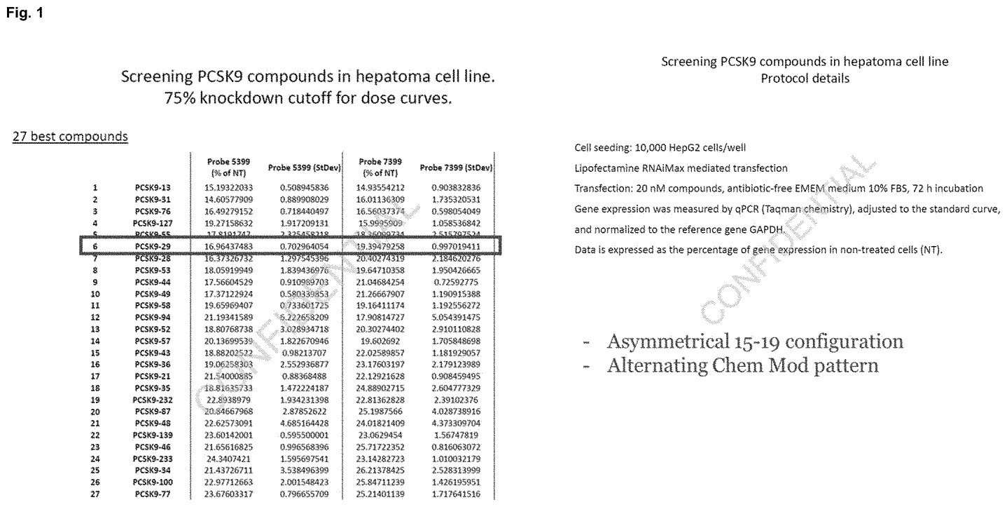

FIG. 1 shows the results (% knockdown) of the primary screen.

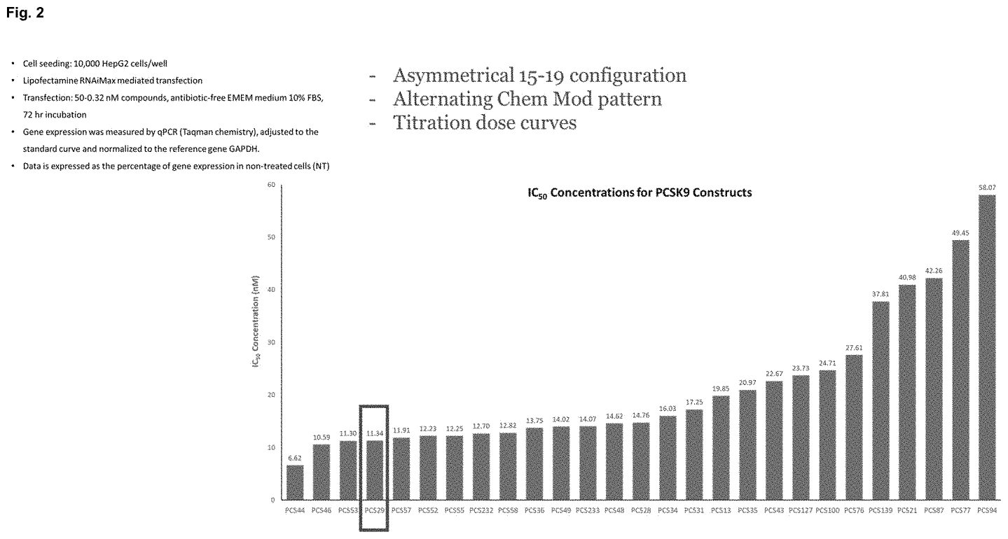

Table 4 below and FIG. 2 show IC50 values (in nM) for the 27 constructs selected in accordance with Example 1. Table discloses SEQ ID NOS 44, 294, 46, 296, 53, 303, 29, 279, 57, 307, 52, 302, 55, 305, 232, 482, 58, 308, 36, 286, 49, 299, 233, 483, 48, 298, 28, 278, 34, 284, 31, 281, 13, 263, 35, 285, 43, 293, 127, 377, 100, 350, 76, 326, 139, 389, 21, 271, 87, 337, 77, 327, 94, and 344, respectively, in order of appearance.

| | | | % k/d at | |
|---|---|---|---|---|
| Sequence | Anti-Sense | SS Sequence | the highest | |
| ID | Sequence (5′ to 3′) | (5′ to 3′) | cone | IC50 |
| PCS44 | UCUUCAAGUUACAAAAGCA | UUUGUAACUUGAAGA | 86.65 | 6.62 |
| PCS46 | UAAAAGCAAAACAGGUCUA | CCJGUUUUGCUUUUA | 79.14 | 10.59 |
| PCS53 | UACAAAAGCAAAACAGGUC | UGUUOUGCUUUUGUA | 86.08 | 11 50 |
| PCS29 | UGCAAAACAGGUCUAGAAA | UAGACCUGUUUUGCA | 80.50 | 13.54 |
| PCS57 | UCAAAAGCAAAACAGGUCU | CUGUUUUGCUUUUGA | 83.24 | 11.91 |
| PCS52 | UAGAAUAAAUAUCUUCAAG | AAGAUAUUUAUUCUA | 83.30 | 12.25 |
| PCS55 | UAAAGCAAAACAGGULUAG | ACCUGUUUUGCUUUA | 72.99 | 12.25 |
| PCS232 | uucuUCAAGUUACAAAAGC | UUGUAACUUGAAGAA | 71.48 | 12.70 |
| PCS55 | UCAAGUUACAAAAGCAAAA | GCUUOUGUAACUUGA | 79.06 | 12.32 |
| PCS36 | UCAAAACAGGUCUAGAAAA | CUAGACCUGUUUUGA | 78.57 | 13.75 |
| PCS49 | UAUAAAUAUCUUCAAGUUA | UUGAAGAUAUUUAUA | 79.11 | 14.02 |
| PCS233 | UUUACAAAAGCAAAACAGG | UUUUSCUUUGUAAA | 72.52 | 14.07 |
| PCS48 | UGUGACACAAAGCAGGUGC | CUGCUUUGUGUCACA | 76.03 | 14 $2 |
| PCS28 | UUUCAAGUUACAAAAGCAA | UUUUGUAACUUGAAA | 77.64 | 14.76 |
| PCS34 | UAAAUAUCUUCAAGUUACA | ACUUGAAGAUAUUUA | 80.55 | 16.03 |
| PCS51 | UAUAAAUGUCUGCUUGCUU | AAGCAGACAUUUAUA | 72.48 | 17.25 |
| PCS13 | UCCGAAUAAACUCCAGGCC | UGGAGUUUAUUCGGA | 71.05 | 19.35 |
| PCS35 | UGGAACCAUUUUAAAGCUC | UUUAAAAUGGUUCCA | 67.12 | 20.37 |
| PCS43 | UCCAGAAUAAAUAUCUUCA | GAUAUUVAUUCUSGA | 68.11 | 22.67 |
| PCS127 | UAUAUCUUCAAGUUACAAA | UAACUUGAAGAUAUA | 70.81 | 23.73 |
| PCS100 | UCUCAGUUCCUGCUGUGUG | CAGCAGGAACUGAGA | 74.42 | 24.71 |
| PCS76 | UCUACAAAACCCAGAAUAA | UCUGGGUUUUGUAGA | 68.90 | 27 SI |
| PCS139 | UCAAAAGAUAAAUGUCUGC | ACAUUUAUCUUUUGA | 58.47 | 37.81 |
| PCS21 | UUUCCGAAUAAACUCCAGG | GAGUUUAUUCGGAAA | 51.24 | 40 99 |
| PCS87 | UCUGGCUUCAGUUCCGCUG | AGGAACUGAGCCAGA | 64.33 | 42.26 |
| PCS77 | UCAGACAGCAUCAUGGCUG | CAUGAUGCUGUCOGA | 51.63 | 49.45 |
| PCS94 | UGGGAUUCCAUCCUUCUUG | GAGCAUGGAAUCCCA | 45.66 | 58.07 |

The IC50 data in the single- to double-digit nanomolar range demonstrate outstanding performance of numerous constructs as described herein.

### Example 3

Optimized Double Stranded Constructs

Full definitions of the constructs are given in Table 5 below. AS=antisense strand (also referred as first region herein); SS=sense strand (also referred to as second region herein).

Notations are explained further above.

| | | |
|---|---|---|
| P29 | AS | 5′ mU*fG*mCfAmAfAmAfCmAfGmGfU*mC*fU*mA*fG*mA*fA* |
| | | rA (SEQ ID NO: 1231) |
| | SS | 5′ mA*fG*mAfCmCfUmGfUmUfUmUfG*mC*fA*/Mono-Galnac-PA/ |
| | | /Mono-Galnac-PA//Mono-Galnac-PA/ (SEQ ID NO: 1232) |
| P57 | AS | 5′ mU*fC*mAfAmAfAmGfCmAfAmAfA*mC*fA*mG*fG*mU*fC* |
| | | rU (SEQ ID NO: 1233) |
| | SS | 5′ mU*fG*mUfUmUfUmGfCmUfUmUfU*mG*fA*/Mono-Galnac-PA// |
| | | Mono-Galnac-PA//Mono-Galnac-PA/ (SEQ ID NO: 1234) |
| P53 | AS | 5′ mU*fA*mCfAmAfAmAfGmCfAmAfA*mA*fC*mA*fG*mG*fU* |
| | | rC (SEQ ID NO: 1235) |
| | SS | 5′ mG*fU*mUfUmUfGmCfUmUfUmUfG*mU*fA*/Mono-Galnac-PA// |
| | | Mono-Galnac-PA//Mono-Galnac-PA/ (SEQ ID NO: 1236) |
| P44 | AS | 5′ mU*fC*mUfUmCfAmAfGmUfUmAfC*mA*fA*mA*fA*mG*fC* |
| | | rA (SEQ ID NO: 1237) |
| | SS | 5′ mU*fU*mGfUmAfAmCfUmUfGmAfA*mG*fA*/Mono-Galnac-PA// |
| | | Mono-Galnac-PA//Mono-Galnac-PA/ (SEQ ID NO: 1238) |
| P29 | AS | [mU][#][fG][#][mC][fA][mA][fA][mA][fC][mA][fG][mG] |
| (15) | | [fU][mC][fU][mA][#][fG][#][mA][#][fA][#][rA] |
| | | (SEQ ID NO: 1239) |
| | SS | [fU][#][mA][#][fG][mA][fC][mC][fU][mG][fU][mU][fU] |
| | | [mU][mG][#][mC][#][mA][#][3XGalNAc SEQ ID NO: 1240) |

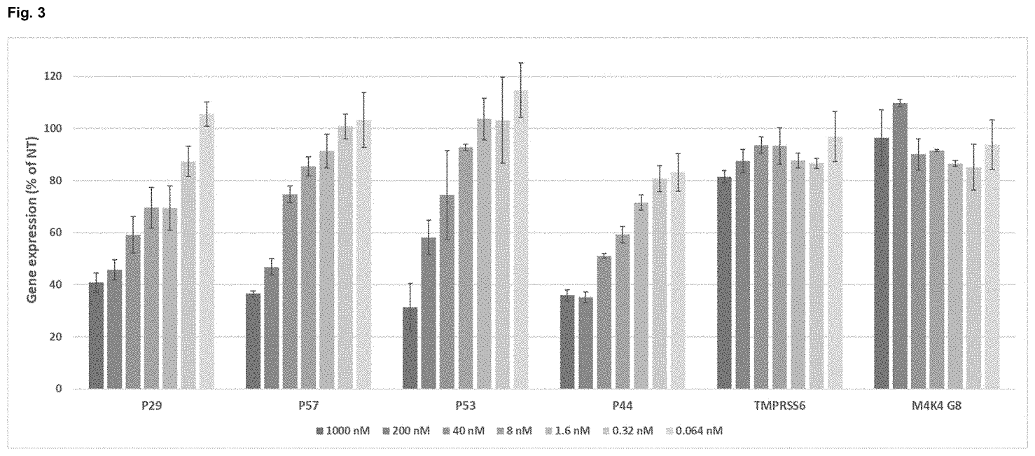

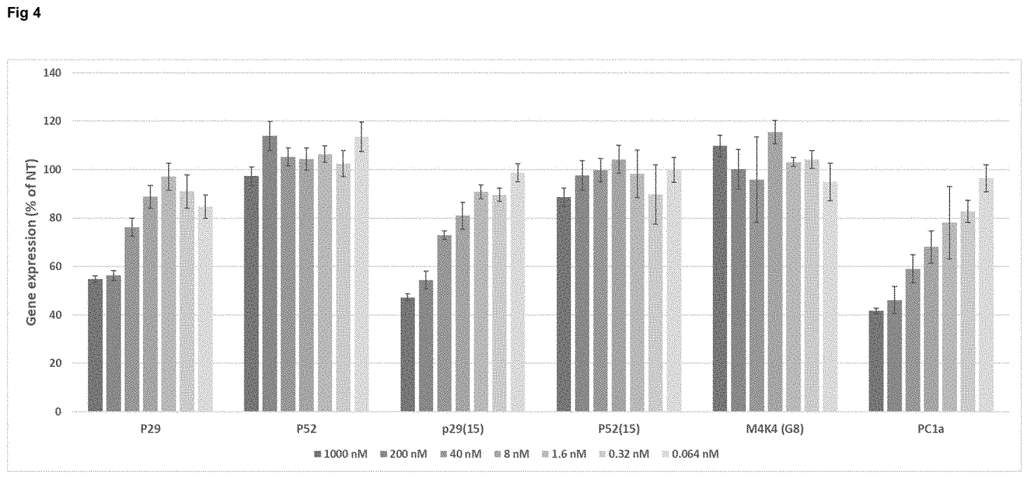

Performance data are shown in FIGS. 3 and 4. Comparison with an inclisiran-type molecule (FIG. 3) shows outstanding performance of the constructs as described herein. Indeed, performance of two constructs as described herein is very similar to a 3xGaINAc (toothbrush) derivative of inclisiran.

### Example 4

Optimized hairpin molecules (mxRNAs)

Full definitions of the molecules are given in Table 6 below.

| Compound name | Structural definition |
|---|---|
| PCS29 | [mU][#][fG][#][mC][fA][mA][fA][mA][fC][mA][fG] |
| (14-5-14) | [mG][fU][mC][fU][#][mA][#][fG][#][mA][#][fA][#] |
| | [mA][#][mA][fG][mA][fC][mC][fU][mG][fU][mU][fU] |
| | [mU][fG][#][mC][#][fA][#][3xGalNAc] |
| | (SEQ ID NO: 587) |
| PCS44 | [mU][#][fC][#][mU][fU][mC][fA][mA][fG][mU][fU] |
| (14-5-14) | [mA][fC][mA][fA][#][mA][#][fA][#][mG][#][fC][#] |
| | [mA][#][mU][fU][mG][fU][mA][fA][mC][fU][mU][fG] |
| | [mA][fA][#][mG][#][fA][#][3xGalNAc] |
| | (SEQ ID NO: 588) |
| PCS53 | [mU][#][fA][#][mC][fA][mA][fA][mA][fG][mC][fA] |
| (14-5-14) | [mA][fA][mA][fC][#][mA][#][fG][#][mG][#][fU][#] |
| | [mC][#][mG][fU][mU][fU][mU][fG][mC][fU][mU][fU] |
| | [mU][fG][#][mU][#][fA][#][3xGalNAc] |
| | (SEQ ID NO: 589) |
| PCS53 | [dT][#][fA][#][mC][fA][mA][fA][mA][fG][mC][fA] |
| (14-5-14) dT | [mA][fA][mA][fC][#][mA][#][fG][#][mG][#][dT][#] |
| | [mC][#][mG][fU][mU][fU][mU][fG][mC][fU][mU][fU] |
| | [mU][fG][#][mU][#][fA][#][3xGalNAc] |
| | (SEQ ID NO: 590) |

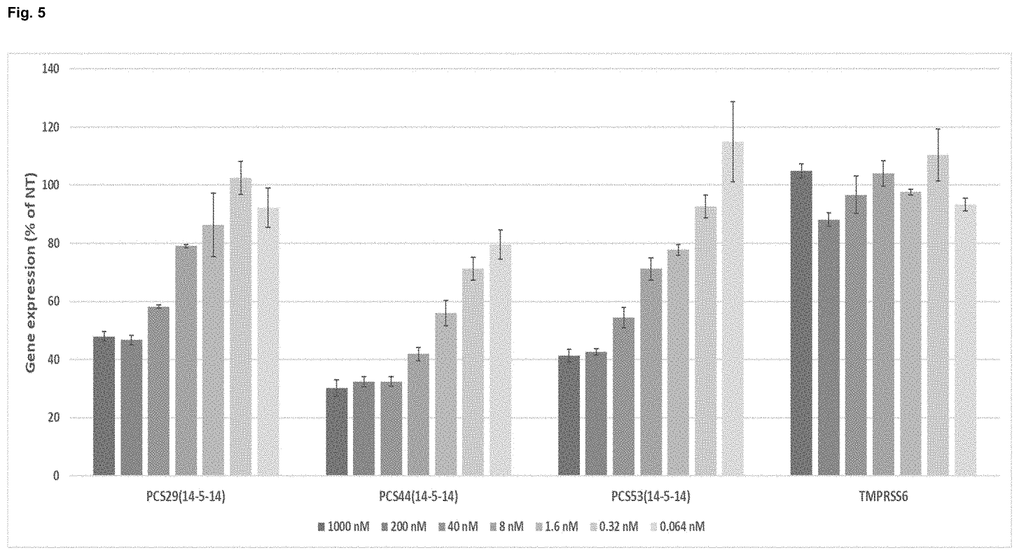

Performance data are shown in FIG. 5.

1. An oligomeric compound capable of inhibiting expression of PCSK9, wherein said compound comprises at least a first region of linked nucleosides having at least a first nucleobase sequence that is at least partially complementary to at least a portion of RNA transcribed from a PCSK9 gene, wherein said first nucleobase sequence is selected from the following sequences, or a portion thereof: sequences of (SEQ ID NOs 1 to 250), or sequences of (SEQ ID NOs 501 to 543), wherein said portion optionally has a length of at least 18 nucleotides.

2. The oligomeric compound according to claim 1, which further comprises at least a second region of linked nucleosides having at least a second nucleobase sequence that is at least partially complementary to said first nucleobase sequence and is selected from the following sequences, or a portion thereof: sequences of (SEQ ID NOs 251 to 500), or sequences of (SEQ ID NOs 544 to 586), wherein said portion optionally has a length of at least 11 nucleotides.

3. The oligomeric compound according to claim 1, wherein said first nucleobase sequence is selected from the following sequences, or a portion thereof: SEQ ID NOs 13, 31, 76, 127, 55, 29, 28, 53, 44, 49, 58, 94, 52, 57, 43, 36, 21, 35, 232, 87, 48, 139, 46, 233, 34, 100, and 77.

4. The oligomeric compound according to claim 3, wherein said second nucleobase sequence is selected from the following sequences, or a portion thereof: SEQ ID NOs 263, 281, 326, 377, 305, 279, 278, 303, 294, 299, 308, 344, 302, 307, 293, 286, 271, 285, 482, 337, 298, 389, 296, 483, 284, 350, and 327, and wherein said portion is advantageously 14 nucleosides long and lacks the 5′-terminal nucleobase as set forth in said SEQ ID NOs.

5. The oligomeric compound according claim 1, wherein said first nucleobase sequence is selected from the following sequences, or a portion thereof: SEQ ID NOs 29, 44, 46, 52, 53, 55, and 57.

6. The oligomeric compound according to claim 5, wherein said second nucleobase sequence is selected from the following sequences, or a portion thereof: SEQ ID NOs 279, 294, 296, 302, 303, 305, and 307, and wherein said portion is optionally 14 nucleosides long and lacks the 5′-terminal nucleobase as set forth in said SEQ ID NOs.

7. The oligomeric compound according to claim 1, wherein said first region of linked nucleosides consists essentially of 18 to 35, 18 to 20, 18 or 19, or 19 linked nucleosides.

8. The oligomeric compound according to claim 2, wherein said second region of linked nucleosides consists essentially of 11 to 35, 11 to 20, 13 to 16, 14 or 15, or 14 linked nucleosides.

9. The oligomeric compound according to claim 2, which comprises at least one complementary duplex region that comprises at least a portion of said first nucleoside region directly or indirectly linked to at least a portion of said second nucleoside region, wherein advantageously said duplex region has a length of 11 to 19, 14 to 19, 14 or 15, or 14 base pairs, wherein optionally there is one mismatch within said duplex region.

10. (canceled)

11. The oligomeric compound according to claim 9, wherein the 5′ region of said first nucleoside region is directly or indirectly linked to the 3′ region of said second nucleoside region, and/or wherein the 3′ region of said first nucleoside region is directly or indirectly linked to the 5′ region of said second nucleoside region, wherein optionally the 5′ terminal nucleoside of said first nucleoside region base pairs with the 3′ terminal nucleoside of said second nucleoside region.

12. The oligomeric compound according to claim 1, which further comprises one or more ligands, wherein said ligand optionally comprises at least one carbohydrate.

13-19. (canceled)

20. The oligomeric compound according to claim 12, wherein said ligand comprises one or more N-Acetyl-Galactosamine moieties.

21-24. (canceled)

25. The oligomeric compound according to claim 9, wherein said oligomeric compound comprises a single strand comprising said first and second nucleoside regions, wherein said single strand dimerises whereby at least a portion of said first nucleoside region is directly or indirectly linked to at least a portion of said second nucleoside region so as to form said at least partially complementary duplex region.

26-27. (canceled)

28. The oligomeric compound according to claim 25, having a nucleobase sequence selected from SEQ ID NOs: 587 to 590.

29. The oligomeric compound according to claim 1, which comprises at least one modified internucleoside linkage.

30-42. (canceled)

43. The oligomeric compound according to claim 1, wherein sugars of the nucleosides at any of positions 2 and 14 downstream from the first nucleoside of the 5′ region of the first nucleoside region, do not contain 2′-O-methyl modifications.

44. The oligomeric compound according to claim 2, wherein sugars of the nucleosides of the second nucleoside region, that correspond in position to any of the nucleosides of the first nucleoside region at any of positions 9 to 11 downstream from the first nucleotide of the 5′ region of the first nucleoside region, do not contain 2′-O-methyl modifications.

45-73. (canceled)

74. The oligomeric compound according to claim 1, wherein the first region of linked nucleotides is selected from the group consisting of Table 2b, Table 3b, Table 1a, and Table 5.

75. The oligomeric compound according to claim 74, wherein the second region of linked nucleotides is selected from the group consisting of Table 2d, Table 3d, Table 1b, and Table 5.

76. A pharmaceutical composition comprising an oligomeric compound according to claim 2, and a physiologically acceptable excipient.

77-81. (canceled)

82. The pharmaceutical composition of claim 76, further comprising a further oligomeric compound which is directed to a target different from PCSK9 and/or a lipid-lowering agent distinct from said oligomeric compound, wherein said lipid-lowering agent optionally is a statin or ezetimib.

83-86. (canceled)

87. A method of treating a disease or disorder comprising administration of an oligomeric compound according to claim 1, to an individual in need of treatment, wherein said disease or disorder is a PCSK9-associated disease or disorder, or a disease or disorder requiring reduction of low-density lipoprotein (LDL) cholesterol, wherein said disease or disorder optionally is selected from the group consisting of dyslipidemia, mixed dyslipidemia, hypercholesterolemia, heterozygous familial hypercholesterolemia, non-familial hypercholesterolemia, atherosclerosis, atherosclerotic cardiovascular disease (ASCVD), myocardial infarction, stroke and peripheral arterial disease.

88-89. (canceled)
